(19) United States
(12) Patent Application Publication
(10) Pub. No.: US 2026/0085077 A1
(43) Pub. Date: Mar. 26, 2026

(54) HETEROCYCLIC DERIVATIVES AS JANUS KINASE INHIBITORS

(71) Applicant: CHIESI FARMACEUTICI S.P.A., Parma (IT)

(72) Inventors: Alessandro ACCETTA, Parma (IT); Alberto CUZZOLIN, Parma (IT); Fabio RANCATI, Parma (IT); Andrea RIZZI, Parma (IT); Ivaylo Jivkov ELENKOV, Parma (IT); Milan MESIC, Parma (IT); Ela RADOŠEVIC, Parma (IT)

(73) Assignee: CHIESI FARMACEUTICI S.P.A., Parma (IT)

(21) Appl. No.: 19/109,331

(22) PCT Filed: Sep. 8, 2023
(86) PCT No.: PCT/EP2023/074691

§ 371 Date: Mar. 6, 2025

(30) Foreign Application Priority Data
Sep. 9, 2022 (EP) 22194839.1

(51) Int. Cl. C07D 519/00; A61K 9/00; A61K 31/519; A61K 31/5377; A61K 31/538; A61K 31/541; A61K 31/5415; A61K 31/553; A61K 45/06; C07D 471/04

(52) U.S. Cl. CPC C07D 519/00; A61K 9/007; A61K 31/519; A61K 31/5377; A61K 31/538; A61K 31/541; A61K 31/5415; A61K 31/553; A61K 45/06; C07D 471/04

(57) ABSTRACT

The present invention relates to a compounds of general formula (I) inhibiting the JAK family of non-receptor tyrosine protein kinases (JAK1, JAK2, JAK3, and TYK2); methods of preparing such compounds, pharmaceutical compositions containing them and therapeutic use thereof. The compounds of the invention may be useful in the treatment of diseases or conditions associated with a dysregulation of the JAK family non-receptor kinases; in particular for the treatment of various inflammatory diseases including asthma, COPD and other respiratory diseases.

### FIELD OF THE INVENTION

The present invention relates to chemical compounds that are derivatives useful as JAK inhibitors, such as JAK 1, useful for the treatment of various inflammatory disease including asthma, COPD and other respiratory diseases.

### BACKGROUND OF THE INVENTION

The JAK family consists of non-receptor tyrosine protein kinases and has four main members, JAK1, JAK2, JAK3, and TYK2. More than 50 cytokines and growth factors bind to type I and II receptors noncovalently associated with different combinations of JAK kinases. The signalling triggered by the ligands consists in tyrosine phosphorylation of receptors by JAK and recruitment of one or more STATs proteins. Tyrosine-phosphorylated STATs dimerize and are then transported into the nucleus through the nuclear membrane to regulate specific genes. JAKs have seven homology domains (the JAK homology domain, JH). Starting from the carboxyl terminus, JH1 is the first JH, known as the kinase domain, and is composed of approximately 250 amino acid residues. JH1 encodes a kinase protein that constitutes the kinase structure domain that phosphorylates a substrate; JH2 is a pseudokinase domain which regulates the activity of the kinase domain. JAK3 is expressed in the bone marrow and lymphatic system, as well as endothelial cells and vascular smooth muscle cells; other members are expressed in almost all tissues (Hu X et al., Signal Transduct Target Ther. 2021, 26; 6(1):402). Many cellular processes are downstream JAK/STAT signalling: hematopoiesis, immune balance, tissue repair, inflammation, apoptosis, and adipogenesis. Different biological responses are regulated by specific pairing of JAK isoforms. JAK1/JAK3 combination mediates IL-2, -4, -7, -9, -15, and -21 signalling that is relevant for growth/maturation of lymphoid cells, differentiation/homeostasis of T-cells/NK cells, B-cell class switching and other inflammatory processes. Combinations of JAK1/TYK2-JAK1/JAK2, regulate the signal associated with the innate immune response, such as IL-6 and the type I interferons, involved into naïve T cell differentiation, T cell homeostasis, granulopoiesis and other inflammatory processes. (Howell M D et al., Front. Immunol. 2019, 10, 2342). JAK2 frequently associates with itself (JAK2/JAK2) controlling the signalling of various cytokines and growth factors, such as IL-3, IL-5, granulocyte macrophage colony-stimulating factor (GM-CSF), erythropoietin (EPO), and thrombopoietin (TPO) (Hodge et al., Clin Exp Rheumatol 2016; 34(2):318-28).

Genetically modified mouse models and human diseases prove the importance of JAK/STAT pathways in immune fitness. In particular, overexpression or mutations involving some JAK isoforms as well as aberrant JAK/STAT signalling drive malignancies of hematopoietic and lymphoid tissues as well as inflammatory disorders. Currently, several Food and Drug Administration (FDA)- and/or EU-approved JAK inhibitors are in clinical use. Two (ruxolitinib and fedratinib) small molecules are in use for hematologic disorders as myelofibrosis and polycythemia vera; six JAK inhibitors (tofacitinib, baricitinib, ruxololitinib, filgotinib, upadicitinib and delgocitinib in Japan) result in use for immune-mediated disorders as rheumatoid arthritis, polyarticular juvenile idiopathic arthritis, atopic dermatitis, ulcerative colitis and acute graft-versus-host disease. Moreover, some of these drugs as well as others are currently under phase II and III of clinical trials for indications that span from autoimmune diseases (lupus, vitiligo, etc.), inflammatory bowel disease to Non-Hodgkin lymphoma and COVID-19 (Hu X. et al., Sig Transduct Target Ther 2021, 6: 402).

The small molecules targeting JAK/STAT represent an attractive option also for the therapy of fibrotic disorders. In fact, inflammatory cytokines (IL-4, IL-3, IL-6, IL-11, IL-31, etc) and growth factors (FGF, VEGF, etc.) involved in the fibrotic processes activate JAK/STAT pathway. Ruxolitinib tested in a bleomycin-induced fibrosis mouse model ameliorated the fibrotic lesions in lung, and reduced levels of fibrotic molecular markers (Zhang, Y et al., Ann. Rheum. Dis. 2017, 76, 1467-1475) while tofacitinib acted as a preventive agent in experimental dermal and pulmonary fibrosis (Wang, W et al., Scleroderma Relat. Disord. 2020, 5, 40-50). In patients, some case reports were studied. A single-case report corroborated the efficacy and safety of tofacitinib in combination with nintedanib in the management of an aggressive interstitial lung disease with poor prognosis (Conca, W et al., Front. Pharmacol. 2020, 11, 5857619). Baricitinib was demonstrated to be a safe immune modulator that reduces the biomarkers' levels of lung fibrosis and inflammation in RA patients, including a subgroup with interstitial lung disease (D'Alessandro M et al., Int. Immunopharmacol. 2020, 86, 106748).

In COVID-19, there are some JAK inhibitors undergoing clinical trials, and they are tofacitinib, baricitinib, and ruxolitinib. Baricitinib and ruxolitinib were associated with a reduced risk of mortality. They reduced the use of invasive mechanical ventilation and had a borderline impact on the admission rate of the intensive care unit and the incidence of acute respiratory distress syndrome (ARDS). (Wijaya, I. et al. Clin. Epidemiol. Glob. Health 2021, 11, 100755). Ruxolitinib also was tested in COVID-19 patients. and improved the clinical symptoms and chest computed tomography images (Cao Y. et al., J. Allergy Clin. Immunol. 2020 146, 137-146).

Asthma can be included in the plethora of immune-mediated diseases for which pathogenesis is characterized by an essential role of JAK/STAT signalling. Asthma is a chronic inflammatory disease of the airways due to a complex interplay between immune response, genetic susceptibility and nonspecific external stimuli like cold, allergens and exercise leading to hyperresponsiveness, remodelling of the airways, ultimately contributing to airflow limitation. Severe asthma affects 5% to 15% of the population with adult asthma (which is 300 million people worldwide) and represents a public health issue associated with increased mortality, increased hospitalizations, significant burden of symptoms, health care costs, and missed work and school (Steve N G et al., J Allergy Clin Immunol 2021; 148:953-63). Severe asthma represents a subset of difficult-to-treat asthma and occurs in patients whose disease remains uncontrolled despite the use of high doses of inhaled corticosteroids (ICSs) combined with long-acting β-agonists or other controllers. To date, four types of biologics are licensed for severe asthma, i.e. omalizumab (anti-immunoglobulin E) antibody, mepolizumab and reslizumab (anti-interleukin [IL]-5antibody), benralizumab (anti-IL-5 receptor a antibody) and dupilumab (anti-IL-4 receptor alpha antibody). Despite their efficacy, many patients continue to experience exacerbations or uncontrolled disease, indicating a need for more novel therapies (Israel E, Reddel H K. N Engl J Med 2017; 377:965-76).

Recently, the better understanding of asthma pathobiology brought to a shift from a phenotypic classification system to the introduction of the “endotype” concept. According to the latter, classification is performed on the basis of pathophysiologic mechanisms and clinical biomarkers associated with a given patient (Wenzel S E et a., Am J Respir Crit Care Med 2021; 203:809-21). There are two major endotypes in asthma: type 2 and non-type 2. The type 2 pathway is defined by activation of cytokines derived from TH2 cells and group 2 innate lymphoid cells (ILC2s); these include IL-4, IL-5, and IL-13 that cause airway inflammation by activating eosinophils, B cells, airway epithelial cells, and other cell types. Biomarkers of type 2 asthma include blood/sputum eosinophilia and elevated levels of fractional exhaled nitric oxide (FENO) and IgE. The type 2-low pathway is characterized by absence of type 2-high cytokines and biomarkers, and it manifests either increased levels of neutrophils in the airways or a paucigranulocytic profile, with normal levels of airway neutrophils and eosinophils. Type 2-low asthma is currently not well understood, and it likely encompasses multiple distinct endotypes. Potential mediators and/or biomarkers of T2 low endotypes under investigation include IL-6, IL-17A/F, IL-23, Type I interferons, CXCL10, TNF, alarmins (TSLP, IL-25, IL-33), IL-1β, IL-8, IFN-γ (Hinks T S C et al., ERJ 2021, 57 (1) 2000528).

Almost all the mediators mentioned above both for T2 and T2-low endotypes activate JAK/STAT pathway, here the rationale for the potential use of JAK inhibitors in both endotypes of severe asthma. Targeting simultaneously several cytokines by JAK inhibitors may offer advantage over the biologics (for no-responder patients) and standard therapies (for patients who remain uncontrolled) considering their administration on top of ICS.

Despite strong rationale of JAK inhibitors in asthma, safety concerns may arise by administration of systemic inhibitors or may limits administration into particular asthma subjects such as children. Considering that Asthma is a lung restricted disease, inhalatory route of administration for a JAK inhibitor may offers the advantage of therapeutic efficacy while limiting systemic exposure and correlated side effects. To date, some companies are developing inhaled JAK inhibitor for asthma treatment. Astrazeneca pipeline include AZD-0449 (completed Phase I clinical trial) and AZD-4604 (ongoing Phase I clinical trial); Theravance Biopharma is starting a new preclinical program on TD-8236 inhaled JAK inhibitor and Kinaset/Vectura is developing VR588 (ongoing Phase I clinical trial) as inhalatory compound. Many preclinical studies sponsored by the companies mentioned above demonstrated the efficacy of JAK inhibitors in the modulation of asthma. In the preclinical phase of drug development, JAK1/3 inhibitor R256 (now referred as AZD0449) orally given showed be effective in decreasing airway resistance, BAL eosinophilia, mucus production and if administered during sensitization, also TH2 cytokine responses (Ashino S et al., J Allergy Clin Immunol 2014; 133:1162-74). iJak-381 from Genentech given as dry powder reduced BAL eosinophilia, CCL11, airway resistance, and Muc5AC in OVA-challenged mice. Moreover, it reduced BAL eosinophilia, neutrophilia, CCL11, and CXCL1 in a in mouse model of chronic exposure to AAH allergens (Dengler H S et al., Sci Transl Med 2018; 10:eaao2151). Moreover, an oral JAK inhibitor as Tofacitinib, formulated for being administered as aerosol, reduced eosinophils count in a house dust mite mouse model of asthma (Younis U S et al., AAPS PharmSci-Tech 2019; 20:167).

Another respiratory disease that could benefit from lung restricted JAK inhibition is Chronic obstructive pulmonary disease (COPD), an inflammatory disease of the lung, most commonly resulting from cigarette smoke exposure, characterised by a largely irreversible and progressive airflow limitation. Despite inflammatory cytokines are drivers of chronic airway inflammation and some of them trigger JAK/STAT activation (IL-6, IFN-γ, IL-2, etc.), the role of this pathway in COPD pathogenesis is poorly characterized. Phosphorylated-STAT4+ cells (Di Stefano A et al., Eur Respir J. 2004 July; 24(1):78-85) were found to be increased in COPD compared to non-smokers healthy controls. In another study, phosphorylated-STAT3+ and phosphorylated-STAT1+ cells counts were higher in lung biopsies of COPD patients than non-smokers controls while it was not possible to reproduce previous data on phosphorylated-STAT4 molecule (Yew-Booth L et al., Eur Respir J 2015; 46(3):843-5). These data might also suggest a therapeutic use of JAK inhibitors also in COPD disease.

In view of the number of pathological responses which are mediated by JAK enzymes, there is a continuing need for inhibitors of JAK enzymes which can be useful in the treatment of many disorders and particularly respiratory diseases.

Thus, the finding of novel and potent JAK inhibitor suitable for local administration to the lungs for treatment of asthma and respiratory disease still remains an important need.

Strong need remains for JAK inhibitors, and particularly inhaled JAK inhibitors that have potential for giving compounds with an improved safety. Despite inhalatory administration, safety concerns may still arise by drug levels reaching systemic circulation following inhalation of a JAKi. In addition to a well suited profile for inhalation, a JAKi should preferably possess additional properties that may further limit the systemic exposure after inhalation.

### SUMMARY OF THE INVENTION

Accordingly, it is one object of the present invention to provide compounds of formula (I)

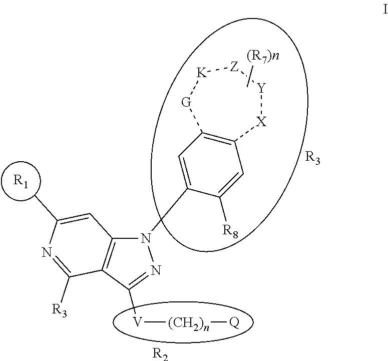

Wherein R₁, R₇, R₈, X, Y, Z, K, G, n, V and Q are as defined in the detailed description of the invention; or a pharmaceutically-acceptable salt thereof, that are useful as JAK kinase inhibitors.

It is another object of the present invention to provide pharmaceutical compositions comprising such compounds, methods of using such compounds to treat respiratory diseases, and processes and intermediates useful for preparing such compounds.

In one aspect, the present invention provides a compound of formula (I) for use as a medicament. In one aspect the present invention provides the use of a compound of the invention for the manufacture of a medicament.

In a further aspect, the present invention provides the use of a compound of the invention for the preparation of a medicament for the treatment of any disease associated with JAK enzyme mechanisms.

In another aspect, the present invention provides a method for prevention and/or treatment of any disease associated with JAK enzyme mechanisms as above defined, said method comprises administering to a patient in need of such treatment a therapeutically effective amount of a compound of the invention.

In a particular aspect the compounds of the invention are used alone or combined with other active ingredients and may be administered for the prevention and/or treatment of a pulmonary disease including asthma, chronic obstructive pulmonary disease (COPD), cystic fibrosis (CF), interstitial lung diseases and idiopathic pulmonary fibrosis (IPF), acute lung injury and acute respiratory distress syndrome (ARDS).

### DETAILED DESCRIPTION OF THE INVENTION

### Definitions

The term “Pharmaceutically acceptable salts” refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts of the invention comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric, hydrobromic, sulfuric, phosphoric, methane sulfonic, camphor sulfonic, acetic, oxalic, maleic, fumaric, succinic and citric acids.

Many organic compounds can form complexes with solvents in which they are reacted or from which they are precipitated or crystallized. These complexes are known as “solvates” which are a further object of the invention. Polymorphs and crystalline forms of compounds of formula (I), or of pharmaceutically acceptable salts, or solvates thereof are a further object of the invention.

The term “Halogen” or “halogen atoms” includes fluorine, chlorine, bromine, and iodine atom; meaning Fluoro, Chloro, Bromo, Iodo as substituent.

The term “(C₁-C₆)Alkyl” refers to straight-chained or branched alkyl groups wherein the number of carbon atoms is in the range 1 to 6. Particular alkyl groups are for example methyl, ethyl, n-propyl, isopropyl, t-butyl, 3-methylbutyl and the like.

The expressions “(C₁-C₆)Haloalkyl” refer to the above defined “(C₁-C₆)alkyl” groups wherein one or more hydrogen atoms are replaced by one or more halogen atoms, which can be the same or different from each other. Examples include halogenated, poly-halogenated and fully halogenated alkyl groups wherein all of the hydrogen atoms are replaced by halogen atoms, e.g. trifluoromethyl or difluoro methyl groups.

By way of analogy, the terms “(C₁-Cₓ) hydroxyalkyl” or “(C₁-Cₓ) aminoalkyl” refer to the above defined “(C₁-Cₓ) alkyl” groups wherein one or more hydrogen atoms are replaced by one or more hydroxy (OH) or amino group respectively. Thus, “(C₁-C₆)hydroxyalkyl” or “(C₁-C₆)aminoalkyl” refers to said hydroxy- or amino-alkyl groups wherein the number of carbon atoms is in the range 1 to 6.

The definition of aminoalkyl encompasses alkyl groups (i.e. “(C₁-C₆)alkyl” groups) substituted by one or more amino groups (—NR₄R₅). An example of aminoalkyl is a mono-aminoalkyl group such as R₄R₅N—(C₁-C₆)alkyl, or —(CH₂)ₘNR₄R₅. Wherein R₄ and R₅ and m are as defined in the detailed description of the invention.

With reference to the substituent R₄ and R₅ as above defined, it is here further explained that when either R₄ and R₅ are taken together with the nitrogen atom they are linked to form a 5 to 6 membered heterocyclic radical, at least one further ring carbon atom in the said heterocyclic radical may be replaced by at least one heteroatom or hetero-group (e.g. N, NH, S or O) or may bear an -oxo (═O) substituent group. The said heterocyclic radical might be further optionally substituted on the available points in the ring, namely on a carbon atom, or on an heteroatom or hetero-group available for substitution. Thus, Examples of said heterocycle radicals are 1-pyrrolidinyl, 1-piperidinyl, 1-piperazinyl, 4-morpholinyl, piperazin-4yl-2-one, 4-methylpiperazine-1-yl.

The term “(C₃-C₁₀)cycloalkyl” likewise “(C₃-C₆)cycloalkyl” refers to saturated cyclic hydrocarbon groups containing the indicated number of ring carbon atoms. Examples include cyclopropyl, cyclobutyl, cyclopentyl, cyclohexyl and cycloheptyl, and polycyclic ring systems such as adamantan-yl.

The expression “Aryl” refers to mono, bi- or tri-cyclic carbon ring systems which have 6 to 20, preferably from 6 to 15 ring atoms, wherein at least one ring is aromatic. The expression “heteroaryl” refers to mono-, bi- or tri-cyclic ring systems with 5 to 20, preferably from 5 to 15 ring atoms, in which at least one ring is aromatic and in which at least one ring atom is a heteroatom (e.g. N, S or O).

Examples of aryl or heteroaryl monocyclic ring systems include, for instance, phenyl, thienyl, pyrrolyl, pyrazolyl, imidazolyl, isoxazolyl, oxazolyl, isothiazolyl, thiazolyl, pyridinyl, pyrimidinyl, pyrazinyl, pyridazinyl, triazinyl, furanyl radicals and the like.

Examples of aryl or heteroaryl bicyclic ring systems include naphthalenyl, biphenylenyl, purinyl, pteridinyl, pyrazolopyrimidinyl, benzotriazolyl, benzoimidazole-yl, quinolinyl, isoquinolinyl, indolyl, isoindolyl, indazolyl, benzothiopheneyl, benzodioxinyl, dihydrobenzodioxinyl, indenyl, dihydro-indenyl, dihydrobenzo[1,4]dioxinyl, benzothiazole-2-yl, dihydrobenzodioxepinyl, benzooxazinyl, 1,2,3,4-tetrahydroisoquinoline-6-yl, 4,5,6,7-tetrahydrothiazolo[4,5-c]pyridine, 4,5,6,7-tetrahydrobenzo[d]thiazol-2-yl, 5,6,7,8-tetrahydro-1,7-naphthyridine, radicals and the like.

Examples of aryl or heteroaryl tricyclic ring systems include fluorenyl radicals as well as benzocondensed derivatives of the aforementioned heteroaryl bicyclic ring systems.

The derived expression “(C₃-C₁₀)heterocycloalkyl” likewise “(C₃-C₆)heterocycloalkyl” refers to saturated or partially unsaturated mono, bi- or tri-cycloalkyl groups of the indicated number of carbons, in which at least one ring carbon atom is replaced by at least one heteroatom (e.g. N, NH, S or O) and/or may bear an -oxo (═O) substituent group (e.g. C(═O), S(═O)₂). Said heterocycloalkyl (i.e. heterocyclic radical or group) is further optionally substituted on the available points in the ring, namely on a carbon atom, or on an heteroatom available for substitution.

Substitution on a carbon atom includes spiro disubstitution as well as substitution on two adjacent carbon atoms, in both cases thus form additional condensed 5 to 6 membered heterocyclic ring.

The derived expression “hydroxyl-(C₃-C₆)heterocycloalkyl” refers to the said heterocycloalkyl groups substituted on the available points in the ring, namely on a carbon atom, or on an heteroatom available for substitution with an hydroxyl group.

Examples of (C₃-C₆) heterocycloalkyl are represented by: oxetanyl, tetrahydro-furanyl, pyrrolidinyl, imidazolidinyl, thiazolidinyl, piperazinyl, piperidinyl, morpholinyl, thiomorpholinyl, dihydro- or tetrahydro-pyridinyl, tetrahydropyranyl, pyranyl, 2H- or 4H-pyranyl, dihydro- or tetrahydrofuranyl, dihydroisoxazolyl, pyrrolidin-2-one-yl, dihydropyrrolyl, 5-oxopyrrolidin-3-yl, (1R,5S,6r)-3-oxabicyclo[3.1.0]hexan-6-yl, 1,1-dioxidothiomorpholino, octahydrocyclopenta[c]pyrrol-5-yl, 4,5,6,7-tetrahydropyrazolo[1,5-a]pyrazin-2-yl; 4,5,6,7-tetrahydrothiazolo[5,4-c]pyridine-2-yl radicals and the like.

Other Examples of said heterocycle radicals are 1-methyl-2-pyrrolidinyl, piperazin-4-yl-2-one, 4-methylpiperazine-1-yl, 1-methylpiperidin-4-yl, 4-metylpiperazine-1-yl-2-one, 7-methyl-2,7-diazaspiro[3.5]nonan-2-yl, 2-methyl-2,9-diazaspiro[5.5]undecan-9-yl, 9-methyl-3,9-diazaspiro[5.5]undecan-3-yl, and (3aR,6aS)-5-methyl-octahydropyrrolo[3,4-c]pyrrol-2-yl, hexahydropyrazino[2,1-c][1,4]oxazin-8 (1H)-yl, 2-oxa-6-azaspiro[3.4]octan-6-yl, 7-oxo-6-oxa-2-azaspiro[3.4]octan-2-yl, 3-hydroxyoxetan-3-yl.

The term “Aryl(C₁-C₆)alkyl” refers to an aryl ring linked to a straight-chained or branched alkyl group wherein the number of constituent carbon atoms is in the range from 1 to 6, e.g. phenylmethyl (i.e. benzyl), phenylethyl or phenylpropyl.

Likewise the term “Heteroaryl(C₁-C₆)alkyl” refers to an heteroaryl ring linked to a straight-chained or branched alkyl group wherein the number of constituent carbon atoms is in the range from 1 to 6, e.g. furanylmethyl.

The term “alkanoyl”, refers to HC(O)— or to alkylcarbonyl groups (e.g. (C₁-C₆)alkylC(O)—) wherein the group “alkyl” has the meaning above defined. Examples include formyl, acetyl, propanoyl, butanoyl.

The term “(C₁-C₁₀) alkoxy” or “(C₁-C₁₀) alkoxyl”, likewise “(C₁-C₆) alkoxy” or “(C₁-C₆) alkoxyl” etc., refers to a straight or branched hydrocarbon of the indicated number of carbons, linked to the rest of the molecule through an oxygen bridge. “(C₁-C₆)Alkylthio” refers to the above hydrocarbon linked through a sulfur bridge.

The derived expression “(C₁-C₆)haloalkoxy” or “(C₁-C₆)haloalkoxyl” refers to the above defined haloalkyl, linked through an oxygen bridge. Example of (C₁-C₆)haloalkoxy is difluoromethoxy, trifluoromethoxy.

Likewise derived expression “(C₃-C₆)heterocycloalkyl-(C₁-C₆)alkyl” and “(C₃-C₆)cycloalkyl-(C₁-C₆)alkyl” refer to the above defined heterocycloalkyl and cycloalkyl groups linked to the rest of the molecule via an alkyl group of the indicated number of carbons, for example piperidin-4-yl-methyl, cyclohexylethyl.

The derived expression “(C₁-C₆)alkoxy (C₁-C₆)alkyl” refers to the above defined alkoxy group linked to the rest of the molecule via an alkyl group of the indicated number of carbons, for example methoxymethyl.

Likewise “(C₁-C₆)haloalkoxy(C₁-C₆)alkyl” refers to the above defined (C₁-C₆)haloalkoxy” group linked to the rest of the molecule via an alkyl group of the indicated number of carbons, for example difluoromethoxypropyl.

Likewise “(C₁-C₆)alkoxycarbonyl” refers to the above defined alkoxy group linked to the rest of the molecule via an carbonyl group.

And “(C₁-C₆)alkoxycarbonyl-(C₁-C₆)alkyl” refers to the above defined alkoxy group linked to the rest of the molecule via an carbonyl group further enchained with an alkyl group of the indicated number of carbons, for example methoxycarbonylmethyl.

And “(C₁-C₆)alkoxycarbonyl-(C₁-C₆)alkylthio consequently refer to enchained groups like methoxycarbonylmethylthio.

An oxo moiety is represented by (O) as an alternative to the other common representation, e.g. (═O). Thus, in terms of general formula, the carbonyl group is herein preferably represented as —C(O)— as an alternative to the other common representations such as —CO—, —(CO)— or —C(═O)—. In general the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

When a numerical index the statement (value) “p is zero” or “p is 0” means that the substituent or group bearing the index p (e.g. Ip) is absent, that is to say no substituent, other than H when needed, is present. Likewise when the index is attached to a bridging divalent group (e.g. (CH₂)n) the statement “n in each occurrence is zero . . . ” or “n is 0” means that the bridging group is absent, that is to say it is a bond.

Whenever basic amino or quaternary ammonium groups are present in the compounds of formula (I), physiological acceptable anions, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate may be present. Likewise, in the presence of acidic groups such as COOH groups, corresponding physiological cation salts may be present as well, for instance including alkaline or alkaline earth metal ions.

Compounds of formula (I) when they contain one or more stereogenic center, may exist as optical stereoisomers.

Where the compounds of the invention have at least one stereogenic center, they may accordingly exist as enantiomers. Where the compounds of the invention possess two or more stereogenic centers, they may additionally exist as diastereoisomers. It is to be understood that all such single enantiomers, diastereoisomers and mixtures thereof in any proportion are encompassed within the scope of the present invention. The absolute configuration (R) or (S) for carbon bearing a stereogenic center is assigned on the basis of Cahn-Ingold-Prelog nomenclature rules based on groups' priorities.

“Single stereoisomer”, “single diastereoisomer” or “single enantiomer”, when reported near the chemical name of a compound indicate that the isomer was isolated as single diastereoisomer or enantiomer (e.g via chiral chromatography) but the absolute configuration at the relevant stereogenic center was not determined/assigned.

Atropisomers result from hindered rotation about single bonds where the steric strain barrier to rotation is high enough to allow for the isolation of the conformers (Bringmann G et al, Angew. Chemie Int. Ed. 44 (34), 5384-5427, 2005. doi:10.1002/anie.200462661).

Oki defined atropisomers as conformers that interconvert with a half-life of more than 1000 seconds at a given temperature (Oki M, Topics in Stereochemistry 14, 1-82, 1983).

Atropisomers differ from other chiral compounds in that in many cases they can be equilibrated thermally whereas in the other forms of chirality isomerization is usually only possible chemically.

Separation of atropisomers is possible by chiral resolution methods such as selective crystallization. In an atropo-enantioselective or atroposelective synthesis one atropisomer is formed at the expense of the other. Atroposelective synthesis may be carried out by use of chiral auxiliaries like a Corey Bakshi Shibata (CBS) catalyst, an asymmetric catalyst derived from proline, or by approaches based on thermodynamic equilibration when an isomerization reaction favors one atropisomer over the other.

Racemic forms of compounds of formula (I) as well as the individual atropisomers (substantially free of its corresponding enantiomer) and stereoisomer-enriched atropisomer mixtures are included in the scope of the present invention.

The invention further concerns the corresponding deuterated derivatives of compounds of formula (I). In the context of the present invention, deuterated derivative means that at least one position occupied by a hydrogen atom is occupied by deuterium in an amount above its natural abundance. Preferably, the percent of deuterium at that position is at least 90%, more preferably at least 95%, even more preferably 99%.

All preferred groups or embodiments described above and here below for compounds of formula (I) may be combined among each other and apply as well mutatis mutandis.

As above mentioned, the present invention provides compounds of general formula (I), acting as JAK inhibitors, to processes for the preparation thereof, pharmaceutical compositions comprising them either alone or in combination with one or more active ingredient, in admixture with one or more pharmaceutically acceptable carriers.

In a first aspect the present invention provides a class of compounds of formula (I)

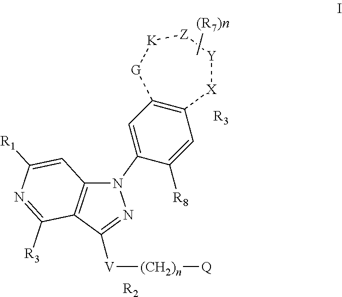

R₁ is a heteroaryl group selected from
imidazo[1,2-b]pyridazin-3-yl, pyrazolo[1,5-a]pyrimidin-3-yl, 3-oxo-(3,4-dihydropyrazin-2-yl)amino;
R₂ is a group of formula

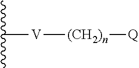

that is a substituent group linked to the molecule scaffold,
wherein
V is absent (meaning it is a bond) or is a divalent group selected from O, S, N(R₆), C(O)N(R₆), N(R₆)C(O), N(R₆)C(O)O, N(R₆)S(O)₂, N(R₆)C(O)N(R₆);
Q is selected from the group consisting of H, (C₁-C₆)alkyl, (C₁-C₆)hydroxyalkyl, (C₁-C₆)alkoxy, —(CH₂)ₘNR₄R₅, (C₃-C₈)cycloalkyl, (C₃-C₁₀)heterocycloalkyl, —S—(C₃-C₆)heterocycloalkyl, and —N(R₆)—(C₃-C₆)heterocycloalkyl; wherein said (C₃-C₈)cycloalkyl and (C₃-C₁₀)heterocycloalkyl are further optionally substituted by one or more substituent selected from the group consisting of —OH, oxo (i.e. (═O)), (C₁-C₁₀)alkyl, (C₁-C₆)alkoxy; halogen, (C₁-C₆)haloalkyl, alkanoyl, (C₁-C₆)hydroxyalkyl, (C₁-C₆)alkoxy(C₁-C₆)alkyl, —N(R₆)(CH₂)ₘC(O)NR₄R₅, —(CO)NR₄R₅, —(CH₂)ₘNR₄R₅, (C₃-C₈)cycloalkyl(C₁-C₆)alkyl; (C₃-C₆)heterocycloalkyl(C₁-C₆)alkyl, (C₃-C₆)heterocycloalkyl and, hydroxyl-(C₃-C₆)heterocycloalkyl;
R₃ is a bicyclic moiety
which is a substituent group linked to the scaffold graphically represented by

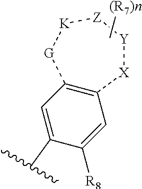

wherein the dashed line --- indicate a single or a double bond
X is selected from N, S, C;
Y is selected from C, N;
Z is selected from C, N, O;
K is absent (meaning a bond) or selected from O, C, S;
G is absent (meaning a bond) or selected from C, O;
(wherein each of X, Y, Z, K and G will be linked in the ring and with the right number of H or substituent according to their valency)

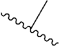

is the point of attachment of a substituent group to the rest of the molecule (meaning the bond linking the substituent group R₁, R₂ or R₃ to the molecule scaffold)
n and m are in each occurrence independently 0 or an integer selected from 1, 2, 3 and 4 (n=0 found in (R₇)ₙ means there is no substituent R₇, i.e. R₇ being H, likewise n=0 or m=0 means there is no linking group —(CH₂)ₙ— or —(CH₂)ₘ, i.e. —(CH₂)ₙ— or —(CH₂)ₘ— being respectively a bond);
R₄ and R₅, the same or different, are selected from the group consisting of
—H,
(C₁-C₆)alkyl,
(C₁-C₆)haloalkyl, and
(C₃-C₆)heterocycloalkyl;
R₆ is in each occurrence independently selected from the group consisting of H, (C₁-C₆)alkyl, (C₁-C₆)hydroxyalkyl;
R₇ is one or more group (preferably 1 or 2) in each occurrence independently selected from the group consisting of —OH, oxo (that is ═O), (C₁-C₆)alkyl, halogen, (C₁-C₆)haloalkyl, (C₁-C₆)hydroxyalkyl, —(CH₂)ₘNR₄R₅, (C₁-C₆)alkyl-S(O)₂— and (C₁-C₆)alkyl-S(O)₂N(R₆)—;
R₈ is selected in the group consisting of
(C₁-C₆)alkoxy,
(C₁-C₆)haloalkoxy;
preferably R₈ is methoxy, fluoromethoxy, difluoromethoxy;
single enantiomers, diastereoisomers and mixtures thereof in any proportion or a pharmaceutically acceptable salt or solvate thereof.

In a preferred embodiment the bicyclic moiety R₃

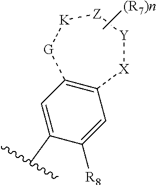

is selected from

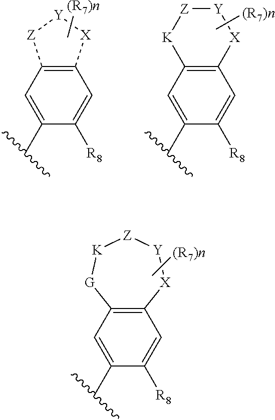

In a further preferred embodiment the bicyclic moiety R₃ is selected in the group consisting of J₁-J₁₃

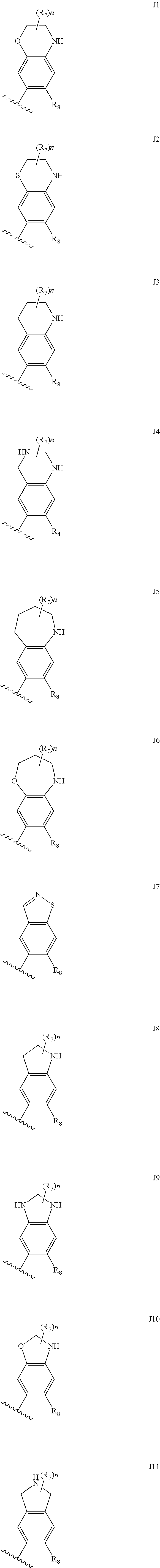

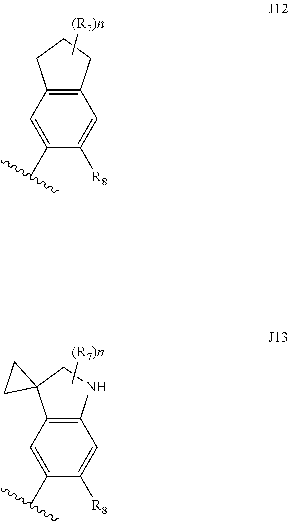

A group of particularly preferred compounds are those of formula I wherein
R₃ is J₁ and R₈ is methoxy
Such preferred group of compounds are represented by the formula Ia

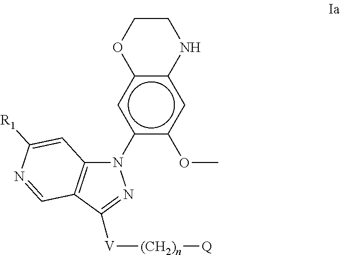

Wherein
R₁ is pyrazolo[1,5-a]pyrimidin-3-yl or (3-oxo-3,4-dihydropyrazin-2-yl)amino
V is a divalent group selected from C(O)N(R₆), N(R₆)C(O)O;
Q is selected from the group consisting of (C₁-C₆)alkyl, (C₁-C₆)alkoxy, —(CH₂)ₘNR₄R₅, (C₃-C₈)cycloalkyl, and (C₃-C₁₂)heterocycloalkyl; wherein said (C₃-C₈)cycloalkyl and (C₃-C₁₂)heterocycloalkyl are further optionally substituted by one or more substituent selected from the group consisting of oxo (i.e. a group (═O)), (C₁-C₁₀)alkyl, halogen, (C₁-C₆)hydroxyalkyl, —(CO)NR₄R₅; (C₃-C₈)cycloalkyl(C₁-C₆)alkyl; (C₃-C₆)heterocycloalkyl;
n and m are in each occurrence independently 0 or an integer selected from 1, 2, 3 and 4;
R₄ and R₅, the same or different, are selected from the group consisting of
—H,
(C₁-C₆)alkyl,
(C₁-C₆)haloalkyl;
single enantiomers, diastereoisomers and mixtures thereof in any proportion
or a pharmaceutically acceptable salt or solvate thereof.
Even more preferred in this group are compounds of the above formula Ia
wherein
V is N(R₆)C(O)O;
Q is selected from the group consisting of (C₁-C₆)alkyl, (C₁-C₆)alkoxy, —(CH₂)ₘNR₄R₅, and (C₃-C₆)heterocycloalkyl; wherein said (C₃-C₆)heterocycloalkyl is a group

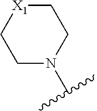

wherein X₁ is selected from CH₂, O, S, NH, NCH₃, (C═O) and S(═O)₂.

Further preferred are compounds of the above formula Ia wherein
V is C(O)N(R₆),
Q is selected from the group consisting of —(CH₂)ₘNR₄R₅, and (C₃-C₆)heterocycloalkyl; wherein said (C₃-C₆)heterocycloalkyl is a group

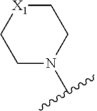

wherein X₁ is selected from the group consisting of CHR₉, O, S, NH, NCH₃, CF₂, (C₁-C₆) alkoxy or (C₁-C₆)alkoxy(C₁-C₆)alkyl, wherein R₉ is H or —(CO)NR₄R₅.

Further preferred are compounds of the above formula Ia wherein
Q is (C₃-C₆)heterocycloalkyl, —S—(C₃-C₆)heterocycloalkyl or —N(R₆)—(C₃-C₆)heterocycloalkyl selected from the group consisting of:

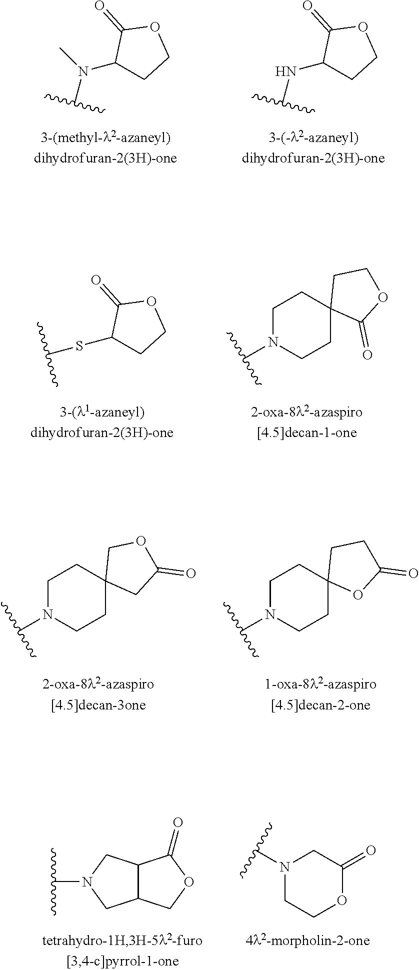

Wherein preferably such tetrahydro-1H,3H-512-furo[3,4-c]pyrrol-1-one is a

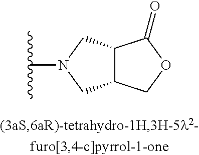 [SMILES: *N1C[C@H]2COC(=O)[C@H]2C1]

According to specific embodiments, the present invention provides the compounds of examples 1 to 86, as listed in the table below, or pharmaceutical acceptable salts and solvates thereof

| Example No | Chemical Name |
|---|---|
| 1 | 6-(difluoromethoxy)-7-(3-methyl-6-(pyrazolo[1,5-a]pyrimidin-3-yl)-1H- |
| | pyrazolo[4,3-c]pyridin-1-yl)-3,4-dihydro-2H-benzo[b][1,4]oxazine |
| 2 | 1-(6-methoxy-2-(methylsulfonyl)isoindolin-5-yl)-3-methyl-6-(pyrazolo[1,5- |
| | a]pyrimidin-3-yl)-1H-pyrazolo[4,3-c]pyridine |
| 3 | 8-methoxy-7-(3-methyl-6-(pyrazolo[1,5-a]pyrimidin-3-yl)-1H-pyrazolo[4,3- |
| | c]pyridin-1-yl)-1,3,4,5-tetrahydro-2H-benzo[b]azepin-2-one |
| 4 | 7-methoxy-6-(3-methyl-6-(pyrazolo[1,5-a]pyrimidin-3-yl)-1H-pyrazolo[4,3- |
| | c]pyridin-1-yl)-3,4-dihydroquinolin-2(1H)-one |
| 5 | 7-methoxy-6-(3-methyl-6-(pyrazolo[1,5-a]pyrimidin-3-yl)-1H-pyrazolo[4,3- |
| | c]pyridin-1-yl)-2,3-dihydroquinolin-4(1H)-one |
| 6 | 6-methoxy-7-(3-methyl-6-(pyrazolo[1,5-a]pyrimidin-3-yl)-1H-pyrazolo[4,3- |
| | c]pyridin-1-yl)-3,4-dihydro-2H-benzo[b][1,4]thiazine |
| 7 | 6-methoxy-7-(3-methyl-6-(pyrazolo[1,5-a]pyrimidin-3-yl)-1H-pyrazolo[4,3- |
| | c]pyridin-1-yl)-2H-benzo[b][1,4]thiazin-3(4H)-one |
| 8 | 6-methoxy-7-(3-methyl-6-(pyrazolo[1,5-a]pyrimidin-3-yl)-1H-pyrazolo[4,3- |
| | c]pyridin-1-yl)-3,4-dihydro-2H-benzo[b][1,4]thiazine 1,1-dioxide |
| 9 | 7-methoxy-8-(3-methyl-6-(pyrazolo[1,5-a]pyrimidin-3-yl)-1H-pyrazolo[4,3- |
| | c]pyridin-1-yl)-2,3,4,5-tetrahydrobenzo[b][1,4]oxazepine |
| 10 | 6-methoxy-5-(3-methyl-6-(pyrazolo[1,5-a]pyrimidin-3-yl)-1H-pyrazolo[4,3- |
| | c]pyridin-1-yl)indolin-2-one |
| 11 | 2,2-difluoro-6-methoxy-7-(3-methyl-6-(pyrazolo[1,5-a]pyrimidin-3-yl)-1H- |
| | pyrazolo[4,3-c]pyridin-1-yl)-2H-benzo[b][1,4]oxazin-3(4H)-one |
| 12 | 7-methoxy-3-methyl-6-(3-methyl-6-(pyrazolo[1,5-a]pyrimidin-3-yl)-1H- |
| | pyrazolo[4,3-c]pyridin-1-yl)-3,4-dihydroquinazolin-2(1H)-one |
| 13 | 1-(6-methoxy-3,4-dihydro-2H-benzo[b][1,4]oxazin-7-yl)-6-(pyrazolo[1,5- |
| | alpyrimidin-3-yl)-N-(1-((tetrahydro-2H-pyran-4-yl)methyl)azetidin-3-yl)- |
| | 1H-pyrazolo[4,3-c]pyridine-3-carboxamide |
| 14 | N-(3-(4,4-difluoropiperidin-1-yl)propyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 15 | 1-(6-methoxy-3,4-dihydro-2H-benzo[b][1,4]oxazin-7-yl)-N-(2-(2-oxo-1-oxa- |
| | 8-azaspiro[4.5]decan-8-yl)ethyl)-6-(pyrazolo[1,5-a]pyrimidin-3-yl)-1H- |
| | pyrazolo[4,3-c]pyridine-3-carboxamide |
| 16 | N-(3-(dimethylamino)propyl)-1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridine-3-carboxamide |
| 17 | 1-(6-methoxy-3,4-dihydro-2H-benzo[b][1,4]thiazin-7-yl)-N-(1- |
| | methylazetidin-3-yl)-6-(pyrazolo[1,5-a]pyrimidin-3-yl)-1H-pyrazolo[4,3- |
| | c]pyridine-3-carboxamide |
| 18 | (R)-1-(6-methoxy-3,4-dihydro-2H-benzo[b][1,4]thiazin-7-yl)-N-((4- |
| | methylmorpholin-2-yl)methyl)-6-(pyrazolo[1,5-a]pyrimidin-3-yl)-1H- |
| | pyrazolo[4,3-c]pyridine-3-carboxamide |
| 19 | 3-(2-oxomorpholino)propyl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 20 | 2-(methyl(2-oxotetrahydrofuran-3-yl)amino)ethyl (1-(6-methoxy-3,4- |
| | dihydro-2H-benzo[b][1,4]thiazin-7-yl)-6-(pyrazolo[1,5-a]pyrimidin-3-yl)- |
| | 1H-pyrazolo[4,3-c]pyridin-3-yl)carbamate |
| 21 | (R)-1-(6-methoxy-3-oxo-3,4-dihydro-2H-benzo[b][1,4]thiazin-7-yl)-N-((4- |
| | methylmorpholin-2-yl)methyl)-6-(pyrazolo[1,5-a]pyrimidin-3-yl)-1H- |
| | pyrazolo[4,3-c]pyridine-3-carboxamide |
| 22 | (S)-1-(6-methoxy-3-oxo-3,4-dihydro-2H-benzo[b][1,4]thiazin-7-yl)-N-((4- |
| | methylmorpholin-2-yl)methyl)-6-(pyrazolo[1,5-a]pyrimidin-3-yl)-1H- |
| | pyrazolo[4,3-c]pyridine-3-carboxamide |
| 23 | N-(3-(dimethylamino)propyl)-1-(6-methoxy-3-oxo-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridine-3-carboxamide |
| 24 | N-(3-(dimethylamino)propyl)-1-(7-methoxy-2-oxo-1,2,3,4- |
| | tetrahydroquinolin-6-yl)-6-(pyrazolo[1,5-a]pyrimidin-3-yl)-1H-pyrazolo[4,3- |
| | c]pyridine-3-carboxamide |
| 25 | N-(3-(dimethylamino)propyl)-1-(7-methoxy-1,2,3,4-tetrahydroquinolin-6- |
| | yl)-6-(pyrazolo[1,5-a]pyrimidin-3-yl)-1H-pyrazolo[4,3-c]pyridine-3- |
| | carboxamide |
| 26 | 7-methoxy-6-(3-(methylamino)-6-(pyrazolo[1,5-a]pyrimidin-3-yl)-1H- |
| | pyrazolo[4,3-c]pyridin-1-yl)-3,4-dihydroquinolin-2(1H)-one |
| 27 | 2-(dimethylamino)ethyl (1-(7-methoxy-2-oxo-1,2,3,4-tetrahydroquinolin-6- |
| | yl)-6-(pyrazolo[1,5-a]pyrimidin-3-yl)-1H-pyrazolo[4,3-c]pyridin-3- |
| | yl)carbamate |
| 28 | 2-(dimethylamino)ethyl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 29 | 2-morpholinoethyl (1-(6-methoxy-3,4-dihydro-2H-benzo[b][1,4]thiazin-7- |
| | yl)-6-(pyrazolo[1,5-a]pyrimidin-3-yl)-1H-pyrazolo[4,3-c]pyridin-3- |
| | yl)carbamate |
| 30 | 3-(4-methylpiperazin-1-yl)propyl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 31 | 2-(piperidin-1-yl)ethyl (1-(6-methoxy-3,4-dihydro-2H-benzo[b][1,4]thiazin- |
| | 7-yl)-6-(pyrazolo[1,5-a]pyrimidin-3-yl)-1H-pyrazolo[4,3-c]pyridin-3- |
| | yl)carbamate |
| 32 | (R)-1-methylpyrrolidin-3-yl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 33 | (S)-1-methylpyrrolidin-3-yl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 34 | (4-(dimethylamino)tetrahydro-2H-pyran-4-yl)methyl (1-(6-methoxy-3,4- |
| | dihydro-2H-benzo[b][1,4]thiazin-7-yl)-6-(pyrazolo[1,5-a]pyrimidin-3-yl)- |
| | 1H-pyrazolo[4,3-c]pyridin-3-yl)carbamate |
| 35 | (R)-2-(3-methoxypyrrolidin-1-yl)ethyl (1-(6-methoxy-3,4-dihydro-2H- |
| | benzo[b][1,4]thiazin-7-yl)-6-(pyrazolo[1,5-a]pyrimidin-3-yl)-1H- |
| | pyrazolo[4,3-c]pyridin-3-yl)carbamate |
| 36 | 3-(1-(6-methoxy-3,4-dihydro-2H-benzo[b][1,4]thiazin-7-yl)-6-(pyrazolo[1,5- |
| | a]pyrimidin-3-yl)-1H-pyrazolo[4,3-c]pyridin-3-yl)-1,1-dimethylurea |
| 37 | 6-methoxy-7-(3-((2-(4-methylpiperazin-1-yl)ethyl)amino)-6-(pyrazolo[1,5- |
| | alpyrimidin-3-yl)-1H-pyrazolo[4,3-c]pyridin-1-yl)-2H-benzo[b][1,4]thiazin- |
| | 3(4H)-one |
| 38 | 7-(3-((2-(dimethylamino)ethyl)amino)-6-(pyrazolo[1,5-a]pyrimidin-3-yl)- |
| | 1H-pyrazolo[4,3-c]pyridin-1-yl)-6-methoxy-2H-benzo[b][1,4]thiazin-3(4H)- |
| | one |
| 39 | 1-(6-methoxybenzo[d]isothiazol-5-yl)-N-(2-morpholinoethyl)-6- |
| | (pyrazolo[1,5-a]pyrimidin-3-yl)-1H-pyrazolo[4,3-c]pyridin-3-amine |
| 40 | 6-methoxy-7-(3-methyl-6-(pyrazolo[1,5-a]pyrimidin-3-yl)-1H-pyrazolo[4,3- |
| | c]pyridin-1-yl)-3,4-dihydro-2H-benzo[b][1,4]oxazine |
| 41 | 1-(6-methoxyindolin-5-yl)-3-methyl-6-(pyrazolo[1,5-a]pyrimidin-3-yl)-1H- |
| | pyrazolo[4,3-c]pyridine |
| 42 | 7-methoxy-6-(3-methyl-6-(pyrazolo[1,5-a]pyrimidin-3-yl)-1H-pyrazolo[4,3- |
| | c]pyridin-1-yl)-1,2,3,4-tetrahydroquinoline |
| 43 | N-(3-(dimethylamino)propyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 44 | 1-(6-methoxy-3,4-dihydro-2H-benzo[b][1,4]oxazin-7-yl)-N-(1- |
| | methylazetidin-3-yl)-6-(pyrazolo[1,5-a]pyrimidin-3-yl)-1H-pyrazolo[4,3- |
| | c]pyridine-3-carboxamide |
| 45 | (R)-1-(6-methoxy-3,4-dihydro-2H-benzo[b][1,4]oxazin-7-yl)-N-((4- |
| | methylmorpholin-2-yl)methyl)-6-(pyrazolo[1,5-a]pyrimidin-3-yl)-1H- |
| | pyrazolo[4,3-c]pyridine-3-carboxamide |
| 46 | 1-(6-methoxy-3,4-dihydro-2H-benzo[b][1,4]oxazin-7-yl)-N-(2-(4- |
| | methoxypiperidin-1-yl)ethyl)-6-(pyrazolo[1,5-a]pyrimidin-3-yl)-1H- |
| | pyrazolo[4,3-c]pyridine-3-carboxamide |
| 47 | 1-(6-methoxy-3,4-dihydro-2H-benzo[b][1,4]oxazin-7-yl)-N-(2-(4- |
| | (methoxymethyl)piperidin-1-yl)ethyl)-6-(pyrazolo[1,5-a]pyrimidin-3-yl)-1H- |
| | pyrazolo[4,3-c]pyridine-3-carboxamide |
| 48 | N-(2-(7-oxa-2-azaspiro[3.5]nonan-2-yl)ethyl)-1-(6-methoxy-3,4-dihydro- |
| | 2H-benzo[b][1,4]oxazin-7-yl)-6-(pyrazolo[1,5-a]pyrimidin-3-yl)-1H- |
| | pyrazolo[4,3-c]pyridine-3-carboxamide |
| 49 | N-(2-(2-oxa-6-azaspiro[3.5]nonan-6-yl)ethyl)-1-(6-methoxy-3,4-dihydro- |
| | 2H-benzo[b][1,4]oxazin-7-yl)-6-(pyrazolo[1,5-a]pyrimidin-3-yl)-1H- |
| | pyrazolo[4,3-c]pyridine-3-carboxamide |
| 50 | 1-(6-methoxy-3,4-dihydro-2H-benzo[b][1,4]oxazin-7-yl)-N-(2-((3aS,6aR)-1- |
| | oxotetrahydro-1H-furo[3,4-c]pyrrol-5(3H)-yl)ethyl)-6-(pyrazolo[1,5- |
| | a]pyrimidin-3-yl)-1H-pyrazolo[4,3-c]pyridine-3-carboxamide |
| 51 | 1-(6-methoxy-3,4-dihydro-2H-benzo[b][1,4]oxazin-7-yl)-N-(2-(3-oxo-2-oxa- |
| | 8-azaspiro[4.5]decan-8-yl)ethyl)-6-(pyrazolo[1,5-a]pyrimidin-3-yl)-1H- |
| | pyrazolo[4,3-c]pyridine-3-carboxamide |
| 52 | 1-(6-methoxy-3,4-dihydro-2H-benzo[b][1,4]oxazin-7-yl)-N-(2-(1-oxo-2-oxa- |
| | 8-azaspiro[4.5]decan-8-yl)ethyl)-6-(pyrazolo[1,5-a]pyrimidin-3-yl)-1H- |
| | pyrazolo[4,3-c]pyridine-3-carboxamide |
| 53 | N-(2-(4-(dimethylcarbamoyl)piperidin-1-yl)ethyl)-1-(6-methoxy-3,4- |
| | dihydro-2H-benzo[b][1,4]oxazin-7-yl)-6-(pyrazolo[1,5-a]pyrimidin-3-yl)- |
| | 1H-pyrazolo[4,3-c]pyridine-3-carboxamide |
| 54 | N1-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3-yl)-N2,N2- |
| | dimethylethane-1,2-diamine |
| 55 | 1-(6-methoxy-3,4-dihydro-2H-benzo[b][1,4]oxazin-7-yl)-N-methyl-6- |
| | (pyrazolo[1,5-a]pyrimidin-3-yl)-1H-pyrazolo[4,3-c]pyridin-3-amine |
| 56 | methyl (1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3-yl)carbamate |
| 57 | 2-(dimethylamino)ethyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 58 | 2-morpholinoethyl (1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridin-3- |
| | yl)carbamate |
| 59 | 3-(4-methylpiperazin-1-yl)propyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 60 | 3-(dimethylamino)propyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 61 | 3-morpholinopropyl (1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridin-3- |
| | yl)carbamate |
| 62 | 2-(1,1-dioxidothiomorpholino)ethyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 63 | 2-(4-methylpiperazin-1-yl)ethyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 64 | 2-thiomorpholinoethyl (1-(6-methoxy-3,4-dihydro-2H-benzo[b][1,4]oxazin- |
| | 7-yl)-6-(pyrazolo[1,5-a]pyrimidin-3-yl)-1H-pyrazolo[4,3-c]pyridin-3- |
| | yl)carbamate |
| 65 | (S)-(4-methylmorpholin-2-yl)methyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 66 | 2-(piperidin-1-yl)ethyl (1-(6-methoxy-3,4-dihydro-2H-benzo[b][1,4]oxazin- |
| | 7-yl)-6-(pyrazolo[1,5-a]pyrimidin-3-yl)-1H-pyrazolo[4,3-c]pyridin-3- |
| | yl)carbamate |
| 67 | 2-((2-oxotetrahydrofuran-3-yl)thio)ethyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 68 | 2-((2-oxotetrahydrofuran-3-yl)amino)ethyl (1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridin-3-yl)carbamate |
| 69 | 2-(dimethylamino)ethyl (1-(7-methoxy-1,2,3,4-tetrahydroquinolin-6-yl)-6- |
| | (pyrazolo[1,5-a]pyrimidin-3-yl)-1H-pyrazolo[4,3-c]pyridin-3-yl)carbamate |
| 70 | 1-(1-(6-methoxy-3,4-dihydro-2H-benzo[b][1,4]oxazin-7-yl)-6-(pyrazolo[1,5- |
| | a]pyrimidin-3-yl)-1H-pyrazolo[4,3-c]pyridin-3-yl)-3-(3- |
| | morpholinopropyl)urea |
| 71 | 6′-methoxy-5′-(3-methyl-6-(pyrazolo[1,5-a]pyrimidin-3-yl)-1H- |
| | pyrazolo[4,3-c]pyridin-1-yl)spiro[cyclopropane-1,3′-indolin]-2′-one |
| 72 | 5-methoxy-6-(3-((2-morpholinoethyl)amino)-6-(pyrazolo[1,5-a]pyrimidin-3- |
| | yl)-1H-pyrazolo[4,3-c]pyridin-1-yl)benzo[d]oxazol-2(3H)-one |
| 73 | N-(3-(dimethylamino)propyl)-6-(imidazo[1,2-b]pyridazin-3-yl)-1-(6- |
| | methoxy-3,4-dihydro-2H-benzo[b][1,4]oxazin-7-yl)-1H-pyrazolo[4,3- |
| | c]pyridine-3-carboxamide |
| 74 | 3-((3-((2-(dimethylamino)ethyl)amino)-1-(6-methoxy-3,4-dihydro-2H- |
| | benzo[b][1,4]oxazin-7-yl)-1H-pyrazolo[4,3-c]pyridin-6-yl)amino)pyrazin- |
| | 2(1H)-one |
| 75 | N-(3-(dimethylamino)propyl)-1-(6-methoxy-3,4-dihydro-2H- |
| | benzo[b][1,4]oxazin-7-yl)-6-((3-oxo-3,4-dihydropyrazin-2-yl)amino)-1H- |
| | pyrazolo[4,3-c]pyridine-3-carboxamide |
| 76 | N-(3-(4,4-difluoropiperidin-1-yl)propyl)-1-(6-methoxy-3,4-dihydro-2H- |
| | benzo[b][1,4]oxazin-7-yl)-6-((3-oxo-3,4-dihydropyrazin-2-yl)amino)-1H- |
| | pyrazolo[4,3-c]pyridine-3-carboxamide |
| 77 | N-(2-(2,2-dimethyl-6-oxomorpholino)ethyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 78 | 1-(6-(difluoromethoxy)-3,4-dihydro-2H-benzo[b][1,4]oxazin-7-yl)-N-(3- |
| | (dimethylamino)propyl)-6-(pyrazolo[1,5-a]pyrimidin-3-yl)-1H-pyrazolo[4,3- |
| | c]pyridine-3-carboxamide |
| 79 | N-(2-(4-carbamoylpiperidin-1-yl)ethyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 80 | (R)-N-(2-(3-carbamoylpyrrolidin-1-yl)ethyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 81 | 1-(6-methoxy-3,4-dihydro-2H-benzo[b][1,4]oxazin-7-yl)-N-(2-(7-oxo-6-oxa- |
| | 2-azaspiro[3.4]octan-2-yl)ethyl)-6-(pyrazolo[1,5-a]pyrimidin-3-yl)-1H- |
| | pyrazolo[4,3-c]pyridine-3-carboxamide |
| 82 | N-(2-(2-oxa-6-azaspiro[3.4]octan-6-yl)ethyl)-1-(6-methoxy-3,4-dihydro-2H- |
| | benzo[b][1,4]oxazin-7-yl)-6-(pyrazolo[1,5-a]pyrimidin-3-yl)-1H- |
| | pyrazolo[4,3-c]pyridine-3-carboxamide |
| 83 | 1-(6-methoxy-3,4-dihydro-2H-benzo[b][1,4]oxazin-7-yl)-N-(2-(2-methyl-6- |
| | oxomorpholino)ethyl)-6-(pyrazolo[1,5-a]pyrimidin-3-yl)-1H-pyrazolo[4,3- |
| | c]pyridine-3-carboxamide |
| 84 | N-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3-yl)-1- |
| | methylpiperidine-4-carboxamide |
| 85 | N-(2-(4-(3-hydroxyoxetan-3-yl)piperidin-1-yl)ethyl)-1-(6-methoxy-3,4- |
| | dihydro-2H-benzo[b][1,4]oxazin-7-yl)-6-(pyrazolo[1,5-a]pyrimidin-3-yl)- |
| | 1H-pyrazolo[4,3-c]pyridine-3-carboxamide |
| 86 | 2-((2-oxotetrahydrofuran-3-yl)thio)ethyl (1-(7-methoxy-1,2,3,4- |
| | tetrahydroquinolin-6-yl)-6-(pyrazolo[1,5-a]pyrimidin-3-yl)-1H-pyrazolo[4,3- |
| | c]pyridin-3-yl)carbamate |

The compounds of the invention, including all the compounds hereabove listed, can be prepared from readily available starting materials using general methods and procedures as described in the experimental part below or by using slightly modified processes readily available to those of ordinary skill in the art. Although a particular embodiment of the present invention may be shown or described herein, those skilled in the art will recognize that all embodiments or aspects of the present invention can be prepared using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures. Processes of preparation described below and reported in the following schemes should not be viewed as limiting the scope of the synthetic methods available for the preparation of the compounds of the invention.

In some cases a step is needed in order to mask or protect sensitive or reactive moieties, generally known protective groups (PG) could be employed, in accordance with general principles of chemistry (Protective group in organic syntheses, 3rd ed. T. W. Greene, P. G. M. Wuts).

Compounds of formula (I), here reported again for clarity, including all here above listed, can be usually prepared according to the procedures shown in the schemes below. Where a specific detail or step differs from the general schemes it has been detailed in the specific examples, and/or in additional schemes.

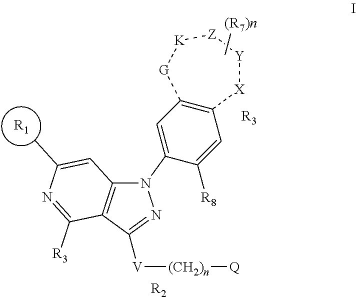

Compounds of formula (I) can be prepared according to scheme 1. Compound IV is an intermediate where general group r₁, r₂, and r₃ can be converted into R₁, R₂ and R₃ respectively by means of procedures well known to those skilled in the art such as protective groups deprotection and/or functional group conversion that may involve more than one step. Said procedures can be applied to one or more of those groups (r₁, r₂, and r₃) to allow the conversion of intermediate IV into compound of general formula I and they are detailed in the experimental section for specific examples. It is apparent that in the case such conversions are not needed (when r₁, r₂, and r₃ correspond to R₁, R₂ and R₃ respectively), any general approach described below for the preparation of intermediate IV will provide a compound of general formula I.

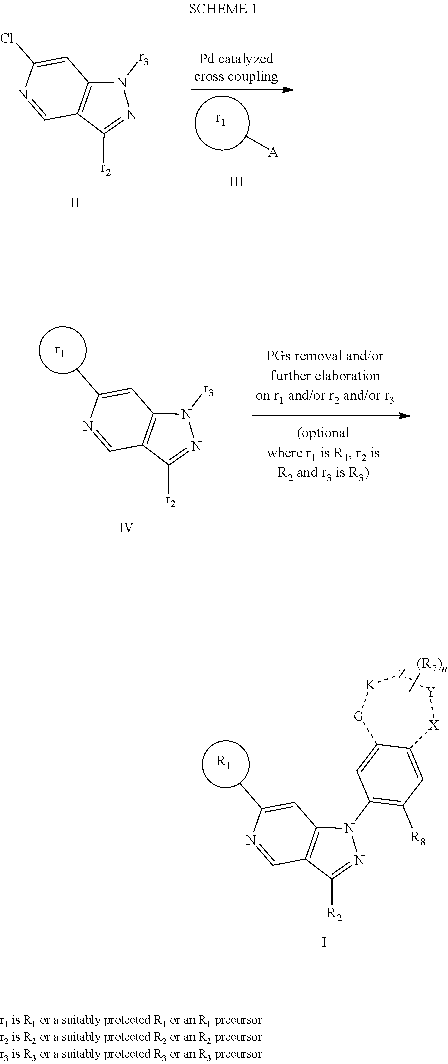

For synthetic convenience, cyclic and/or exocyclic NH moieties that may be present in r₂ and/or r₃ may need to be protected during the synthetic sequences. Suitable protective groups for protecting such NH moieties can be carbamate type protective groups such as Boc (tert-butoxycarbonyl) and/or hemiaminals such as SEM (2-(Trimethylsilyl)ethoxymethyl). A Boc group can be inserted by reacting the NH derivative with Boc₂O in the presence of a base like for example DMAP or pyridine, and in an organic solvent like THF or DMF at room temperature. A SEM group can be inserted by reacting the NH derivative with SEM-Cl (2-(Trimethylsilyl)ethoxymethyl chloride) in a suitable organic solvent like DMF and in the presence of a strong base like NaH or LiHMDS (lithium hexamethyldisilazide), at low temperature like 0° C.

A Boc group can be easily removed by treating Boc protected intermediate IV (or from other convenient upstream intermediates) in acidic conditions with an organic or an inorganic strong acid. For example, Boc group can be cleaved by treating the intermediates with trifluoroacetic acid neat or in mixture with an organic solvent such as DCM, DCE, THF or similar, typically at room temperature for a few hours (typically 1 to 3 h). A SEM group can be removed by treating intermediate IV (or from other convenient upstream intermediates) in acidic conditions similarly to Boc group removal. In some cases, acidic treatment may lead incomplete SEM removal, therefore a further treatment with concentrated ammonia may be necessary to completely remove formaldehyde adducts resistant to acidic treatment. It is understood that insertion and removal of NH protective groups in r₂/r₃ can be done where it is more convenient in the synthetic sequence.

Compound of formula I (or intermediate IV) can be obtained by direct introduction of R₁ (or r₁) through a metal/palladium catalysed cross coupling reaction such as Suzuki coupling, Stille coupling, Buchwald-Hartwig or similar (Strategic application of named reactions in organic synthesis, L. Kurti, B. Czako, Ed. 2005) by reaction of intermediate II with intermediate III. For example, a suitable palladium catalysed cross coupling for introducing R₁, when it is an pyrazolo[1,5-a]pyrimidin-3-yl, is a Suzuki coupling. Suzuki coupling can be performed by reacting intermediate II with the suitable boronic acid or boron pinacolate derivative (intermediate III, where r₁ is pyrazolo[1,5-a]pyrimidin-3-yl and A is dihydroxyboryl or 4,4,5,5-tetramethyl-1,3,2-dioxaborolanyl) in the presence of a Pd catalyst such as tetrakistriphenylphosphinepalladium(0), PdCl₂(dppf)₂, or a ligand-palladacycle precatalyst for example XPhos-Pd-G3 [(2-Dicyclohexylphosphino-2′,4′,6′-triisopropyl-1,1′-biphenyl)[2-(2′-amino-1,1′-biphenyl)]palladium(II) methanesulfonate], in an organic solvent such as 1,4-dioxane, THF, 1,2-dimethoxyethane, 2-propanol or DMF, with or without water, in the presence of an inorganic base such as an alkaline carbonate (for example Cs₂CO₃ or K₂CO₃) or an inorganic phosphate (for example K₃PO₄), under heating (typically in the range of 50-100° C.) for a few hours (typically 1 to 3 h). Boronic acid and boronic pinacolate esters are generally commercially available or may be readily prepared by those skilled in the art starting from commercially available reagents.

A suitable palladium catalyzed cross coupling for introducing R₁ when it is an imidazo[1,2-b]pyridazin-3-yl, is a Stille coupling that can be performed by reacting intermediate II with the suitable stannane reagent (intermediate III, where r₁ is imidazo[1,2-b]pyridazin-3-yl and A is tributylstannyl or a trimethylstannyl) in the presence of an appropriate palladium catalyst (such as Pd(PPh₃)₂Cl₂) in a polar organic solvent (for example DMF or dioxane with or without additives (like base or lithium salt). Stannanes are generally commercially available or may be readily prepared by those skilled in the art starting from commercially available reagents.

A suitable palladium catalyzed cross coupling for introducing r₁, when it is an (3-oxo-3,4-dihydropyrazin-2-yl)amino, is a Buchwald-Hartwig coupling. For synthetic convenience the lactam group of (3-oxo-3,4-dihydropyrazin-2-yl)amino needs to be masked as an alkoxyimmino group (such as methoxy immino, —C(OMe)=N—) and that is deprotected at the end of the synthesis from intermediate IV. Intermediate II and intermediate III (where r₁ is 3-methoxypyrazin-2-aminyl and A is H) can be reacted to give intermediate IV (where r₁ is 3-methoxypyrazin-2-aminyl) in the presence of a suitable ligand palladacyle system such as XPhos-Pd-G3 (2-Dicyclohexylphosphino-2′,4′,6′-triisopropyl-1,1′-biphenyl)[2-(2′-amino-1,1′-biphenyl)]palladium(II) methanesulfonate) or RuPhos-Pd-G3 (2-Dicyclohexylphosphino-2′,6′-diisopropoxy-1,1′-biphenyl)[2-(2′-amino-1,1′-biphenyl)]palladium(II) methanesulfonate) or in general a suitable Pd source (for example Pd₂(dba)₃ or Pd(OAc)₂) with a suitable phosphine ligand such as biphenylphosphine ligand type (RuPhos, X-Phos, or similar), in the presence of a strong organic base such as sodium tert-butoxide or an inorganic base such as Cs₂CO₃, in an organic solvent such 1,4-dioxane, THF or toluene, under heating at high temperature (typically 80-120° C.), for a few hours (typically overnight). Preparation of compound of formula I wherein R₁ is 3-oxo-3,4-dihydropyrazin-2-yl)amino can be prepared by mean of demethylation of the corresponding methoxy-immino derivative by treatment of the protected precursor with TMS-Cl (trimethylsilyl chloride) and sodium iodide in acetonitrile for 1 to 5 h at 60-100° C.; these condition can also lead to Boc/SEM groups deprotection eventually present in r₂/r₃.

The above described process may provide at least one non limiting synthetic route for the preparation of examples of the invention starting from the appropriate intermediate II.

A possible approach for the preparation of intermediate II is reported in scheme 2. Intermediate II can be prepared by means of N-arylation of intermediate V with an halide intermediate VI by using a copper catalyzed Ullmann type reaction. An Ullmann reaction between a NH heteroaryl and an aryl/heteroaryl halide (bromide or iodide) can be performed in the presence of a suitable copper(I) catalyst/promoter such as Cut, Cu₂O or CuTC (copper thiophene carboxylate), ligandless or with a suitable ligand such as N,N-dimethylglicine, proline, phenantroline or dimethylcyclohexane-1,2-diamine (DMCHA), in the presence of an inorganic base such as K₂CO₃ or Cs₂CO₃, by heating (typically 90-150° C.) in a polar organic solvent such as DMSO, DMF or DMA, overnight or longer.

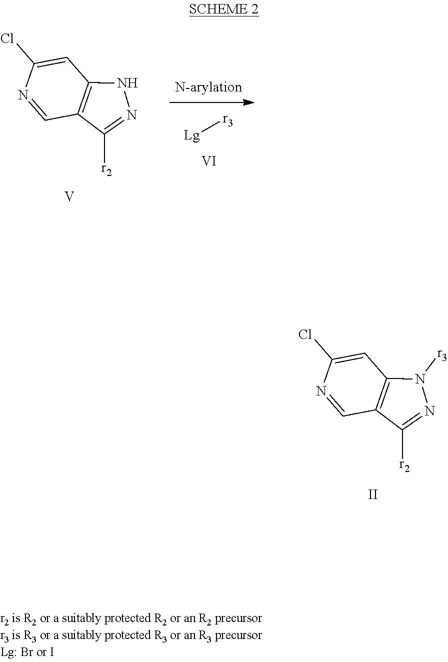

In another approach, intermediate II when r₃/R₃ is J₂, indicated as IIⁱ (for n:0) and IIⁱⁱ (for n:1, R₇: 3-oxo) can be synthesized by means of a multistep synthesis according to scheme 3. Intermediate V and intermediate VIIa can undergo an aromatic nucleophilic substitution (SNAr) to give intermediate VIIIa, for example by reacting them in an organic solvent like DMF, DMSO or 1,4-dioxane, in the presence of an organic base like DBU or DIPEA or an inorganic base like K₂CO₃ or Cs₂CO₃, at RT or higher (up to 120° C.), for a few hours (typically 1 to 4 hours). Similarly, intermediate VIIIb and VIIIc can be made from V by reaction with VIIb and VIIc respectively. Intermediate VIIIa can be reacted with mercaptoethanol (r₅-SH, r₅ is ethanol-2yl) under Pd-catalyzed C—S coupling conditions to give intermediate IXa. C—S coupling can be performed by reacting aryl bromide VIIIa and mercaptoethanol in the presence of a suitable catalytic system like as Pd₂(dba)₃/Xantphos or another suitable palladium source/phosphine source, in an organic solvent as 1,4-dioxane, toluene or DMA, in the presence of an organic base like DIPEA or DBU, at temperature up to 100° C. for a few hours (typically 3-5 hours).

Intermediate IXb can be synthesized by aromatic nucleophilic substitution (SNAr) of intermediate VIIIa with methyl 2-sulfanylacetate (r₅-SH, r₅ is methyl ethanoat-2-yl) by reacting them in an organic solvent like acetonitrile or dioxane, under heating at high temperature (typically 120° C.), for a few hours (typically 3 to 6 hours).

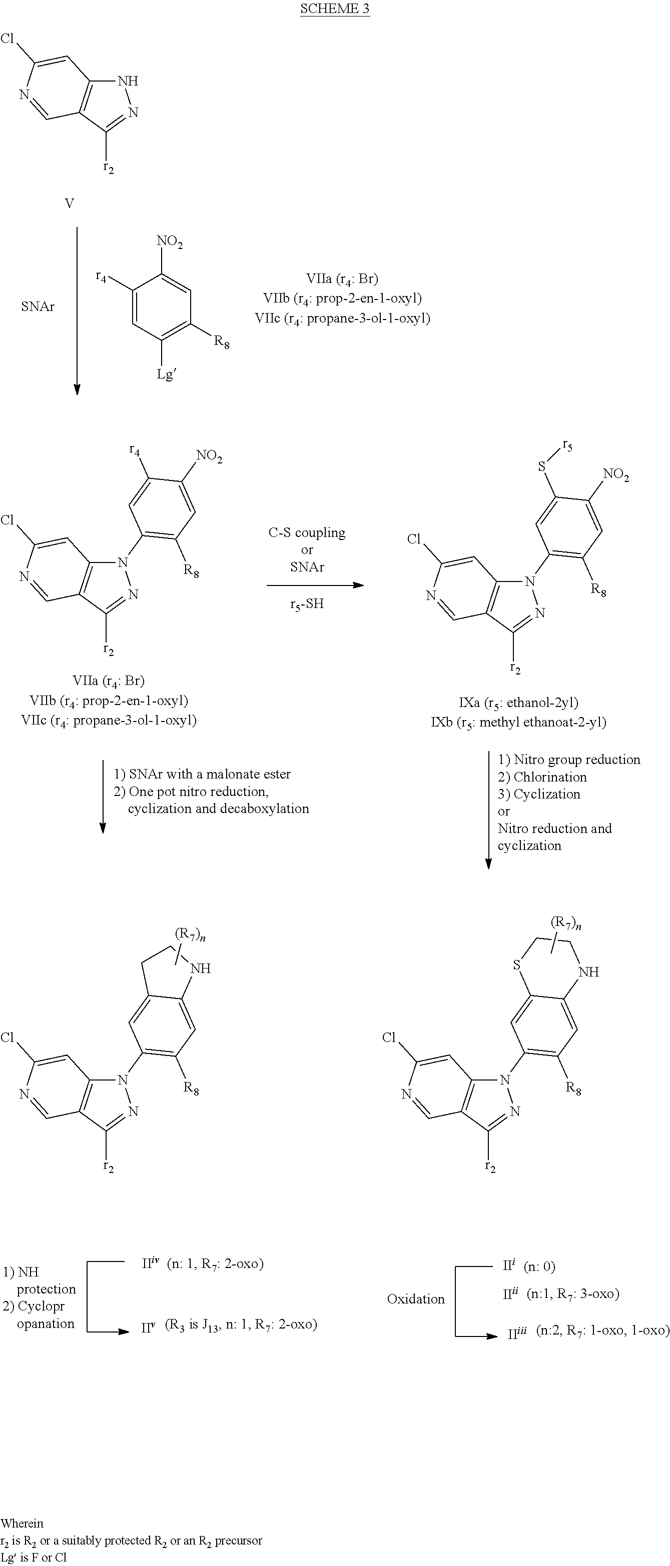

Intermediate IXa can be converted into intermediate II by a three steps process that involves: 1) nitro reduction, 2) chlorination and cyclization. Nitro reduction can be performed by treating intermediate IXa with a reducing agent like iron powder or zinc powder, in an organic solvent like methanol or ethanol, in the presence of an aqueous solution of a weak inorganic acid salt like ammonium chloride, by heating at temperature up to 80° C. for a few hours (typically 4-5 hours). Subsequent conversion of alcohol to the corresponding chloride can be achieved by reaction with neat thionyl chloride or oxalyl chloride at low temperature (around 0° C.). Chloride intermediate can be cyclized to give intermediate Ili by heating (typically 80-100° C.) in an appropriate organic solvent like DMF or 1,4-dioxane, in presence of an inorganic base like potassium carbonate or sodium carbonate and in the presence of an additive like sodium iodide.

Intermediate IXb can be converted into intermediate IIⁱⁱ by a process that involves nitro reduction and cyclization to lactam. Nitro reduction of intermediate IXb can be obtained similarly to what described for intermediate IXa. Cyclization can occur spontaneously during nitro reduction step or promoted in a separate step by treatment with a strong organic acid like TFA.

In an alternative approach, intermediate IIⁱⁱ can be converted into intermediate IIⁱ by reduction of lactam with borane like BH₃*THF complex into the corresponding lactam.

In another approach, intermediate II can be converted into intermediate IIⁱⁱⁱ by oxidation of sulfide to sulfone using an oxidizing agent like m-CPBA, in an organic solvent like DCM, at temperature around 0° C.

In another way, intermediates II when r₃/R₃ is J8, indicated as II^{iv} in scheme 3, can be prepared by a multistep process starting from intermediate VIIIa. In the first step, intermediate VIIIa can undergo aromatic nucleophilic substitution with a malonate ester like diethyl malonate, by heating reagents (typically 60-70° C.) in an organic solvent like DMSO or DMF. In the second step, treatment with iron powder at high temperature (for example 90° C.) in acetic acid as a solvent can promote one pot nitro reduction, cyclization and decarboxylation to give intermediate II^{iv}.

In another approach, intermediate II^{iv} can be converted to II^{v} by a two step process that involves 1) NH protection with SEM group and 2) cyclopropanation with a sulphur vinyl ylide like vinyl diphenyl sulfonium triflate.

In a different way, intermediate II when r₃/R₃ is J₆ (with n:0) indicated as intermediate II^{vi} and intermediate II when r₃/R₃ is J1 (with n:3, R₇: 3-oxo, 1,1-difluoro) indicated as intermediate II^{vii}, can be prepared according to scheme 4.

Intermediate VIIIb can be converted into intermediate X by a two step process that involves 1) de-allylation and 2) nitro reduction. De-allylation reaction can be performed by treating (typically at 50-70° C.) intermediate VIIIb with an inorganic base like potassium carbonate or cesium carbonate, in an organic solvent like methanol or ethanol and in the presence of a Pd catalyst such as tetrakis(triphenylphosphine)palladium(0) or palladium chloride (II). Subsequent nitro reduction can be performed similarly to what described in scheme 3 for the reduction of intermediate IXa. Intermediate X can be selectively acylated on the anilinic nitrogen to form the corresponding amide, by transamidation of methyl 2-2,2-dibromo-difluoroacetate promoted by DABAL-Me₃ at high temperature (typically 130° C.) in an organic solvent like THF. Subsequent cyclization of amide intermediate, promoted by an organic base like DBU or DIPEA by heating (100-120° C.) in an organic solvent like THF or dioxane, can give intermediate II^{vii}.

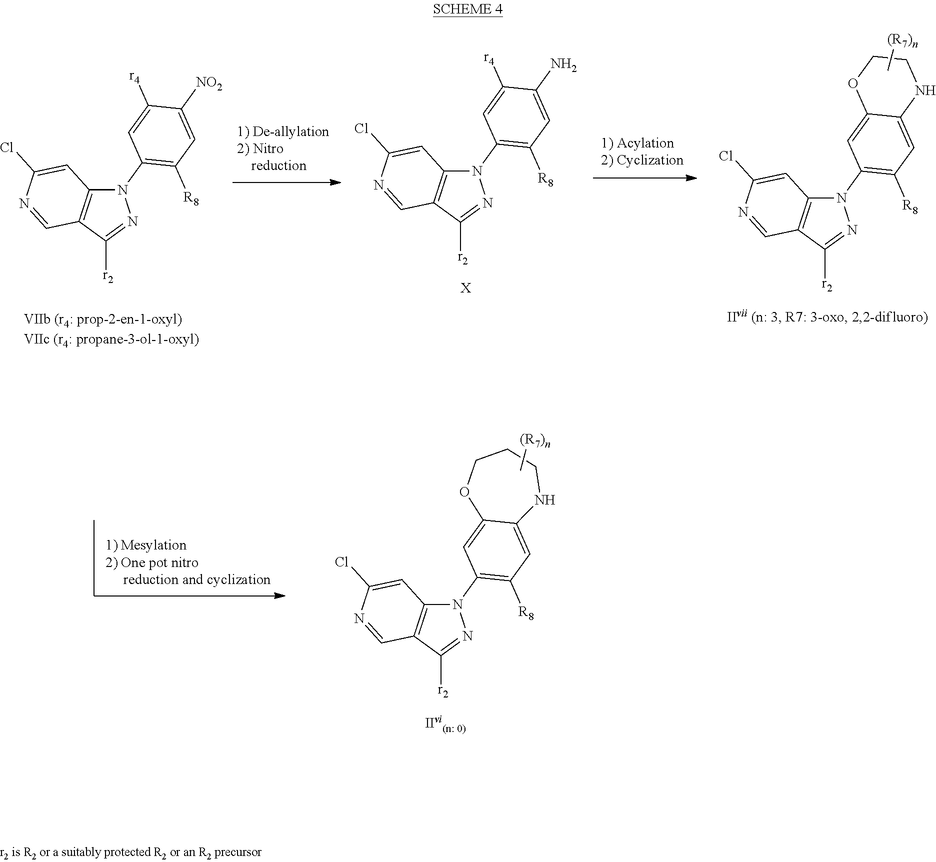

In another approach, intermediate II^{vi} can be obtained from intermediate VIIc by a two step process that involve mesylation of the free hydroxyl group in r₄ (when r₄ is propane-3-ol-1-oxyl) and followed by one pot nitro reduction and cyclization. Mesylation can be carried out by treatment of alcohol with methanesulfonyl chloride in an organic solvent like DMF at room temperature, followed by one pot nitro reduction and cyclization by heating intermediate (around 80° C.) intermediate with iron, in an organic solvent like ethanol, in the presence of a weak inorganic acid like aqueous ammonium chloride.

In a different way, intermediate II when r₂ is —NR₆(CH₂)ₙQ′, indicated as IIa, can be made according to scheme 5. Q′ is a group that, if differs from Q, can be easily converted into Q by means of procedures well known to those skilled in the art such as protective groups deprotection and/or functional group conversion that may involve more than one step.

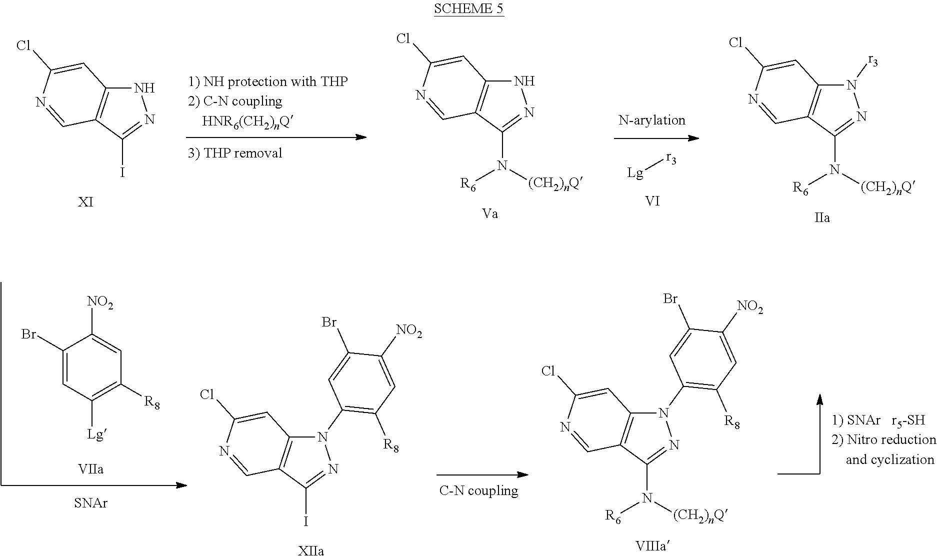

Intermediate XI can be converted into intermediate Va by a multistep process that involves 1) NH protection, 2) C—N coupling with HNR₆(CH₂)ₙQ′ and 3) N-PG deprotection. A suitable protective group for this NH of intermediate XI is for example a THP group (tetrahydropyranyl). THP introduction can be achieved by heating around 40° C. intermediate XI with dihydropyran in an organic solvent like THF, DCM or mixture and in the presence of a sulfonic acid like methanesulfonic acid for several hours (12 h or more). A C—N coupling between THP protected intermediate XI and HNR₆(CH₂)ₙ-Q′ may be performed by heating (typically 60-100° C.) the heteroaromatic iodide and the amine in an organic solvent like DMF or DMSO, in the presence of copper(I) catalyst/promoter such as CuI, Cu₂O or CuTC (copper thiophene carboxylate), ligandless or with a suitable ligand such as proline, N,N-dimethylglicine, dimethylcyclohexane-1,2-diamine (DMCHA), and in the presence of an inorganic base as K₂CO₃ or Cs₂CO₃. Finally, THP removal can be performed by acidic treatment with an organic acid like TFA or methanesulphonic acid, in the presence of a silane scavenger like triethylsilane, in an organic solvent like DMF or THF for a few hours (around 2 h) at room temperature. Conversion of intermediate Va to intermediate IIa can be performed using similar conditions to what described in scheme 2 for the conversion of intermediate V into intermediate II.

In another way, intermediate XI and intermediate VIIa can be converted by SNAr into intermediate XIIa similarly to what described in scheme 3 for the conversion of V and VIIa to VIIIa. Conversion of intermediate XIIa into VIIIa′ can be done using similar conditions that were reported above for the C—N coupling. Finally, conversion of VIIa′ to intermediate IIa (when r₃/R₃ is J2) can be done applying similar conditions already reported in scheme 3 for the conversion of IXb to IIⁱⁱ.

In another approach, intermediates II can be obtained from further elaboration of r₂ group by general accepted methods and in accordance with principles of chemistry. In the following schemes, the most common transformations that can be used to obtain specific intermediates II have been detailed. For sake of clarity they were labelled with an additional letter index.

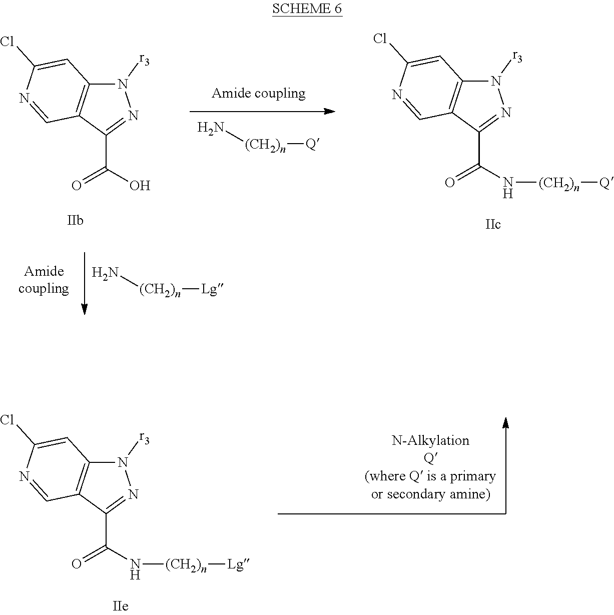

In one of these alternative approaches, intermediate IIc (intermediate II when r₂ is —C(O)N(R₆)—(CH₂)ₙ-Q′) can be prepared according to scheme 6 starting from intermediate IIb (intermediate II when r₂ is —C(O)OH) by amide coupling with HNR₆(CH₂)ₙQ′. An amide coupling can be performed by reacting the amine and the acid in an organic solvent like DMF, DCM, or THF, in the presence of a coupling agent like HATU ((1-[Bis(dimethylamino)methylene]-1H-1,2,3-triazolo[4,5-b]pyridinium 3-oxide hexafluorophosphate), HBTU (0-(Benzotriazol-1-yl)-N,N,N′,N′-tetramethyluronium hexafluorophosphate) or COMU ((1-Cyano-2-ethoxy-2-oxoethylidenaminooxy)dimethylamino-morpholino-carbenium) and in the presence of an organic base like DIPEA, TEA, or pyridine.

In a different way, when for synthetic convenience intermediate IIb is synthesized as methyl ester, it can be converted directly to amide by a transamidation reaction with the corresponding amine promoted by DABAL-Me₃ at high temperature (typically 130° C.) in an organic solvent like THF.

In another approach, intermediate IIc can be prepared from intermediate IIe by displacement of Lg″ by alkylation with primary/secondary nitrogen present in Q′ moiety by treating at room temperature or higher (for example 40° C.) for a few hours to overnight the amine Q′ and halide IIe in an organic solvent like DMF, acetone or 1,4-dioxane, in the presence of an organic base like DIPEA, TEA or an inorganic base like sodium carbonate together with an inorganic iodide like sodium iodide. Intermediate IIe can be obtained from intermediate IIb and HNR₆(CH₂)ₙLg″ by an amide coupling reaction using similar conditions to those reported above in this scheme for the conversion of intermediate IIb to intermediate IIc.

Intermediate IIb can be made by using conditions reported above for general synthesis of intermediate II in scheme 2, scheme 3 and scheme 4 starting from a suitable intermediate V where r₂ is —COOH or —COOMe.

In another approach, reported in scheme 7, intermediate IIg (or IIh) may be prepared from intermediate IIf and HO(CH₂)ₙQ′ (or HNR₆(CH₂)ₙQ′) by a two steps/one pot process that involve: 1) isocyanate formation and 2) reaction with alcohol (or amine) to form the corresponding carbamate (or urea). Intermediate IIf can be reacted with bis(trichloromethyl) carbonate in an organic solvent as DCM, THF or Me-THF, at low temperature like dry ice temperature (−78° C.), in the presence of an organic base like TEA or DIPEA for times up to 1 or 2 hours to form the corresponding isocyanate; addition of intermediate alcohol (or amine) and reaction at room temperature lead to the formation of carbamate of formula IIg (or urea of formula IIh). An alternative method to form the isocyanate intermediate from IIf can use carbonyldiimidazole (CDI) in an organic solvent like 2-Me-THF by heating at temperature around 90° C. overnight.

In a different approach, carbamate IIg can be obtained by reaction of amine IIf can be reacted with the corresponding alcohol chloroformate (Cl—C(O)O(CH₂)ₙQ′) in an organic solvent like DCM or THF and in the presence of a base like pyridine or DIPEA.

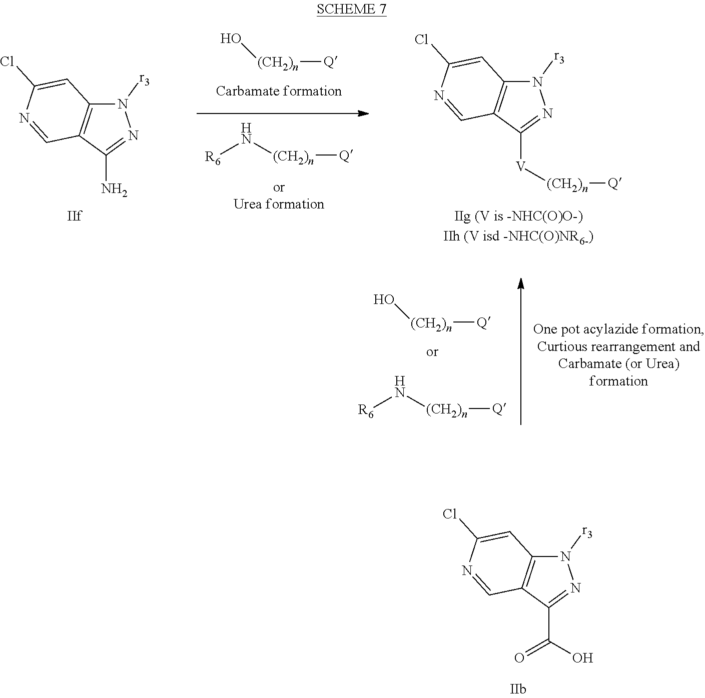

In an alternative method, intermediate of formula IIg (and IIh) may be obtained from intermediate IIb and alcohol intermediate HO(CH₂)ₙQ′ (or amine intermediate HNR₆(CH₂)ₙQ′) through a two steps/one pot process that involves: 1) acylazide formation and Curtius rearrangement to give the corresponding isocyanate intermediate and 2) one pot reaction of isocyanate with alcohol (or amine) to form the corresponding carbamate (or urea). The corresponding acyl azide of intermediate IIb can be prepared by reaction of IIb with an azide source as azido(trimethyl)silane, in the presence of a suitable coupling agent such as an alkyl phosphonic anhydride like T3P (2,4,6-Tripropyl-1,3,5,2,4,6-trioxatriphosphorinane-2,4,6-trioxide solution), with an organic base like TEA or DIPEA, and in an organic solvent such as 2-methyl-THF, DMF or toluene. The following Curtius rearrangement can be done by thermal degradation around solvent reflux temperature (generally from 50° C. to 100° C.) for few hours (generally 1-3 hours) to give the corresponding isocyanate; after isocyanate formation, addition of alcohol intermediate HO(CH₂)ₙQ′ (or amine HNR₆(CH₂)ₙQ′) and continuing reflux overnight can lead to the formation of the corresponding carbamate of formula IIg (or urea IIh).

Intermediate IIf can be made by using conditions reported above for general synthesis of intermediate II in scheme 2, scheme 3 and following scheme 4 starting from a suitable intermediate V where r₂ is —NH₂.

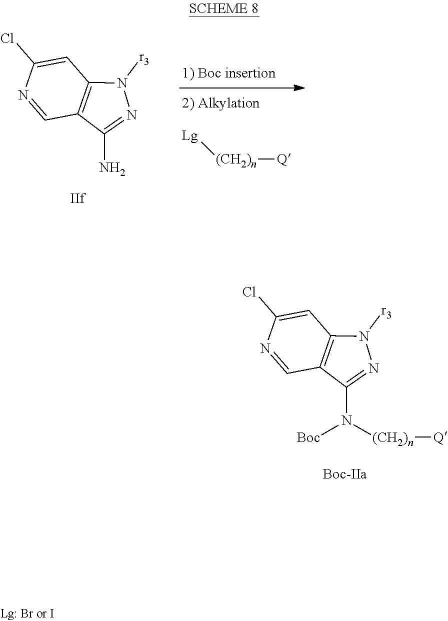

In an alternative approach reported in scheme 8, intermediate IIa when exocyclic NH is protected by a Boc group, indicated as Boc-IIa, can be prepared by a two step process that involves 1) Boc insertion at exocyclic amine and 2) alkylation with Lg-(CH₂)ₙ-Q′. Boc protection can be performed by reaction of IIf with Boc₂O in an organic solvent like THF or 1,4-dioxane and in the presence of a strong base like LiHMDS (lithium hexamethyldisilazide) or NaH, at low temperature like 0° C. Alkylation can be performed by reacting Boc intermediate with Lg-(CH₂)ₙ-Q′ in an organic solvent like DMF or THF, in the presence of a strong base like NaH, at low temperature like 0° C.

In a different approach, intermediate II can be obtained by further elaboration of Q′ group to Q″ by means of the functional group transformation summarized in table 1, full details of transformation were provided in the experimental section. Q″ is a group that, if differs from Q, can be easily converted into Q by means of procedures well known to those skilled in the art such as protective groups deprotection and/or functional group conversion that may involve more than one step.

TABLE 1

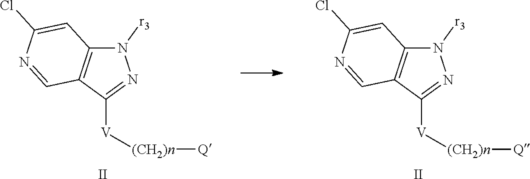

| —V(CH₂)ₙQ′ | —V(CH₂)ₙQ″ | Reaction type/steps |
|---|---|---|
| 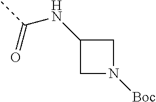 | 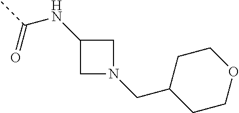 | 1) Boc deprotection 2) Alkylation |
| 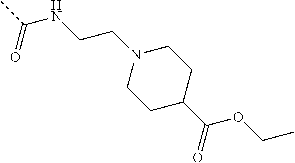 | 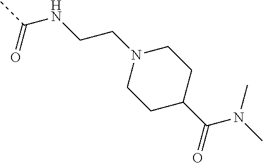 | 1) Ethyl ester hydrolysis 2) Amide coupling |
| 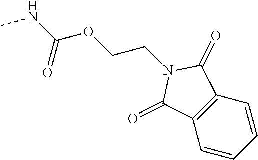 | 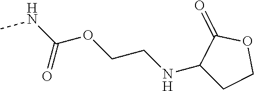 | 1) Deprotection of phthalimide with hydrazine 2) Alkylation |
| 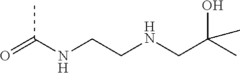 | 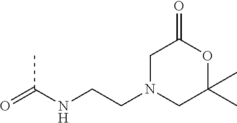 | 1) Boc deprotection on r₃ 3) Condensation with oxaldehyde |
| 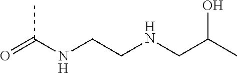 | 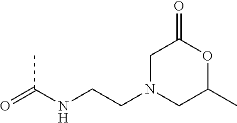 | Tandem alkylation and transesterification with ethylbromoacetate |

In a different approach, intermediate IV can be obtained by further elaboration of Q′ group to Q″ by means of the functional group transformation summarized in table 2, full details of transformation were provided in the experimental section. Q″ is a group that, if differs from Q, can be easily converted into Q by means of procedures well known to those skilled in the art such as protective groups deprotection and/or functional group conversion that may involve more than one step.

TABLE 2

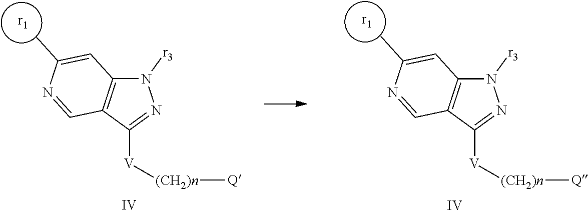

| —V(CH₂)ₙQ′ | —V(CH₂)ₙQ″ | Reaction type/steps |
|---|---|---|
| 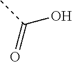 | 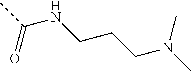 | Amide coupling |
| 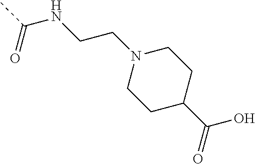 | 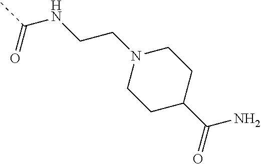 | Amide coupling |
| 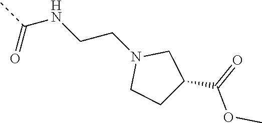 | 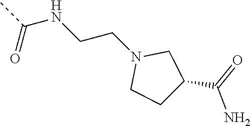 | 1) Hydrolysis 2) Amide coupling |

As herein described in details, the compounds of the invention are inhibitors of kinase activity, in particular. inhibiting JAK kinase activity for the treatment of JAK-dependent diseases.

In one aspect the invention provides compounds according to the invention, i.e. a compound of formula (I) or a pharmaceutical composition thereof, for use as a medicament, preferably for the prevention and/or treatment of respiratory and specifically pulmonary disease.

In a further aspect the invention provides the use of a compound (I), or a pharmaceutically acceptable salt thereof, in the manufacture of a medicament for the treatment of disorders associated with JAK mechanisms, particularly for the treatment of disorders such as respiratory and pulmonary diseases.

In particular, the invention provides compounds of formula (I) for use in the prevention and/or treatment of pulmonary disease selected from the group consisting of asthma, chronic obstructive pulmonary disease COPD, idiopathic pulmonary fibrosis (IPF) acute lung injury and acute respiratory distress syndrome (ARDS).

Moreover, the invention provides a method for the prevention and/or treatment of disorders associated with JAK mechanisms, said method comprising administering to a patient in need of such treatment a therapeutically effective amount of a compound of the invention.

In particular, the invention provides methods for the prevention and/or treatment wherein the disorder is a respiratory disease selected from asthma, chronic obstructive pulmonary disease (COPD), idiopathic pulmonary fibrosis (IPF), acute lung injury and acute respiratory distress syndrome (ARDS).

Preferred is the use of the compounds of the invention for the prevention of the aforesaid disorders.

Equally preferred is the use of the compounds of the invention for the treatment of the aforesaid disorders.

Generally speaking, compounds which are JAK inhibitors may be useful in the treatment of many disorders associated with JAK enzyme mechanisms.

In one embodiment, the disorder that can be treated by the compound of the present invention is selected from the group consisting of asthma, chronic obstructive pulmonary disease (COPD) and interstitial lung disease such as idiopathic pulmonary fibrosis (IPF), acute lung injury and acute respiratory distress syndrome (ARDS).

In a further embodiment, the disorder is selected from asthma and chronic obstructive pulmonary disease (COPD).

The methods of treatment of the invention comprise administering an effective amount of a compound of formula (I) or a pharmaceutically acceptable salt thereof to a patient in need thereof. As used herein, “effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

The invention also provides pharmaceutical compositions of compounds of formula (I) in admixture with one or more pharmaceutically acceptable carrier or excipient, for example those described in Remington's Pharmaceutical Sciences Handbook, XVII Ed., Mack Pub., N.Y., U.S.A.

The present invention is also directed to use of the compounds of the invention and their pharmaceutical compositions for various route of administration.

Administration of the compounds of the invention and their pharmaceutical compositions may be accomplished according to patient needs, for example, orally, nasally, parenterally (subcutaneously, intravenously, intramuscularly, intrasternally and by infusion), by inhalation, rectally, vaginally, topically, locally, transdermally, and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like. Time release capsules, tablets and gels are also advantageous.

Various liquid oral dosage forms can also be used for administering compounds of the invention, including aqueous and non-aqueous solutions, emulsions, suspensions, syrups, and elixirs. Such dosage forms can also contain suitable known inert diluents such as water and suitable known excipients such as preservatives, wetting agents, sweeteners, flavorants, as well as agents for emulsifying and/or suspending the compounds of the invention. The compounds of the present invention may be formulated as injectable composition, for example to be injected intravenously, in the form of an isotonic sterile solution. Other preparations are also possible.

Suppositories for rectal administration of the compounds of the invention can be prepared by mixing the compound with a suitable excipient such as cocoa butter, salicylates and polyethylene glycols.

Formulations for vaginal administration can be in the form of cream, gel, paste, foam, or spray formula containing, in addition to the active ingredient, such as suitable carriers, are also known.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Compounds of the invention may exhibit profile suitable for oral route administration. Optimization of drugs for oral delivery needs certain characteristics that allow orally administered compound to be absorbed by GI (gastrointestinal) tract and to be poorly cleared in order to give a good bioavailability (F %), thus to maintain a sufficient concentration in plasma and target tissues for a time adequate to sustain pharmacological effect. To enhance oral bioavailability, one or more features of the compounds need to be optimized such as, and not limited to, maximizing membrane permeability and reducing metabolic hot spots (optimizing in-vitro clearance).

For the treatment of the diseases of the respiratory tract, the compounds according to the invention, as above said, may also preferably be administered by inhalation.

Some preferred compounds of the invention exhibit profile suitable for inhalatory route administration.

Drugs optimized for inhaled delivery require certain characteristics that allow the compound, when administered to the lung to maintain a sufficient local concentration (lung retention) to exert a pharmacological effect of the desired duration, with minimal drug absorption in the GI tract for the swallowed fraction and in general non-relevant levels in unwanted compartments (i.e. plasma). For this purpose, one or more features of a compounds were optimized such as, and not limited to, membrane permeability, dissolution rate and the compound's basicity to enhance its binding to the phospholipid-rich lung tissue or through lysosomal trapping. In some embodiments, compounds of invention show one or more of the features above in the range desirable for an inhaled compound. An inhaled JAKi should preferably possess additional properties that may further limit the systemic exposure after inhalation.

A way to limit systemic exposure, after local administration, might be to design soft-drug that means the introduction of specific moieties, like for example cyclic esters or lactones, in the present invention, which favour a controlled rapid systemic metabolism (in the liver and/or in the blood) into predicted functionally less active, metabolites compared to parent compound. For this purpose, one way resulted in the optimization of ‘suitably designed lactones derivatives’ which can be substrates of liver and/or blood esterases that may be beneficial for achieving an enhanced clearance in-vivo.

Thus, preferred compounds of the invention showed one or more of the following properties: high biochemical activity on target, strong functional activity (like cell based activity) and a rapid clearance in representative assays (stability in liver microsomes and/or hepatocytes, plasma stability) so that they have potential for giving compounds with an improved safety.

For the treatment of the diseases of the respiratory tract, the compounds according to the invention, as above said, may be administered by inhalation.

Inhalable preparations include inhalable powders, propellant-containing metering aerosols or propellant-free inhalable formulations and may be administered through a suitable inhalation device which may be respectively selected from dry powder inhaler, pressurized metered dosed inhaler, or a nebulizer.

For administration as a dry powder, single- or multi-dose inhalers known from the prior art may be utilized. In that case the powder may be filled in gelatine, plastic or other capsules, cartridges or blister packs or in a reservoir.

A diluent or carrier, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in the form of solutions or suspensions in an aqueous, alcoholic or hydroalcoholic medium and they may be delivered by jet or ultrasonic nebulizers known from the prior art or by soft-mist nebulizers such as Respimat®, a registered trademark of Boehringer Ingelheim Pharmaceuticals (Wachtel, H., Kattenbeck, S., Dunne, S. et al. Pulm Ther (2017) 3: 19.

The compounds of the invention, regardless of the route of administration, can be administered as the sole active agent or in combination (i.e. as co-therapeutic agents administered in fixed dose combination or in combined therapy of separately formulated active ingredients) with other pharmaceutical active ingredients.

The compounds of the invention can be administered as the sole active agent or in combination with other pharmaceutical active ingredients including those currently used in the treatment of respiratory disorders, and known to the skilled person, such as beta2-agonists, antimuscarinic agents, corticosteroids, mitogen-activated kinases (P38 MAP kinases) inhibitors, PI3K inhibitors (phosphoinositide 3-kinases), nuclear factor kappa-B kinase subunit beta inhibitors (IKK2), Rho kinase inhibitors (ROCKi), human neutrophil elastase (HNE inhibitors, phosphodiesterase 4 (PDE4) inhibitors, leukotriene modulators, non-steroidal anti-inflammatory agents (NSAIDs) and mucus regulators).

The invention is also directed to a kit comprising the pharmaceutical compositions of compounds of the invention alone or in combination with or in admixture with one or more pharmaceutically acceptable carriers and/or excipients and a device which may be a single- or multi-dose dry powder inhaler, a metered dose inhaler or a nebulizer.

The dosages of the compounds of the invention depend upon a variety of factors including the particular disease to be treated, the severity of the symptoms, the route of administration, the frequency of the dosage interval, the particular compound utilized, the efficacy, toxicology profile, and pharmacokinetic profile of the compound.

A pharmaceutical composition comprising a compound of the invention suitable to be administered by inhalation is in various respirable forms, such as inhalable powders (DPI), propellant-containing metering aerosols (PMDI) or propellant-free inhalable formulations (e.g. UDV).

The invention is also directed to a device comprising the pharmaceutical composition comprising a compound according to the invention, which may be a single- or multi-dose dry powder inhaler, a metered dose inhaler and a nebulizer particularly soft mist nebulizer.

The following examples illustrate the invention in more detail.

The features of the invention will become apparent in the course of the following descriptions of exemplary embodiments which are given for illustration of the invention and are not intended to be limiting thereof.

### PREPARATION OF INTERMEDIATES AND EXAMPLES

### General Experimental Details

Chemical Names of the compounds were generated with Structure To Name Enterprise 10.0 Cambridge Software or latest.

When reactions are conducted at a temperature above solvent boiling point, it is intended a reaction caried out in a closed vessel by conventional heating or by microwave heating.

Purification by ‘chromatography’ or ‘flash chromatography’ refers to purification using a Biotage SP1, or Interchim puriFlash purification system, or Biotage Isolera Four purification system equipped with Biotage Dalton 2000 mass detector, or equivalent MPLC using a pre-packed polypropylene column containing stationary phase (cartridge). Where products were purified using a Si cartridge, this refers to an Interchim (or equivalent) pre-packed polypropylene column containing unbounded activated silica with spherical particles with average size of 15 μm or Isolute® (or equivalent) pre-packed polypropylene column containing unbounded activated silica with irregular particles with average size of 50 μm. Fractions containing the desired product (identified by TLC and/or LCMS analysis) were pooled and concentrated in vacuo. Purification by ‘reverse phase chromatography’ or ‘reverse phase flash chromatography’ refers to purification on MPLC instrument with C18 functionalized silica cartridges, as Biotage Sfar C₁₈ or equivalents. Where an SCX-2 cartridge was used, ‘SCX-2 cartridge’ refers to a Bond Elut® pre-packed polypropylene column containing a non-end-capped propylsulphonic acid functionalised silica strong cation exchange sorbent. Where preparative HPLC-MDAP was used for purification (MDAP: mass directed automatic purification) fractions containing the desired product were pooled and the solvent removed by evaporation or alternatively lyophilised.

### NMR Methods

NMR spectra were obtained on a Bruker Avance III 600 (5 mm RT inverse probe head), Bruker DRX 500, Bruker Avance AV 400 (5 mm RT direct probehead) or Bruker DPX 300 spectrometers using standard Bruker pulse sequences. Alternatively, NMR spectra were recorded with Varian MR-400 Mhz spectrometer operating at 400 Mhz or a Varian Unity Inova 400 spectrometer with a 5 mm inverse detection triple resonance probe operating at 400 MHz. DMSO-d₆ or CDCl₃ were used as solvents. Chemical shifts are given in relative to internal standard tetramethylsilane or solvent residual peak. All experiments were recorded at 298 K, unless stated differently. Coupling constants, (J values) are given in hertz (Hz) and multiplicities are reported using the following abbreviation: s=singlet, d=doublet, t=triplet, q=quartet, m=multiplet, br=broad, nd=not determined.

### LCMS Methods

### Method 1

Acquity UPLC coupled with SQD mass spectrometer; Column: Acquity UPLC BEH C₁₈ (50 mm×2.1 mm i.d., 1.7 μm packing diameter), mobile phase A: 0.1% formic acid in water, mobile phase B: 0.1% formic acid in acetonitrile;

| Gradient - Time | Flow (mL/min) | A % | B % |
|---|---|---|---|
| 0.00 | 0.9 | 97 | 3 |
| 1.50 | 0.9 | 3 | 97 |
| 1.90 | 0.9 | 3 | 97 |
| 2.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 2

Acquity UPLC coupled with SQD mass spectrometer; Column: Acquity UPLC BEH C₁₈ (50 mm×2.1 mm i.d., 1.7 μm packing diameter), mobile phase A: 10 mM aqueous solution of ammonium bicarbonate (adjusted to pH 10 with ammonia), mobile phase B: acetonitrile;

| Gradient-Time | Flow (mL/min) | A % | B % |
|---|---|---|---|
| 0.00 | 0.9 | 97 | 3 |
| 1.50 | 0.9 | 3 | 97 |
| 1.90 | 0.9 | 3 | 97 |
| 2.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 3

Acquity UPLC coupled with SQD mass spectrometer; Column: Acquity UPLC BEH C18 (50 mm×2.1 mm i.d., 1.7 μm packing diameter), mobile phase A: 0.1% v/v formic acid in water, mobile phase B: 0.1% v/v formic acid in acetonitrile;

| Gradient-Time | Flow (mL/min) | A % | B % |
|---|---|---|---|
| 0.00 | 0.6 | 97 | 3 |
| 0.50 | 0.6 | 97 | 3 |
| 7.00 | 0.6 | 3 | 97 |
| 7.50 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1500 AMU.

### Method 4

AGILENT LC 1260 Infinity with SFC and Agilent 6540 UHD Accurate-Mass Q-TOF LC/MS; Column: Acquity UPLC BEH C₁₈ (100 mm×2.1 mm i.d., 1.7 μm packing diameter), mobile phase A: 0.05% aqueous ammonia, mobile phase B: acetonitrile;

| Gradient-Time | Flow (mL/min) | A % | B % |
|---|---|---|---|
| 0.00 | 0.5 | 97 | 3 |
| 8.00 | 0.5 | 0 | 100 |
| 10.00 | 0.5 | 97 | 3 |
| 12.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 5

Shimadzu LCMS-2020 Single Quadrupole Liquid Chromatograph Mass Spectrometer; Column: Acquity UPLC BEH C₁₈ (100 mm×2.1 mm i.d., 1.7 μm packing diameter), mobile phase A: 0.1% formic acid in water, mobile phase B: 0.1% formic acid in acetonitrile;

| Gradient-Time | Flow (mL/min) | A % | B % |
|---|---|---|---|
| 0.10 | 0.5 | 80 | 20 |
| 2.00 | 0.5 | 5 | 95 |
| 2.70 | 0.5 | 5 | 95 |
| 2.80 | 0.5 | 80 | 20 |
| 3.00 | 0.5 | 80 | 20 |

Column temperature: 25° C.; UV detection: 215 nm and 254 nm; MS conditions: Ionisation Mode: alternate-scan Positive and Negative Electrospray (ES⁺/ES−), Scan Range: 100 to 1000 AMU.

### HPLC-MDAP

### Method 1

Agilent 1290 Infinity II Purification System; Column: Waters XBridge® (C18, 100 mm×19 mm i.d., 5 μm), mobile phase A: 0.1% (v/v) formic acid in water, mobile phase B: acetonitrile;

| Gradient - Time | Flow (mL/min) | A % | B % |
|---|---|---|---|
| 0.00 | 30 | 90 | 10 |
| 0.80 | 30 | 90 | 10 |
| 5.00 | 30 | 70 | 30 |
| 6.01 | 30 | 0 | 100 |
| 6.95 | 30 | 0 | 100 |
| 6.96 | 30 | 10 | 90 |

### Abbreviations Used

AIBN=Azobisisobutyronitrile; aq.=aqueous; Boc₂O=Di-tert-butyl dicarbonate; CDI=carbonyl diimidatzole; DABAL-Me₃=Bis(trimethylaluminum)-1,4-diazabicyclo[2.2.2]octane adduct; DBU=1,8-Diazabicyclo[5.4.0]undec-7-ene; DCC=Dicyclohexylcarbodiimine; DCE=1,2-Dichloroethane; DCM=Dichloromethane; DIPEA=N,N-Diisopropylethylamine; DMAP=4-dimethylaminopyridine; DMCHDA=trans-N,N′-Dimethylcyclohexane-1,2-diamine; DMF=N,N-Dimethylformamide; DMSO=Dimethylsulfoxide; EtOAc=Ethyl acetate; HATU=(1-[Bis(dimethylamino)methylene]-1H-1,2,3-triazolo[4,5-b]pyridinium 3-oxid hexafluorophosphate); LCMS=Liquid chromatography-mass spectrometry; LiHMDS=Lithium bis(trimethylsilyl)amide; NBS═N-Bromosuccinimide; μW=microwave; ¹H-NMR=Proton nuclear magnetic resonance; RM=Reaction mixture; Rt=Retention time; RT=Room temperature; sat.=saturated; T3P®=Propylphosphonic anhydride; TEA=Triethylamine; TFA—Trifluoroacetic acid; THF=Tetrahydrofuran; Xphos-Pd-G3-(2-Dicyclohexylphosphino-2′,4′,6′-triisopropyl-1,1′-biphenyl)[2-(2′-amino-1,1′-biphenyl)]palladium(II) methanesulfonate.

In the procedures that follow, some of the starting materials are identified through an “Intermediate” or “Example” number with indications on Step number. This is provided merely for assistance to the skilled chemist.

A “similar” or “analogous” procedure means that such a procedure may involve minor variations, for example reaction temperature, reagent/solvent amount, reaction time, work-up conditions and/or chromatographic purification conditions.

The stereochemistry of the compounds in the Examples, where indicated, has been assigned on the assumption that absolute configuration at resolved stereogenic centres of starting materials is maintained throughout any subsequent reaction conditions.

Unless otherwise stated, where absolute configuration (R) or (S) is reported in the compound name, ee % has to be considered equal or greater than 90%.

### Preparation of Intermediates

### Intermediate 1

### Step 1

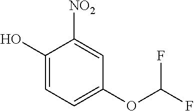 [SMILES: O=[N+]([O-])c1cc(OC(F)F)ccc1O]

### 4-(Difluoromethoxy)-2-nitrophenol (Intermediate 1-1)

A solution of 4-(difluoromethoxy)phenol (385 μL, 3.12 mmol) in DCE (3 mL) was cooled at 0° C. Concentrated nitric acid 63% w/w (456 μL) was added dropwise and RM stirred at 0° C. for 3 h. RM was poured on ice/water mix and extracted with DCE (10 mL). Organic layer was separated, dried and evaporated in vacuo to afford the desired product (630 mg) that was used in the next steps without further purification.

LCMS (Method 1): Rt=0.40 min

¹H-NMR (500 MHz, DMSO-d₆) δ: 11.09 (bs, 1H), 7.73 (d, J=2.9 Hz, 1H), 7.42 (dd, J=9.0, 2.9 Hz, 1H), 7.18 (t, J=73.1 Hz, 1H), 7.17 (d, J=9.0 Hz, 1H).

### Step 2

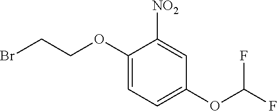 [SMILES: O=[N+]([O-])c1cc(OC(F)F)ccc1OCCBr]

### 1-(2-Bromoethoxy)-4-(difluoromethoxy)-2-nitrobenzene (Intermediate 1-2)

A mixture of intermediate 1-1 (630 mg, 3.07 mmol) and K₂CO₃ (1.27 g, 9.21 mmol) in DMF (10 mL) and 1,2-dibromoethane (265 μL, 3.07 mmol) was stirred at 60° C. overnight. After cooling to RT, RM was diluted with water and extracted twice with EtOAc. Combined organics were washed with sat aq. NaHCO₃, dried and concentrated in vacuo. The crude material was purified by flash chromatography on Si cartridge by eluting with 0-20% DCM/MeOH/NH₄OH (90:9:0.5) in DCM to give the title product (466 mg).

LCMS (Method 1): Rt=1.15 min, No MS data

¹H-NMR (500 MHz, DMSO-d₆) δ: 7.80 (d, J=2.4 Hz, 1H), 7.44-7.52 (m, 2H), 7.23 (t, J=73.1 Hz, 1H), 4.50 (t, J=5.2 Hz, 2H), 3.79 (t, J=5.4 Hz, 2H).

### Step 3

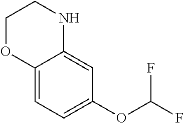 [SMILES: FC(F)Oc1ccc2c(c1)NCCO2]

### 6-(Difluoromethoxy)-3,4-dihydro-2H-benzo[b][1,4]oxazine (Intermediate 1-3)

Intermediate 1-2 (466 mg, 0.78 mmol) was dissolved in ethanol (42 mL) and heated at 80° C., then a solution of NH₄Cl (166 mg, 3.10 mmol) in water (5 mL) was added and followed by the addition of iron (434 mg, 7.80 mmol). RM was stirred at 80° C. for 3 h. After cooling to RT, RM was diluted with water and extracted twice with DCM. Combined organic layers were dried over Na₂SO₄ and concentrated in vacuo. The residue was purified by flash chromatography on Si cartridge by eluting with 0-20% EtOAc in cyclohexane to afford the title product (88 mg).

LCMS (Method 1): Rt=0.99 min, ES⁺ m/z 202.1 [M+H]⁺.

### Step 4

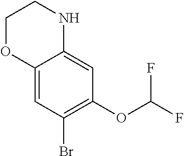 [SMILES: FC(F)Oc1cc2c(cc1Br)OCCN2]

### 7-Bromo-6-(difluoromethoxy)-3,4-dihydro-2H-benzo[b][1,4]oxazine (Intermediate 1)

To a 0° C. cooled solution of intermediate 1-3 (44.0 mg, 0.22 mol) in EtOAc (1 mL), NBS (38.9 mg, 0.22 mmol) was added and RM stirred at RT for 5 h. RM was diluted with EtOAc and quenched with sat. aq. NaHCO₃. The layers were separated and aqueous layer additionally extracted with EtOAc. Combined organic layers were washed with sat.aq. NaCl, passed through a phase separator and concentrated in vacuo to afford the desired product (63 mg) that was used in the next synthetic steps without further purification.

LCMS (Method 1): Rt=1.14 min, ES⁺ m/z 280.0/282.1 [M+H]⁺.

### Intermediate 2

### Step 1

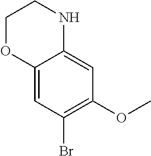

### 7-Bromo-6-methoxy-3,4-dihydro-2H-benzo[b][1,4]oxazine (Intermediate 2-1)

A solution of 6-methoxy-3,4-dihydro-2H-1,4-benzoxazine (3.0 g, 18.20 mmol) in EtOAc (30.0 mL) was cooled to 0° C. 1,3-Dibromo-5,5-dimethyl-imidazolidine-2,4-dione (2.6 g, 9.08 mmol) was added portion-wise during 15 minutes. RM was stirred for additional 30 min at 0° C. and quenched with an aqueous K₂CO₃ solution (10% w/w; 60 mL). The organic layer was separated, washed with sat. aq. NaCl and concentrated in vacuo. The residue was purified by flash chromatography on Si cartridge by eluting with 0-30% EtOAc in cyclohexane to afford the title product (3.5 g).

LCMS (Method 1): Rt=0.97 min, ES' m/z 243.9/245.9 [M+H]⁺.

Step 2

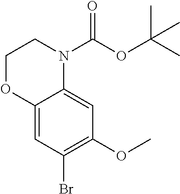 [SMILES: COc1cc2c(cc1Br)OCCN2C(=O)OC(C)(C)C]

### tert-Butyl 7-bromo-6-methoxy-2,3-dihydro-4H-benzo[b][1,4]oxazine-4-carboxylate (Intermediate 2)

THF (15 mL) was added to a mixture of intermediate 2-1 (1.4 g, 5.74 mmol), DMAP (840.9 mg, 6.88 mmol) and Boc₂O (2.80 g, 13.19 mmol), then RM stirred at RT overnight. RM was partitioned between EtOAc (50 mL)/water (30 mL). The organic layer was washed with 2M aq. citric acid (2×20 mL), sat. aq. NaCl (20 mL) and evaporated in vacuo. The residue was purified by flash chromatography on Si cartridge by eluting with 0-10% EtOAc in cyclohexane to afford the title compound (1.26 g).

LCMS (Method 1): Rt=1.27 min

¹H-NMR (300 MHz, CDCl₃) δ: 7.56 (brs, 1H), 7.05 (s, 1H), 4.14-4.18 (m, 2H), 3.82 (s, 3H), 3.78-3.82 (m, 2H), 1.53 (s, 9H).

### Intermediate 3

### Step 1

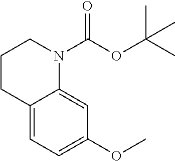 [SMILES: COc1ccc2c(c1)N(C(=O)OC(C)(C)C)CCC2]

tert-Butyl 7-methoxy-3,4-dihydroquinoline-1 (2H)-carboxylate (Intermediate 3-1) A solution of 7-methoxy-1,2,3,4-tetrahydroquinoline (500 mg, 3.06 mmol), DMAP (449 mg, 3.68 mmol) and Boc₂O (1.54 g, 7.05 mmol) in THF (10 mL) was stirred at RT overnight. A further equivalent of Boc₂O was added and stirring proceed at RT. RM was partitioned between EtOAc (50 mL) and water (30 mL). The organic layer was washed with aq. 2M citric acid (2×15 mL), sat. aq. NaCl (20 mL) and solvent evaporated in vacuo. The residue was purified by flash chromatography on Si cartridge by eluting with 0-10% EtOAc in cyclohexane to afford the title product (228 mg).

LCMS (Method 1): Rt=1.35 min

¹H-NMR (300 MHz, DMSO-d₆) δ: 7.18 (d, J=2.5 Hz, 1H), 6.98 (d, J=8.6 Hz, 1H), 6.57 (dd, J=8.5, 2.6 Hz, 1H), 3.69 (s, 3H), 3.56-3.60 (m, 2H), 2.63 (t, J=6.46 Hz, 2H), 1.74-1.82 (m, 2H), 1.45 (s, 9H).

Step 2

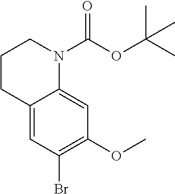 [SMILES: COc1cc2c(cc1Br)CCCN2C(=O)OC(C)(C)C]

### tert-Butyl 6-bromo-7-methoxy-3,4-dihydroquinoline-1 (2H)-carboxylate (Intermediate 3)

A solution of intermediate 3-1 (228 mg, 0.86 mmol) in EtOAc (10 mL) was cooled to 0° C. 1,3-dibromo-5,5-dimethyl-imidazolidine-2,4-dione (124 mg, 43.3 mmol) was added portionwise over 15 min. RM was stirred at 0° C. for 20 min, then quenched with 10% (w/w) aq. K₂CO₃ (20 mL). The organic layer was separated, washed with sat. aq. NaCl (20 mL) and concentrated in vacuo. The residue was purified by flash chromatography on Si cartridge by eluting with 0-15% EtOAc in cyclohexane to afford the title product (240 mg).

LCMS (Method 1): Rt=1.46 min, ES⁺ m/z 285.9/287.9 [M+H]⁺.

### Intermediate 4

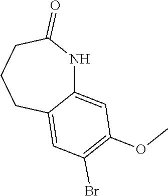

### 7-Bromo-8-methoxy-1,3,4,5-tetrahydro-2H-benzo[b]azepin-2-one (Intermediate 4)

1,3-dibromo-5,5-dimethyl-imidazolidine-2,4-dione (123 mg, 0.43 mmol) was added portionwise over 10 minutes to a solution of 8-methoxy-1,3,4,5-tetrahydro-1-benzazepin-2-one (165 mg, 0.86 mmol) in EtOAc (4 mL) at 0° C. and. RM was stirred at RT overnight. RM was quenched with sat. aq. NaHCO₃ and extracted twice with EtOAc. Combined organic layers were washed with sat. aq. NaCl, passed through a phase separator and concentrated in vacuo to give the title product (230 mg) that was used in the next synthetic steps without further purification.

LCMS (Method 1): Rt=0.93, ES' m/z 270.0/272.0 [M+H]⁺.

### Intermediate 5

### Step 1

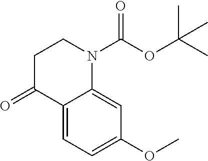 [SMILES: COc1ccc2c(c1)N(C(=O)OC(C)(C)C)CCC2=O]

### tert-Butyl 7-methoxy-4-oxo-3,4-dihydroquinoline-1 (2H)-carboxylate (Intermediate 5-1)

A solution of 7-methoxy-2,3-dihydro-1H-quinolin-4-one (100.0 mg, 0.56 mmol), DMAP (20.7 mg, 0.17 mmol) and BoC₂O (147.8 mg, 0.68 mmol) in THF (1.0 mL) was stirred at RT for 2 h. RM was partitioned between EtOAc and water. The organic layer was washed with sat. aq. NaCl, passed through a phase separator and concentrated in vacuo. The residue was purified by flash chromatography on Si cartridge by eluting with 0-10% EtOAc in cyclohexane to afford the title product (138 mg).

LCMS (Method 1): Rt=1.11 min

¹H-NMR (500 MHz, DMSO-d₆) δ: 7.79 (d, J=8.6 Hz, 1H), 7.27 (d, J=2.0 Hz, 1H), 6.79 (dd, J=8.4, 2.0 Hz, 1H), 4.07 (t, J=6.1 Hz, 2H), 3.82 (s, 3H), 2.66 (t, J=6.1 Hz, 2H), 1.51 (s, 9H).

### Step 2

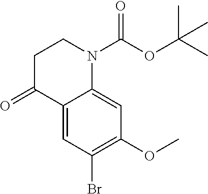 [SMILES: COc1cc2c(cc1Br)C(=O)CCN2C(=O)OC(C)(C)C]

### tert-Butyl 6-bromo-7-methoxy-4-oxo-3,4-dihydroquinoline-1 (2H)-carboxylate (Intermediate 5)

A solution of intermediate 5-1 (100.0 mg, 0.36 mmol) in EtOAc (2.0 mL) was cooled to 0° C. prior the addition of NBS (64.2 mg, 0.36 mmol). RM was warmed to RT and heated at 65° C. for 30 h. After cooling to RT, RM was diluted sat. aq. NaHCO₃ and extracted twice with EtOAc. Combined organic layers were washed with sat. aq. NaCl and passed through a phase separator. Solvent was removed in vacuo. The residue was purified by flash chromatography on Si cartridge by eluting with 0-40% EtOAc in cyclohexane to give the title product (153 mg).

LCMS (Method 1): Rt=1.30 min.

¹H-NMR (500 MHz, DMSO-d₆) δ: 7.93 (s, 1H), 7.46 (s, 1H), 4.08 (t, J=6.0 Hz, 2H), 3.93 (s, 3H), 2.69 (t, J=6.0 Hz, 2H), 1.52 (s, 9H).

### Intermediate 6

### Step 1

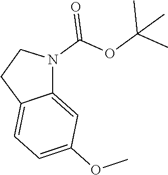 [SMILES: COc1ccc2c(c1)N(C(=O)OC(C)(C)C)CC2]

tert-Butyl 6-methoxyindoline-1-carboxylate (Intermediate 6-1) A solution of 6-methoxyindoline (1.0 g, 6.70 mmol), BoC₂O (1.76 mg, 8.04 mmol) and DMAP (819 mg, 6.70 mmol) in THF (20 mL) was stirred at RT for 6 h. RM was partitioned between EtOAc (50 mL) and water (30 mL) and organic layer washed with aq. 10% w/w citric acid (2×50 mL), sat. aq. NaHCO₃ (50 mL) and sat. aq. NaCl (50 mL). Organic phase was evaporated in vacuo to dryness. The residue was purified by flash chromatography on Si cartridge by eluting with 0-15% EtOAc in cyclohexane to afford the title product (1.16 g).

LCMS (Method 1): Rt=1.25 min, ES⁺ m/z 250.0 [M+H]⁺.

### Step 2

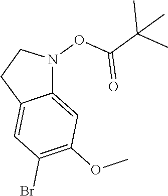 [SMILES: COc1cc2c(cc1Br)CCN2OC(=O)C(C)(C)C]

### tert-Butyl 5-bromo-6-methoxyindoline-1-carboxylate (Intermediate 6)

1,3-dibromo-5,5-dimethyl-imidazolidine-2,4-dione (0.66 g, 2.3 mmol) was added portion-wise over 15 minutes to a solution of intermediate 6-1 (1.16 g, 4.65 mmol) in EtOAc (70 mL) at 0° C. RM was stirred at RT for 2 h, then quenched with 10% w/w aq. K₂CO₃ (75 mL). Organic layer was separated, washed with sat. aq. NaCl (35 mL) and concentrated in vacuo to afford the desired product (1.52 g) that was used in the next synthetic steps without further purification.

LCMS (Method 1): Rt=1.40 min, ES⁺ m/z 228.0/230.0 [M+H]⁺.

### Intermediate 7

### Step 1

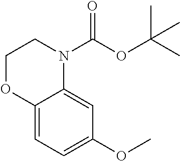 [SMILES: COc1ccc2c(c1)N(C(=O)OC(C)(C)C)CCO2]

### tert-butyl 6-methoxy-2,3-dihydro-4H-benzo[b][1,4]oxazine-4-carboxylate (Intermediate 7-1)

The title compound was prepared in a similar manner of intermediate 3—step 1 starting from 6-methoxy-3,4-dihydro-2H-1,4-benzoxazine.

LCMS (Method 5): Rt=2.5 min

¹H-NMR (300 MHz, DMSO-d₆) δ: 7.47-7.38 (m, 1H), 6.77 (d, J=8.9 Hz, 1H), 6.57 (dd, J=8.9, 3.0 Hz, 1H), 4.18-4.09 (m, 2H), 3.80-3.72 (m, 2H), 3.68 (s, 3H), 1.50 (s, 9H).

### Step 2

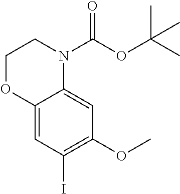 [SMILES: COc1cc2c(cc1I)OCCN2C(=O)OC(C)(C)C]

### tert-butyl 7-iodo-6-methoxy-2,3-dihydro-4H-benzo[b][1,4]oxazine-4-carboxylate (Intermediate 7)

Intermediate 7-1 (5.9 g, 22.1 mmol) was dissolved in DMF (60 mL), then N-Iodohydroxysuccinimide (12.7 g, 111 mmol) added and RM stirred at 40° C. overnight. RM was quenched in cold water and extracted with EtOAc. Combined organic layers were evaporated to dryness and the residue purified by chromatography on silica gel by gradient eluation from 1:1 to 3:2 DCM-Hexane to afford the title compound (7.38 g).

LCMS (Method 1): Rt=2.8 min

¹H-NMR (300 MHz, DMSO-d₆) δ: 7.50 (s, 1H), 7.24 (s, 1H), 4.14 (dd, J=5.3, 3.8 Hz, 2H), 3.80-3.75 (m, 2H), 3.74 (s, 3H), 1.51 (s, 9H).

### Intermediate 8

### Step 1

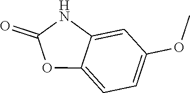

### 5-Methoxybenzo[d]oxazol-2 (3H)-one (Intermediate 8-1)

CDI (2.40 g, 14.8 mmol) was added portionwise over 1 h to a refluxing solution of 2-amino-4-methoxy-phenol (1.00 g, 14.4 mmol) in THF (20 mL) and RM refluxed for further 1 h. RM was cooled to RT and solvent removed in vacuo. The residue was dissolved in EtOAc (100 mL), washed with water (3×30 mL) and sat. aq. NaCl (2×30 mL). Organic layer was dried over MgSO₄ and solvent removed in vacuo. The crude material was purified by flash chromatography on Si cartridge by eluting with 0-80% DCM/MeOH/NH₄OH (90:5:0.5) in DCM to afford the title product (700 mg).

LCMS (Method 1): Rt=0.70 min, ES⁺ m/z 166.1 [M+H]⁺.

### Step 2

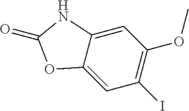

### 6-Iodo-5-methoxybenzo[d]oxazol-2 (3H)-one (Intermediate 8-2)

A solution of intermediate 8-1 (400 mg, 2.42 mmol) and NIS (817 mg, 3.63 mmol) in DMF (5 mL) was stirred at RT for 1 h. reaction was quenched with water (30 mL) and extracted with EtOAc (3×20 mL). Combined organic layers were washed with water (3×20 mL) and sat. aq. NaCl (2×10 mL). Organic phase was evaporated in vacuo to afford the title product (626 mg) that was used in the next synthetic steps without further purification.

LCMS (Method 1): Rt=0.93 min, ES⁺ m/z 291.9 [M+H]⁺.

### Step 3

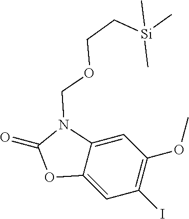 [SMILES: COc1cc2c(cc1I)oc(=O)n2COCC[Si](C)(C)C]

### 6-Iodo-5-methoxy-3-((2-(trimethylsilyl)ethoxy)methyl)benzo[d]oxazol-2 (3H)-one (Intermediate 8)

A solution intermediate 8-2 (300 mg, 1.03 mmol) in DMF (9 mL) was added to a 0° C. cooled suspension of NaH (60.0% in mineral oil, 41.2 mg, 1.03 mmol) in DMF (5 mL). RM was stirred for 30 min reaching RT. After cooling RM to 0° C., 2-(chloromethoxy)ethyl-trimethyl-silane (192 μL, 1.03 mmol) was added dropwise. RM was stirred for 2 h reaching RT. RM was quenched with sat. aq. NaHCO₃ (15 mM) and extracted with EtOAc (3×15 mL). Combined organic layers were washed with water (20 mL), sat. aq. NaCl (20 mL), dried over MgSO₄ and concentrated in vacuo to afford the desired product (300 mg) that was used in the next synthetic steps without further purification.

LCMS (Method 1): Rt=1.49 min.

¹H-NMR (300 MHz, DMSO-d₆) δ: 7.78 (s, 1H), 7.13 (s, 1H), 5.25 (s, 2H), 3.83 (s, 3H), 3.61 (t, J=8.2 Hz, 2H), 0.86 (t, J=8.0 Hz, 2H), −0.06 (s, 9H).

### Intermediate 9

### Step 1

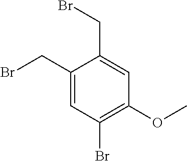

### 1-Bromo-4,5-bis(bromomethyl)-2-methoxybenzene (Intermediate 9-1)

1-Bromo-2-methoxy-4,5-dimethyl-benzene (200 mg, 0.93 mmol) was dissolved in α,α,α-trifluorotoluene (10.0 mL). NBS (331 mg, 1.86 mmol) and AIBN (30.5 mg, 0.19 mmol) were added and RM was stirred at 90° C. for 3 h. After cooling to RT, RM was diluted with EtOAc, washed with water, and sat. aq. NaCl, dried over phase separator and solvent removed in vacuo. The residue was purified by flash chromatography on Si cartridge by eluting with 0-30% EtOAc in cyclohexane to afford the title product (246 mg).

LCMS (Method 1): Rt=1.34 min.

¹H-NMR (300 MHz, DMSO-d₆) δ: 7.74 (s, 1H), 7.26 (s, 1H), 4.80 (s, 2H), 4.79 (s, 2H), 3.87 (s, 3H).

### Step 2

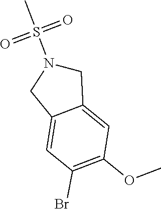 [SMILES: COc1cc2c(cc1Br)CN(S(C)(=O)=O)C2]

### 5-Bromo-6-methoxy-2-(methylsulfonyl)isoindoline (Intermediate 9)

NaH (60.0%, 56.6 mg, 1.42 mmol) was added to a solution of methanesulfonamide (135 mg, 1.42 mmol) in DMF (10.0 mL) and RM stirred for 1 h. Intermediate 9-1 (240 mg, 0.64 mmol) in DMF (10.0 mL) was added dropwise and the resulting solution stirred at 50° C. for 3 h and at RT overnight. RM was quenched with water and extracted with EtOAc. Combined organic layers were washed with water and sat. aq. NaCl, passed through a phase separator and concentrated in vacuo. The residue was purified by flash chromatography on Si cartridge by eluting with 0-30% EtOAc in cyclohexane to afford the title product (160 mg).

LCMS (Method 1): Rt=1.34 min.

¹H-NMR (300 MHz, DMSO-d₆) δ: 7.56 (s, 1H), 7.12 (s, 1H), 4.57 (s, 2H), 4.55 (s, 2H), 3.83 (s, 3H), 2.96 (s, 3H).

### Intermediate 10

### Step 1

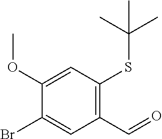 [SMILES: COc1cc(SC(C)(C)C)c(C=O)cc1Br]

### 5-Bromo-2-(tert-butylthio)-4-methoxybenzaldehyde (Intermediate 10-1)

A mixture of 5-bromo-2-fluoro-4-methoxy-benzaldehyde (1.0 g, 4.29 mmol), 2-methyl-2-propanethiol (726 μL, 6.44 mmol) and K₂CO₃ (949 mg, 6.87 mmol) in DMF (15 mL) was stirred at 80° C. for 3 h. RM was cooled to RT and poured in water (30 mL). The formed precipitate was collected by filtration, washed with water (2×10 mL), and dried at 45° C. for 3 h to afford the title product (1 g).

LCMS (Method 1): Rt=1.40 min.

¹H-NMR (300 MHz, CDCl₃) δ:10.6 (s, 1H), 8.20 (s, 1H), 7.08 (s, 1H), 3.98 (s, 3H), 1.32 (s, 9H).

### Step 2

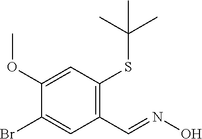 [SMILES: COc1cc(SC(C)(C)C)c(/C=N/O)cc1Br]

### 5-Bromo-2-(tert-butylthio)-4-methoxybenzaldehyde oxime (Intermediate 10-2)

A mixture of intermediate 10-1 (0.9 g, 0.3 mmol), hydroxylamine chloride (413 mg, 0.59 mmol) and sodium acetate (584 mg, 7.12 mmol) in EtOH (9 mL) was stirred at RT for 1 h. RM was filtered to remove residual solids and filtrate evaporated in vacuo. The residue was dissolved in EtOAc (25 mL), washed with water (15 mL), sat. aq. NaCl (15 mL), dried over MgSO₄ and solvent removed in vacuo to afford the desired product (997 mg) that was used in the next synthetic steps without further purification.

LCMS (Method 1): Rt=1.26 min.

¹H-NMR (300 MHz, DMSO-d₆) δ: 11.34 (s, 1H), 8.90 (s, 1H), 7.99 (s, 1H), 7.16 (s, 1H), 3.89 (s, 3H), 1.24 (s, 9H).

### Step 3

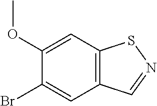

5-Bromo-6-methoxybenzo[d]isothiazole (Intermediate 10)

A solution of intermediate 10-2 (0.87 g, 2.73 mmol) and TsOH (52 mg, 0.27 mmol) in i-PrOH (8 mL) was stirred at 100° C. overnight, then in a μW reactor at 110° C. for 30 min. A second equivalent of TsOH was added and RM further stirred in μW reactor at 120° C. for 1 h for two cycle. After cooling to RT, the formed precipitate was collected by filtration and dried at 45° C. to afford the desired product (280 mg).

LCMS (Method 1): Rt=1.10 min.

¹H-NMR (500 MHz, DMSO-d₆) δ: 8.92 (s, 1H), 8.46 (s, 1H), 7.93 (s, 1H), 3.96 (s, 3H).

### Intermediate 11

### Step 1

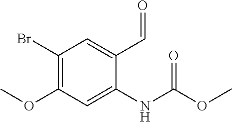

### Methyl (4-bromo-2-formyl-5-methoxyphenyl)carbamate (Intermediate 11-1)

Methyl chloroformate (170 μL, 2.2 mmol) was added dropwise to a 0° C. cooled solution of 2-amino-5-bromo-4-methoxy-benzaldehyde (200 mg, 0.87 mmol) and DIPEA (0.61 mL, 3.5 mmol) in DCM (5 mL). RM was stirred at 40° C. for 2 days. RM was evaporated in vacuo and the residue purified by flash chromatography on Si cartridge, by eluting with 0-25% DCM/MeOH (9:1) in DCM, to afford the title product (190 mg).

LCMS (Method 1): Rt=1.16 min, ES⁺ m/z 288.0/290.0 [M+H]⁺

### Step 2

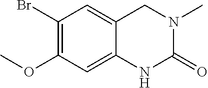

### 6-Bromo-7-methoxy-3-methyl-3,4-dihydroquinazolin-2 (1H)-one (Intermediate 11)

A solution of intermediate 11-1 (185 mg, 0.64 mmol) and methanolic methyl amine (9.8 M, 97 μL, 0.95 mmol) in AcOH (2 mL) was stirred in a μW reactor at 130° C. for 15 min. RM was cooled to RT and formic acid (1.2 mL, 32 mmol) added. RM was stirred at 150 C° for 30 min. RM was evaporated in vacuo and the residue purified by flash chromatography on a Si cartridge, by eluting with 0-50% DCM/MeOH/NH₄OH (90:5:0.5) in DCM, to afford the title product (154 mg).

LCMS (Method 1): Rt=0.88 min, ES⁺ m/z 271.1/273. [M+H]⁺

### Intermediate 12

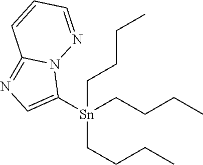 [SMILES: CCC[CH2][Sn]([CH2]CCC)([CH2]CCC)[c]1cnc2cccnn12]

### 3-(Tributylstannyl)imidazo[1,2-b]pyridazine (Intermediate 12)

A solution of 3-bromoimidazo[1,2-b]pyridazine (400 mg, 2.0 mmol) in THF (5 mL) was cooled to 0° C. then i-PrMgCl LiCl complex (1.3 M solution in THF, 4.7 mL, 6.1 mmol) added dropwise over 5 min. RM was stirred at 0° C. for 15 min then tri-n-butyltin chloride (685 μL, 2.5 mmol) added dropwise, and RM further stirred at RT for 30 min RM was cooled in an ice bath and quenched with water (5 mL), added with aq. sat. NH₄Cl (10 mL) and extracted with EtOAc (15 mL). Organic layer was washed with aq. sat. NaCl (10 mL), dried over MgSO₄ and evaporated in vacuo. The residue was purified by chromatography on neutral allumina (Al₂O₃) by eluting with mixtures of cyclohexane and EtOAc to afford the title product (285 mg).

LCMS (Method 2): Rt=1.82 min, ES⁺ m/z (most abundant isotope) 410.0 [M+H]⁺

### Intermediate 13

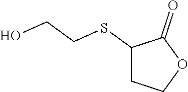

### 3-((2-Hydroxyethyl)thio)dihydrofuran-2 (3H)-one (Intermediate 13)

A solution of 3-bromotetrahydrofuran-2-one (822 μL, 9.09 mmol), 2-sulfanylethanol (2.55 mL, 36.4 mmol), and DIPEA (1.74 mL, 10.0 mmol) in THF (4 mL) was stirred at RT overnight. RM was evaporated in vacuo and the residue purified by flash chromatography on Si cartridge by eluting with 0-50% EtOAc in DCM to afford the title product (1.43 g).

¹H-NMR (300 MHz, CDCl₃) δ: 4.42 (dt, J=8.9, 7.4 Hz, 1H), 4.31 (ddd, J=9.1, 8.0, 4.8 Hz, 1H), 3.82-3.87 (m, 2H), 3.63 (dd, J=8.7, 5.5 Hz, 1H), 3.07 (ddd, J=14.3, 5.9, 4.8, 1H), 2.75-2.78 (m, 1H), 2.67 (ddd, J=15.9, 13.8, 8.0, 1H), 2.08-2.16 (m, 1H).

### Intermediate 14

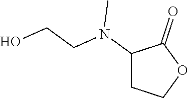

### 3-((2-Hydroxyethyl)(methyl)amino)dihydrofuran-2 (3H)-one (Intermediate 14)

A solution of 3-bromotetrahydrofuran-2-one (782 μL, 8.65 mmol), 2-(methylamino)ethanol (1.39 mL, 17.3 mmol) in THF (4 mL) was stirred at RT overnight. Solvent was removed in vacuo and the residue purified by flash chromatography on Si cartridge by eluting with 0-50% DCM/MeOH (9:1) in DCM to afford the title product (295 mg).

¹H-NMR (300 MHz, CDCl₃) δ: 4.37 (dt, J=9.1, 2.1 Hz, 1H), 4.18 (ddd, J=10.2, 9.6, 6.6 Hz, 1H), 3.73 (dd, J=10.9, 8.9 Hz, 1H), 3.63 (t, J=5.3, 2H), 2.76-2.81 (m, 2H), 2.42 (s, 3H), 2.11-2.39 (m, 1H).

### Intermediate 15

### Step 1

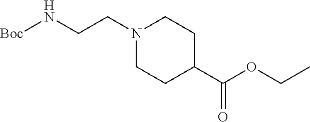 [SMILES: CCOC(=O)C1CCN(CCNC(=O)OC(C)(C)C)CC1]

### Ethyl 1-(2-((tert-butoxycarbonyl)amino)ethyl)piperidine-4-carboxylate (Intermediate 15-1)

A suspension of tert-butyl N-(2-bromoethyl)carbamate (3 g, 13.0 mmol), ethyl piperidine-4-carboxylate (2.1 mL, 13.0 mmol) and K₂CO₃ (3.7 g, 27 mmol) in DMF (30.0 mL) was stirred at 65° C. for 24 h. After cooling to RT, RM was diluted with water (80 mL) and extracted with EtOAc (3×80 mL). Combined organics were washed with sat. aq. NaHCO₃ (80 mL) and sat. aq. NaCl (50 mL). Organic solvent was removed in vacuo and residue was purified by flash chromatography on Si cartridge by eluting with 0-50% DCM:MeOH (93:7) in DCM to afford the title product (3.24 g).

LCMS (Method 2): Rt=1.05 min, ES' m/z 301.3 [M+H]⁺.

### Step 2

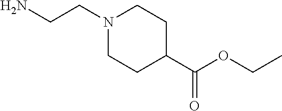

### Ethyl 1-(2-aminoethyl)piperidine-4-carboxylate (Intermediate 15)

A solution of intermediate 15-1 (1.77 g, 5.9 mmol) and TFA (8.8 mL, 118 mmol) in DCM (14 mL) was stirred at RT overnight. Volatiles were removed in vacuo and crude product passed through an SCX cartridge, washed with EtOH (500 mL) and eluted with 15% aq. NH₃ in EtOH (100 mL) to afford the title product (1.16 g).

¹H-NMR (300 MHz, CDCl₃) δ: 4.13 (q, J=6.9 Hz, 2H), 2.84-2.88 (m, 2H), 2.78 (t, J=6.2 Hz, 2H), 2.39 (t, J=6.2 Hz, 2H), 2.24-230 (m, 1H), 2.02 (t, J=11.5 Hz, 2H), 1.87-1.91 (m, 2H), 1.71-1.79 (m, 2H), 1.25 (t, J=6.9 Hz, 3H).

### Intermediate 16

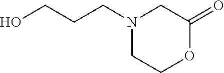 [SMILES: O=C1CN(CCCO)CCO1]

### 4-(3-Hydroxypropyl)morpholin-2-one (Intermediate 16)

A mixture of 3-(2-hydroxyethylamino)propan-1-ol (393 mg, 3.30 mmol), methyl 2-bromoacetate (344 μL, 3.63 mmol) and K₂CO₃ (502 mg, 3.63 mmol) in dry acetonitrile (14 mL) was stirred at RT overnight. RM was filtered and concentrated in vacuo. Residue was dissolved in DCM (20 mL) and washed with water (10 mL). Combined organic layers were passed through phase separator and solvent removed in vacuo to afford the desired product (120 mg) that was used in the next synthetic steps without further purification.

¹H-NMR (300 MHz, CDCl₃) δ: 4.37-4.40 (m, 2H), 3.67-3.82 (m, 2H), 3.34 (s, 2H), 3.05 (m, 1H), 2.73 (t, J=5.1 Hz, 2H), 2.62 (t, J=6.2 Hz, 2H), 1.74 (quint, J=5.5 Hz, 2H).

### Intermediate 17

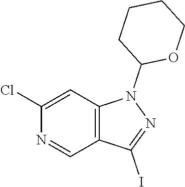 [SMILES: Clc1cc2c(cn1)c(I)nn2C1CCCCO1]

### 6-Chloro-3-iodo-1-(tetrahydro-2H-pyran-2-yl)-1H-pyrazolo[4,3-c]pyridine (Intermediate 17)

Dihydropyran (9.79 mL, 107 mmol) and methanesulfonic acid (464 μL, 7.16 mmol) were added to 6-chloro-3-iodo-1H-pyrazolo[4,3-c]pyridine (10 g, 35.8 mmol) in DCM (100 mL) and THF (50 mL). RM was stirred at 40° C. for 4 h, then at RT overnight. RM was evaporated to dryness and the residue purified by flash chromatography on a Si cartridge by eluting with 0-40% of EtOAc in cyclohexane to afford the title product (7.5 g).

LCMS (Method 2): Rt=1.22 min, ES⁺ m/z 364.0/366.0 [M+H]⁺.

### Intermediate 18a

### Step 1

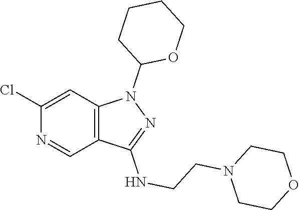 [SMILES: Clc1cc2c(cn1)c(NCCN1CCOCC1)nn2C1CCCCO1]

### 6-Chloro-N-(2-morpholinoethyl)-1-(tetrahydro-2H-pyran-2-yl)-1H-pyrazolo[4,3-c]pyridin-3-amine (Intermediate 18a-1)

A degassed mixture of intermediate 17 (900 mg, 2.48 mmol), 2-morpholinoethanamine (1.30 mL, 12.7 mmol), K₂CO₃ (2.05 g, 14.9 mmol), proline (85.5 mg, 0.74 mmol) and CuI (94.3 mg, 0.50 mmol) in DMF (10 mL) was stirred under nitrogen at 100° C. for 2 h. After cooling to RT, RM was diluted with water (30 mL) and extracted with EtOAc (4×15 mL). Combined organics were washed with aq. ammonia 10% w/w (2×15 mL), water (2×20 mL) and sat. aq. NaCl (40 mL). Organic phase was evaporated in vacuo and the residue purified by flash chromatography on Si cartridge by eluting with 0-80% DCM/MeOH/NH₄OH (90:5:0.5) in DCM to afford the title product (750 mg).

LCMS (Method 2): Rt=0.94 min, ES⁺ m/z 366.3/368.3 [M+H]⁺.

Step 2

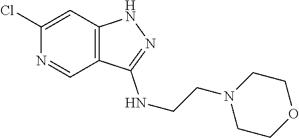 [SMILES: Clc1cc2[nH]nc(NCCN3CCOCC3)c2cn1]

### 6-Chloro-N-(2-morpholinoethyl)-1H-pyrazolo[4,3-c]pyridin-3-amine (Intermediate 18a)

A solution of intermediate 18a-1 (635 mg, 1.88 mmol), TFA (2.66 mL, 34.7 mmol) and triethylsilane (0.9 mL, 5.64 mmol) in DCM (20 mL) was stirred at RT for 2 h. RM was quenched with water (15 mL) and pH adjusted to 9.5. Aqueous phase was further extracted with DCM (3×20 mL), and combined organic layers were evaporated in vacuo. The residue was purified by flash chromatography on Si cartridge by eluting with 0-100% DCM/MeOH/NH₄OH (90:5:0.5) in DCM to give the title product (250 mg).

LCMS (Method 1): Rt=0.42 min, ES⁺ m/z 282.0/283.9 [M+H]⁺

### Intermediate 18b

### Step 1

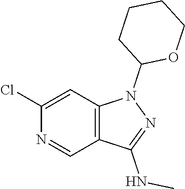 [SMILES: CNc1nn(C2CCCCO2)c2cc(Cl)ncc12]

6-Chloro-N-methyl-1-(tetrahydro-2H-pyran-2-yl)-1H-pyrazolo[4,3-c]pyridin-3-amine (Intermediate 18b-1)

The title product was prepared on a similar manner to intermediate 18a-1 (step 1) starting from intermediate 17 (230 mg, 0.63 mmol) and methylamine hydrochloride (171 mg, 2.5 mmol). DMSO was used as reaction solvent instead of DMF.

LCMS (Method 2): Rt=0.94 min, ES m/z 267.1/269.1 [M+H]⁺.

### Step 2

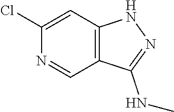

### 6-chloro-N-methyl-1H-pyrazolo[4,3-c]pyridin-3-amine (Intermediate 18b)

The title compound was prepared similarly to intermediate 18a (step 2) starting from intermediate 18b-1.

LCMS (Method 2): Rt=0.51 min, ES⁺ m/z 183.1/185.1 [M+H]⁺

### Intermediate 19

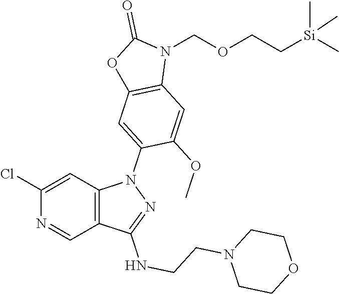 [SMILES: COc1cc2c(cc1-n1nc(NCCN3CCOCC3)c3cnc(Cl)cc31)oc(=O)n2COCC[Si](C)(C)C]

### 6-(6-Chloro-3-((2-morpholinoethyl)amino)-1H-pyrazolo[4,3-c]pyridin-1-yl)-5-methoxy-3-((2-(trimethylsilyl)ethoxy)methyl)benzo[d]oxazol-2 (3H)-one (Intermediate 19)

A degassed mixture of intermediate 18a (102 mg, 0.36 mmol), intermediate 8 (85.0 mg, 0.18 mmol), K₂CO₃ (151 mg, 1.09 mmol), proline (6.27 mg, 0.05 mmol) and CuI (6.9 mg, 36 μmol) in DMF (2 mL) was stirred under nitrogen at 100° C. for 8 h. After cooling to RT, RM was diluted with water (10 mL) and extracted with EtOAc (5×5 mL). Combined organic layers were washed with aq. Ammonia 10% w/w (2×10 mL), water (2×20 mL) and sat. aq. NaCl (30 mL). Organic phase was evaporated in vacuo and the residue purified by flash chromatography on Si cartridge by eluting with 0-80% DCM/MeOH/NH₄OH (90:5:0.5) in DCM to afford the title product (15 mg).

LCMS (Method 1): Rt=1.07 min, ES⁺ m/z 575.4/577.4 [M+H]⁺.

### Intermediate 20

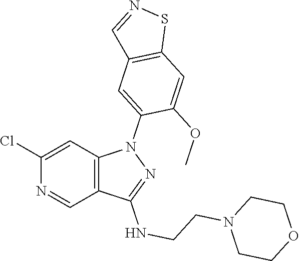 [SMILES: COc1cc2sncc2cc1-n1nc(NCCN2CCOCC2)c2cnc(Cl)cc21]

### 6-Chloro-1-(6-methoxybenzo[d]isothiazol-5-yl)-N-(2-morpholinoethyl)-1H-pyrazolo[4,3-c]pyridin-3-amine (Intermediate 20)

A degassed mixture of intermediate 18a (120 mg, 0.43 mmol), intermediate 10 (156 mg, 0.64 mmol), CuI (40.6 mg, 0.21 mmol), N,N-dimethylglycine (43.9 mg, 0.43 mmol) and K₂CO₃ (118 mg, 0.85 mmol) in DMSO (2.5 mL) was heated overnight at 100° C. under nitrogen. After cooling to RT, RM was diluted with EtOAc (15 mL), washed with water (2×10 mL) and sat. aq.

NaCl (10 mL). RM was evaporated in vacuo and the residue purified by flash chromatography on Si cartridge by eluting with 0-80% DCM/MeOH/NH₄OH (90:5:0.5) in DCM to give the title product (105 mg).

LCMS (Method 1): Rt=0.99 min, ES⁺ m/z 445.2/447.2 [M+H]⁺

### Intermediate 21

### Step 1

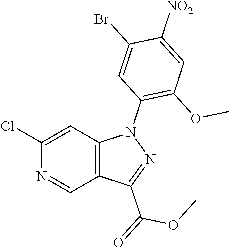 [SMILES: COC(=O)c1nn(-c2cc(Br)c([N+](=O)[O-])cc2OC)c2cc(Cl)ncc12]

### Methyl 1-(5-bromo-2-methoxy-4-nitrophenyl)-6-chloro-1H-pyrazolo[4,3-c]pyridine-3-carboxylate (Intermediate 21-1)

6-Chloro-1H-pyrazolo[4,3-c]pyridine-3-carboxylic acid (3.00 g, 15.2 mmol) was added to a solution of 1-bromo-5-fluoro-4-methoxy-2-nitro-benzene (3.80 g, 20.2 mmol) and DBU (5.88 mL, 45.6 mmol) in DMF (48 mL). RM was stirred at RT for 4 h. Iodomethane (3.78 mL, 60.7 mmol) was added, and RM stirred at RT overnight. RM was quenched into water (200 mL), the formed precipitate was collected by filtration and dried. The crude material was triturated in EtOAc and washed with EtOAc to give the title product (5.83 g) that was used in the next synthetic step without further purification.

LCMS (Method 1): Rt=1.08 min, ES⁺ m/z 426.9/428.9/430.9 [M+H]⁺.

### Step 2

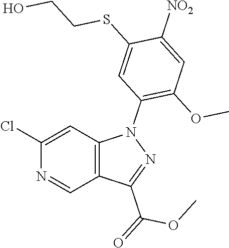 [SMILES: COC(=O)c1nn(-c2cc(SCCO)c([N+](=O)[O-])cc2OC)c2cc(Cl)ncc12]

### Methyl 6-chloro-1-(5-((2-hydroxyethyl)thio)-2-methoxy-4-nitrophenyl)-1H-pyrazolo[4,3-c]pyridine-3-carboxylate (Intermediate 21-2)

A degassed mixture of intermediate 21-1 (5.0 g, 11 mmol), 2-sulfanylethanol (1.13 mL, 12 mmol), DIPEA (5.91 mL, 20 mmol), Xantphos (820 mg, 1.4 mmol) and Pd₂(dba)₃ (330 mg, 0.57 mmol) in 1,4-dioxane (100 mL) was stirred at 100° C. under argon for 3 h. After cooling to RT, reaction was quenched with water. The formed precipitate was collected by filtration, washed with a small amount of EtOAc and dried to afford the title compound (5.3 g) that was used in the next synthetic step without further purification.

LCMS (Method 1): Rt=1.08 min, ES⁺ m/z 439.0/441.0 [M+H]⁺.

### Step 3

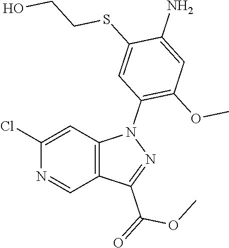 [SMILES: COC(=O)c1nn(-c2cc(SCCO)c(N)cc2OC)c2cc(Cl)ncc12]

### Methyl 1-(4-amino-5-((2-hydroxyethyl)thio)-2-methoxyphenyl)-6-chloro-1H-pyrazolo[4,3-c]pyridine-3-carboxylate (Intermediate 21-3)

A solution of intermediate 21-2 (5.3 g, 12 mmol) in MeOH (200 mL) was stirred at 80° C., then followed by the addition of a solution of NH₄Cl (6.5 g, 121 mmol) in water (20 mL) and iron powder (6.7 g, 121 mmol). RM was stirred at 80° C. for 4.5 h, then filtered while still hot and the filtrate concentrated in vacuo. The crude material was diluted with sat. aq NaHCO₃, the resulting suspension sonicated, and the solids collected by filtration. The solid material was washed several times with water and dried to afford the title product (3.13 g).

LCMS (Method 1): Rt=0.92 min, ES⁺ m/z 409.1/411.1 [M+H]⁺.

### Step 4

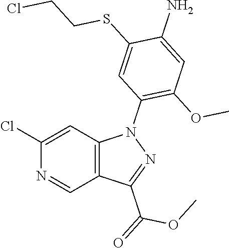 [SMILES: COC(=O)c1nn(-c2cc(SCCCl)c(N)cc2OC)c2cc(Cl)ncc12]

### Methyl 1-(4-amino-5-((2-chloroethyl)thio)-2-methoxyphenyl)-6-chloro-1H-pyrazolo[4,3-c]pyridine-3-carboxylate (Intermediate 21-4)

Intermediate 21-3 (4.4 g, 11 mmol) was mixed with thionyl chloride (12 mL, 165 mmol) at 0° C. RM was warmed to RT and stirred for 2 h, then volatiles removed in vacuo and the residue taken with sat. aq. NaHCO₃. The formed slurry was sonicated and solid collected by filtration, washed with water, and dried. The resulting crude was purified by flash chromatography on Si cartridge by eluting with 0-10% EtOAc in DCM to afford the title product (2.7 g).

LCMS (Method 1): Rt=1.24 min, ES⁺ m/z 427.0/429.0/431.0 [M+H]⁺.

### Step 5

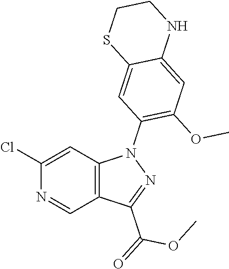 [SMILES: COC(=O)c1nn(-c2cc3c(cc2OC)NCCS3)c2cc(Cl)ncc12]

### Methyl 6-chloro-1-(6-methoxy-3,4-dihydro-2H-benzo[b][1,4]thiazin-7-yl)-1H-pyrazolo[4,3-c]pyridine-3-carboxylate (Intermediate 21-5)

A mixture of intermediate 21-4 (2.70 g, 6.32 mmol), K₂CO₃ (2.62 g, 19 mmol) and NaI (189 mg, 1.26 mmol) in DMF (80 mL) was stirred at 90° C. overnight. After cooling to RT, RM was diluted with water (200 mL). The formed precipitate was filtered, washed several times with water and dried. The resulting material was purified by flash chromatography on Si cartridge by eluting with 0-100% DCM/Acetonitrile (9:1) in DCM to afford the title product (730 mg).

LCMS (Method 1): Rt=1.13 min, ES⁺ m/z 391.1/393.1 [M+H]⁺.

### Step 6

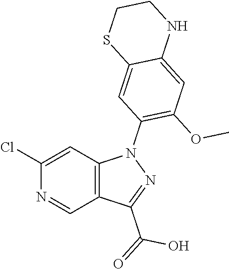 [SMILES: COc1cc2c(cc1-n1nc(C(=O)O)c3cnc(Cl)cc31)SCCN2]

### 6-Chloro-1-(6-methoxy-3,4-dihydro-2H-benzo[b][1,4]thiazin-7-yl)-1H-pyrazolo[4,3-c]pyridine-3-carboxylic acid (Intermediate 21)

Intermediate 21-5 (730 mg, 1.87 mmol) was suspended in THF (15 mL), and added with a solution of LiOH (1.0 M in water, 9 mL, 9 mmol). RM was stirred at 40° C. for 2.5 h. Organic solvent was removed in vacuo and the residue diluted with water. The pH was adjusted to 2.5 using aq. 1M HCl. The formed precipitate was filtered, washed several times with water and dried to afford the title compound (698 mg).

LCMS (Method 1): Rt=0.95 min, ES m/z 377.1/379.0 [M+H]⁺.

### Intermediate 22

### Step 1

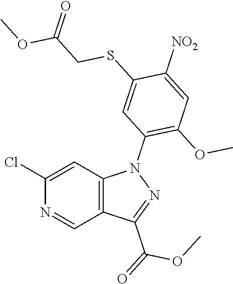

### Methyl 6-chloro-1-(2-methoxy-5-((2-methoxy-2-oxoethyl)thio)-4-nitrophenyl)-1H-pyrazolo[4,3-c]pyridine-3-carboxylate (Intermediate 22-1)

Intermediate was prepared similarly to intermediate 21-2 starting from intermediate 21-1 and methyl 2-sulfanylacetate.

LCMS (Method 1): Rt=1.18 min, ES m/z 467.1/469.1 [M+H]⁺.

Step 2

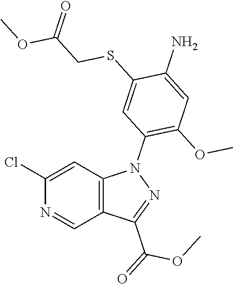

### Methyl 1-(4-amino-2-methoxy-5-((2-methoxy-2-oxoethyl)thio)phenyl)-6-chloro-1H-pyrazolo[4,3-c]pyridine-3-carboxylate (Intermediate 22-2)

A solution of intermediate 22-1 (265 mg, 0.51 mmol) in ethanol (6.0 mL) was stirred at 80° C., then added with a solution of NH₄Cl (109 mg, 2.0 mmol) in water (3.0 mL) and iron (340 mg, 6.1 mmol). RM was stirred at 80° C. for 16 h. After cooling to RT, RM was filtered, and the filter further washed with EtOAc. Combined organic layers were evaporated in vacuo to afford the desired product (1.03 g) that was used in the next synthetic steps without further purification.

LCMS (Method 1): Rt=1.09 min, ES⁺ m/z 437.2/439.1 [M+H]⁺.

### Step 3

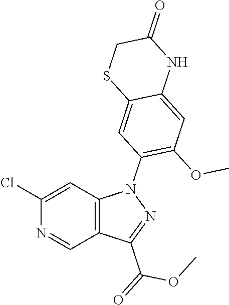 [SMILES: COC(=O)c1nn(-c2cc3c(cc2OC)NC(=O)CS3)c2cc(Cl)ncc12]

### Methyl 6-chloro-1-(6-methoxy-3-oxo-3,4-dihydro-2H-benzo[b][1,4]thiazin-7-yl)-1H-pyrazolo[4,3-c]pyridine-3-carboxylate (Intermediate 22-3)

TFA (1.3 mL, 17.0 mmol) was added to a suspension of intermediate 22-2 (1.0 g, 1.7 mmol) in DCM (12.0 mL). RM was stirred at RT for 24 h. RM was evaporated in vacuo and the residue purified by flash chromatography on Si cartridge by eluting with 0-60% DCM/MeOH (20:1) in DCM to give the title product (341.5 mg).

LCMS (Method 1): Rt=0.99 min, ES⁺ m/z 405.2/407.1 [M+H]⁺.

### Step 4

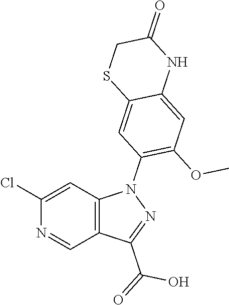 [SMILES: COc1cc2c(cc1-n1nc(C(=O)O)c3cnc(Cl)cc31)SCC(=O)N2]

### 6-Chloro-1-(6-methoxy-3-oxo-3,4-dihydro-2H-benzo[b][1,4]thiazin-7-yl)-1H-pyrazolo[4,3-c]pyridine-3-carboxylic acid (Intermediate 22)

An aqueous solution of LiOH (1.0 M, 7.5 mL, 7.5 mmol) was added to a suspension of intermediate 22-3 (340 mg, 0.84 mmol) in THF (10 mL). RM was stirred at RT for 2 h. THF was removed in vacuo. The residue was diluted with water and acidified to pH 2.5 by using aq.1M HCl. The formed precipitate was collected by filtration, washed with water and dried to afford the title product (320 mg).

LCMS (Method 2): Rt=0.48 min, ES⁺ m/z 391.0/393.0 [M+H]⁺.

### Intermediate 23a

### Step 1

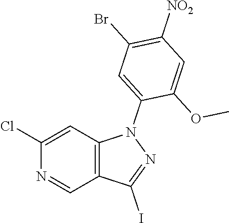 [SMILES: COc1cc([N+](=O)[O-])c(Br)cc1-n1nc(I)c2cnc(Cl)cc21]

### 1-(5-bromo-2-methoxy-4-nitrophenyl)-6-chloro-3-iodo-1H-pyrazolo[4,3-c]pyridine (Intermediate 23a-1)

A mixture of 6-chloro-3-iodo-1H-pyrazolo[4,3-c]pyridine (1.50 g, 5.37 mmol), 1-bromo-5-fluoro-4-methoxy-2-nitro-benzene (1.34 g, 5.37 mmol) and K₂CO₃ (2.23 g, 16.1 mmol) in DMF (30 mL) was stirred at 65° C. for 2 h. Reaction was repeated on 17.4 mmol scale and combined with previous after cooling to RT. Combined RM were poured into water (400 mL), and the precipitate collected by filtration. Collected solids were washed thoroughly with water and azeotroped with toluene to afford title product (11.1 g).

LCMS (Method 2): Rt=1.36 min, ES⁺ m/z 508.9/510.9/512.8 [M+H]⁺.

### Step 2

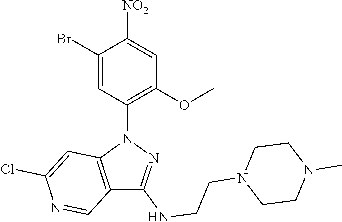 [SMILES: COc1cc([N+](=O)[O-])c(Br)cc1-n1nc(NCCN2CCN(C)CC2)c2cnc(Cl)cc21]

### 1-(5-Bromo-2-methoxy-4-nitrophenyl)-6-chloro-N-(2-(4-methylpiperazin-1-yl)ethyl)-1H-pyrazolo[4,3-c]pyridin-3-amine (Intermediate 23a-2)

A degassed mixture of intermediate 23a-1 (800 mg, 1.57 mmol), 2-(4-methylpiperazin-1-yl)ethanamine (366 μL, 2.83 mmol), proline (90.4 mg, 0.79 mmol), K₂CO₃ (1.3 g, 9.42 mmol) and CuI (89.7 mg, 0.47 mmol) in dry DMSO (6 mL) was stirred at 75° C. for 5 h. A second equivalent of CuI and proline was added. After cooling to RT, solvent was removed in vacuo. The residue was partitioned between EtOAc and water. Aqueous layer was further extracted with AcOEt. Combined organic layers were washed with sat. aq. NaCl (20 mL) and concentrated in vacuo. The residue was purified twice by flash chromatography on Si cartridge by eluting with DCM/MeOH/NH₄OH (90:15:1.5) to afford the title product (243 mg).

LCMS (Method 2): Rt=1.08 min, ES⁺ m/z 524.1/526.1/528.1 [M+H]⁺.

### Step 3

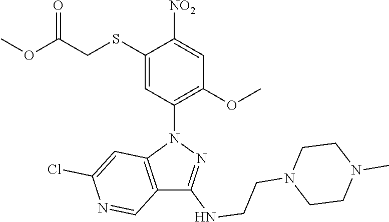

### Methyl 2-((5-(6-chloro-3-((2-(4-methylpiperazin-1-yl)ethyl)amino)-1H-pyrazolo[4,3-c]pyridin-1-yl)-4-methoxy-2-nitrophenyl)thio)acetate (Intermediate 23a-3)

A solution of intermediate 23a-2 (240 mg, 0.46 mmol), DIPEA (199 μL, 1.1 mmol) and methyl 2-sulfanylacetate (102 μL, 1.1 mmol) in dry acetonitrile (14 mL) was stirred at 120° C. overnight. The solvent was removed in vacuo. The residue was purified by flash chromatography on Si cartridge by eluting with 0-100% DCM/MeOH/NH₄OH (90:15:1.5) in DCM to afford the title product (87 mg).

LCMS (Method 2): Rt=0.97 min, ES⁺ m/z 550.2/552.2 [M+H]⁺.

### Step 4

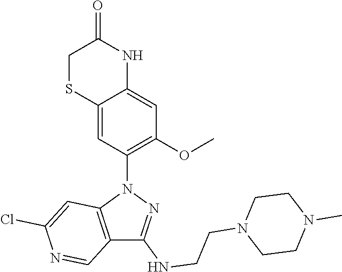 [SMILES: COc1cc2c(cc1-n1nc(NCCN3CCN(C)CC3)c3cnc(Cl)cc31)SCC(=O)N2]

### 7-(6-Chloro-3-((2-(4-methylpiperazin-1-yl)ethyl)amino)-1H-pyrazolo[4,3-c]pyridin-1-3l)-6-methoxy-2H-benzo[b][1,4]thiazin-3 (4H)-one (Intermediate 23a)

A solution of intermediate 23a-3 (85 mg, 0.15 mmol) in EtOH (20 mL) was stirred at 80° C., then added with a solution of NH₄Cl (70 mg, 1.3 mmol) in water (5 mL) and iron (0.16 g, 2.9 mmol). RM was stirred at 80° C. for 4.5 h. A second equivalent of iron and NH₄Cl were added and RM further stirred at 80° C. for 1 h. RM was quenched with aq. 2 N HCl (1 mL) and stirred at 80° C. for 2 h. After cooling to RT, RM was filtered and filter cake washed with EtOH. The filtrate was evaporated in vacuo. The residue was treated with sat. aq. NaHCO₃ (10 mL) and extracted with EtOAc (2×20 mL). Combined organic layers were washed with sat. aq. NaCl (10 mL), dried over Na₂SO₄, and solvent evaporated in vacuo. The residue was purified by flash chromatography on Si cartridge by eluting with 0-100% DCM/MeOH/NH₄OH (90:15:1.5) in DCM to afford the title product (40 mg).

LCMS (Method 2): Rt=0.80 min, ES⁺ m/z 488.2/490.2 [M+H]⁺.

### Intermediate 23b

### Step 1

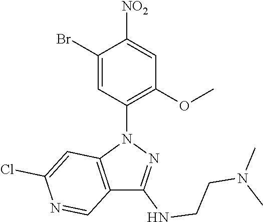 [SMILES: COc1cc([N+](=O)[O-])c(Br)cc1-n1nc(NCCN(C)C)c2cnc(Cl)cc21]

### N¹-(1-(5-Bromo-2-methoxy-4-nitrophenyl)-6-chloro-1H-pyrazolo[4,3-c]pyridin-3-yl)-N²,N²-dimethylethane-1,2-diamine (Intermediate 23b-1)

The title intermediate was prepared on a similar manner to intermediate 23a-2 starting from intermediate 23a-1 and N′,N′-dimethylethane-1,2-diamine.

LCMS (Method 2): Rt=1.18 min, ES⁺ m/z 469.1/471.1/473.1 [M+H]⁺

### Step 2

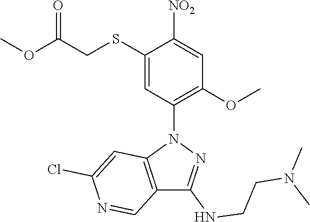

### Methyl 2-((5-(6-chloro-3-((2-(dimethylamino)ethyl)amino)-1H-pyrazolo[4,3-c]pyridin-1-yl)-4-methoxy-2-nitrophenyl)thio)acetate (Intermediate 23b-2)

The title intermediate was prepared on a similar manner to intermediate 23a-3 starting from intermediate 23b-1 and methyl 2-sulfanylacetate to give the title product (122 mg).

LCMS (Method 2): Rt=1.12 min ES⁺ m/z 495.1/497.1 [M+H]⁺

### Step 3

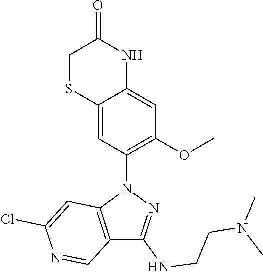 [SMILES: COc1cc2c(cc1-n1nc(NCCN(C)C)c3cnc(Cl)cc31)SCC(=O)N2]

### 7-(6-Chloro-3-((2-(dimethylamino)ethyl)amino)-1H-pyrazolo[4,3-c]pyridin-1-yl)-6-methoxy-2H-benzo[b][1,4]thiazin-3 (4H)-one (Intermediate 23b)

The title compound was prepared on a similar manner to intermediate 23a-4 starting from intermediate 23b-2.

LCMS (Method 2): Rt=1.04 min, ES⁺ m/z 433.2/434.2 [M+H]⁺

### Intermediate 24

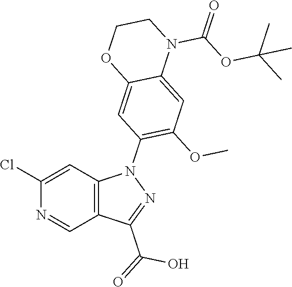 [SMILES: COc1cc2c(cc1-n1nc(C(=O)O)c3cnc(Cl)cc31)OCCN2C(=O)OC(C)(C)C]

### 1-(4-(tert-Butoxycarbonyl)-6-methoxy-3,4-dihydro-2H-benzo[b][1,4]oxazin-7-yl)-6-chloro-1H-pyrazolo[4,3-c]pyridine-3-carboxylic acid (Intermediate 24)

### Method 1

A degassed mixture of 6-Chloro-1H-pyrazolo[4,3-c]pyridine-3-carboxylic acid (400 mg, 2.02 mmol), intermediate 2 (733 mg, 2.13 mmol), Cs₂CO₃ (2.31 g, 7.09 mmol) and thiophene-2-carbonyloxycopper (278 mg, 1.46 mmol) in anhydrous DMSO (7 mL) was stirred under nitrogen at 110° C. overnight. After cooling to RT, RM was filtered. The filtrate was diluted with MeCN (50 mL) thus the formed precipitate collected by filtration and triturated with MeOH. The crude material was purified by flash chromatography on Si cartridge by eluting with 0-100% MeOH in EtOAc to afford the title product (150 mg).

### Method 2

A degassed mixture of 6-Chloro-1H-pyrazolo[4,3-c]pyridine-3-carboxylic acid (9.4 g, 47.5 mmol), intermediate 7 (18.6 g, 47.5 mmol), Cs₂CO₃ (54 g, 166.3 mmol) and thiophene-2-carbonyloxycopper (9 g, 47.5 mmol) in DMSO (100 mL) was stirred under argon at 110° C. for 24 h. RM was quenched in water and extracted with DCM. Combined organic layers was washed with aq 10% w/w citric acid, aq. sat. NaCl and evaporated to dryness. The residue was purified by chromatography on silica gel by eluting with DCM to DCM (1% v/v MeOH+1% AcOH v/v). The material thus obtained was triturated in ethyl ether to afford the title compound (3.14 g).

LCMS (Method 1): Rt=1.22 min, ES⁺ m/z 461.2/463.2 [M+H]⁺.

### Intermediate 25

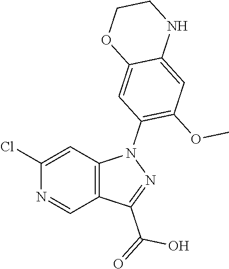 [SMILES: COc1cc2c(cc1-n1nc(C(=O)O)c3cnc(Cl)cc31)OCCN2]

### 6-Chloro-1-(6-methoxy-3,4-dihydro-2H-benzo[b][1,4]oxazin-7-yl)-1H-pyrazolo[4,3-c]pyridine-3-carboxylic acid (Intermediate 25)

The title intermediate was prepared in a similar manner to intermediate 24 method 1 starting 6-Chloro-1H-pyrazolo[4,3-c]pyridine-3-carboxylic acid and intermediate 2-1.

LCMS (Method 1): Rt=0.86 min, ES⁺ m/z 360.9/362.8 [M+H]⁺.

### Intermediate 26

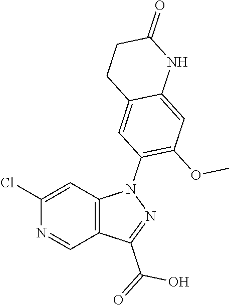 [SMILES: COc1cc2c(cc1-n1nc(C(=O)O)c3cnc(Cl)cc31)CCC(=O)N2]

### 6-Chloro-1-(7-methoxy-2-oxo-1,2,3,4-tetrahydroquinolin-6-yl)-1H-pyrazolo[4,3-c]pyridine-3-carboxylic acid (Intermediate 26)

The title intermediate was prepared on similar manner like intermediate 24 method 1 starting from 6-Chloro-1H-pyrazolo[4,3-c]pyridine-3-carboxylic acid and 6-bromo-7-methoxy-3,4-dihydro-1H-quinolin-2-one.

LCMS (Method 1): Rt=0.77 min, ES⁺ m/z 373.1/375.1 [M+H]⁺.

### Intermediate 27a

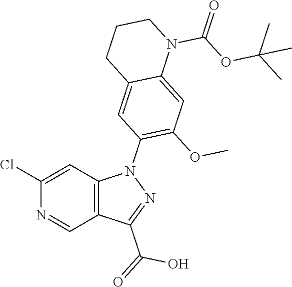 [SMILES: COc1cc2c(cc1-n1nc(C(=O)O)c3cnc(Cl)cc31)CCCN2C(=O)OC(C)(C)C]

### 1-(1-(tert-Butoxycarbonyl)-7-methoxy-1,2,3,4-tetrahydroquinolin-6-yl)-6-chloro-1H-pyrazolo[4,3-c]pyridine-3-carboxylic acid (Intermediate 27a)

A mixture of intermediate 3 (1.04 g, 3.04 mmol), 6-Chloro-1H-pyrazolo[4,3-c]pyridine-3-carboxylic acid (300 mg, 1.52 mmol), thiophene-2-carbonyloxycopper (290 mg, 1.52 mmol) and Cs₂CO₃ (1.48 g, 4.56 mmol) in DMSO (7 mL) was stirred for 72 h at 100° C. under nitrogen. After cooling to RT, RM was added dropwise to water under stirring. The pH was adjusted to 4 (with aq. HCl 1M) and the formed precipitate filtered, washed with water, and dried. The crude material was purified by flash chromatography on Si cartridge by eluting with 0-50% DCM:MeOH:HCO₂H (90:10:0.3) in DCM to afford the title product (330 mg).

LCMS (Method 1): Rt=1.24 min, ES⁺ m/z 458.9/460.9 [M+H]⁺.

### Intermediate 27b

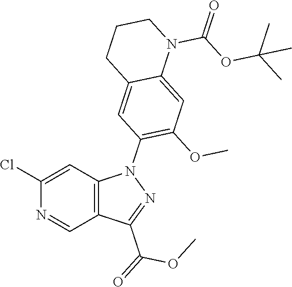 [SMILES: COC(=O)c1nn(-c2cc3c(cc2OC)N(C(=O)OC(C)(C)C)CCC3)c2cc(Cl)ncc12]

### tert-Butyl 6-(6-chloro-3-(methoxycarbonyl)-1H-pyrazolo[4,3-c]pyridin-1-yl)-7-methoxy-3,4-dihydroquinoline-1 (2H)-carboxylate (Intermediate 27b)

A mixture of intermediate 3 (346 mg, 1.01 mmol), 6-Chloro-1H-pyrazolo[4,3-c]pyridine-3-carboxylic acid (100 mg, 0.506 mmol), thiophene-2-carbonyloxycopper (97 mg, 0.5 mmol) and Cs₂CO₃ (0.49 g, 1.52 mmol) in DMSO (2.3 mL) was stirred for 72 h at 100° C. under nitrogen. After cooling to RT, iodomethane (126 μl, 2.02 mmol) was added and the mixture stirred at RT for further 2 h more. RM was quenched with water and extracted three times with DCM. Combined organic layers were dried over Na₂SO₄, filtered, and evaporated to dryness. The residue was purified by flash chromatography on Si cartridge by eluting with 0-10% cyclohexane/EtOAc (1:1) in cyclohexane to afford the title product (118 mg).

LCMS (Method 1): Rt=1.41 min, ES⁺ m/z 473.0/475.0 [M+H]⁺.

### Intermediate 28

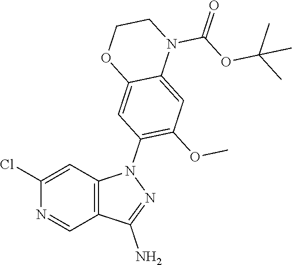 [SMILES: COc1cc2c(cc1-n1nc(N)c3cnc(Cl)cc31)OCCN2C(=O)OC(C)(C)C]

### tert-Butyl 7-(3-amino-6-chloro-1H-pyrazolo[4,3-c]pyridin-1-yl)-6-methoxy-2,3-dihydro-4H-benzo[b][1,4]oxazine-4-carboxylate (Intermediate 28)

A degassed mixture of intermediate 2 (408 mg, 1.19 mmol), 6-chloro-1H-pyrazolo[4,3-c]pyridin-3-amine (200 mg, 1.19 mmol), Cs₂CO₃ (980 mg, 3.01 mmol), DMCHDA (93.5 μL, 0.59 mmol) and CuI (113 mg, 0.59 mmol) in DMSO (4.9 mL) was stirred under argon at 110° C. overnight. After cooling to RT, RM was quenched with sat. aq. NaHCO₃ (50 mL) and extracted with EtOAc (5×50 mL). Combined organic layers were washed with sat. aq. NaCl, dried over Na₂SO₄ and solvent removed in vacuo. The crude residue was purified by flash chromatography on Si cartridge by eluting with 0-50% DCM/MeOH/NH₄OH (90:5:0.5) in DCM to afford the title product (282 mg).

LCMS (Method 1): Rt=1.15 min, ES⁺ m/z 431.9/433.9 [M+H]⁺.

### Intermediate 29

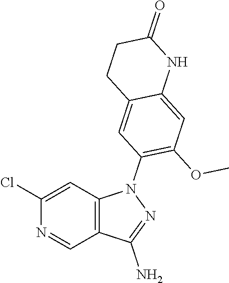 [SMILES: COc1cc2c(cc1-n1nc(N)c3cnc(Cl)cc31)CCC(=O)N2]

### 6-(3-Amino-6-chloro-1H-pyrazolo[4,3-c]pyridin-1-yl)-7-methoxy-3,4-dihydroquinolin-2 (1H)-one (Intermediate 29)

A degassed solution of 6-bromo-7-methoxy-3,4-dihydro-1H-quinolin-2-one (304 mg, 1.19 mmol), 6-chloro-1H-pyrazolo[4,3-c]pyridin-3-amine (100 mg, 0.59 mmol), K₂CO₃ (246 mg, 1.78 mmol), NN-dimethylglycine (93.5 μL, 0.59 mmol) and CuI (56.5 mg, 0.30 mmol) in DMSO (3.6 mL) was stirred under argon at 110° C. overnight. After cooling to RT, RM was quenched with sat. aq. NaHCO₃ (50 mL) and extracted with EtOAc (5×50 mL). Combined organic layers were washed with sat. aq. NaCl, dried over Na₂SO₄ and solvent removed in vacuo. The residue was purified by flash chromatography on Si cartridge by eluting with 0-100% DCM/MeOH/NH₄OH (90:5:1) in DCM to give the title product (68.6 mg).

LCMS (Method 2): Rt=0.68 min, ES⁺ m/z 344.1/345.9 [M+H]⁺.

### Intermediate 30

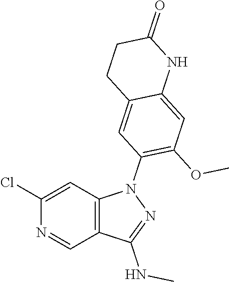 [SMILES: CNc1nn(-c2cc3c(cc2OC)NC(=O)CC3)c2cc(Cl)ncc12]

### 6-(6-Chloro-3-(methylamino)-1H-pyrazolo[4,3-c]pyridin-1-yl)-7-methoxy-3,4-dihydroquinolin-2 (1H)-one (Intermediate 30)

The title compound was prepared similarly to intermediate 29 starting from 6-bromo-7-methoxy-3,4-dihydroquinolin-2 (1H)-one and intermediate 18b.

LCMS (Method 2): Rt=0.79 min, ES⁺ m/z 358.1/360.1 [M+H]⁺.

### Intermediate 31a

### Step 1

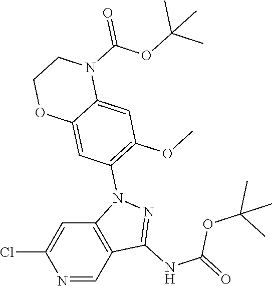 [SMILES: COc1cc2c(cc1-n1nc(NC(=O)OC(C)(C)C)c3cnc(Cl)cc31)OCCN2C(=O)OC(C)(C)C]

### tert-Butyl 7-(3-((tert-butoxycarbonyl)amino)-6-chloro-1H-pyrazolo[4,3-c]pyridin-1-yl)-6-methoxy-2,3-dihydro-4H-benzo[b][1,4]oxazine-4-carboxylate (Intermediate 31a-1)

LiHMDS (1.30 M in THF, 463 μL, 0.60 mmol) was added to a solution of intermediate 28 (130 mg, 0.30 mmol) in THF (7 mL) at 0° C. under nitrogen. RM was stirred for 15 min. A solution of Boc₂O (131 mg, 0.60 mmol) in THF (1 mL) was added dropwise and RM stirred at RT for 3 h. RM was diluted with water and extracted with EtOAc. Combined organic layers were washed with sat. aq. NaCl, dried over Na₂SO₄ and solvent removed in vacuo. The residue was purified by flash chromatography on Si cartridge by eluting with 0-50% EtOAc in cyclohexane. The isolated product was dissolved in methanol (1 mL), added with K₂CO₃ (76.5 mg, 0.55 mmol) and stirred at RT overnight. RM was diluted with sat. aq. NaHCO₃ and extracted with DCM. Combined organic layers were washed with sat. aq. NaCl, dried over Na₂SO₄ and solvent removed in vacuo to afford the title product (107 mg) that was used in the next synthetic step without further purification.

LCMS (Method 2): Rt=1.49 min ES⁺ m/z 532.3/534.3 [M+H]⁺.

### Step 2

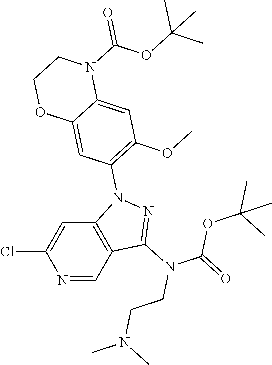 [SMILES: COc1cc2c(cc1-n1nc(N(CCN(C)C)C(=O)OC(C)(C)C)c3cnc(Cl)cc31)OCCN2C(=O)OC(C)(C)C]

### tert-Butyl 7-(3-((tert-butoxycarbonyl)(2-(dimethylamino)ethyl)amino)-6-chloro-1H-pyrazolo[4,3-c]pyridin-1-yl)-6-methoxy-2,3-dihydro-4H-benzo[b][1,4]oxazine-4-carboxylate (Intermediate 31a)

A solution of intermediate 31a-1 (85.0 mg, 0.16 mmol) in dry DMF (1.0 mL) was cooled under argon at 0° C. NaI (23.9 mg, 0.16 mmol) and NaH, (60% dispersion in mineral oil, 19.2 mg, 0.48 mmol) were added and RM stirred at 0° C. for 30 min. 2-bromo-N,N-dimethyl-ethanamine hydrobromide (55.8 mg, 0.24 mmol) was added and stirring proceeded at RT for 60 h. RMs was diluted with water and extracted with EtOAc (3×10 mL). Combined organics were passed through a phase separator cartridge and solvent was evaporated in vacuo. The residue was purified by flash chromatography on Si cartridge by eluting with 0-100% DCM/MeOH/NH₄OH (90:4:1) in DCM to afford the title product.

LCMS (Method 2): Rt=1.54 min, ES⁺ m/z 603.3/605.3 [M+H]⁺.

### Intermediate 31b

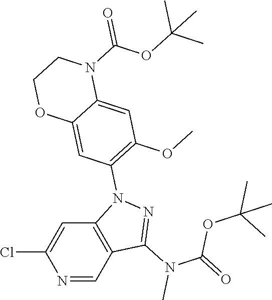 [SMILES: COc1cc2c(cc1-n1nc(N(C)C(=O)OC(C)(C)C)c3cnc(Cl)cc31)OCCN2C(=O)OC(C)(C)C]

### tert-Butyl 7-(3-((tert-butoxycarbonyl)(methyl)amino)-6-chloro-1H-pyrazolo[4,3-c]pyridin-1-yl)-6-methoxy-2,3-dihydro-4H-benzo[b][1,4]oxazine-4-carboxylate (Intermediate 31b)

The title compound was prepared similarly to intermediate 31a starting from intermediate 31a-1 and iodomethane.

LCMS (Method 2): Rt=1.54 min, ES⁺ m/z 546.2/548.2 [M+H]⁺.

### Intermediate 32a

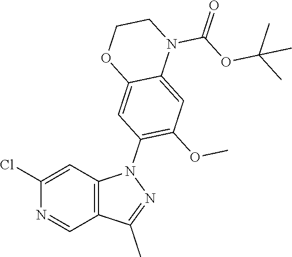 [SMILES: COc1cc2c(cc1-n1nc(C)c3cnc(Cl)cc31)OCCN2C(=O)OC(C)(C)C]

### tert-Butyl 7-(6-chloro-3-methyl-1H-pyrazolo[4,3-c]pyridin-1-yl)-6-methoxy-2,3-dihydro-4H-benzo[b][1,4]oxazine-4-carboxylate (Intermediate 32a)

To a degassed mixture of intermediate 2 (657 mg, 1.9 ol), 6-chloro-3-methyl-1H-pyrazolo[4,3-c]pyridine (200 mg, 1.2 mmol), CuI (114 mg, 0.6 mmol), N,N-dimethylglycine (123 mg, 1.2 mmol) and K₂CO₃ (330 mg, 2.4 mmol) was added DMSO (5 mL). RMV was stirred at 100° C. overnight. After cooling to RT, RM was diluted with EtOAc (25 mL) and washed with 15% w/w aq. NH₄OH (3×15 mL) and sat. aq. NaCl (15 mL). The organic layers were dried over Na₂SO₄ and concentrated in vacuo. The residue was purified by flash chromatography on Si cartridge by eluting with 0-15% EtOAc in DCM to afford the title product (312 mg).

LCMS (Method 2): Rt=1.32 min, ES⁺ m/z 431.1/433.1 [M+H]⁺.

### Intermediates 32b to 32i

The following intermediates were prepared in a similar manner to intermediate 32a from the indicated starting materials. It is additionally stated if base, ligand/catalyst, solvent and/or temperature were varied.

| Intermediate | Structure | Starting material | LCMS |
|---|---|---|---|
| Intermediate 32b | 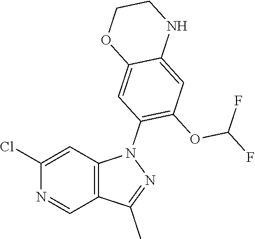 [SMILES: Cc1nn(-c2cc3c(cc2OC(F)F)NCCO3)c2cc(Cl)ncc12] | Intermediate 1/6- chloro-3-methyl-1H- pyrazolo[4,3- c]pyridine ligand: DMCHA solvent: DMF temp.: 110° C. | (Method 1): Rt = 1.08 min, ES⁺ m/z 367.1/369.1 [M + H]⁺ |
| Intermediate 32c | 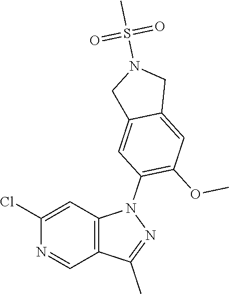 [SMILES: COc1cc2c(cc1-n1nc(C)c3cnc(Cl)cc31)CN(S(C)(=O)=O)C2] | Intermediate 9/6- chloro-3-methyl-1H- pyrazolo[4,3- c]pyridine | (Method 1): Rt = 0.96 min, ES⁺ m/z 393.1/ 395.2 [M + H]⁺ |
| Intermediate 32d | 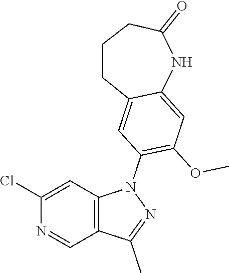 [SMILES: COc1cc2c(cc1-n1nc(C)c3cnc(Cl)cc31)CCCC(=O)N2] | Intermediate 4/6- chloro-3-methyl-1H- pyrazolo[4,3- c]pyridine | (Method 1): Rt = 0.90 min, ES⁺ m/z 357.1/ 359.1 [M + H]⁺ |
| Intermediate 32e | 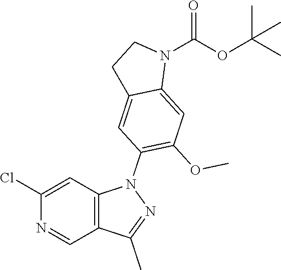 [SMILES: COc1cc2c(cc1-n1nc(C)c3cnc(Cl)cc31)CCN2C(=O)OC(C)(C)C] | Intermediate 6/6- chloro-3-methyl-1H- pyrazolo[4,3- c]pyridine | (Method 1): Rt = 1.36 min, ES⁺ m/z 415.2/ 417.2 [M + H]⁺ |
| Intermediate 32f | 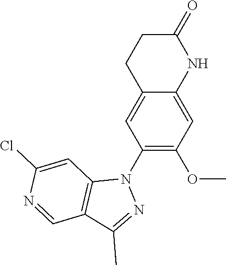 [SMILES: COc1cc2c(cc1-n1nc(C)c3cnc(Cl)cc31)CCC(=O)N2] | 6-bromo-7-methoxy- 3,4-dihydro-1H- quinolin-2-one/6- chloro-3-methyl-1H- pyrazolo[4,3- c]pyridine | (Method 1): Rt = 0.86 min, ES⁺ m/z 343.0/345.0 [M + H]⁺ |
| Intermediate 32g | 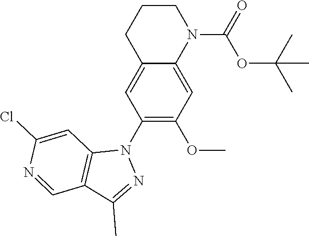 [SMILES: COc1cc2c(cc1-n1nc(C)c3cnc(Cl)cc31)CCCN2C(=O)OC(C)(C)C] | Intermediate 3/6- chloro-3-methyl-1H- pyrazolo[4,3- c]pyridine | (Method 1): Rt = 1.37 min, ES⁺ m/z 428.9/430.8 [M + H]⁺ |
| Intermediate 32h | 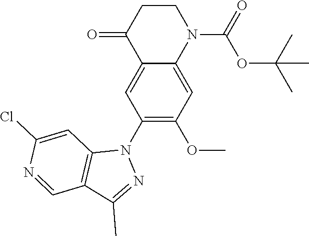 [SMILES: COc1cc2c(cc1-n1nc(C)c3cnc(Cl)cc31)C(=O)CCN2C(=O)OC(C)(C)C] | Intermediate 5/6- chloro-3-methyl-1H- pyrazolo[4,3- c]pyridine | (Method 1): Rt = 1.29 min, ES⁺ m/z 443.1/445.1 [M + H]⁺ |
| Intermediate 32i | 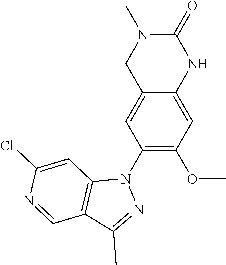 [SMILES: COc1cc2c(cc1-n1nc(C)c3cnc(Cl)cc31)CN(C)C(=O)N2] | Intermediate 11/6- chloro-3-methyl-1H- pyrazolo[4,3- c]pyridine | (Method 1): Rt = 0.94 min, ES⁺ m/z 358.2/360.2 [M + H]⁺ |

### Intermediate 32i

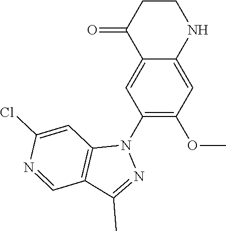 [SMILES: COc1cc2c(cc1-n1nc(C)c3cnc(Cl)cc31)C(=O)CCN2]

### 6-(6-Chloro-3-methyl-1H-pyrazolo[4,3-c]pyridin-1-yl)-7-methoxy-2,3-dihydroquinolin-4 (1H)-one (Intermediate 32i)

TFA (173 μL, 2.26 mmol) was added to a solution of intermediate 32 h (40.0 mg, 0.09 mmol) in DCM (1.0 mL). RM was stirred at RT overnight, then concentrated in vacuo. The residue was loaded on SCX cartridge, washed with methanol, and eluted with 2M methanolic ammonia to afford the title product (32 mg).

LCMS (Method 1): Rt=0.87 min, ES m/z 342.9/344.9 [M+H]⁺

### Intermediate 33

### Step 1

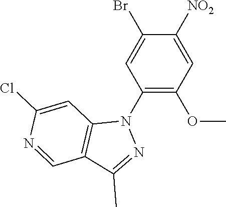 [SMILES: COc1cc([N+](=O)[O-])c(Br)cc1-n1nc(C)c2cnc(Cl)cc21]

### 1-(5-Bromo-2-methoxy-4-nitrophenyl)-6-chloro-3-methyl-1H-pyrazolo[4,3-c]pyridine (Intermediate 33-1)

A mixture of 6-chloro-3-methyl-1H-pyrazolo[4,3-c]pyridine (300 mg, 1.79 mmol), 1-bromo-5-fluoro-4-methoxy-2-nitro-benzene (448 g, 1.79 mmol) and K₂CO₃ (742 g, 5.37 mmol) was suspended in DMF (6.0 mL) and stirred at 80° C. for 1 h. After cooling to RT, RM was diluted with water (80 mL). The formed precipitate was collected by filtration, washed with water (3×30 mL) and dried to afford the title product (620 mg) that was used in the next synthetic steps without further purification.

LCMS (Method 1): Rt=1.26 min, ES m/z 397.1/399.1/401.1 [M+H]⁺

### Step 2

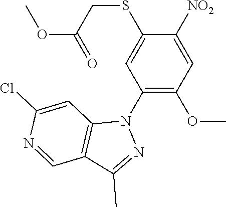 [SMILES: COC(=O)CSc1cc(-n2nc(C)c3cnc(Cl)cc32)c(OC)cc1[N+](=O)[O-]]

### Methyl 2-((5-(6-chloro-3-methyl-1H-pyrazolo[4,3-c]pyridin-1-yl)-4-methoxy-2-nitrophenyl)thio)acetate (Intermediate 33-2)

A mixture of intermediate 33-1 (350 mg, 0.88 mmol), methyl 2-sulfanylacetate (118 μL, 1.3 mmol) and DIPEA (230 μL, 1.3 mmol) in dry acetonitrile (15.0 mL) was stirred at 120° C. in a μM reactor for 3 h. A new equivalent of methyl 2-sulfanylacetate (16 μL, 0.17 mmol) was added and RM was further stirred at 120° C. for 2 h. After cooling to RT, solvent was removed in vacuo. The residue was purified by flash chromatography on Si cartridge by eluting with 0-80% (DCM/MeOH (9:1) in DCM to afford the desired product (170 mg).

LCMS (Method 1): Rt=1.16 min, ES⁺ m/z 423.2/425.2 [M+H]⁺

### Step 3

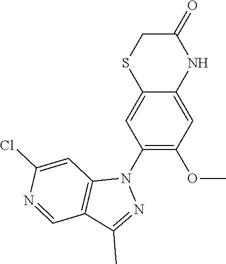 [SMILES: COc1cc2c(cc1-n1nc(C)c3cnc(Cl)cc31)SCC(=O)N2]

### 7-(6-Chloro-3-methyl-1H-pyrazolo[4,3-c]pyridin-1-yl)-6-methoxy-2H-benzo[b][1,4]thiazin-3 (4H)-one (Intermediate 33-3)

To a stirring solution of intermediate 33-2 (160 mg, 0.38 mmol) in ethanol (5 mL) w at 80° C., a solution of NH₄Cl (81 mg, 1.5 mmol) in water (2 mL) and iron (0.25 g, 4.5 mmol) were added. RM was stirred at 80° C. for 6 h. After cooling to RT, RM was diluted with EtOAc (20 mL), filtered through a bed of diacematous earth and washed thoroughly with EtOAc. The filtrate was concentrated in vacuo. The residue was purified by flash chromatography on Si cartridge by eluting with 0-35% DCM/MeOH (95:5) in DCM to afford the title product (50 mg).

LCMS (Method 1): Rt=0.96 min, ES⁺ m/z 361.2/363.2 [M+H]⁺

### Step 4

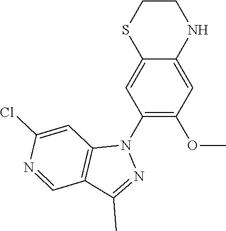 [SMILES: COc1cc2c(cc1-n1nc(C)c3cnc(Cl)cc31)SCCN2]

### 7-(6-Chloro-3-methyl-1H-pyrazolo[4,3-c]pyridin-1-yl)-6-methoxy-3,4-dihydro-2H-benzo[b][1,4]thiazine (Intermediate 33-4)

To a 0° C. cooled solution of BH₃·THF (1.0 M in THF, 166.3 μL, 0.16 mmol), a solution of intermediate 33-3 (30.00 mg, 0.08 mmol) in THF (1 mL) was added and RM stirred at 25° C. for 2 h. A second equivalent of BH₃·THF was added and RM further stirred for 2 h. RM was quenched with sat aq. NH₄Cl (20 mL) and extracted with EtOAc (2×10 mL). Combined organic layers were washed with sat. aq. NaHCO₃ (3×10 mL) and concentrated in vacuo. The residue was purified by flash chromatography on Si cartridge by eluting with 0-100% DCM/MeOH (98:2) in DCM to afford the title product (18 mg).

LCMS (Method 1): Rt=1.11 min, ES⁺ m/z 347.2/349.2 [M+H]⁺

### Step 5

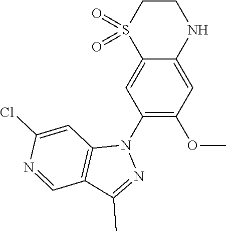 [SMILES: COc1cc2c(cc1-n1nc(C)c3cnc(Cl)cc31)S(=O)(=O)CCN2]

### 7-(6-Chloro-3-methyl-1H-pyrazolo[4,3-c]pyridin-1-yl)-6-methoxy-3,4-dihydro-2H-benzo[b][1,4]thiazine 1,1-dioxide (Intermediate 33)

To a 0° C. cooled solution of intermediate 33-4 (25.0 mg, 67.8 μmol) in dry DCM (1 mL), m-CPBA (70.0%, 33.4 mg, 0.136 mmol) was added and RM stirred at 0° C. for 1 h. After warming to RT, RM was diluted with DCM and washed with sat. aq. NaHCO₃. Organic phase was evaporated in vacuo. The residue was purified by flash chromatography on Si cartridge by eluting with 0-60% DCM/MeOH/NH₄OH (90:5:0.5) in DCM to give the title product (13 mg).

LCMS (Method 1): Rt=0.82 min, ES⁺ m/z 378.9/380.8 [M+H]⁺

### Intermediate 34

### Step 1

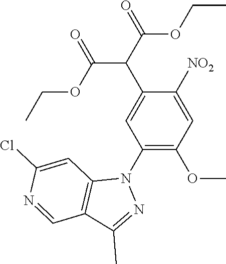 [SMILES: CCOC(=O)C(C(=O)OCC)c1cc(-n2nc(C)c3cnc(Cl)cc32)c(OC)cc1[N+](=O)[O-]]

### Diethyl 2-(5-(6-chloro-3-methyl-1H-pyrazolo[4,3-c]pyridin-1-yl)-4-methoxy-2-nitrophenyl)malonate (Intermediate 34-1)

To a mixture of NaH (60% suspension in mineral oil, 80 mg, 2.0 mmol) in DMSO (4.0 mL), diethyl malonate (186 μL, 1.3 mmol) was slowly added and RM stirred at 65° C. After 20 min, intermediate 33-1 (195 mg, 0.49 mmol) was added and RM was at 100° C. for 1 h. After cooling to RT, RM was poured on ice/water and was extracted with EtOAc (4×10 mL). Combined organic layers were washed with water (10×10 mL) and evaporated in vacuo. The residue was purified by flash chromatography on Si cartridge by eluting with 0-45% DCM/MeOH (98:2) in DCM to afford the title product (75 mg).

LCMS (Method 1): Rt=1.26 min, ES⁺ m/z 477.2/479.2 [M+H]⁺

### Step 2

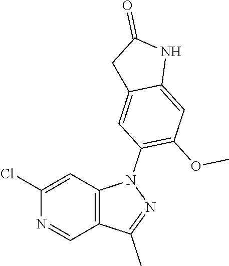 [SMILES: COc1cc2c(cc1-n1nc(C)c3cnc(Cl)cc31)CC(=O)N2]

### 5-(6-chloro-3-methyl-1H-pyrazolo[4,3-c]pyridin-1-yl)-6-methoxyindolin-2-one (Intermediate 34-2)

To a stirring solution of intermediate 34-1 (75 mg, 0.16 mmol) in acetic acid (4.0 mL) at 90° C., iron (97 mg, 1.7 mmol) was added in portions and RM stirred at 90° C. for 80 min. After cooling to RT, RM was filtered, filtrate was diluted with water (5 mL) and extracted EtOAc (3×10 mL). Combined organics were washed with sat. aq. NaHCO₃ (10 mL), dried over Na₂SO₄ and concentrated in vacuo. The residue was purified by flash chromatography on Si cartridge by eluting with 0-75% DCM/MeOH/NH₄OH (90:5:0.5) in DCM to afford the title product (21.4 mg).

LCMS (Method 1): Rt=0.82 min, ES⁺ m/z 329.2/331.2 [M+H]⁺

### Step 3

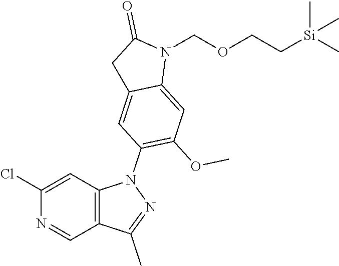 [SMILES: COc1cc2c(cc1-n1nc(C)c3cnc(Cl)cc31)CC(=O)N2COCC[Si](C)(C)C]

### 5-(6-Chloro-3-methyl-1H-pyrazolo[4,3-c]pyridin-1-yl)-6-methoxy-1-((2-(trimethylsilyl)ethoxy)methyl)indolin-2-one (Intermediate 34-3)

To a stirring suspension of NaH (60.0%, 14.9 mg, 0.37 mmol) in dry DMF (0.3 mL), a solution of intermediate 34-2 (180 mg, 0.37 mmol) in DMF (3 mL) was added and RM stirred for 30 min reaching RT. 2-(chloromethoxy)ethyl-trimethyl-silane (139 μL, 0.75 mmol) was added dropwise at 0° C. RM was stirred overnight at RT, then quenched with sat. aq. NaHCO₃ (15 mL), and extracted with EtOAc (3×15 mL). Combined organic layers were washed with water (20 mL), sat. aq. NaCl (20 mL) and solvent removed in vacuo. The residue was purified by flash chromatography on Si cartridge by eluting with 0-40% EtOAc/DCM (1:9) in DCM to afford the title product (100 mg).

LCMS (Method 2): Rt=1.35 min, ES⁺ m/z 459.2/461.2 [M+H]⁺

### Step 4

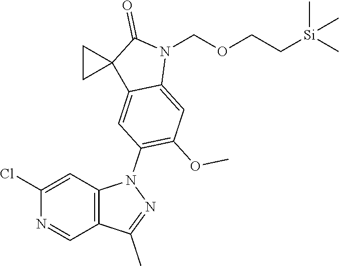 [SMILES: COc1cc2c(cc1-n1nc(C)c3cnc(Cl)cc31)C1(CC1)C(=O)N2COCC[Si](C)(C)C]

### 5′-(6-Chloro-3-methyl-1H-pyrazolo[4,3-c]pyridin-1-yl)-6′-methoxy-1′-((2-(trimethylsilyl)ethoxy)methyl)spiro[cyclopropane-1,3′-indolin]-2′-one (Intermediate 34)

To a stirring mixture of intermediate 34-3 (60.0 mg, 0.12 mmol) diphenyl(vinyl)sulfonium trifluoromethanesulfonate (51.7 mg, 0.14 mmol) and zinc trifluoromethanesulfonate (43.2 mg, 0.12 mmol) in dry DMF (3 mL), DBU (53.3 μL, 0.36 mmol) was added. RM was stirred at RT for 2 h. RM was quenched with sat. aq. NH₄Cl (10 mL) and extracted with EtOAc (3×15 mL). Combined organic layers were washed with water (2×10 mL), dried over Na₂SO₄ and solvent removed in vacuo. The residue was purified by flash chromatography on Si cartridge by eluting with 0-25% EtOAc/DCM (1:9) in DCM to afford the title product (48 mg).

LCMS (Method 2): Rt=1.44 min ES⁺ m/z 485.2/487.2 [M+H]⁺

### Intermediate 35

### Step 1

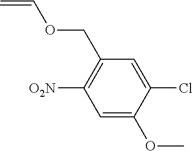 [SMILES: C=COCc1cc(Cl)c(OC)cc1[N+](=O)[O-]]

### 1-(Allyloxy)-5-chloro-4-methoxy-2-nitrobenzene (Intermediate 35-1)

Suspension of 5-chloro-4-methoxy-2-nitro-phenol (1.00 g, 4.91 mmol), 3-bromoprop-1-ene (509 μL, 5.89 mmol) and K₂CO₃ (1018 mg, 7.37 mmol) in acetonitrile (10 mL) was stirred at 75° C. overnight. RM was cooled to RT and diluted with water and DCM. Layers were separated and aqueous layer was washed with DCM. Combined organics were dried and concentrated to get a crude product that was used in the next synthetic step without further purification (1.08 g).

LCMS (Method 1): Rt=1.18 min

¹H-NMR (300 MHz, CDCl₃) δ: 7.48 (s, 1H), 7.13 (s, 1H), 5.95-6.07 (m, 1H), 5.47 (dq, J=17.3, 1.3 Hz, 1H), 5.33 (dq, J=10.4, 1.3 Hz, 1H), 4.62 (dt, J=5.0, 1.3 Hz, 2H), 3.90 (s, 3H).

### Step 2

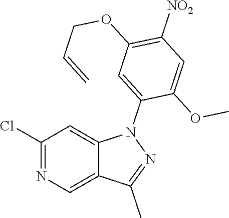 [SMILES: C=CCOc1cc(-n2nc(C)c3cnc(Cl)cc32)c(OC)cc1[N+](=O)[O-]]

### 1-(5-(Allyloxy)-2-methoxy-4-nitrophenyl)-6-chloro-3-methyl-1H-pyrazolo[4,3-c]pyridine (Intermediate 35-2)

A suspension of 6-chloro-3-methyl-1H-pyrazolo[4,3-c]pyridine (700 mg, 4.18 mmol), intermediate 35-1 (1.08 g, 4.42 mmol) and K₂CO₃ (1.73 g, 12.5 mmol) in DMSO (10.0 mL) was stirred at 120° C. for 5 h. RM was cooled to RT and diluted with water. The formed precipitate was collected by filtration, washed with water, and dried to afford the desired product (950 mg) that was used in the next synthetic steps without further purification.

LCMS (Method 1): Rt=1.19 min, ES⁺ m/z 374.9/376.9 [M+H]⁺.

### Step 3

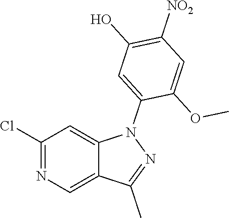 [SMILES: COc1cc([N+](=O)[O-])c(O)cc1-n1nc(C)c2cnc(Cl)cc21]

### 5-(6-Chloro-3-methyl-1H-pyrazolo[4,3-c]pyridin-1-yl)-4-methoxy-2-nitrophenol (Intermediate 35-3)

To a degassed mixture of intermediate 35-2 (500 mg, 1.33 mmol) and K₂CO₃ (553 mg, 4.00 mmol) in MeOH (15 mL), Pd(PPh₃)₄ (50.0 mg, 0.04 mmol) was added and RM stirred at 60° C. for 30 min. After cooling to RT, RM was concentrated and suspended in water, acidified to pH 3 and extracted with DCM. Combined organic layers were dried over Na₂SO₄, and evaporated to dryness. The residue was triturated in DCM/MeOH to afford the desired product (200 mg) that was used in the next synthetic steps without further purification.

LCMS (Method 1): Rt=1.09 min, ES⁺ m/z 335.1/337.1 [M+H]⁺.

### Step 4

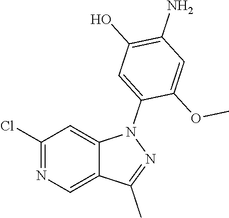 [SMILES: COc1cc(N)c(O)cc1-n1nc(C)c2cnc(Cl)cc21]

### 2-Amino-5-(6-chloro-3-methyl-1H-pyrazolo[4,3-c]pyridin-1-yl)-4-methoxyphenol (Intermediate 35-4)

To a warm refluxing solution of intermediate 35-3 (500 mg, 1.49 mmol) in MeOH (30 mL), a solution of NH₄Cl (320 mg, 5.98 mmol) in water (5 mL) and iron (834 mg, 14.9 mmol) were added. RM was heated at reflux temperature for 2 days. After cooling to RT, RM was filtered through a pad of diacematous earth. Filtrate was evaporated in vacuo and the residue taken in DCM, washed with water. The aqueous phase was back-extracted with DCM (3×50 mL) and combined organic layers were washed with sat. aq. NaCl and passed through a phase separator. Organic phase was evaporated in vacuo to afford the desired product (126.9 mg) that was used in the next steps without further purification.

LCMS (Method 1): Rt=0.72 min, ES⁺ m/z 305.0/306.9 [M+H]⁺.

### Step 5

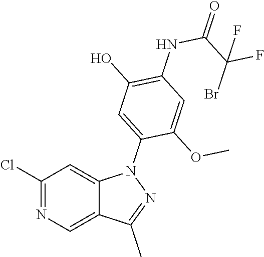 [SMILES: COc1cc(NC(=O)C(F)(F)Br)c(O)cc1-n1nc(C)c2cnc(Cl)cc21]

### 2-Bromo-N-(4-(6-chloro-3-methyl-1H-pyrazolo[4,3-c]pyridin-1-yl)-2-hydroxy-5-methoxyphenyl)-2,2-difluoroacetamide (Intermediate 35-5)

A solution of intermediate 35-4 (125 mg, 0.34 mmol), DABAL-Me₃ (131 mg, 0.51 mmol) and methyl 2-bromo-2,2-difluoro-acetate (71 mg, 0.37 mmol) in THF (3 mL) was stirred under nitrogen and microwave irradiation at 130° C. for 15 min. After cooling to RT, reaction was cautiously quenched with 4M HCl in 1,4-dioxane (2 mL). The resulting mixture was poured into sat. aq. NaHCO₃ and extracted with DCM. Organic phase was evaporated in vacuo and the residue purified by flash chromatography on Si cartridge by eluting with 0-90% DCM/MeOH (20:1) in DCM to afford the title product (25 mg).

LCMS (Method 1): Rt=1.09 min, ES⁺ m/z 461.0/463.1/465.0 [M+H].

### Step 6

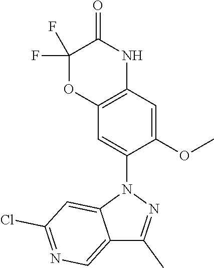 [SMILES: COc1cc2c(cc1-n1nc(C)c3cnc(Cl)cc31)OC(F)(F)C(=O)N2]

### 7-(6-Chloro-3-methyl-1H-pyrazolo[4,3-c]pyridin-1-yl)-2,2-difluoro-6-methoxy-2H-benzo[b][1,4]oxazin-3 (4H)-one (Intermediate 35)

A solution of intermediate 35-5 (24 mg, 52 μmol) and DBU (13.0 μL, 0.10 mmol) in THF (15 mL) was stirred at 110° C. for 16 h. After cooling to RT, RM was evaporated in vacuo. The residue was purified by flash chromatography on Si cartridge by eluting with 0-50% DCM/MeOH (20:1) in DCM to afford the title product (12.4 mg).

LCMS (Method 1): Rt=1.09 min, ES⁺ m/z 381.1/383.1 [M+H]⁺.

### Intermediate 36

### Step 1

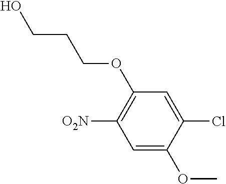 [SMILES: COc1cc([N+](=O)[O-])c(OCCCO)cc1Cl]

### 3-(5-Chloro-4-methoxy-2-nitrophenoxy)propan-1-ol (Intermediate 36-1)

5-Chloro-4-methoxy-2-nitro-phenol (1.00 g, 4.91 mmol), 3-iodopropan-1-ol (685 μL, 7.37 mmol) and K₂CO₃ (2.04 mg, 14.7 mmol) were suspended in DMF (15 mL) and RM stirred at 70° C. for 5 h. After cooling to RT, RM was diluted with water (30 mL) and extracted with EtOAc (4×20 mL). Combined organic layers were washed with water (40 mL) and sat. aq. NaCl (40 mL), and solvent removed in vacuo. The residue was purified by flash chromatography on Si cartridge by eluting with 0-40% DCM/MeOH (99:1) in DCM to afford the title product (1.08 g).

LCMS (Method 1): Rt=0.93 min, ES⁺ m/z 262.1 [M+H]⁺.

### Step 2

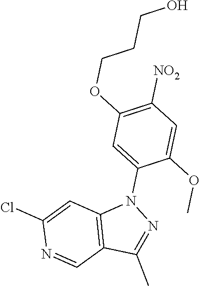 [SMILES: COc1cc([N+](=O)[O-])c(OCCCO)cc1-n1nc(C)c2cnc(Cl)cc21]

### 3-(5-(6-chloro-3-methyl-1H-pyrazolo[4,3-c]pyridin-1-yl)-4-methoxy-2-nitrophenoxy)propan-1-ol (Intermediate 36-2)

A suspension of intermediate 36-1 (1.01 g, 3.86 mmol), 6-chloro-3-methyl-1H-pyrazolo[4,3-c]pyridine (610 mg, 3.64 mmol), and K₂CO₃ (1.51 g, 10.9 mmol) in DMSO (10.0 mL) was stirred at 120° C. for 5 h. After cooling to RT, RM was diluted with water. The formed precipitate was collected by filtration, washed with water, and dried to afford the title product (700 mg) that was used in the next synthetic steps without further purification.

LCMS (Method 1): Rt=1.00 min, ES⁺ m/z 393.2/395.2 [M+H]⁺.

### Step 3

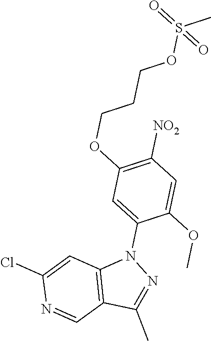 [SMILES: COc1cc([N+](=O)[O-])c(OCCCOS(C)(=O)=O)cc1-n1nc(C)c2cnc(Cl)cc21]

### 3-(5-(6-Chloro-3-methyl-1H-pyrazolo[4,3-c]pyridin-1-yl)-4-methoxy-2-nitrophenoxy)propyl methanesulfonate (Intermediate 36-3)

To a 0° C. cooled solution of intermediate 36-2 (200 mg, 0.51 mmol) in DMF (5 mL), methanesulfonyl chloride (59 μL, 0.76 mmol) was added dropwise and RM stirred at RT for 2 h. RM was poured into water (20 mL) and the formed precipitate collected by filtration, washed with water, and dried to afford the title product (225 mg) that was used in the next synthetic steps without further purification.

LCMS (Method 1): Rt=1.11 min, ES⁺ m/z 471.1/473.1 [M+H]⁺.

Step 4

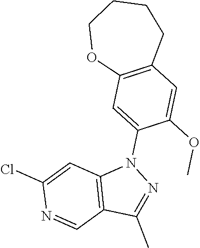 [SMILES: COc1cc2c(cc1-n1nc(C)c3cnc(Cl)cc31)OCCCC2]

### 8-(6-Chloro-3-methyl-1H-pyrazolo[4,3-c]pyridin-1-yl)-7-methoxy-2,3,4,5-tetrahydrobenzo[b][1,4]oxazepine (Intermediate 36)

To a stirring solution of intermediate 36-3 (210 mg, 0.42 mmol) in ethanol (10 mL) at 80° C., a solution of NH₄Cl (91 mg, 1.7 mmol) in water (5 mL) and Fe (0.28 g, 5.10 mmol) were added. RM was stirred at 80° C. overnight. After cooling to RT, RM was diluted with EtOAc (20 mL), filtered through a bed of diacematous earth and washed thoroughly with EtOAc. Filtrate was concentrated in vacuo. The residue was purified by flash chromatography on Si cartridge by eluting with 0-50% DCM/MeOH (95:5) in DCM to afford the title product (65 mg).

LCMS (Method 1): Rt=0.99 min, ES⁺ m/z 345.2 [M+H]⁺.

### Intermediate 37a

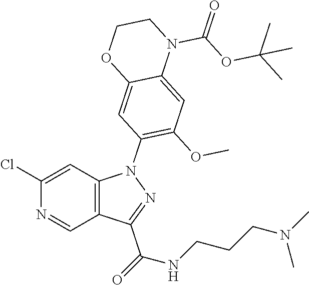 [SMILES: COc1cc2c(cc1-n1nc(C(=O)NCCCN(C)C)c3cnc(Cl)cc31)OCCN2C(=O)OC(C)(C)C]

### tert-Butyl 7-(6-chloro-3-((3-(dimethylamino)propyl)carbamoyl)-1H-pyrazolo[4,3-c]pyridin-1-yl)-6-methoxy-2,3-dihydro-4H-benzo[b][1,4]oxazine-4-carboxylate (Intermediate 37a)

To a solution of intermediate 24 (100 mg, 0.22 mmol), DIPEA (76 μL, 0.43 mmol) and HATU (91 mg, 0.26 mmol) in DMF (2.0 mL), N′,N′-dimethylpropane-1,3-diamine (33 μL, 0.26 mmol) was added and RM stirred at RT for 1 h. RM was diluted with EtOAc (20 mL) and washed with sat. NaHCO₃ (4×4 mL), and sat. aq. NaCl (10 mL). Combined organic layers were concentrated in vacuo. The residue was purified by flash chromatography on Si cartridge by eluting with 0-85% DCM/MeOH/NH₄OH (95:9:1.5) in DCM to afford the title product (63.6 mg).

LCMS (Method 1): Rt=0.92 min, ES⁺ m/z 545.4/547.3 [M+H]⁺.

### Intermediates 37b to 37k

The following intermediates were prepared in a similar manner to intermediate 37a from the indicated starting materials.

| | | Starting | |
|---|---|---|---|
| Intermediate | Structure | materials | LCMS |
| Intermediate 37b | 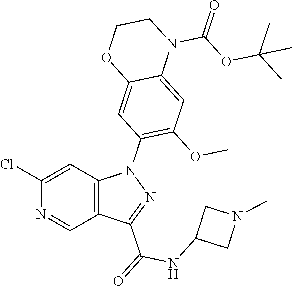 [SMILES: COc1cc2c(cc1-n1nc(C(=O)NC3CN(C)C3)c3cnc(Cl)cc31)OCCN2C(=O)OC(C)(C)C] | Intermediate 24 and 1- methylazetidin- 3-amine dihydrochloride | (Method 1): Rt = 0.93 min, ES⁺ m/z 529.3/531.3 [M + H]⁺ |
| Intermediate 37c | 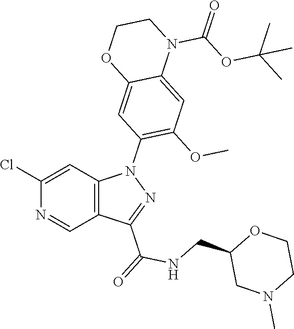 [SMILES: COc1cc2c(cc1-n1nc(C(=O)NC[C@@H]3CN(C)CCO3)c3cnc(Cl)cc31)OCCN2C(=O)OC(C)(C)C] | Intermediate 24 and [(2R)-4- methylmorpholin- 2- yl]methanamine | (Method 1): Rt = 0.93 min, ES⁺ m/z 573.4/575.4 [M + H]⁺ |
| Intermediate 37d | 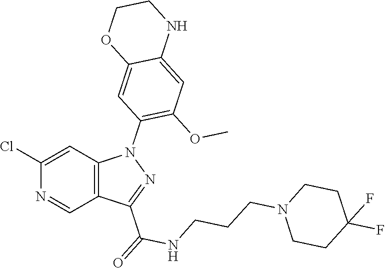 [SMILES: COc1cc2c(cc1-n1nc(C(=O)NCCCN3CCC(F)(F)CC3)c3cnc(Cl)cc31)OCCN2] | Intermediate 25 and 3-(4,4- difluoropiperidin- 1-yl)propan-1- amine | (Method 2): Rt = 1.08 min, ES⁺ m/z 521.3/523.3 [M + H]⁺ |
| Intermediate 37e | 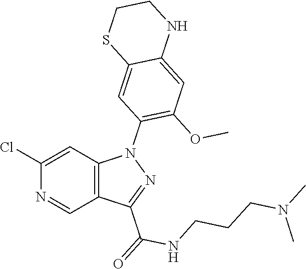 [SMILES: COc1cc2c(cc1-n1nc(C(=O)NCCCN(C)C)c3cnc(Cl)cc31)SCCN2] | Intermediate 21 and N′,N′- dimethylpropane- 1,3-diamine | (Method 1): Rt = 0.81 min, ES⁺ m/z 461.1/463.1 [M + H]⁺ |
| Intermediate 37f | 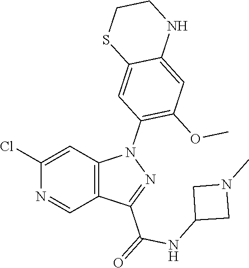 [SMILES: COc1cc2c(cc1-n1nc(C(=O)NC3CN(C)C3)c3cnc(Cl)cc31)SCCN2] | Intermediate 21 and 1- methylazetidin- 3-amine dihydrochloride | (Method 2): Rt = 0.79 min, ES⁺ m/z 445.1/447.0 [M + H]⁺ |
| Intermediate 37g | 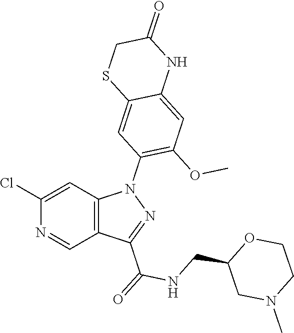 [SMILES: COc1cc2c(cc1-n1nc(C(=O)NC[C@@H]3CN(C)CCO3)c3cnc(Cl)cc31)SCC(=O)N2] | Intermediate 22 and (R)-(4- methylmorpholin- 2- yl)methanamine | (Method 2): Rt = 0.86 min, ES⁺ m/z 503.2/505.1 [M + H]⁺ |
| Intermediate 37h | 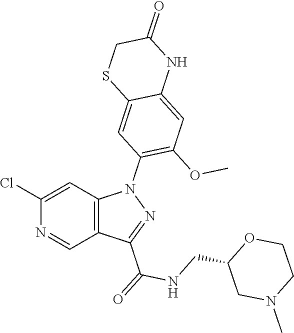 [SMILES: COc1cc2c(cc1-n1nc(C(=O)NC[C@H]3CN(C)CCO3)c3cnc(Cl)cc31)SCC(=O)N2] | Intermediate 22 and (S)-(4- methylmorpholin- 2- yl)methanamine | (Method 2): Rt = 0.86 min, ES⁺ m/z 503.2/505.1 [M + H]⁺ |
| Intermediate 37i | 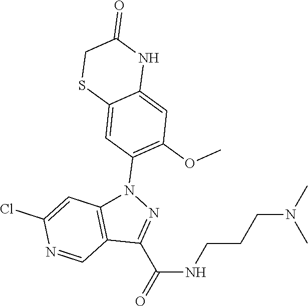 | Intermediate 22 and N′,N′- dimethylpropane- 1,3-diamine | (Method 1): Rt = 0.81 min, ES⁺ m/z 461.1/463.1 [M + H]⁺ |
| Intermediate 37j | 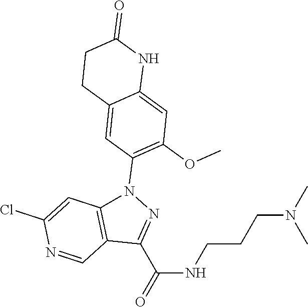 [SMILES: COc1cc2c(cc1-n1nc(C(=O)NCCCN(C)C)c3cnc(Cl)cc31)CCC(=O)N2] | Intermediate 26 and N′,N′- dimethylpropane- 1,3-diamine | (Method 1): Rt = 0.65 min, ES⁺ m/z 457.2/459.1 [M + H]⁺ |
| Intermediate 37k | 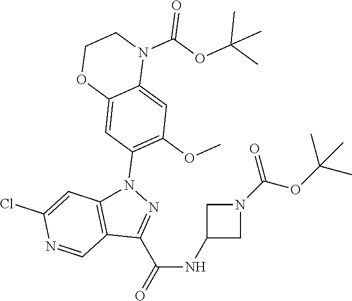 [SMILES: COc1cc2c(cc1-n1nc(C(=O)NC3CN(C(=O)OC(C)(C)C)C3)c3cnc(Cl)cc31)OCCN2C(=O)OC(C)(C)C] | Intermediate 24 and tert-butyl 3- aminoazetidine- 1-carboxylate | (Method 1): Rt = 1.43 min, ES⁺ m/z 615.3/617.3 [M + H]⁺ |
| Intermediate 37l | 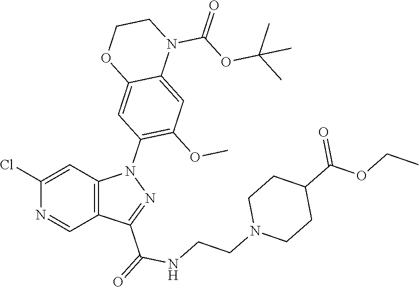 [SMILES: CCOC(=O)C1CCN(CCNC(=O)c2nn(-c3cc4c(cc3OC)N(C(=O)OC(C)(C)C)CCO4)c3cc(Cl)ncc23)CC1] | Intermediate 24 and Intermediate 15 | (Method 1): Rt = 1.02 min, ES⁺ m/z 643.4/645.4 [M + H]⁺ |

### Intermediate 37m

### Step 1

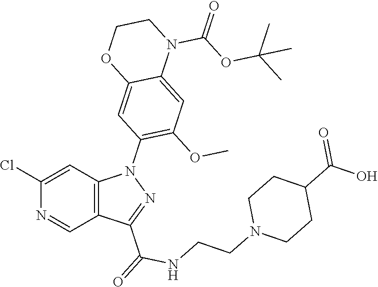 [SMILES: COc1cc2c(cc1-n1nc(C(=O)NCCN3CCC(C(=O)O)CC3)c3cnc(Cl)cc31)OCCN2C(=O)OC(C)(C)C]

### 1-(2-(L-(4-(tert-butoxycarbonyl)-6-ethoxy-3,4-dihydro-2H-benzo[b][1,4]oxazin-7-yl)-6-chloro-1H-pyrazolo[4,3-c]pyridine-3-carboxamido)ethyl)piperidine-4-carboxylic acid (Intermediate 37m-1)

To a solution of intermediate 371 (1.0 g, 1.48 mmol) in THF (10 mL), an aq. solution 1M of LiOH (10.6 mL, 11.8 mmol) was added and RMV stirred at RT for 16 h. RMV was evaporated in vacuo and the residue taken with water and the pH adjusted to 6.5 using aq. 1M HCl. The aqueous phase was extracted with DCM (3×10 mL), then combined organic layers were evaporated to dryness in vacuo to afford the title product (756 mg).

LCMS (Method 1): Rt=0.99 min, ES⁺ m/z 615.1/617.1 [M+H]⁺

### Step 2

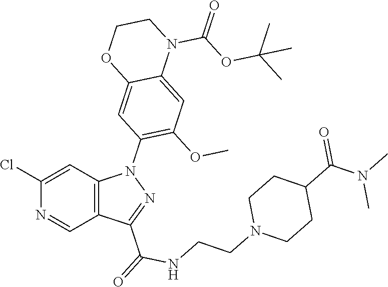 [SMILES: COc1cc2c(cc1-n1nc(C(=O)NCCN3CCC(C(=O)N(C)C)CC3)c3cnc(Cl)cc31)OCCN2C(=O)OC(C)(C)C]

### tert-butyl 7-(6-chloro-3-((2-(4-(dimethylcarbamoyl)piperidin-1-yl)ethyl)carbamoyl)-1H-pyrazolo[4,3-c]pyridin-1-yl)-6-methoxy-2,3-dihydro-4H-benzo[b][1,4]oxazine-4-carboxylate (Intermediate 37m)

A mixture of intermediate 37m-1 (34.0 mg, 55.3 μmol), dimethylamine hydrochloride (9.11 mg, 111 μmol), HATU (25.2 mg, 66.3 μmol) and DIPEA (48.1 μL, 0.28 mmol) in DMF (1 mL) was stirred at RT for 1.5 h. RM was quenched with water (20 mL) and extracted twice with EtOAc. Combined organic layers were washed with sat. aq. NaCl (20 mL) and solvent removed in vacuo. The residue was purified by flash chromatography on Si cartridge by eluting with 0-70% DCM/MeOH/NH₄OH (90:5:0.5) in DCM to afford the title product (26 mg).

LCMS (Method 2): Rt=0.95 min, ES⁺ m/z 642.4/644.4 [M+H]⁺

### Intermediate 37n

### Step 1

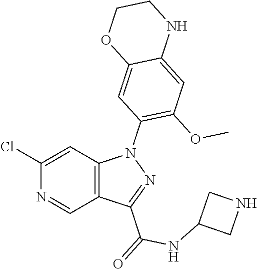 [SMILES: COc1cc2c(cc1-n1nc(C(=O)NC3CNC3)c3cnc(Cl)cc31)OCCN2]

### N-(azetidin-3-yl)-6-chloro-1-(6-methoxy-3,4-dihydro-2H-benzo[b][1,4]oxazin-7-yl)-1H-pyrazolo[4,3-c]pyridine-3-carboxamide (Intermediate 37n-1)

A solution of intermediate 37k (100.0 mg, 0.16 mmol) and TFA (362 μL, 4.88 mmol) in DCM (4 mL) was stirred at RT for 4.5 h. RM was evaporated in vacuo. The residue was dissolved in MeOH and passed through a on a SCX cartridge, washed with methanol and eluted with methanolic ammonia (7M) to afford the title product (63 mg).

LCMS (Method 2): Rt=0.68 min. ES⁺ m/z 415.2/4.17.2 [M+H]⁺

### Step 2

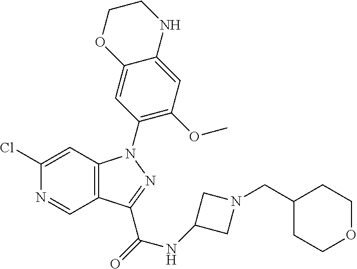 [SMILES: COc1cc2c(cc1-n1nc(C(=O)NC3CN(CC4CCOCC4)C3)c3cnc(Cl)cc31)OCCN2]

### 6-Chloro-1-(6-methoxy-3,4-dihydro-2H-benzo[b][1,4]oxazin-7-yl)-N-(1-((tetrahydro-2H-pyran-4-yl)methyl)azetidin-3-yl)-1H-pyrazolo[4,3-c]pyridine-3-carboxamide (Intermediate 37n)

A mixture of intermediate 37n-1 (50 mg, 0.12 mmol), 4-(bromomethyl)tetrahydropyran (33.3 mg, 0.13 mmol) and K₂CO₃ (33.3 mg, 0.24 mmol) in DMF (1.5 mmol) was stirred at 80° C. for 7 h. A second equivalent of 4-(bromomethyl)tetrahydropyran was added and RM further stirred for 48 hours. After cooling to RT, reaction was quenched with water (10 mL) and extracted with EtOAc (2×15 mL). Combined organic layers were washed with sat. aq. NaCl (10 mL) and evaporated in vacuo. The residue was purified by flash chromatography on Si cartridge by eluting with 0-15% DCM/MeOH/NH₄OH (90:9:0.5) in DCM to afford the title product (48 mg).

LCMS (Method 2): Rt=0.72, ES' m/z 557.2/559.2.

### Intermediate 370

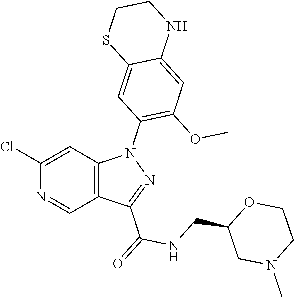 [SMILES: COc1cc2c(cc1-n1nc(C(=O)NC[C@@H]3CN(C)CCO3)c3cnc(Cl)cc31)SCCN2]

### (R)-6-chloro-1-(6-methoxy-3,4-dihydro-2H-benzo[b][1,4]thiazin-7-yl)-N-((4-methylmorpholin-2-yl)methyl)-1H-pyrazolo[4,3-c]pyridine-3-carboxamide (Intermediate 37o)

A mixture of intermediate 21-5 (30 mg, 77 μmol), DABAL-Me₃ (30 mg, 0.12 mmol), THF (1 mL) and [(2R)-4-methylmorpholin-2-yl]methanamine (15 mg, 0.12 mmol) was heated under argon atmosphere, at 130° C. for 45 min, under μW irradiation. After cooling to RT, RM was quenched with 4M HCl in dioxane (2 mL) and poured into sat. aq. NaHCO₃. The mixture was extracted with DCM and combined organic layers evaporated in vacuo. The residue was purified by flash chromatography on Si cartridge by eluting with 0-50% DCM/MeOH (9:1) in DCM to afford the title product (26 mg).

LCMS (Method 1): Rt=0.81 min, ES⁺ m/z 489.2/491.2 [M+H]⁺

### Intermediate 37p

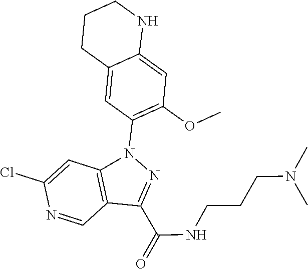 [SMILES: COc1cc2c(cc1-n1nc(C(=O)NCCCN(C)C)c3cnc(Cl)cc31)CCCN2]

### 6-Chloro-N-(3-(dimethylamino)propyl)-1-(7-methoxy-1,2,3,4-tetrahydroquinolin-6-yl)-1H-pyrazolo[4,3-c]pyridine-3-carboxamide (Intermediate 37p)

The title compound was prepared on a similar manner to intermediate 37o starting from intermediate 27b and N′,N′-dimethylpropane-1,3-diamine.

LCMS (Method 1): Rt=0.77 min, ES⁺ m/z 443.0/445.0 [M+H]⁺.

### Intermediate 37p

### Step 1

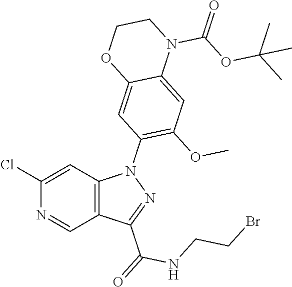 [SMILES: COc1cc2c(cc1-n1nc(C(=O)NCCBr)c3cnc(Cl)cc31)OCCN2C(=O)OC(C)(C)C]

### tert-Butyl 7-(3-((2-bromoethyl)carbamoyl)-6-chloro-1H-pyrazolo[4,3-c]96yridine-1-yl)-6-methoxy-2,3-dihydro-4H-benzo[b][1,4]oxazine-4-carboxylate (Intermediate 37 g-1)

Intermediate 37q-1 was prepared in a similar manner to intermediate 37a starting from intermediate 24 and 2-bromoethylamine hydrochloride.

LCMS (Method 2): Rt=1.38 min, ES⁺ m/z 566.6/568.1/570.1 [M+H]⁺

### Step 2

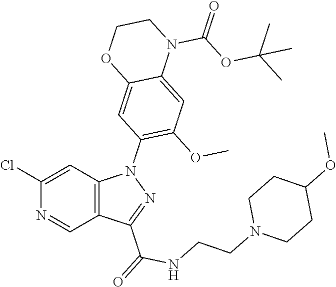 [SMILES: COc1cc2c(cc1-n1nc(C(=O)NCCN3CCC(OC)CC3)c3cnc(Cl)cc31)OCCN2C(=O)OC(C)(C)C]

### tert-Butyl 7-(6-chloro-3-((2-(4-methoxypiperidine-1-yl)ethyl)carbamoyl)-1H-pyrazolo[4,3-c]97yridine-1-yl)-6-methoxy-2,3-dihydro-4H-benzo[b][1,4]oxazine-4-carboxylate (Intermediate 373)

A solution o of intermediate 37q-1 (80.0 mg, 0.14 mmol), 4-methoxypiperidine (19.5 mg, 0.17 mmol) and DIPEA (98.3 μL, 0.57 mmol) in dry DMF (1.3 mL) was stirred under argon, overnight at RT. RMV was quenched by the addition of sat. aq. NH₄Cl, then extracted with EtOAc (×2). Combined organic layers were washed with water and sat. aq. NaCl, dried over Na₂SO₄ and evaporated to dryness. The residue was purified by flash chromatography on Si cartridge by eluting with DCM/MeOH mixture (from 20:1 to 10:1) to afford the title product (50.0 mg).

LCMS (Method 1): Rt=0.95 min, ES⁺ m/z 601.0/603.2 [M+H]⁺

### Intermediates 37q to 37w

The following intermediates were prepared in a similar manner to Intermediate 37q from the indicated starting materials.

| Intermediate | Structure | Starting materials | LCMS |
|---|---|---|---|
| Intermediate 37r | 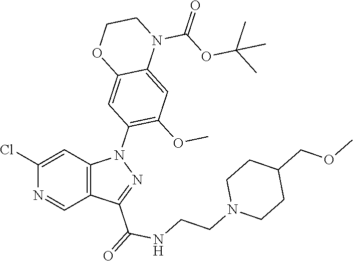 [SMILES: COCC1CCN(CCNC(=O)c2nn(-c3cc4c(cc3OC)N(C(=O)OC(C)(C)C)CCO4)c3cc(Cl)ncc23)CC1] | Intermediate 37q- 1 and 4-(methoxy- methyl)piperidine | (Method 1): Rt = 0.95 min, ES⁺ m/z 615.3/617.3 [M + H]⁺ |
| Intermediate 37s | 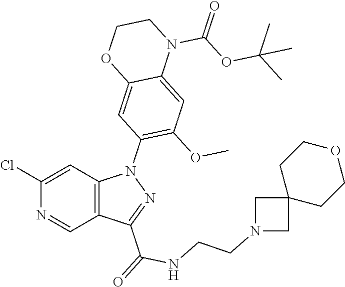 [SMILES: COc1cc2c(cc1-n1nc(C(=O)NCCN3CC4(CCOCC4)C3)c3cnc(Cl)cc31)OCCN2C(=O)OC(C)(C)C] | Intermediate 37q- 1 and 7-oxa-2-aza- spiro[3.5]nonane | (Method 1): Rt = 0.94 min, ES⁺ m/z 613.4/615.3 [M + H]⁺ |
| Intermediate 37t | 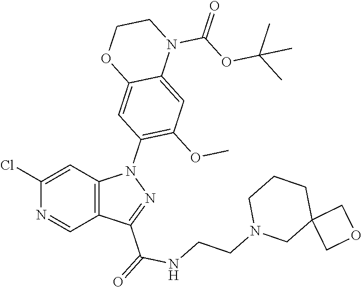 [SMILES: COc1cc2c(cc1-n1nc(C(=O)NCCN3CCCC4(COC4)C3)c3cnc(Cl)cc31)OCCN2C(=O)OC(C)(C)C] | Intermediate 37q- 1 and 2-oxa-8-aza- spiro[3.5]nonane | (Method 1): Rt = 0.94 min, ES⁺ m/z 613.4/615.3 [M + H]⁺ |
| Intermediate 37u | 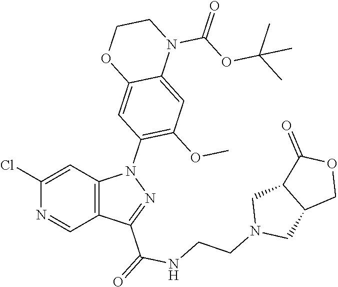 [SMILES: COc1cc2c(cc1-n1nc(C(=O)NCCN3C[C@H]4COC(=O)[C@H]4C3)c3cnc(Cl)cc31)OCCN2C(=O)OC(C)(C)C] | Intermediate 37q- 1 and (3aR,6aS)- 1,3a,4,5,6,6a-hexa- hydrofuro[3,4-c] pyrrol-3-one hydrochloride | (Method 1): Rt = 0.94 min, ES⁺ m/z 613.4/615.3 [M + H]⁺ |
| Intermediate 37v | 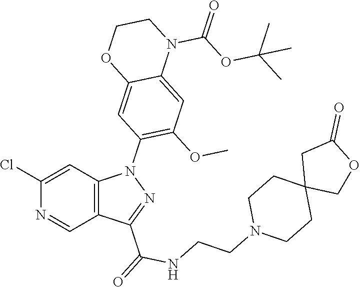 [SMILES: COc1cc2c(cc1-n1nc(C(=O)NCCN3CCC4(CC3)COC(=O)C4)c3cnc(Cl)cc31)OCCN2C(=O)OC(C)(C)C] | Intermediate 37q- 1 and 2-oxa-8-aza- spiro[4.5]decan-3- one hydrochloride | (Method 1): Rt = 0.98 min, ES⁺ m/z 641.4/643.4 [M + H]⁺ |
| Intermediate 37w | 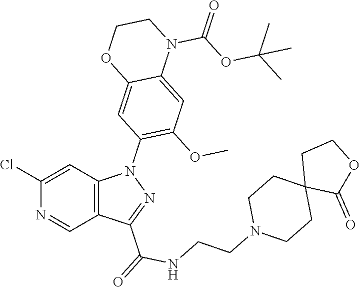 [SMILES: COc1cc2c(cc1-n1nc(C(=O)NCCN3CCC4(CCOC4=O)CC3)c3cnc(Cl)cc31)OCCN2C(=O)OC(C)(C)C] | Intermediate 37q- 1 and 2-oxa-8-aza- spiro[4.5]decan-1- one hydrochloride | (Method 1): Rt = 0.99 min, ES⁺ m/z 641.4/643.4 [M + H]⁺ |

### Intermediate 37x

### Step 1

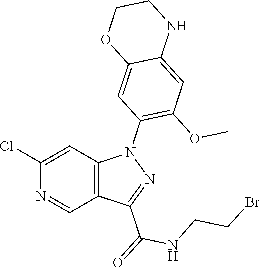 [SMILES: COc1cc2c(cc1-n1nc(C(=O)NCCBr)c3cnc(Cl)cc31)OCCN2]

### N-(2-bromoethyl)-6-chloro-1-(6-methoxy-3,4-dihydro-2H-benzo[b][1,4]oxazin-7-3yl)-1H-pyrazolo[4,3-c]pyridine-3-carboxamide (Intermediate 37x-1)

A solution of intermediate 37q-1 (215.0 mg, 0.37 mmol) and TFA (1.38 mL, 18.6 mol) in DCM (2.0 mL) was stirred at 16° C. for 16 h. RM was neutralized with sat. aq. NaHCO₃ (20 mL) and layers were separated. Organic phase was washed with sat. aq. NaHCO₃, passed through a phase separator and evaporated in vacuo to afford the desired product (223.6 mg) that was used in the next synthetic steps without further purification.

LCMS (Method 1): Rt=1.07 min, ES⁺ m/z 466.1/468.1/470.0 [M+H]⁺

### Step 2

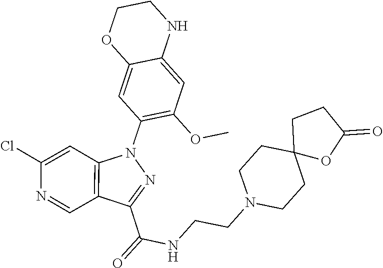 [SMILES: COc1cc2c(cc1-n1nc(C(=O)NCCN3CCC4(CCC(=O)O4)CC3)c3cnc(Cl)cc31)OCCN2]

### 6-Chloro-1-(6-methoxy-3,4-dihydro-2H-benzo[b][1,4]oxazin-7-yl)-N-(2-(2-oxo-1-oxa-8-azaspiro[4.5]decan-8-yl)ethyl)-1H-pyrazolo[4,3-c]pyridine-3-carboxamide (Intermediate 37x)

To a mixture of intermediate 37x-1 (40.0 mg, 81 μmol) in acetone (0.5 mL), 1-oxa-8-azaspiro[4.5]decan-2-one hydrochloride (49 mg, 240 μmol), Na₂CO₃ (8.6 mg, 81 μmol) and NaI (1.3 mg, 8.1 μmol) in acetone (1.0 mL) were added and RM stirred at 40° C. for 24 h. RM was evaporated in vacuo and the residue partitioned between water (10 mL) and DCM (10 mL). After additional extraction of aqueous phase with DCM (3×10 mL), combined organic layers were passed through a phase separator and solvent evaporated in vacuo. The residue was purified by flash chromatography on Si cartridge by eluting with 0-50% DCM/MeOH/NH₄OH (90:5:0.5) in DCM to afford the title product (22 mg).

LCMS (Method 1): Rt=0.71 min, ES⁺ m/z 541.1/543.1 [M+H]⁺

### Intermediate 38a

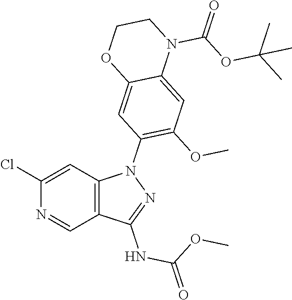 [SMILES: COC(=O)Nc1nn(-c2cc3c(cc2OC)N(C(=O)OC(C)(C)C)CCO3)c2cc(Cl)ncc12]

### tert-Butyl 7-(6-chloro-3-((methoxycarbonyl)amino)-1H-pyrazolo[4,3-c]pyridin-1-yl)-6-methoxy-2,3-dihydro-4H-benzo[b][1,4]oxazine-4-carboxylate (Intermediate 38a)

To a 0° C. cooled solution of intermediate 28 (60 mg, 0.14 mmol) in DCM (0.9 mL), pyridine (15 μL, 0.18 mmol) was added followed by methyl chloroformate (13 μL, 0.17 mmol). RM was stirred at 0° C. for 30 min and at RT for 1 h. RM was quenched with sat aq. NaHCO₃ and extracted with DCM (4×15 mL). Combined organic layers were washed with sat. aq. NaHCO₃ and sat. aq. NaCl, dried over Na₂SO₄ and evaporated in vacuo. The residue was purified by flash chromatography on Si cartridge by eluting with 0-50% DCM/MeOH/NH₄OH (90:9:1.5) in DCM to afford the title product (30.6 mg).

LCMS (Method 1): Rt=1.27 min, ES⁺ m/z 490.1/492.0 [M+H]⁺

### Intermediate 38b

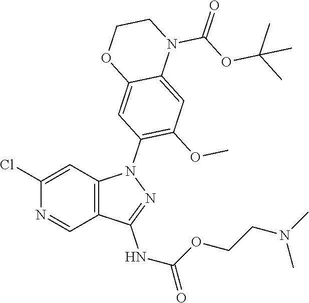 [SMILES: COc1cc2c(cc1-n1nc(NC(=O)OCCN(C)C)c3cnc(Cl)cc31)OCCN2C(=O)OC(C)(C)C]

### tert-Butyl 7-(6-chloro-3-(((2-(dimethylamino)ethoxy)carbonyl)amino)-1H-pyrazolo[4,3-c]pyridin-1-yl)-6-methoxy-2,3-dihydro-4H-benzo[b][1,4]oxazine-4-carboxylate (Intermediate 38b)

To a −78° C. cooled solution of intermediate 28 (250 mg, 0.58 mmol) and TEA (242 μL, 1.74 mmol) in DCM (15 mL), bis(trichloromethyl) carbonate (172 mg, 0.58 mmol) was added in one portion. RM was stirred at −78° C. for 1 h, followed by the addition of a solution of 2-(dimethylamino)ethanol (293 μL, 2.89 mmol) and TEA (161 μL, 1.16 mmol) in DCM (7.5 mL). RM was stirred at −78° C. for further 1 h, then allowed to warm to RT and quenched with sat. aq. NaHCO₃. Aqueous phase was further extracted with DCM (3×25 mL) and combined organic layers were dried over Na₂SO₄ and evaporated in vacuo. The residue was purified by flash chromatography on Si cartridge by eluting with 0-100% DCM/MeOH/NH₄OH (90:9:1) in DCM to afford the title product (310 mg).

LCMS (Method 2): Rt=1.23 min, ES⁺ m/z 547.3/549.2 [M+H]⁺

### Intermediates 38c to 38k

The following intermediates were prepared in a similar manner to intermediate 38b from the indicated starting materials. It is additionally stated if base, solvent or temperature were varied.

| Intermediate | Structure | Starting materials | LCMS |
|---|---|---|---|
| Intermediate 38c | 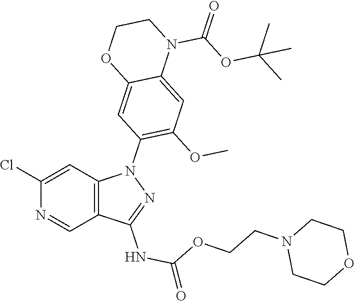 [SMILES: COc1cc2c(cc1-n1nc(NC(=O)OCCN3CCOCC3)c3cnc(Cl)cc31)OCCN2C(=O)OC(C)(C)C] | Intermediate 28 and 2-morpholino- ethanol dissolved in DCM/DMF | (Method 2): Rt = 1.21 min, ES⁺ m/z 589.3/591.3 [M + H]⁺ |
| Intermediate 38d | 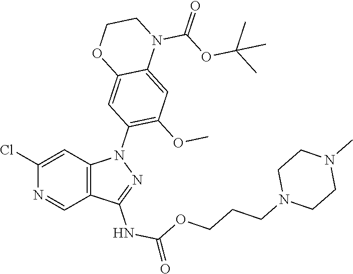 [SMILES: COc1cc2c(cc1-n1nc(NC(=O)OCCCN3CCN(C)CC3)c3cnc(Cl)cc31)OCCN2C(=O)OC(C)(C)C] | Intermediate 28 and 3-(4-methyl- piperazin-1-yl)- propan-1-ol | (Method 2): Rt = 1.18 min, ES⁺ m/z 616.3/618.3 [M + H]⁺ |
| Intermediate 38e | 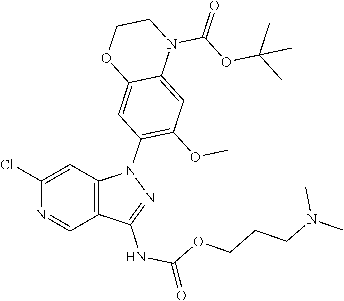 [SMILES: COc1cc2c(cc1-n1nc(NC(=O)OCCCN(C)C)c3cnc(Cl)cc31)OCCN2C(=O)OC(C)(C)C] | Intermediate 28 and 3-(dimethyl- amino)propan-1- ol dissolved in DCM/DMF | (Method 2): Rt = 1.27 min, ES⁺ m/z 561.4/563.3 [M + H]⁺ |
| Intermediate 38f | 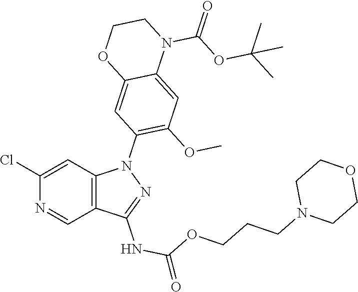 [SMILES: COc1cc2c(cc1-n1nc(NC(=O)OCCCN3CCOCC3)c3cnc(Cl)cc31)OCCN2C(=O)OC(C)(C)C] | Intermediate 28 and 3-morpholino- propan-1-ol | (Method 2): Rt = 1.21 min, ES⁺ m/z 601.4/603.4 [M + H]⁺ |
| Intermediate 38g | 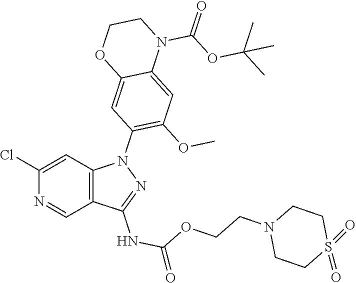 [SMILES: COc1cc2c(cc1-n1nc(NC(=O)OCCN3CCS(=O)(=O)CC3)c3cnc(Cl)cc31)OCCN2C(=O)OC(C)(C)C] | Intermediate 28 and 2-(1,1-dioxo- 1,4-thiazinan-4- yl)ethanol | (Method 2): Rt = 1.19 min, ES⁺ m/z 637.3/369.3 [M + H]⁺ |
| Intermediate 38h | 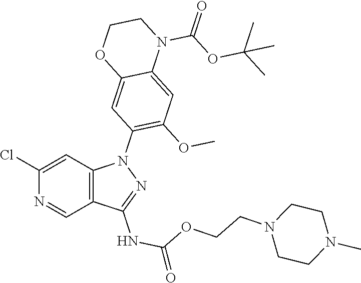 [SMILES: COc1cc2c(cc1-n1nc(NC(=O)OCCN3CCN(C)CC3)c3cnc(Cl)cc31)OCCN2C(=O)OC(C)(C)C] | Intermediate 28 and 2-(4-methyl- piperazin-1-yl)- ethanol | (Method 2): Rt = 1.18 min, ES⁺ m/z 602.3/604.2 [M + H]⁺ |
| Intermediate 38i | 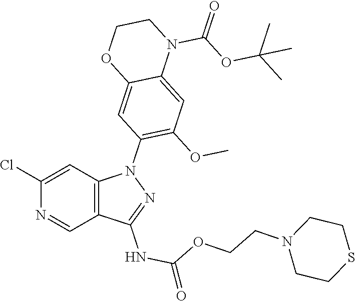 [SMILES: COc1cc2c(cc1-n1nc(NC(=O)OCCN3CCSCC3)c3cnc(Cl)cc31)OCCN2C(=O)OC(C)(C)C] | Intermediate 28 and 2-thiomor- pholinoethanol | (Method 2): Rt = 1.31 min, ES⁺ m/z 605.3/607.2 [M + H]⁺ |
| Intermediate 38j | 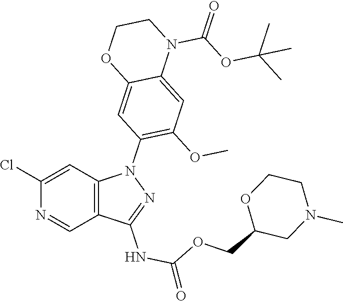 [SMILES: COc1cc2c(cc1-n1nc(NC(=O)OC[C@@H]3CN(C)CCO3)c3cnc(Cl)cc31)OCCN2C(=O)OC(C)(C)C] | Intermediate 28 and [(2S)-4-meth- ylmorpholin-2-yl] methanol | (Method 2): Rt = 1.25 min, ES⁺ m/z 589.3/591.2 [M + H]⁺ |
| Intermediate 38k | 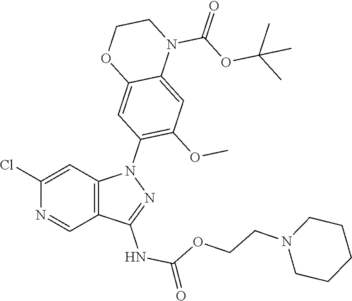 | Intermediate 28 and 2-(1-piperid- yl)ethanol | (Method 2): Rt = 1.14 min, ES⁺ m/z 475.1/477.1 [M + H]⁺ |

### Intermediate 38l

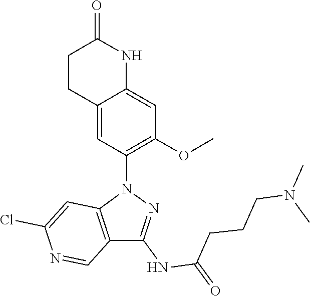 [SMILES: COc1cc2c(cc1-n1nc(NC(=O)CCCN(C)C)c3cnc(Cl)cc31)CCC(=O)N2]

### 2-(Dimethylamino)ethyl (6-chloro-1-(7-methoxy-2-oxo-1,2,3,4-tetrahydroquinolin-6-yl)-1H-pyrazolo[4,3-c]pyridin-3-yl)carbamate (Intermediate 38l)

A solution of CDI (75 mg, 0.47 mmol), intermediate 29 (40 mg, 0.12 mmol) and imidazole (24 mg, 0.35 mmol) in 2-Me-THF (1.3 mL) was stirred under argon at 90° C. overnight. 2-(dimethylamino)ethanol (35 μL, 0.35 mmol) was added and RM stirred at 90° C. for further 5 h. After cooling to RT, RM was diluted with water and extracted with DCM/iPrOH (3×15 mL). Combined organic layers were washed with sat. aq. NaCl, dried over Na₂SO₄ and evaporated in vacuo. The residue was purified by flash chromatography on Si cartridge by eluting with 0-100% DCM/MeOH/NH₄OH (90:9:0.5) in DCM to afford the title product (19.5 mg).

LCMS (Method 2): Rt=0.94 min, ES⁺ m/z 459.1/461.1 [M+H]⁺

### Intermediate 38m

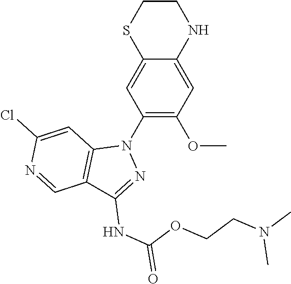 [SMILES: COc1cc2c(cc1-n1nc(NC(=O)OCCN(C)C)c3cnc(Cl)cc31)SCCN2]

### 2-(Dimethylamino)ethyl (6-chloro-1-(6-methoxy-3,4-dihydro-2H-benzo[b][1,4]thiazin-7-yl)-1H-pyrazolo[4,3-c]pyridin-3-yl)carbamate (Intermediate 38m)

A mixture of intermediate 21 (200 mg, 0.53 mmol), T3P® (50%, in EtOAc, 620 μL, 1.1 mmol), azido(trimethyl)silane (141 μL, 1.1 mmol) and TEA (222 μL, 1.6 mmol) in 2-Me THF (20 mL) was refluxed for 1 h. A solution of 2-(dimethylamino)ethanol (95 mg, 1.1 mmol) in 2-Me THF (1 mL) was added and RM refluxed for further 4 h. After cooling to RT, RM was diluted with EtOAc (35 mL), and washed with sat. aq. NaHCO₃ (3×10 mL) and sat. aq. NaCl (10 mL). Organic layer was dried over Na₂SO₄ and concentrated in vacuo. The residue was purified by flash chromatography on Si cartridge by eluting with 0-50% DCM/MeOH/NH₄OH (90:9:1.5) in DCM to afford the title product (116 mg).

LCMS (Method 1): Rt=0.75 min, ES⁺ m/z 463.2/465.2 [M+H]⁺

### Intermediates 38n to 38x

The following intermediates were prepared in a similar manner to intermediate 38m from the indicated starting materials.

| Intermediate | Structure | Starting materials | LCMS |
|---|---|---|---|
| Intermediate 38n | 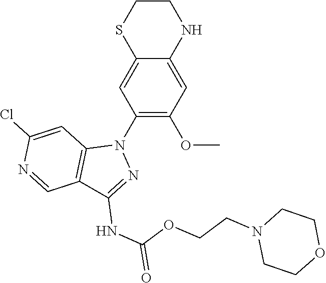 [SMILES: COc1cc2c(cc1-n1nc(NC(=O)OCCN3CCOCC3)c3cnc(Cl)cc31)SCCN2] | Intermediate 21 and 2-morpholino- ethanol | (Method 1): Rt = 0.76 min, ES⁺ m/z 505.2/507.2 [M + H]⁺ |
| Intermediate 38o | 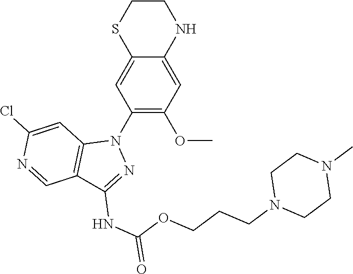 [SMILES: COc1cc2c(cc1-n1nc(NC(=O)OCCCN3CCN(C)CC3)c3cnc(Cl)cc31)SCCN2] | Intermediate 21 and 3-(4-methyl- piperazin-1-yl)- propan-1-ol | (Method 1): Rt = 0.81 min, ES⁺ m/z 532.2/534.2 [M + H]⁺ |
| Intermediate 38p | 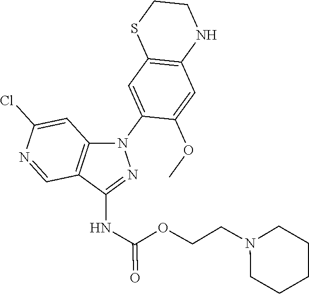 [SMILES: COc1cc2c(cc1-n1nc(NC(=O)OCCN3CCCCC3)c3cnc(Cl)cc31)SCCN2] | Intermediate 21 and 2-(1-piperid- yl)ethanol | (Method 1): Rt = 0.87 min, ES⁺ m/z 503.2/505.1 [M + H]⁺ |
| Intermediate 38q | 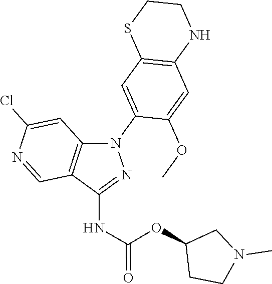 [SMILES: COc1cc2c(cc1-n1nc(NC(=O)O[C@@H]3CCN(C)C3)c3cnc(Cl)cc31)SCCN2] | Intermediate 21 and (R)-1-methyl- pyrrolidin-3-ol | (Method 1): Rt = 0.75 min, ES⁺ m/z 475.2/477.1 [M + H]⁺ |
| Intermediate 38r | 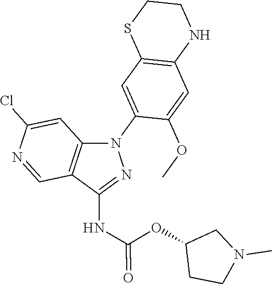 [SMILES: COc1cc2c(cc1-n1nc(NC(=O)O[C@H]3CCN(C)C3)c3cnc(Cl)cc31)SCCN2] | Intermediate 21 and (S)-1-methyl- pyrrolidin-3-ol | (Method 1): Rt = 0.75 min, ES⁺ m/z 475.2/477.1 [M + H]⁺ |
| Intermediate 38s | 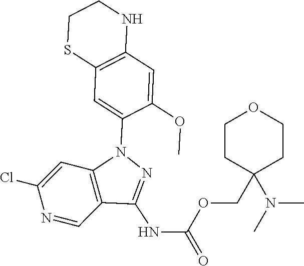 [SMILES: COc1cc2c(cc1-n1nc(NC(=O)OCC3(N(C)C)CCOCC3)c3cnc(Cl)cc31)SCCN2] | Intermediate 21 and [4-(dimethyl- amino)tetrahydro- pyran-4-yl]methan- ol hydrochloride | (Method 1): Rt = 0.78 min, ES⁺ m/z 533.1/534.9 [M + H]⁺ |
| Intermediate 38t | 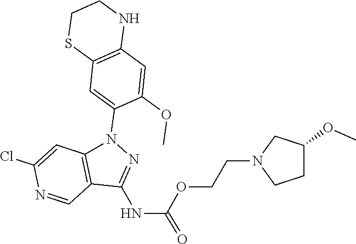 [SMILES: COc1cc2c(cc1-n1nc(NC(=O)OCCN3CC[C@@H](OC)C3)c3cnc(Cl)cc31)SCCN2] | Intermediate 21 and (R) 2-(3-meth- oxypyrrolidin-1- yl)ethanol | (Method 1): Rt = 0.79 min, ES⁺ m/z 519.1/521.1 [M + H]⁺ |
| Intermediate 38u | 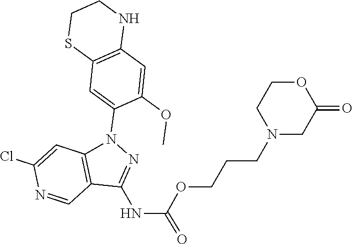 [SMILES: COc1cc2c(cc1-n1nc(NC(=O)OCCCN3CCOC(=O)C3)c3cnc(Cl)cc31)SCCN2] | Intermediate 21 and Intermediate 16 | (Method 1): Rt = 0.88 min, ES⁺ m/z 533.1/535.1 [M + H]⁺ |
| Intermediate 38v | 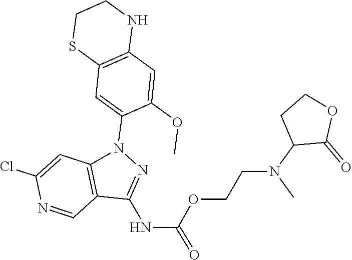 [SMILES: COc1cc2c(cc1-n1nc(NC(=O)OCCN(C)C3CCOC3=O)c3cnc(Cl)cc31)SCCN2] | Intermediate 21 and Intermediate 14 | (Method 1): Rt = 0.84 min, ES⁺ m/z 533.1/535.1 [M + H]⁺ |
| Intermediate 38w | 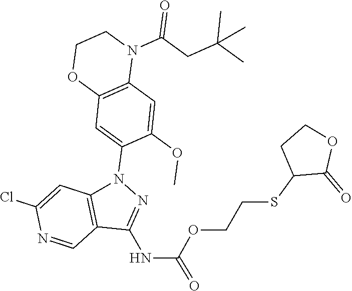 [SMILES: COc1cc2c(cc1-n1nc(NC(=O)OCCSC3CCOC3=O)c3cnc(Cl)cc31)OCCN2C(=O)CC(C)(C)C] | Intermediate 24 and Intermediate 13 | (Method 1): Rt = 1.27 min, ES⁺ m/z 620.1/622.0 [M + H]⁺ |
| Intermediate 38x | 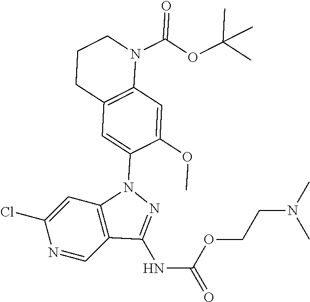 [SMILES: COc1cc2c(cc1-n1nc(NC(=O)OCCN(C)C)c3cnc(Cl)cc31)CCCN2C(=O)OC(C)(C)C] | Intermediate 27a and 2-(dimethyl- amino)ethanol | (Method 1): Rt = 0.95 min, ES⁺ m/z 545.1/547.1 [M + H]⁺ |

### Intermediate 38y

### Step 1

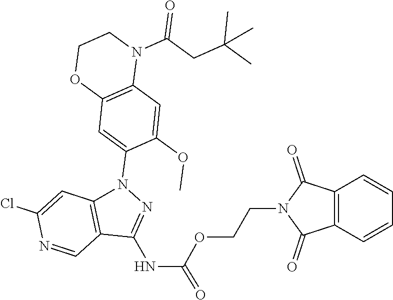 [SMILES: COc1cc2c(cc1-n1nc(NC(=O)OCCN3C(=O)c4ccccc4C3=O)c3cnc(Cl)cc31)OCCN2C(=O)CC(C)(C)C]

### tert-butyl 7-(6-chloro-3-(((2-(1,3-dioxoisoindolin-2-yl)ethoxy)carbonyl)amino)-1H-pyrazolo[4,3-c]pyridin-1-yl)-6-methoxy-2,3-dihydro-4H-benzo[b][1,4]oxazine-4-carboxylate (Intermediate 38y-1)

To a −78° C. cooled solution of intermediate 28 (100 mg, 0.23 mmol) in DCM (5 mL), TEA (97 μL, 0.70 mmol) and bis(trichloromethyl) carbonate (68.7 mg, 0.23 mmol) were added and RM stirred at −78° C. for 1 h. 2-(2-Hydroxyethyl)isoindoline-1,3-dione (88 μL, 0.926 mmol) and TEA (65 μL, 0.46 mmol) were added and RM stirred at RT overnight. RM was quenched with sat. aq. NaHCO₃ (10 mL) and extracted with DCM (15 mL). Combined organic layers were washed with sat. aq. NaCl and evaporated in vacuo. The residue was purified by flash chromatography on Si cartridge by eluting with 0-10% EtOAc in DCM to afford the title product (182 mg).

LCMS (Method 2): Rt=1.32 min, ES m/z 649.3/661.3 [M+H]⁺

### Step 2

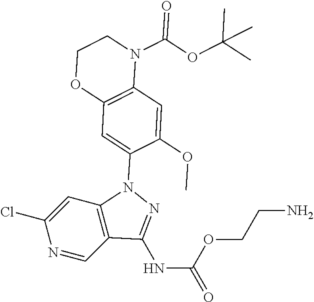 [SMILES: COc1cc2c(cc1-n1nc(NC(=O)OCCN)c3cnc(Cl)cc31)OCCN2C(=O)OC(C)(C)C]

### tert-Butyl 7-(3-(((2-aminoethoxy)carbonyl)amino)-6-chloro-1H-pyrazolo[4,3-c]pyridin-1-yl)-6-methoxy-2,3-dihydro-4H-benzo[b][1,4]oxazine-4-carboxylate (Intermediate 38y-2)

A solution of intermediate 38y-1 (155 mg, 0.24 mmol) and hydrazine hydrate (310 μL, 6.39 mmol) in EtOH (5 mL) was stirred at RT for 2 h. The precipitate removed by filtration and the filtrate partitioned between EtOAc (30 mL) and water (10 mL). Combined organic layers were washed with sat. aq. NaCl (10 mL) and solvent removed in vacuo to afford the desired product (81.7 mg) that was used in the next synthetic steps without further purification.

LCMS (Method 2): Rt=0.92 min, ES⁺ m/z 519.2/521.2 [M+H]⁺

### Step 3

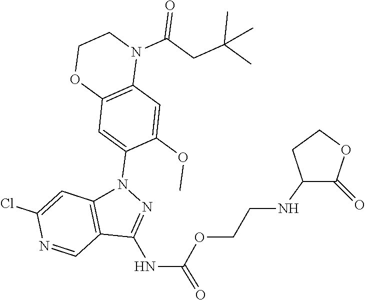 [SMILES: COc1cc2c(cc1-n1nc(NC(=O)OCCNC3CCOC3=O)c3cnc(Cl)cc31)OCCN2C(=O)CC(C)(C)C]

### tert-Butyl 7-(6-chloro-3-(((2-((2-oxotetrahydrofuran-3-yl)amino)ethoxy)carbonyl)amino)-1H-pyrazolo[4,3-c]pyridin-1-yl)-6-methoxy-2,3-dihydro-4H-benzo[b][1,4]oxazine-4-carboxylate (Intermediate 38y)

A solution of intermediate 38y-2 (75.0 mg, 0.145 mmol), 3-bromotetrahydrofuran-2-one (14.4 μL, 0.16 mmol) and DIPEA (101 μL, 0.58 mmol) in DMF (3 mL) was stirred at RT for 72 h. RM was diluted with EtOAc (15 mL) and washed with water (2×10 mL), and sat. aq. NaCl (15 mL). Solvents were removed in vacuo. The residue was purified by flash chromatography on Si cartridge by eluting with 0-5% DCM/MeOH/NH₄OH (90:5:0.5) in DCM to afford the title product (30 mg)

LCMS (Method 2): Rt=0.95 min, ES⁺ m/z 603.3/605.3 [M+H]⁺

### Intermediate 39a

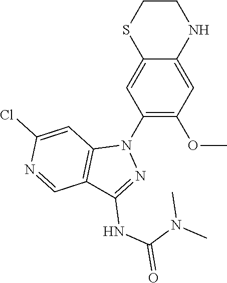 [SMILES: COc1cc2c(cc1-n1nc(NC(=O)N(C)C)c3cnc(Cl)cc31)SCCN2]

### 3-(6-chloro-1-(6-methoxy-3,4-dihydro-2H-benzo[b][1,4]thiazin-7-yl)-1H-pyrazolo[4,3-c]pyridin-3-yl)-1,1-dimethylurea (Intermediate 39a)

A solution of intermediate 21 (40.0 mg, 0.11 mmol), T3P® (50.0% solution in DMF, (124 μL, 0.21 mmol), azido(trimethyl)silane (28.2 μL, 0.21 mmol) and TEA (44.4 μL, 0.32 mmol) in 2-Me THF (0.5 ml) was refluxed overnight. After cooling to RT, RM was diluted with EtOAc (25 mL) and washed with sat. aq. NaHCO₃ (3×15 mL) and sat. aq. NaCl (15 mL). Organic layer was dried over Na₂SO₄ and concentrated in vacuo. The residue was purified by flash chromatography on Si cartridge by eluting with 0-30% DCM/MeOH (90:1) in DCM to afford the title product (10 mg).

LCMS (Method 1): Rt=1.01 min, ES⁺ m/z 419.1/421.1 [M+H]⁺

### Intermediate 39b

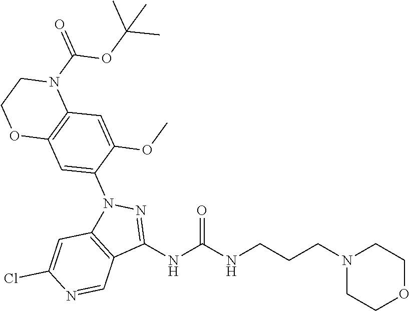 [SMILES: COc1cc2c(cc1-n1nc(NC(=O)NCCCN3CCOCC3)c3cnc(Cl)cc31)OCCN2C(=O)OC(C)(C)C]

### tert-Butyl 7-(6-chloro-3-(3-(3-morpholinopropyl)ureido)-1H-pyrazolo[4,3-c]pyridin-1-yl)-6-methoxy-2,3-dihydro-4H-benzo[b][1,4]oxazine-4-carboxylate (Intermediate 39b)

To a −78° C. cooled solution of intermediate 28 (50.0 mg, 0.12 mmol) in DCM (3 mL), DIPEA (48.4 μL, 0.35 mmol) and bis(trichloromethyl) carbonate (34.4 mg, 0.12 mmol) were added and RM stirred for 2 h. A solution of 3-morpholinopropan-1-amine (59.2 μL, 0.40 mmol) and DIPEA (32.3 μL, 0.23 mmol) in DCM (2 mL) was added dropwise and RM stirred at −78° C. for further 2 h. RM was allowed to warm to RT and diluted with sat. aq. NaHCO₃. Aqueous layer was further extracted with DCM (3×15 mL), and combined organic layers dried over Na₂SO₄ and evaporated in vacuo. The residue was purified by flash chromatography on Si cartridge by eluting with 0-100% DCM/MeOH/NH₄OH (90:9:0.5) in DCM to afford the title product (19.4 mg).

LCMS (Method 2): Rt=1.24 min, ES⁺ m/z 602.3/604.3 [M+H]⁺

### Intermediate 40

### Step 1

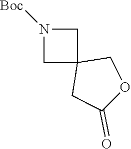 [SMILES: CC(C)(C)OC(=O)N1CC2(COC(=O)C2)C1]

### tert-Butyl 7-oxo-6-oxa-2-azaspiro[3.4]octane-2-carboxylate (Intermediate 40-1)

To a solution of tert-butyl 6-oxo-2-azaspiro[3.3]heptane-2-carboxylate (1 g, 4.64 mmol) in DCM (10 mL) at 0° C., NaHCO₃ (1.68 g) and m-CPBA (1.43 g, 7.89 mmol) were added and RM stirred at RT for 3 h. RM was diluted with DCM (10 mL) and sat. aq. NaHCO₃ (25 mL). The phases were separated, and aqueous layer extracted with DCM (2×25 mL). Combined organic layers were washed with brine (20 mL) and solvent removed in vacuo. Residue was slurried in a mixture DCM/MeOH, filtered to remove insoluble solids and the filtrate evaporated in vacuo to afford the title product (1.11 g).

¹H-NMR (500 MHz, DMSO-d₆) δ: 4.39 (s, 2H), 3.87 (bs, 4H), 2.83 (s, 2H), 1.37 (s, 9H).

### Step 2

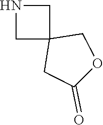

### 6-Oxa-2-azaspiro[3.4]octan-7-one trifluoroacetate (intermediate 40-2)

A solution of Intermediate 40-1 (200 mg, 0.88 mmol) and TFA (9.81 mL, 132.0 mmol) in DCM (10.0 mL) was stirred at RT for 3 h. The volatiles were evaporated in vacuo and residue triturated in diethyl ether to afford the title product (198.9 mg).

¹H-NMR (500 MHz, DMSO-d₆) δ: 8.80 (bs, 2H), 4.40 (s, 2H), 4.04 (bs, 4H), 2.90 (s, 2H).

Step 3

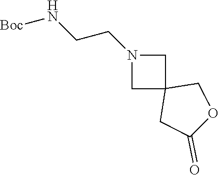 [SMILES: CC(C)(C)OC(=O)NCCN1CC2(COC(=O)C2)C1]

### tert-Butyl (2-(7-oxo-6-oxa-2-azaspiro[3.4]octan-2-yl)ethyl)carbamate (Intermediate 40-3

A mixture of N-Boc-2-aminoacetaldehyde (109.0 mg, 0.68 mmol, Intermediate 40-2 (128.0 mg, 0.57 mmol), TEA (79.2 μL, 0.57 mmol) and powdered molecular sieves (4 Å, 200 mg) in methanol (4.0 mL) was stirred at RT for 1 h. Na(CN)BH₃ (143.0 mg, 2.27 mmol) and acetic acid (48.8 μL, 0.85 mmol) were added and RM stirred at RT for 16 h. RM was quenched with water (10 mL), diluted with EtOAc (10 mL), filtered and layers separated through a phase separator. Organic layer was evaporated in vacuo. The residue was purified by flash chromatography on Si cartridge by eluting with 0-100% DCM/MeOH (10:1) in DCM to afford the title product.

LCMS (Method 2): Rt=0.75 min. ES⁺ m/z 271.2 [M+H]⁺

### Step 3

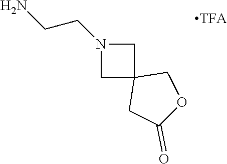 [SMILES: NCCN1CC2(COC(=O)C2)C1]

### 2-(2-Aminoethyl)-6-oxa-2-azaspiro[3.4]octan-7-one trifluoroacetate (Intermediate 40)

A Solution of Intermediate 40-3 (50 mg, 0.185 mmol) and TFA (2.06 mL, 27.7 mmol) in DCM (2.0 mL) was stirred at RT for 16 h. RM was evaporated to dryness and triturated in diethyl ether to afford the desired product (93 mg) that was used in the next synthetic step without further purifications.

LCMS (Method 2): Rt=0.34 min, ES⁺ m/z 171.2 [M+H]⁺

### Intermediate 41

### Step 1

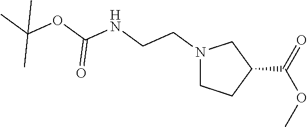 [SMILES: COC(=O)[C@@H]1CCN(CCNC(=O)OC(C)(C)C)C1]

### Methyl (R)-1-(2-((tert-butoxycarbonyl)amino)ethyl)pyrrolidine-3-carboxylate (Intermediate 41-1)

A mixture of (R)-pyrrolidine-3-carboxylate hydrochloride (406 mg, 2.5 mmol), K₂CO₃ (925 mg, 6.7 mmol) and tert-butyl N-(2-bromoethyl)carbamate (500 mg, 2.2 mmol) in MeCN (2.5 mL) was stirred at 70° C. overnight. After cooling to RT, RM was filtered and solvent evaporated. The crude was dissolved in EtOAc and washed with water. Aqueous solution was extracted with EtOAc (2×15 mL) and combined organic layers washed further with water (3×15 mL), sat aq NH₄Cl (15 mL) and sat. aq. NaCl (15 mL). Organic layer was passed through a phase separator and solvent evaporated to afford the title product (422 mg) that was used in the next synthetic steps without further purifications.

¹H-NMR (300 MHz, CDCl₃) δ: 4.95 (brs, 1H), 3.66 (s, 3H), 3.18-3.23 (m, 2H), 2.94-3.04 (m, 1H), 2.82 (t, J=7.8 Hz, 1H), 2.49-2.72 (m, 5H), 2.01-2.10 (m, 2H), 1.24 (s, 9H).

### Step 2

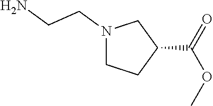

### Methyl (R)-1-(2-aminoethyl)pyrrolidine-3-carboxylate (Intermediate 41)

To a solution of Intermediate 41-1 (420 mg, 1.54 mmol) in dry DCM (3 ml) at 0° C., TFA (1.77 mL, 23.1 mmol) was added dropwise. RM was warmed to RT and stirred for 2 h. RM was loaded onto a preconditioned SCX column, washed with MeOH and eluted with 2N methanolic ammonia (50 mL). Evaporation of methanolic fractions afforded the desired product (263 mg).

¹H-NMR (300 MHz, CDCl₃) δ: 3.66 (s, 3H), 2.95-3.06 (m, 1H), 2.76-2.87 (m, 3H), 2.61-2-68 (m, 2H), 2.43-2.58 (m, 3H), 2.02-2.16 (m, 2H).

### Intermediates 42a-b

The following intermediates were prepared in a similar manner to intermediate 37a from the indicated starting materials.

| Intermediate | Structure | Starting materials | LCMS |
|---|---|---|---|
| Intermediate 42a | 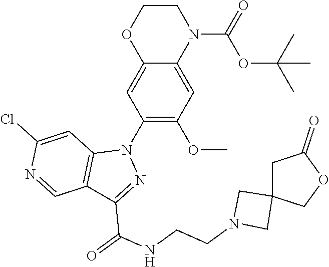 [SMILES: COc1cc2c(cc1-n1nc(C(=O)NCCN3CC4(COC(=O)C4)C3)c3cnc(Cl)cc31)OCCN2C(=O)OC(C)(C)C] | Intermediate 24 and Intermediate 40 | (Method 2): Rt = 1.16 min, ES⁺ m/z 613.4/615.4 [M + H]⁺ |
| Intermediate 42b | 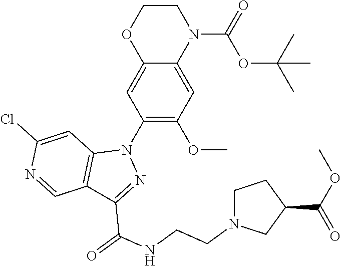 [SMILES: COC(=O)[C@@H]1CCN(CCNC(=O)c2nn(-c3cc4c(cc3OC)N(C(=O)OC(C)(C)C)CCO4)c3cc(Cl)ncc23)C1] | Intermediate 24 and Intermediate 41 | (Method 2): Rt = 1.29 min, ES⁺ m/z 615.3/617.3 [M + H]⁺ |

### Intermediate 43

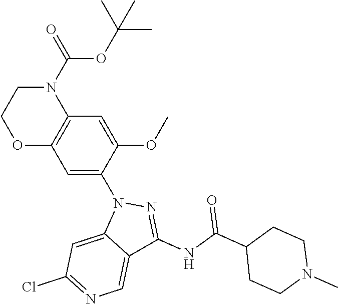 [SMILES: COc1cc2c(cc1-n1nc(NC(=O)C3CCN(C)CC3)c3cnc(Cl)cc31)OCCN2C(=O)OC(C)(C)C]

### tert-Butyl 7-(6-chloro-3-(1-methylpiperidine-4-carboxamido)-H-pyrazolo[4,3-c]pyridin-1-yl)-6-methoxy-2,3-dihydro-4H-benzo[b][1,4]oxazine-4-carboxylate (Intermediate 43)

A solution of Intermediate 28 (50.0 mg, 0.12 mmol), TEA (97 μL, 0.70 mmol) and 1-methylpiperidine-4-carbonyl chloride hydrochloride (34.4 mg, 0.17 mmol) in DCM (2 mL) was stirred at RT for 2 h. A second equivalent of 1-methylpiperidine-4-carbonyl chloride hydrochloride was added and stirring proceeded overnight. RM was diluted with DCM (10 mL), washed with sat. aq. NaHCO₃ and sat. aq. NH₄Cl (10 mL each). Organic layer was separated through a phase separator and solvent removed in vacuo. The residue was purified by flash chromatography on Si cartridge by eluting with 0-40% DCM/MeOH/NH₄OH (90:9:1.5) in DCM to afford the desired product (44 mg).

LCMS (Method 2): Rt=1.21 min, ES⁺ m/z 557.3/559.3 [M+H]⁺

### Intermediate 44

### Step 1

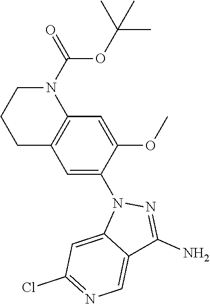 [SMILES: COc1cc2c(cc1-n1nc(N)c3cnc(Cl)cc31)CCCN2C(=O)OC(C)(C)C]

### tert-Butyl 6-(3-amino-6-chloro-1H-pyrazolo[4,3-c]pyridin-1-yl)-7-methoxy-3,4-dihydroquinoline-1 (2H)-carboxylate (Intermediate 44-1)

A Solution of Intermediate 27a (500 mg, 1.1 mmol), T3P® (50% in EtOAc, 1.27 mL, 2.2 mmol), azido(trimethyl)silane (289 μL, 2.2 mmol) and TEA (456 μL, 3.3 mmol) in 2-Me THF (20 ml) was refluxed for 30 min. Water (2.51 mL, 139 mmol) was added and RM was refluxed overnight. After cooling to RT, RM was diluted with EtOAc (25 mL) and sat. aq. NaHCO₃. Aqueous layer was extracted with EtOAc (3×25 mL). Combined organic layers were washed with sat. aq. NaCl, dried over Na₂SO₄ and concentrated in vacuo. The residue was purified by flash chromatography on Si cartridge by eluting with 0-35% DCM/MeOH/NH₄OH (90:9:1.5) in DCM to afford the title product (279 mg).

LCMS (Method 1): Rt=1.20 min. ES⁺ m/z 430.2/432.2 [M+H]⁺

### Step 2

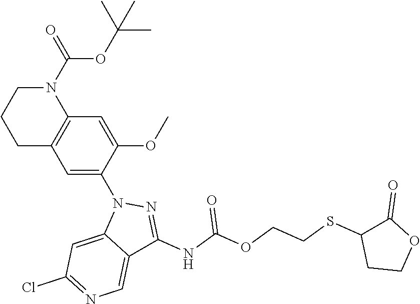 [SMILES: COc1cc2c(cc1-n1nc(NC(=O)OCCSC3CCOC3=O)c3cnc(Cl)cc31)CCCN2C(=O)OC(C)(C)C]

### tert-Butyl 6-(6-chloro-3-(((2-((2-oxotetrahydrofuran-3-yl)thio)ethoxy)carbonyl)amino)-1H-pyrazolo[4,3-c]pyridin-1-yl)-7-methoxy-3,4-dihydroquinoline-1 (2H)-carboxylate (Intermediate 44)

Solution of Intermediate 44-1 (100 mg, 0.23 mmol), TEA (97.3 μL, 0.70 mmol) in DCM (5 mL) was cooled −78° C. and bis(trichloromethyl) carbonate (69.0 mg, 0.23 mmol) was added in one portion. RM was stirred under the same conditions for 1 h. A solution of Intermediate 13 (151 mg, 0.93 mmol) and TEA (64.8 μL, 0.47 mmol) in DCM (2.5 mL) was added to RM at −78° C. and stirring proceeded for 2 h under cooling and overnight at RT. Reaction was quenched with sat. aq. NaHCO₃ and added with DCM and water. Layers were separated, and aqueous one extracted with DCM (3×20 mL). Combined organic layers were washed with sat. aq. NaCl, dried over Na₂SO₄ and solvent removed in vacuo. The residue was purified by flash chromatography on Si cartridge by eluting with 0-35% DCM/EtOH/NH₄OH (90:4:1) in DCM to afford the title product (177 mg).

LCMS (Method 2): Rt=1.28 min, ES⁺ m/z 618.2/620.2 [M+H]⁺

### Intermediates 45a to 45d

The following intermediates were prepared in a similar manner to step 2 of Intermediate 37q from the indicated starting materials.

| Intermediate | Structure | Starting materials | LCMS |
|---|---|---|---|
| Intermediate 45a | 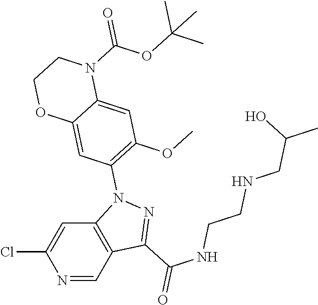 [SMILES: COc1cc2c(cc1-n1nc(C(=O)NCCNCC(C)O)c3cnc(Cl)cc31)OCCN2C(=O)OC(C)(C)C] | Intermediate 37q-1 and 1-aminopropan- 2-ol | (Method 2): Rt = 1.15 min, ES⁺ m/z 561.2/563.2 [M + H]⁺ |
| Intermediate 45b | 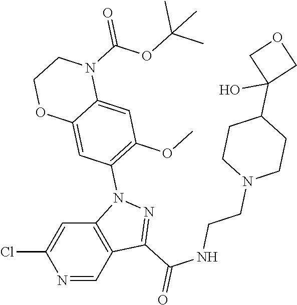 [SMILES: COc1cc2c(cc1-n1nc(C(=O)NCCN3CCC(C4(O)COC4)CC3)c3cnc(Cl)cc31)OCCN2C(=O)OC(C)(C)C] | Intermediate 37q-1 and 1-aminopro3-(4- piperidyl)oxetan-3- olpan-2-ol | (Method 2): Rt = 1.14 min, ES⁺ m/z 643.4/645.4 [M + H]⁺ |
| Intermediate 45c | 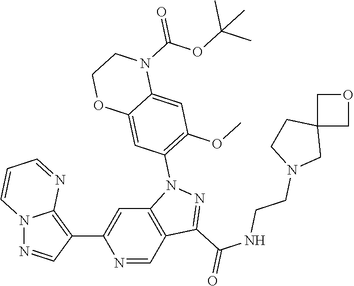 | Intermediate 37q-1 and 2-oxa-7-aza- spiro[3.4]octane | (Method 2): Rt = 1.22 min, ES⁺ m/z 599.3/601.3 [M + H]⁺ |
| Intermediate 45d | 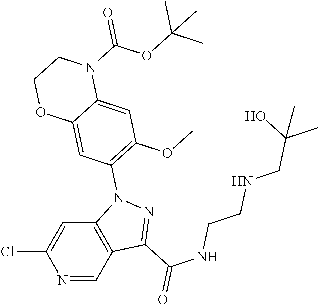 [SMILES: COc1cc2c(cc1-n1nc(C(=O)NCCNCC(C)(C)O)c3cnc(Cl)cc31)OCCN2C(=O)OC(C)(C)C] | Intermediate 37q-1 and 1-amino-2- methyl-propan-2-ol | (Method 1): Rt = 0.94 min, ES⁺ m/z 575.4/577.2 [M + H]⁺ |

### Intermediate 45e

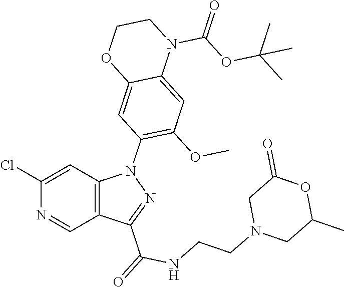 [SMILES: COc1cc2c(cc1-n1nc(C(=O)NCCN3CC(=O)OC(C)C3)c3cnc(Cl)cc31)OCCN2C(=O)OC(C)(C)C]

### tert-Butyl 7-(6-chloro-3-((2-(2-ethyl-6-oxomorpholino)ethyl)carbamoyl)-1H-pyrazolo[4,3-c]pyridin-1-yl)-6-methoxy-2,3-dihydro-4H-benzo[b][1,4]oxazine-4-carbonylate (Intermediate 45e)

Mixture of Intermediate 45a (42.0 mg, 0.07 mmol), K₂CO₃ (11.4 mg, 0.08 mmol) and ethyl 2-bromoacetate (9.1 μL, 0.08 mmol) in dry MeCN (9 mL) was stirred at RT overnight, then heated in pressure vial at 110° C. for 9 h. After cooling to RT, solvent was removed in vacuo and residue taken with DCM (20 mL), washed with sat. aq. NaHCO₃ (3×10 mL) and sat. aq. NaCl (10 mL). Organic layer evaporated to afford the title product (45 mg,) that was used in the next step without further purifications.

LCMS (Method 2): Rt=1.25 min ES⁺ m/z 601.3/603.3 [M+H]⁺

### Intermediate 45f

### Step 1

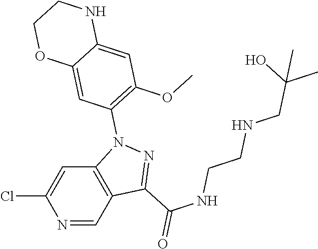 [SMILES: COc1cc2c(cc1-n1nc(C(=O)NCCNCC(C)(C)O)c3cnc(Cl)cc31)OCCN2]

### 6-Chloro-N-(2-((2-hydroxy-2-methylpropyl)amino)ethyl)-1-(6-methoxy-3,4-dihydro-2H-benzo[b][1,4]oxazin-7-yl)-1H-pyrazolo[4,3-c]pyridine-3-carboxamide (Intermediate 45f-1)

A solution of Intermediate 45d (80 mg, 0.14 mmol) and TFA (517 μL, 7.0 mmol) in DCM (9.5 mL) was stirred at RT overnight. Solvent was removed in vacuo and residue purified on a SCX column by elution with methanol and methanolic ammonia to afford the title compound (55.1 mg) that was used in the next step without further purification.

LCMS (Method 1): Rt=0.70 min ES⁺ m/z 475.2/477.1 [M+H]⁺

### Step 2

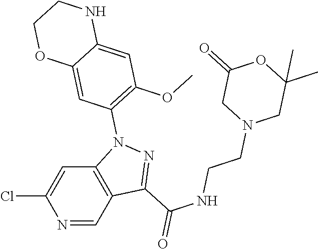 [SMILES: COc1cc2c(cc1-n1nc(C(=O)NCCN3CC(=O)OC(C)(C)C3)c3cnc(Cl)cc31)OCCN2]

### 6-Chloro-N-(2-(2,2-dimethyl-6-oxomorpholino)ethyl)-1-(6-methoxy-3,4-dihydro-2H-benzo[b][1,4]oxazin-7-yl)-1H-pyrazolo[4,3-c]pyridine-3-carboxamide (Intermediate 45f)

A solution of Intermediate 45f-1 (55 mg, 0.12 mmol) in toluene/acetonitrile was added to a precooled mixture of oxaldehyde (40%, 15 μL, 0.12 mmol) in toluene (0.3 mL) at 10° C. RM was stirred for 3 h at 10° C. and RT overnight. RM was diluted with water and extracted with DCM (4×10 mL). Combined organic layers were passed through phase separator and solvent was evaporated in vacuo. The residue was purified by flash chromatography on Si cartridge by eluting with 0-50% DCM/MeOH/NH₄OH (90:5:0.5) in DCM to afford the title product (32 mg).

LCMS (Method 2): Rt=0.99 min, ES⁺ m/z 515.2/517.1 [M+H]⁺

### PREPARATION OF EXAMPLES

### Example 1

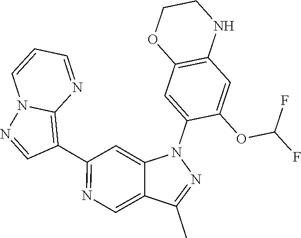 [SMILES: Cc1nn(-c2cc3c(cc2OC(F)F)NCCO3)c2cc(-c3cnn4cccnc34)ncc12]

### 6-(Difluoromethoxy)-7-(3-methyl-6-(pyrazolo[1,5-a]pyrimidin-3-yl)-1H-pyrazolo[4,3-c]pyridin-1-yl)-3,4-dihydro-2H-benzo[b][1,4]oxazine (Example 1)

To a degassed solution of intermediate 32b (33.0 mg, 0.09 mmol), 3-(4,4,5,5-tetramethyl-1,3,2-dioxaborolan-2-yl)pyrazolo[1,5-a]pyrimidine (30.9 mg, 0.13 mmol) and K₃PO₄ (38.2 mg, 0.18 mmol) THF (0.5 mL) and water (0.2 mL), XPhos Pd G3 (7.62 mg, 9.0 μmol) was added and RM stirred at 75° C. for 2 h. After cooling to RT, RM was diluted with water and extracted with DCM (3×5 mL). Combined organic layers were passed through phase separator and evaporated in vacuo. The residue was purified by flash chromatography on Si cartridge by eluting with 0-50% DCM/MeOH/NH₄OH (90:9:0.5) in DCM, and purified through an SCX column to afford the title compound (17 mg).

LCMS (Method 4): Rt=4.42 min, ES⁺ m/z 450.1 [M+H]⁺

¹H-NMR (500 MHz, DMSO-d₆) δ: 9.22 (br d, J=7.0 Hz, 1H), 9.12 (s, 1H), 8.89 (s, 1H), 8.72 (d, J=2.7 Hz, 1H), 8.18 (s, 1H), 7.16 (m, 1H), 6.88 (s, 1H), 6.88 (t, J=72.4 Hz, 1H), 6.65 (s, 1H), 6.57 (s, 1H), 4.19 (br s, 2H), 3.39 (br s, 2H), 2.61 (s, 3H).

### Examples 2 to 39

The following examples were prepared in a similar manner to Example 1 from the indicated intermediate.

| Example | Structure/Name | Intermediate | ¹H-NMR | LCMS |
|---|---|---|---|---|
| Example 2 | 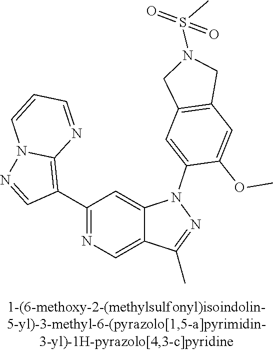 | Intermediate 32c | (500 MHz, DMSO- d₆) δ: 9.22 (dd, J = 6.9, 1.4 Hz, 1H); 9.15 (s, 1H); 8.89 (s, 1H); 8.69 (dd, J = 4.0, 1.5 Hz, 1H); 8.22 (s, 1H); 7.49 (s, 1H); 7.37 (s, 1H); 7.15 (dd, J = 7.0, 4.0 Hz, 1H); 4.75 (s, 2H); 4.65 (s, 2H); 3.83 (s, 3H); 3.03 (s, 3H); 2.65 (s, 3H) | (Method 4): Rt = 3.92 min, ES⁺ m/z 398.2 [M + H]⁺ |
| Example 3 | 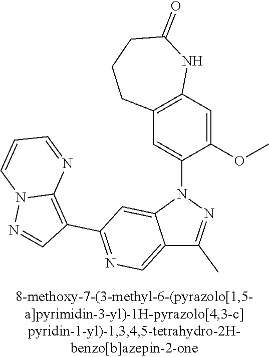 [SMILES: COc1cc2c(cc1-n1nc(C)c3cnc(-c4cnn5cccnc45)cc31)CCCC(=O)N2] | Intermediate 32d | (500 MHz, DMSO- d₆) δ: 9.70 (s, 1H); 9.22 (d, J = 6.7 Hz, 1H); 9.14 (s, 1H); 8.88 (s, 1H); 8.69 (d, J = 2.4 Hz, 1H); 8.24 (s, 1H); 7.41 (s, 1H); 7.20-7.10 (m, 1H); 6.94 (s, 1H); 3.79 (s, 3H); 2.78-2.68 (m, 2H); 2.64 (s, 3H); 2.32- 2.22 (m, 2H); 2.20- 2.06 (m, 2H) | (Method 4): Rt = 3.95 min, ES⁺ m/z 440.1 [M + H]⁺ |
| Example 4 | 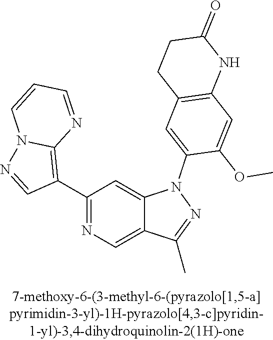 [SMILES: COc1cc2c(cc1-n1nc(C)c3cnc(-c4cnn5cccnc45)cc31)CCC(=O)N2] | Intermediate 32f | (500 MHz, DMSO- d₆) δ: 10.26 (s, 1H); 9.22 (d, J = 7.02 Hz, 1H); 9.13 (s, 1H); 8.88 (s, 1H); 8.71 (d, J = 4.27 Hz, 1H); 8.20 (s, 1H); 7.32 (s, 1H); 7.15 (dd, J = 6.87, 4.12 Hz, 1H); 6.81 (s, 1H); 3.73 (s, 3H); 2.90 (t, J = 7.48 Hz, 2H); 2.63 (s, 3H); 2.50 (t, 2 H, overlapped with DMSO) | (Method 4): Rt = 3.77 min, ES⁺ m/z 426.2 [M + H]⁺ |
| Example 5 | 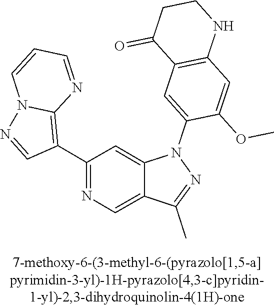 [SMILES: COc1cc2c(cc1-n1nc(C)c3cnc(-c4cnn5cccnc45)cc31)C(=O)CCN2] | Intermediate 32j | (600 MHz, DMSO- d₆) δ: 9.22 (dd, J = 6.97, 1.65 Hz, 1H); 9.12 (d, J = 1.10 Hz, 1H); 8.89 (s, 1H); 8.71 (dd, J = 4.22, 1.65 Hz, 1H); 8.16 (d, J = 1.10 Hz, 1H); 7.62 (s, 1H); 7.22 (br s, 1H); 7.16 (dd, J = 6.97, 4.22 Hz, 1H); 6.55 (s, 1H); 3.77 (s, 3H); 3.56- 3.52 (m, 2H); 2.63 (s, 3H); 2.58-2.54 (m, 2H) | (Method 4): Rt =3.77 min, ES⁺ m/z 426.2 [M + H]⁺ |
| Example 6 | 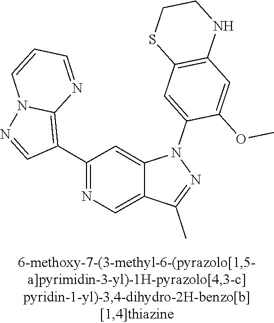 [SMILES: COc1cc2c(cc1-n1nc(C)c3cnc(-c4cnn5cccnc45)cc31)SCCN2] | Intermediate 33-4 | (500 MHz, DMSO- d₆) δ: 9.22 (d, J = 7.5, 1H); 9.09 (s, 1H); 8.88 (s, 1H); 8.72 (d, J = 4.0 Hz, 1H); 8.14(s, 1H); 7.15 (dd, J = 7.2, 4.3 Hz, 1H); 6.93 (s, 1H); 6.54 (s, 1H); 6.42 (s, 1H); 3.63 (s, 3H); 3.56-3.61 (m, 2H); 2.96-3.01 (m, 2H); 2.60 (s, 3H). | (Method 4): Rt = 4.63, ES⁺ m/z 430.2 [M + H]⁺ |
| Example 7 | 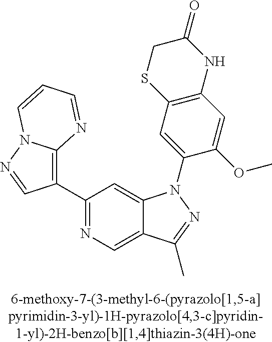 [SMILES: COc1cc2c(cc1-n1nc(C)c3cnc(-c4cnn5cccnc45)cc31)SCC(=O)N2] | Intermediate 33-3 | (500 MHz, DMSO- d₆) δ: 10.76 (s, 1H); 9.22 (dd, J = 7.1, 1.7 Hz, 1H); 9.13 (d, J = 1.1 Hz, 1H); 8.88 (s, 1H); 8.71 (dd, J = 4.0, 1.7 Hz, 1H); 8.21 (d, J = 1.0 Hz, 1H); 7.46 (s, 1H); 7.16 (dd, J = 6.8, 4.2 Hz, 1H); 6.98 (s, 1H); 3.76 (s, 3H); 3.54 (s, 2H); 2.63 (s, 3H, over- lapped with DMSO) | (Method 4): Rt = 4.08, ES⁺ m/z 444.1 [M + H]⁺ |
| Example 8 | 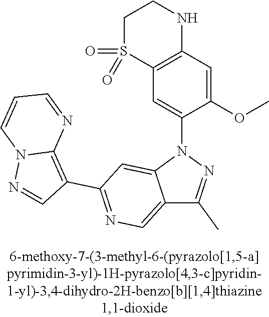 [SMILES: COc1cc2c(cc1-n1nc(C)c3cnc(-c4cnn5cccnc45)cc31)S(=O)(=O)CCN2] | Intermediate 33 | (500 MHz, DMSO- d₆) δ: 9.22 (dd, J = 7.2, 1.4 Hz, 1H); 9.09 (s, 1H); 8.88 (s, 1H); 8.72 (dd, J = 4.1, 1.5 Hz, 1H); 8.17(s, 1H); 7.53 (s, 1H); 7.45 (s, 1H); 7.15 (dd, J = 7.2, 4.3 Hz, 1H); 6.57 (s, 1H); 3.80-3.86 (2H, m); 3.76 (s, 3H); 3.42-3.47 (m, 2H); 2.62 (s, 3H) | (Method 4): Rt = 3.85, ES⁺ m/z 462.1 [M + H]⁺ |
| Example 9 | 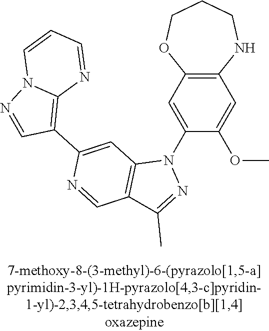 [SMILES: COc1cc2c(cc1-n1nc(C)c3cnc(-c4cnn5cccnc45)cc31)OCCCN2] | Intermediate 36 | (500 MHz, DMSO- d₆) δ: 9.22 (dd, J = 7.4, 1.3 Hz, 1H), 9.09 (s, 1H), 8.88 (s, 1H), 8.72 (dd, J = 4.2, 1.7 Hz, 1H), 8.18 (s, 1H), 7.15 (dd, J = 7.0, 4.0 Hz, 1H), 6.90 (s, 1H), 6.73 (s, 1H), 5.81 (t, J = 3.1 Hz, 1H), 4.02 (t, J = 4.0 Hz, 2H), 3.66 (s, 3H), 3.18- 3.24 (m, 2H), 2.61 (s, 3H), 1.89-1.95 (m, 2H) | (Method 4): Rt = 4.20, ES⁺ m/z 428.1 [M + H]⁺ |
| Example 10 | 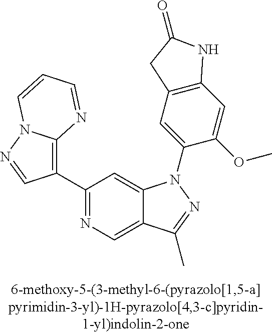 [SMILES: COc1cc2c(cc1-n1nc(C)c3cnc(-c4cnn5cccnc45)cc31)CC(=O)N2] | Intermediate 34-2 | (500 MHz, DMSO- d₆) δ: 10.65 (s, 1H), 9.20 (dd, J = 6.8 Hz, J = 1.8 Hz, 1H), 9.12 (s, 1H), 8.88 (s, 1H), 8.69 (dd, J = 4.6 Hz, J = 2.0 Hz, 1H), 8.16 (s, 1H), 7.30 (s, 1H), 7.13 (dd, J = 6.9 Hz, J = 3.8 Hz, 1H), 6.75 (s, 1H), 3.76 (s, 3H), 3.52 (s, 2H), 2.63 (s, 3H). | (Method 4): Rt = 3.54, ES⁺ m/z 412.1 [M + H]⁺ |
| Example 11 | 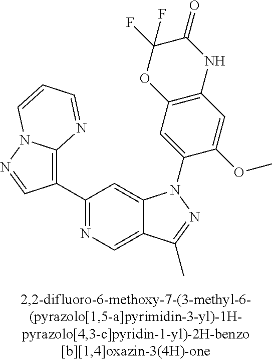 [SMILES: COc1cc2c(cc1-n1nc(C)c3cnc(-c4cnn5cccnc45)cc31)OC(F)(F)C(=O)N2] | Intermediate 35 | (500 MHz, DMSO- d₆) δ: 12.16 (br s, 1H), 9.23 (dd, J = 7.0, 1.5 Hz, 1H), 9.15 (s, 1H), 8.89 (s, 1H), 8.71 (dd, J = 4.0, 1.5 Hz, 1H), 8.24 (s, 1H), 7.56 (s, 1H), 7.16 (dd, J = 7.0, 4.0 Hz, 1H), 6.99 (s, 1H), 3.81 (s, 3H), 2.64 (s, 3H) | (Method 4): Rt = 4.60, ES⁺ m/z 464.1 [M + H]⁺ |
| Example 12 | 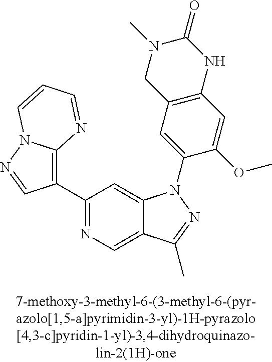 [SMILES: COc1cc2c(cc1-n1nc(C)c3cnc(-c4cnn5cccnc45)cc31)CN(C)C(=O)N2] | Intermediate 32i | (600 MHz, CDCl₃) δ: 9.08 (d, J = 1.1 Hz, 1H), 8.93 (s, 1H), 8.75 (dd, J = 6.7, 1.6, 1H), 8.58 (dd, J = 4.0, 1.7 Hz, 1H), 8.21 (d, J = 1.1 Hz, 1H), 7.31 (bs, 1H), 7.22 (s, 1H), 6.9 (dd, J = 7.1, 4.0 Hz, 1H), 6.46 (s, 1H), 3.82 (s, 3H), 3.06 (s, 3H), 2.72 (s, 3H) | (Method 4): Rt = 3.75, ES⁺ m/z 441.2 [M + H]⁺ |
| Example 13 | 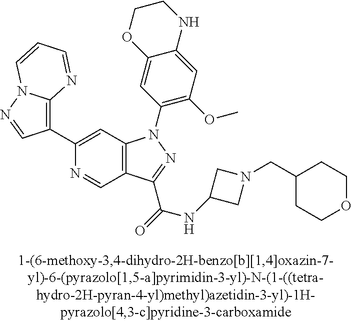 [SMILES: COc1cc2c(cc1-n1nc(C(=O)NC3CN(CC4CCOCC4)C3)c3cnc(-c4cnn5cccnc45)cc31)OCCN2] | Intermediate 37n | (600 MHz, DMSO- d₆) δ: 9.44 (d, J = 1.2 Hz, 1H), 9.24 (dd, J = 7.0, 1.7 Hz, 1H), 8.96 (d, J = 7.2 Hz, 1H), 8.91 (s, 1H), 8.74 (dd, J = .41, 1.7 Hz, 1H), 8.25 (d, J = 1.2 Hz, 1H), 7,18 (dd, J = 7.0, 4.1 Hz, 1H), 6.91 (s, 1H), 6.50 (s, 1H), 6.39 (bt, J = 2.2 Hz, 1H), 4.57 (sext, J = 7.0 Hz, 1H), 4.15 (d, J = 4.5 Hz, 1H), 3.80-3.82 (m, 2H), 3.64 (s, 3H), 3.57- 3.59 (m, 2H), 3.39- 3.41 (m, 2H), 3.24- | (Method 4): Rt = 4.02, ES⁺ m/z 596.3 [M + H]⁺ |
| | | | 328 (m, 2H), 3.06- | |
| | | | 3.08 (m, 2H), 2.30 | |
| | | | (d, J = 6.9 Hz, 2H), | |
| | | | 1.56-1.59 (m, 2H), | |
| | | | 1.47-1.53 (m, 1H), | |
| | | | 1.09-1.16 (m, 2H) | |
| Example 14 | 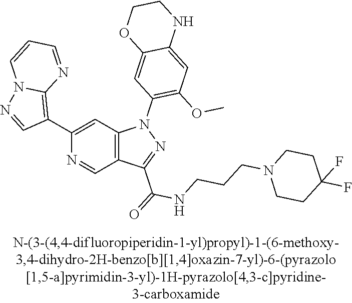 [SMILES: COc1cc2c(cc1-n1nc(C(=O)NCCCN3CCC(F)(F)CC3)c3cnc(-c4cnn5cccnc45)cc31)OCCN2] | Intermediate 37d | (300 MHz , DMSO- d₆) δ: 9.45 (d, J = 0.7 Hz, 1H), 9.22 (dd, J = 7.0, 1.6 Hz, 1H), 8.90 (s, 1H), 8.79 (t, J = 5.6 Hz, 1H), 8.72 (dd, J = 4.1, 1.7 Hz, 1H), 8.22 (d, J = 0.9 Hz, 1H), 7.16 (dd, J = 7.1, 4.3 Hz, 1H), 6.85 (s, 1H), 6.47 (s, 1H), 6.35 (s, 1H), 4.07-4.17 (m, 2H), 3.61 (s, 3H), 3.38 (m, 4H, under water), 2.49 (m, 6H, overlapped with DMSO), 1.83-2.01 (m, 4H), 1.67-1.78 | (Method 4): Rt = 4.14, ES⁺ m/z 604.2 [M + H]⁺ |
| | | | (m, 2H) | |
| Example 15 | 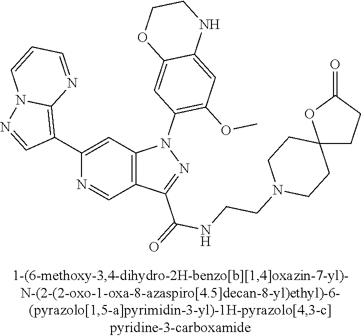 [SMILES: COc1cc2c(cc1-n1nc(C(=O)NCCN3CCC4(CCC(=O)O4)CC3)c3cnc(-c4cnn5cccnc45)cc31)OCCN2] | Intermediate 37x | (600 MHz, DMSO- d₆) 9.47 (d, J = 1.2 Hz, 1H), 9.24 (dd, J = 6.9, 1.7 Hz, 1H), 8.92 (s, 1H), 8.74 (dd, J = 4.1, 1.7 Hz, 1H), 8.46 (t, J = 5.8 Hz, 1H), 8.25 (d, J = 1.2 Hz, 1H), 7.17 (dd, J = 7.0, 4.0 Hz, 1H), 6.89 (s, 1H), 6.50 (s, 1H), 6.39 (br s, 1H), 4.14 (t, J = 4.4 Hz, 2H), 3.64 (s, 3H), 3.46 (q, J = 6.4 Hz, 2H), 3.41-3.37 (m, 2H), 2.60-2.53 (m, 6H), 2.48-2.41 (m, 2H), 1.99 (t, J = 8.2 Hz, 2H), 1.82- 1.76 (m, 2H), 1.75- | (Method 4): Rt = 3.76, ES⁺ m/z 624.3 [M + H]⁺ |
| | | | 1.69 (m, 2H) | |
| Example 16 | 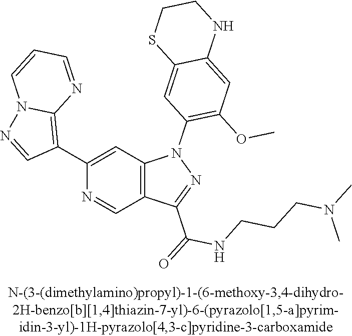 [SMILES: COc1cc2c(cc1-n1nc(C(=O)NCCCN(C)C)c3cnc(-c4cnn5cccnc45)cc31)SCCN2] | Intermediate 37e | (600 MHz, DMSO- d₆) δ: 9.46 (d, J = 1.3 Hz, 1H), 9.24 (dd, J = 7.0, 1.7 Hz, 1H), 8.91 (s, 1H), 8.74 (dd, J = 4.1, 1.7 Hz, 1H), 8.69 (t, J = 5.9 Hz, 1H), 8.25 (d, J = 1.1 Hz, 1H), 7.18 (dd, J = 7.0, 4.0 Hz, 1H), 7.09 (s, 1H), 6.66 (t, J = 2.8 Hz, 1H), 6.45 (s, 1H), 3.66 (s, 3H), 3.58- 3.63 (m, 2H), 3.33- 3.38 (m, 2H), 2.97- 3.03 (m, 2H), 2.27 (t, J = 7.0 Hz, 2H), 2.14 (s, 6H), 1.70 (quin, J = 7.1 Hz, 2H) | (Method 4): Rt = 3.99, ES⁺ m/z 544.2 [M + H]⁺ |
| Example 17 | 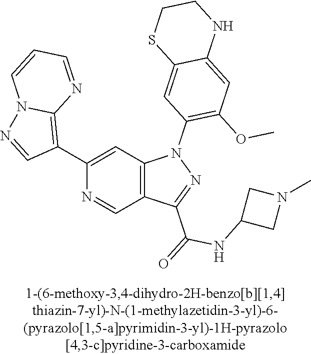 [SMILES: COc1cc2c(cc1-n1nc(C(=O)NC3CN(C)C3)c3cnc(-c4cnn5cccnc45)cc31)SCCN2] | Intermediate 37f | (600 MHz, DMSO- d₆) δ: 9.44 (bs, 1H), 9.24 (dd, J = 7.0, 1.7 Hz, 1H), 8.97 (d, J = 7.3 Hz, 1H), 8.91 (s, 1H), 8.75 (dd, J = 4.1, 1.7 Hz, 1H), 8.24 (bs, 1H), 7.18 (dd, J = 7.1, 4.1 Hz, 1H), 7.11 (s, 1H), 6.67-6.66 (m, 1H), 6.46 (s, 1H), 4.53 (sxt, J = 7.1 Hz, 1H), 3.65 (s, 3H), 3.62-3.57 (m, 4H), 3.11-3.06 (m, 2H), 3.02-2.98 (m, 2H), 2.26 (s, 3H) | (Method 4): Rt = 4.01, ES⁺ m/z 528.2 [M+H]⁺ |
| Example 18 | 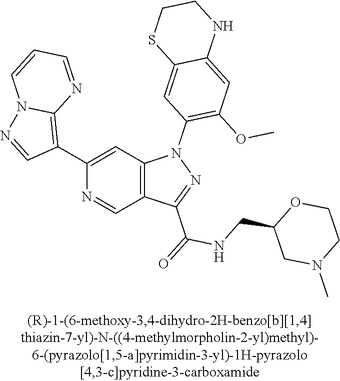 [SMILES: COc1cc2c(cc1-n1nc(C(=O)NC[C@@H]3CN(C)CCO3)c3cnc(-c4cnn5cccnc45)cc31)SCCN2] | Intermediate 37o | (300 MHz, DMSO- d₆) δ: 9.42-9.46 (m, 1H), 9.23 (dd, J = 7.0, 1.4 Hz, 1H), 8.90 (s, 1H), 8.73 (dd, J = 3.9, 1.5 Hz, 1H), 8.53 (br t, J = 5.8 Hz, 1H), 8.20- 8.25 (m, 1H), 7.16 (dd, J = 6.9, 4.3 Hz, 1H), 7.09 (s, 1H), 6.62-6.72 (m, 1H), 6.44 (s, 1H), 3.78 (br d, J = 11.3 Hz, 1H), 3.64-3.69 (m, 1H), 3.64 (s, 3H), 3.55- 3.62 (m, 2H), 3.42- 3.53 (m, 1H), 3.34- 3.41 (m, 2H), 2.92- 3.04 (m, 2H), 2.67- 2.78 (m, 1H), 2.51- 2.60 (m, 1H), 2.16 (s, 3H), 1.90-2.04 | (Method 4): Rt = 4.04, ES⁺ m/z 572.2 [M + H]⁺ |
| | | | (m, 1H), 1.75 (br t, | |
| | | | J = 10.6 Hz, 1H) | |
| Example 19 | 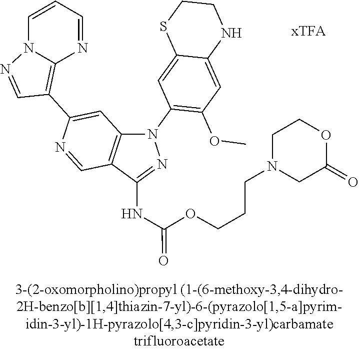 [SMILES: COc1cc2c(cc1-n1nc(NC(=O)OCCCN3CCOC(=O)C3)c3cnc(-c4cnn5cccnc45)cc31)SCCN2] | Intermediate 38u | (600 MHz, DMSO- d₆) δ: 10.83 (s, 1H), 9.45 (s, 1H), 9.29- 9.32 (m, 1H), 8.99 (s, 1H), 8.79-8.83 (m, 1H), 8.16 (s, 1H), 7.26 (dd, J = 6.9, 3.9 Hz, 1H), 7.00 (s, 1H), 6.44 (s, 1H), 4.42-4.48 (m, 2H), 4.27 (t, J = 6.3 Hz, 2H), 3.67-3.75 (m, 2H), 3.66 (s, 3H), 3.57-3.61 (m, 2H), 3.02-3.17 (m, 2H), 2.96-3.01 (m, 2H), 2.81-2.92 (m, 2H), 1.94-2.03 (m, 2H). | (Method 4): Rt = 4.31, ES⁺ m/z 616.1 [M + H]⁺ |
| Example 20 | 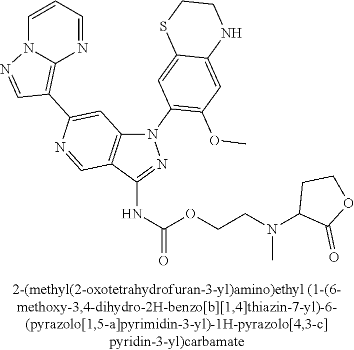 [SMILES: COc1cc2c(cc1-n1nc(NC(=O)OCCN(C)C3CCOC3=O)c3cnc(-c4cnn5cccnc45)cc31)SCCN2] | Intermediate 38v | (600 MHz, DMSO- d₆) δ: 10.46 (br s, 1H), 9.24-9.27 (m, 1H), 9.22 (dd, J = 7.0, 1.5 Hz, 1H), 8.87 (s, 1H), 8.72 (dd, J = 4.0, 1.5 Hz, 1H), 8.09 (d, J = 0.6 Hz, 1H), 7.16 (dd, J = 6.9, 4.1 Hz, 1H), 6.94 (s, 1H), 6.56- 6.59 (m, 1H), 6.42 (s, 1H), 4.28-4.33 (m, 1H), 4.22-4.28 (m, 2H), 4.10-4.18 (m, 1H), 3.91 (br t, J = 9.5 Hz, 1H), 3.64 (s, 3H), 3.56-3.61 (m, 2H), 2.96-3.01 (m, 2H), 2.81-2.95 (m, 2H), 2.35 (s, | (Method 4): Rt = 4.13, ES⁺ m/z 616.2 [M + H]⁺ |
| | | | 3H), 2.24-2.32 (m, | |
| | | | 1H), 2.14-2.24 (m, | |
| | | | 1H) | |
| Example 21 | 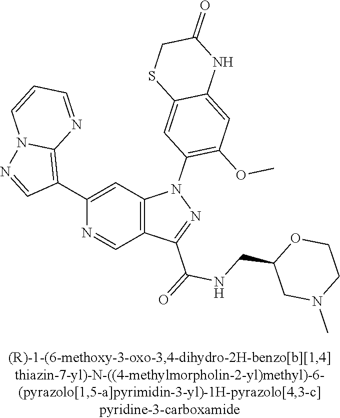 [SMILES: COc1cc2c(cc1-n1nc(C(=O)NC[C@@H]3CN(C)CCO3)c3cnc(-c4cnn5cccnc45)cc31)SCC(=O)N2] | Intermediate 37g | (300 MHz, DMSO- d₆) δ: 10.80 (s, 1H), 9.47 (d, J = 1.0 Hz, 1H), 9.23 (dd, J = 7.0, 1.7 Hz, 1H), 8.91 (s, 1H), 8.72 (dd, J = 4.0, 1.7 Hz, 1H), 8.61 (t, J = 5.9 Hz, 1H), 8.28 (d, J = 1.0 Hz, 1H), 7.64 (s, 1H), 7.17 (dd, J = 7.1, 4.1 Hz, 1H), 6.98 (s, 1H), 3.78- 3.84 (m, 1H), 3.76 (s, 3H), 3.61-3.71 (m, 1H), 3.56 (s, 2H), 3.44-3.53 (m, 1H), 3.35-3.44 (m, 2H), 2.68-2.77 (m, 1H), 2.53-2.59 (m, 1H), 2.16 (s, 3H), 1.96 (td, J = 11.2, 2.9 Hz, 1H), 1.75 (t, J = 10.5 Hz, 1H) | (Method 4): Rt = 3.72, ES⁺ m/z 586.0 [M + H]⁺ |
| Example 22 | 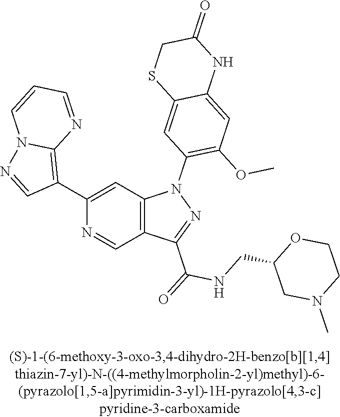 [SMILES: COc1cc2c(cc1-n1nc(C(=O)NC[C@H]3CN(C)CCO3)c3cnc(-c4cnn5cccnc45)cc31)SCC(=O)N2] | Intermediate 37h | (300 MHz, DMSO- d₆) δ: 10.80 (s, 1H), 9.47 (d, J = 1.0 Hz, 1H), 9.23 (dd, J = 7.0, 1.6 Hz, 1H), 8.91 (s, 1H), 8.72 (dd, J = 4.0, 1.7 Hz, 1H), 8.61 (t, J = 5.8 Hz, 1H), 8.28 (d, J = 1.0 Hz, 1H), 7.64 (s, 1H), 7.17 (dd, J = 7.1, 4.1 Hz, 1H), 6.98 (s, 1H), 3.78-3.82 (m, 1H), 3.76 (s, 3H), 3.60- 3.71 (m, 1H), 3.56 (s, 2H), 3.44-3.53 (m, 1H), 3.36-3.44 (m, 2H), 2.68-2.77 (m, 1H), 2.50-2.60 (m, 1H), 2.16 (s, 3H), 1.96 (td, J = 11.1, 3.3 Hz, 1H), 1.75 (t, J = 10.5 Hz, 1H) | (Method 4): Rt = 3.72, ES⁺ m/z 586.0 [M + H]⁺ |
| Example 23 | 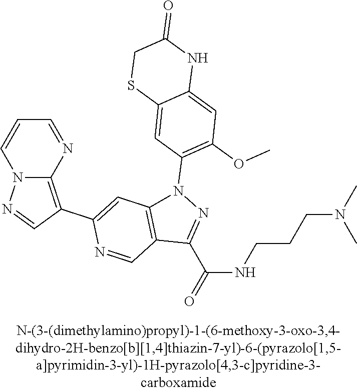 [SMILES: COc1cc2c(cc1-n1nc(C(=O)NCCCN(C)C)c3cnc(-c4cnn5cccnc45)cc31)SCC(=O)N2] | Intermediate 37i | (500 MHz, DMSO- d₆) δ: 10.81 (s, 1H), 9.45-9.52 (m, 1H), 9.24 (dd, J = 7.0, 1.5 Hz, 1H), 8.92 (s, 1H), 8.76 (t, J = 5.8 Hz, 1H), 8.73 (dd, J = 4.0, 1.5 Hz, 1H), 8.28-8.32 (m, 1H), 7.63 (s, 1H), 7.18 (dd, J = 6.9, 4.1 Hz, 1H), 6.99 (s, 1H), 3.78 (s, 3H), 3.57 (s, 2H), 3.33-3.39 (m, 2H), 2.28 (t, J = 6.9 Hz, 2H), 2.14 (s, 6H), 1.70 (quin, J = 7.1 Hz, 2H) | (Method 4): Rt = 3.68, ES⁺ m/z 558.1 [M + H]⁺ |
| Example 24 | 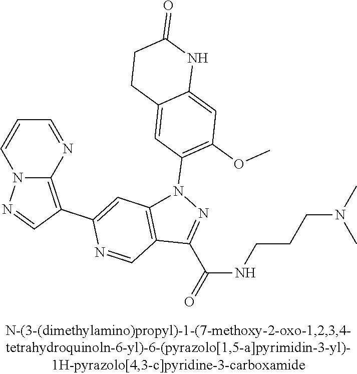 [SMILES: COc1cc2c(cc1-n1nc(C(=O)NCCCN(C)C)c3cnc(-c4cnn5cccnc45)cc31)CCC(=O)N2] | Intermediate 37j | (300 MHz, DMSO- d₆) δ: 10.33 (s, 1H), 9.48 (d, J = 1.0 Hz, 1H), 9.23 (dd, J = 7.0, 1.7 Hz, 1H), 8.91 (s, 1H), 8.68- 8.77 (m, 2H), 8.27 (d, J = 1.2 Hz, 1H), 7.44 (s, 1H), 7.16 (dd, J = 7.0, 4.2 Hz, 1H), 6.83 (s, 1H), 3.73 (s, 3H), 3.33- 3.40 (m, 2H), 2.90 (t, J = 7.4 Hz, 2H), 2.50-2.57 (m, 2H), 2.23-2.33 (m, 2H), 2.14 (s, 6H), 1.69 (quin, J = 7.1 Hz, 2H) | (Method 4): Rt = 3.43, ES⁺ m/z 540.1 [M + H]⁺ |
| Example 25 | 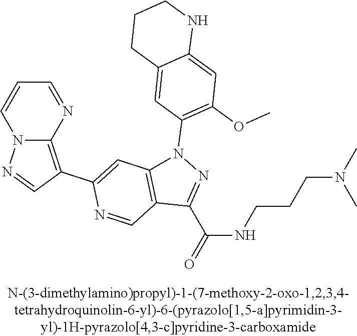 [SMILES: COc1cc2c(cc1-n1nc(C(=O)NCCCN(C)C)c3cnc(-c4cnn5cccnc45)cc31)CCCN2] | Intermediate 37p | (600 MHz, DMSO- d₆) δ: 9.46 (d, J = 1.1 Hz, 1H), 9.23 (dd, J = 7.0 Hz, J = 1.7 Hz, 1H), 8.91 (s, 1H), 8.74 (dd, J = 4.1 Hz, J = 1.7 Hz, 1H), 8.67 (t, J = 5.9 Hz, 1H), 8.24 (d, J = 1.3 Hz, 1H), 7.17 (dd, J = 7.0 Hz, J = 4.2 Hz, 1H), 7.02 (s, 1H), 6.33 (s, 1H), 6.25 (s, 1H), 3.63 (s, 3H), 3.33-3.37 (m, overlapped with HDO, 2H), 3.27 (m, 2H), 2.66 (t, J = 6.1 Hz, 2H), 2.28 (t, J = 7.0 Hz, 2H), 2.14 (s, 6H), 1.83 (m, 2H), | (Method 4): Rt = 3.99, ES⁺ m/z 526.2 [M + H]⁺ |
| | | | 1.70 (quin, J = 7.1 | |
| | | | Hz, 2H) | |
| Example 26 | 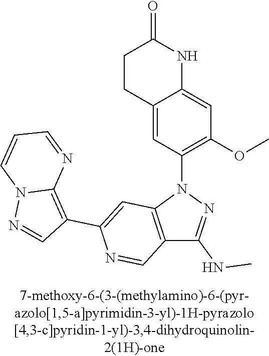 [SMILES: CNc1nn(-c2cc3c(cc2OC)NC(=O)CC3)c2cc(-c3cnn4cccnc34)ncc12] | Intermediate 30 | (500 MHz, DMSO- d₆) δ: 10.19 (s, 1H), 9.19 (dd, J = 7.1, 1.7 Hz, 1H), 8.99 (d, J = 0.9 Hz, 1H), 8.83 (s, 1H), 8.68 (dd, J = 4.1, 1.7 Hz, 1H), 8.01 (d, J = 0.7 Hz, 1H), 7.28 (s, 1H), 7.12 (dd, J = 7.0, 4.0 Hz, 1H), 6.77 (s, 1H), 6.62-6.73 (m, 1H), 3.74 (s, 3H), 2.82- 2.94 (d, J = 4.88 Hz, 3H), 2.83-2.88 (m, 2H), 2.48-2.55 (m, 2H) | (Method 4): Rt = 3.71, ES⁺ m/z 441.1 [M + H]⁺ |
| Example 27 | 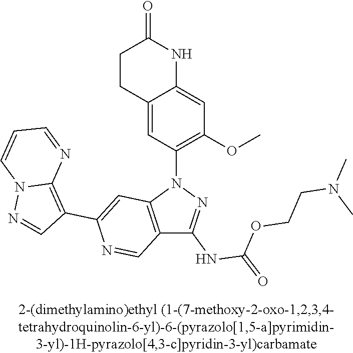 [SMILES: COc1cc2c(cc1-n1nc(NC(=O)OCCN(C)C)c3cnc(-c4cnn5cccnc45)cc31)CCC(=O)N2] | Intermediate 38l | (500 MHz, DMSO- d₆) δ: 10.50 (bs, 1H), 10.27 (s, 1H), 9.27 (s, 1H), 9.22 (dd, J = 7.0, 1.7 Hz, 1H), 8.87 (s, 1H), 8.71 (dd, J = 4.1, 1.7 Hz, 1H), 8.14 (s, 1H), 7.31 (s, 1H), 7.15 (dd, J = 7.0, 4.1 Hz, 1H), 6.82 (bs, 1H), 4.27 (t, J = 5.8 Hz, 2H), 3.74 (s, 3H), 2.89 (t, J = 7.5 Hz, 2H), 2.56 (t, J = 5.8 Hz, 2H), 2.54- 2.51 (m, 2H, over- lapped with DMSO), 2.22 (s, 6H) | (Method 4): Rt = 3.16, ES⁺ m/z 542.2 [M + H]⁺ |
| Example 28 | 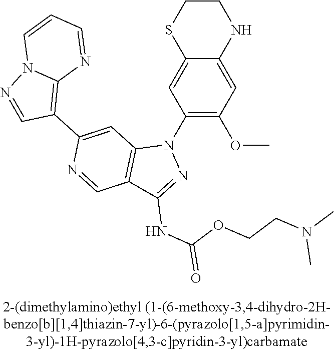 [SMILES: COc1cc2c(cc1-n1nc(NC(=O)OCCN(C)C)c3cnc(-c4cnn5cccnc45)cc31)SCCN2] | Intermediate 38m | (500 MHz, DMSO- d₆) δ: 10.44 (s, 1H), 9.24 (bs, 1H), 9.21 (dd, J = 7.0, 1.8 Hz, 1H), 8.87 (s, 1H), 8.71 (dd, J = 3.9, 1.7 Hz, 1H), 8.08 (s, 1H), 7.14 (dd, J = 7.5, 4.0 Hz, 1H), 6.94 (s, 1H), 6.57 (t, J = 2.8 Hz, 1H), 6.43 (s, 1H), 4.25 (t, J = 5.1 Hz, 2H), 3.64 (s, 3H), 3.56-3.61 (m, 2H), 2.96-3.00 (m, 2H), 2.54 (t, J = 6.1 Hz, 2H), 2.22 (s, 6H) | (Method 4): Rt = 3.73, ES⁺ m/z 546.2 [M + H]⁺ |
| Example 29 | 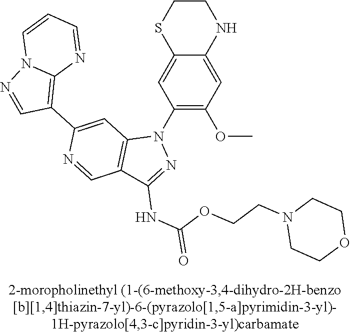 [SMILES: COc1cc2c(cc1-n1nc(NC(=O)OCCN3CCOCC3)c3cnc(-c4cnn5cccnc45)cc31)SCCN2] | Intermediate 38n | (600 MHz, DMSO- d₆) δ: 10.44 (bs, 1H), 9.26 (d, J = 1.1 Hz, 1H), 9.22 (dd, J = 7.1, 1.7 Hz, 1H), 8.87 (s, 1H), 8.72 (dd, J = 4.1, 1.7 Hz, 1H), 8.08 (d, J = 1.1 Hz, 1H), 7.16 (dd, J = 7.2, 4.2 Hz, 1H), 6.94 (s, 1H), 6.57 (t, J = 2.9 Hz, 1H), 6.42 (s, 1H), 4.29 (t, J = 5.7 Hz, 2H), 3.64 (s, 3H), 3.62-3.57 (m, 6H), 3.00-2.97 (m, 2H), 2.63 (t, J = 5.7 Hz, 2H), 2.47 (bs, 4H) | (Method 4): Rt = 3.74, ES⁺ m/z 588.2 [M + H]⁺ |
| Example 30 | 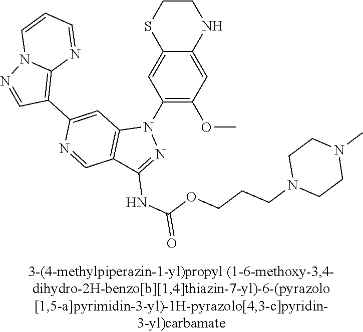 [SMILES: COc1cc2c(cc1-n1nc(NC(=O)OCCCN3CCN(C)CC3)c3cnc(-c4cnn5cccnc45)cc31)SCCN2] | Intermediate 38o | (500 MHz, DMSO) δ: 10.38 (s, 1H), 9.24-9.20 (m, 2H), 8.87 (s, 1H), 8.72 (dd, J = 4.2, 1.5 Hz, 1H), 8.08 (s, 1H), 7.15 (dd, J = 7.3, 4.5 Hz, 1H), 6.93 (s, 1H), 6.55-6.58 (m, 1H), 6.43 (s, 1H), 4.18 (t, J = 6.4 Hz, 2H), 3.62 (s, 3H), 3.56-3.60 (m, 2H), 2.94-3.00 (m, 2H), 2.18-2.42 (m, 10H), 2.13 (s, 3H), 1.81 (quin, J = 6.3 Hz, 2H). | (Method 4): Rt = 3.68, ES⁺ m/z 615.2 [M + H]⁺ |
| Example 31 | 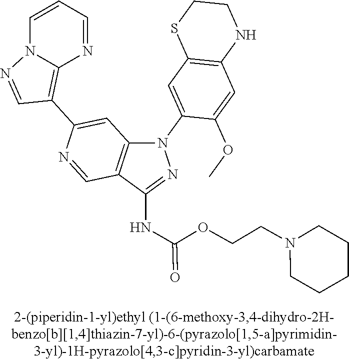 [SMILES: COc1cc2c(cc1-n1nc(NC(=O)OCCN3CCCCC3)c3cnc(-c4cnn5cccnc45)cc31)SCCN2] | Intermediate 38p | (500 MHz, DMSO- d₆) δ: 10.43 (br s, 1H), 9.25 (s, 1H), 9.20-9.24 (dd, J = 7.0 Hz, J = 1.5 Hz, 1H), 8.87 (s, 1H), 8.72 (dd, J = 4.0 Hz, J = 1.5 Hz, 1H), 8.08 (s, 1H), 7.15 (dd, J = 7.0, 4.3 Hz, 1H), 6.93 (s, 1H), 6.57 (br s, 1H), 6.43 (s, 1H), 4.26 (t, J = 5.8 Hz, 2H), 3.64 (s, 3H), 3.57-3.61 (m, 2H), 2.97-3.01 (m, 2H), 2.60 (t, J = 5.5 Hz, 2H), 2.42 (br s, 4H), 1.52 (quin, J = 5.5 Hz, 4H), δ: 1.39 (br d, J = 5.2 Hz, 2H) | (Method 4): Rt = 3.86, ES⁺ m/z 586.2 [M + H]⁺ |
| Example 32 | 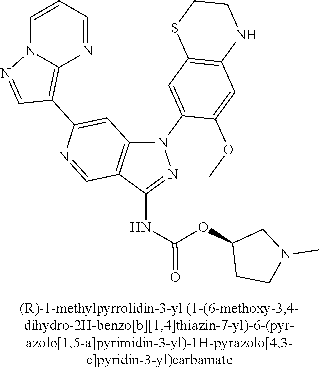 [SMILES: COc1cc2c(cc1-n1nc(NC(=O)O[C@@H]3CCN(C)C3)c3cnc(-c4cnn5cccnc45)cc31)SCCN2] | Intermediate 38q | (500 MHz, DMSO- d₆) δ: 10.03 (br s, 1H), 9.23 (d, J = 0.9 Hz, 1H), 9.12 (dd, J = 7.0 Hz, J = 1.5 Hz, 1H), 8.84 (s, 1H), 8.69 (dd, J = 4.1 Hz, J = 1.7 Hz, 1H), 8.10 (d, J = 1.2 Hz, 1H), 7.10 (dd, J = 7.0 Hz, J = 4.3 Hz, 1H), 6.92 (s, 1H), 6.47 (s, 1H), 6.29 (br s, 1H), 5.19-5.24 (m, 1H), 3.67 (s, 3H), 3.59-3.62 (m, 2H), 2.99-3.02 (m, 2H), 2.67-2.78 (m, 3H), 2.35-2.41 (m, 1H), 2.30 (s, 3H), 2.23- 2.30 (m, 1H), 1.83- 1.91 (m, 1H) | (Method 4): Rt = 3.70, ES⁺ m/z 558.1 [M + H]⁺ |
| Example 33 | 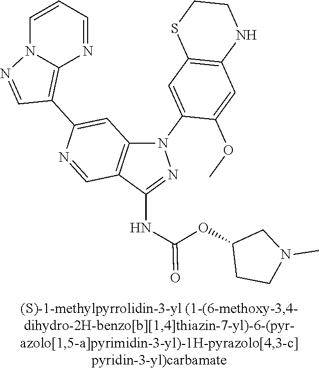 [SMILES: COc1cc2c(cc1-n1nc(NC(=O)O[C@H]3CCN(C)C3)c3cnc(-c4cnn5cccnc45)cc31)SCCN2] | Intermediate 38r | (500 MHz, DMSO- d₆) δ: 10.07 (br s, 1H), 9.23 (s, 1H), 9.11-9.15 (m, 1H), 8.84 (s, 1H), 8.67- 8.71 (m, 1H), 8.09 (s, 1H), 7.09-7.13 (m, 1H), 6.92 (s, 1H), 6.47 (s, 1H), 6.32 (br s, 1H), 5.18-5.24 (m, 1H), 3.67 (s, 3H), 3.57-3.64 (m, 2H), 2.98-3.03 (m, 2H), 2.66-2.77 (m, 3H), 2.34-2.40 (m, 1H), 2.30 (s, 3H), 2.22-2.29 (m, 1H), 1.82-1.90 (m, 1H) | (Method 4): Rt = 3.72, ES⁺ m/z 558.2 [M + H]⁺ |
| Example 34 | 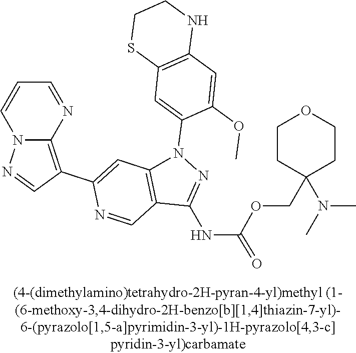 [SMILES: COc1cc2c(cc1-n1nc(NC(=O)OCC3(N(C)C)CCOCC3)c3cnc(-c4cnn5cccnc45)cc31)SCCN2] | Intermediate 38s | (500 MHz, DMSO- d₆) δ: 10.45 (br s, 1H), 9.21-9.24 (m, 2H), 8.87 (s, 1H), 8.72 (dd, J = 3.8, 1.4 Hz, 1H), 8.09 (s, 1H), 7.16 (dd, J = 7.0, 4.3 Hz, 1H), 6.94 (s, 1H), 6.57 (br s, 1H), 6.43 (s, 1H), 4.24 (s, 2H), 3.65- 3.70 (m, 2H), 3.64 (s, 3H), 3.57-3.61 (m, 2H), 3.51-3.56 (m, 2H), 2.95-3.03 (m, 2H), 2.30 (s, 6H), 1.68-1.77 (m, 2H), 1.57-1.67 (m, 2H) | (Method 4): Rt = 3.87, ES⁺ m/z 616.2 [M + H]⁺ |
| Example 35 | 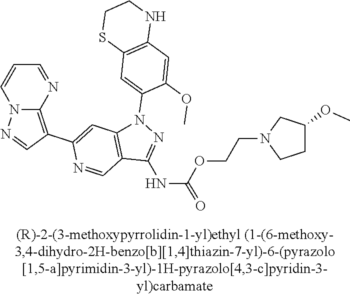 [SMILES: COc1cc2c(cc1-n1nc(NC(=O)OCCN3CC[C@@H](OC)C3)c3cnc(-c4cnn5cccnc45)cc31)SCCN2] | Intermediate 38t | (500 MHz, DMSO- d₆) δ:10.42-10.49 (br s, 1H), 9.24 (d, J = 0.9 Hz, 1H), 9.22 (dd, J = 7.0, 1.5 Hz, 1H), 8.87 (s, 1H), 8.72 (dd, J = 4.1, 1.7 Hz, 1H), 8.08 (d, J = 0.9 Hz, 1H), 7.16 (dd, J = 6.9, 4.1 Hz, 1H), 6.93 (s, 1H), 6.57 (br s, 1H), 6.42 (s, 1H), 4.26 (t, J = 5.6 Hz, 2H), 3.86- 3.90 (m, 1H), 3.64 (s, 3H), 3.56-3.61 (m, 2H), 3.17 (s, 3H), 2.95-3.02 (m, | (Method 4): Rt = 3.76, ES⁺ m/z 602.2 [M + H]⁺ |
| | | | 2H), 2.68-2.78 (m, | |
| | | | 3H), 2.60-2.67 (m, | |
| | | | 2H), 2.54 (m, over- | |
| | | | lapped with DMSO, | |
| | | | 1H), 1.95-2.02 (m, | |
| | | | 1H), 1.64-1.68 (m, | |
| | | | 1H) | |
| Example 36 | 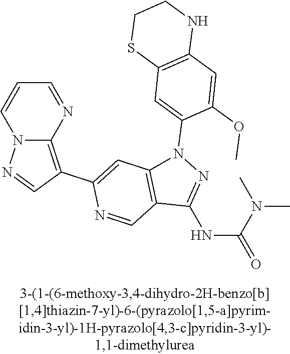 [SMILES: COc1cc2c(cc1-n1nc(NC(=O)N(C)C)c3cnc(-c4cnn5cccnc45)cc31)SCCN2] | Intermediate 39a | (500 MHz, DMSO- d₆) δ: 9.33 (bs, 1H), 9.21 (dd, J = 7.0, 1.5 Hz, 1H), 9.11 (bs, 1H), 8.86 (s, 1H), 8.71 (dd, J = 4.0, 1.5 Hz, 1H), 8.06 (bs, 1H), 7.15 (dd, J = 7.0, 4.0 Hz, 1H), 6.94 (s, 1H), 6.53- 6.57 (m, 1H), 6.43 (s, 1H), 3.65 (s, 3H), 3.55-3.61 (m, 2H), 3.01 (s, 6H), 2.96- 3.00 (m, 2H). | (Method 4): Rt = 4.49, ES⁺ m/z 502.2 [M + H]⁺ |
| Example 37 | 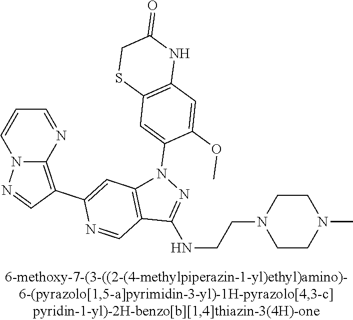 [SMILES: COc1cc2c(cc1-n1nc(NCCN3CCN(C)CC3)c3cnc(-c4cnn5cccnc45)cc31)SCC(=O)N2] | Intermediate 23a | (500 MHz, 353 K, DMSO-d₆) δ: 10.37 (s, 1H), 9.10 (dd, J = 7.0 Hz, J = 1.3 Hz, 1H), 9.04 (d, J = 0.8 Hz, 1H), 8.82 (s, 1H), 8.66 (dd, J = 4.1 Hz, J = 1.7 Hz, 1H), 8.21 (s, 0.4H), 8.07 (d, J = 0.8 Hz, 1H), 7.37 (s, 1H), 7.09 (dd, J = 7.1 Hz, J = 4.1 Hz, 1H), 6.96 (s, 1H), 6.28-6.32 (m, 1H), 3.82 (s, 3H), 3.49 (s, 2H), 3.48 (q, J = 6.2 Hz, 2H), 2.65 (t, J = 6.7 Hz, 2H), 2.45-2.53 (m, 4H), 2.34-2.38 | (Method 4): Rt = 3.22 min, ES⁺ m/z 571.2 [M + H]⁺ |
| | | | (m, 4H), 2.17 (s, 3H) | |
| Example 38 | 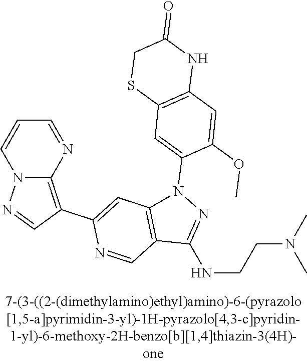 [SMILES: COc1cc2c(cc1-n1nc(NCCN(C)C)c3cnc(-c4cnn5cccnc45)cc31)SCC(=O)N2] | Intermediate 23b | (500 MHz, DMSO- d₆) δ: 10.68 (bs, 1H), 9.21 (dd, J = 7.1, 1.3 Hz, 1H), 9.07 (s, 1H), 8.85 (s, 1H), 8.70 (dd, J = 4.1, 1.3 Hz, 1H), 8.07 (s, 1H), 7.40 (s, 1H), 7.14 (dd, J = 7.1, 4.1 Hz, 1H), 6.93 (s, 1H), 6.66 (t, J = 5.5 Hz, 1H), 3.79 (s, 3H), 3.52 (s, 2H), 3.42-3-37 (m, 2H, overlapped with HDO), 2.54 (t, J = 6.5 Hz, 2H, over- lapped with DMSO), 2.22 (s, 6H) | (Method 4): Rt = 3.20 min, ES⁺ m/z 516.1 [M + H]⁺ |
| Example 39 | 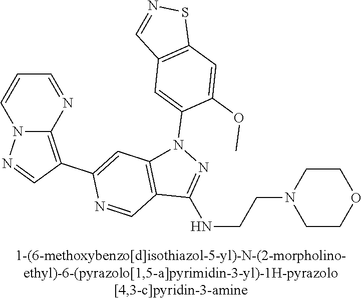 [SMILES: COc1cc2sncc2cc1-n1nc(NCCN2CCOCC2)c2cnc(-c3cnn4cccnc34)cc21] | Intermediate 20 | (500 MHz, DMSO- d₆) δ: 9.20 (dd, J = 1.5, 7.0 Hz, 1H), 9.11(d, J = 0.9 Hz, 1H), 9.06 (s, 1H), 8.86 (s, 1H), 8.67 (dd, J = 1.7, 4.1 Hz, 1H), 8.29 (s, 1H), 8.14 (d, J = 0.6 Hz, 1H), 8.12 (s, 1H), 7.13 (dd, J = 4.3, 7.0 Hz, 1H), 6.78 (t, J = 5.5 Hz, 1H), 3.98 (s, 3H), 3.60 (t, J = 4.6 Hz, 4H), 3.48 (q, J = 6.4 Hz, 2H), 2.65 (t, J = 6.9 Hz, 2H), 2.47 (br s, 4H) | (Method 4): Rt = 4.15 min, ES⁺ m/z 528.2 [M + H]⁺ |

### Example 40

### Step 1

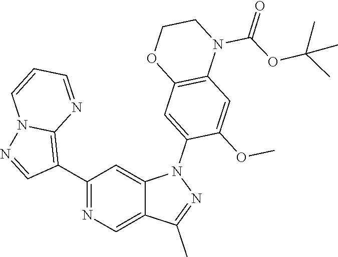 [SMILES: COc1cc2c(cc1-n1nc(C)c3cnc(-c4cnn5cccnc45)cc31)OCCN2C(=O)OC(C)(C)C]

### tert-Butyl 6-ethoxy-7-(3-ethyl-6-(pyrazolo[1,5-a]pyrimidin-3-yl)-1H-pyrazolo[4,3-c]pyridin-1-yl)-2,3-dihydro-4H-benzo[b][1,4]oxazine-4-carboxylate (Example 40—Step 1)

A degassed mixture of intermediate 32a (172 mg, 0.40 mol), 3-(4,4,5,5-tetramethyl-1,3,2-dioxaborolan-2-yl)pyrazolo[1,5-a]pyrimidine (117 mg, 0.48 mmol), K₃PO₄ (169 mg, 0.80 mmol) and XPhos PdG3 (17 mg, 20 μmol) in water (1 mL)/THF (2 mL) was stirred under nitrogen at 55° C. for 1.5 h. After cooling to RT, RM was diluted with water (5 mL) and extracted with DCM (3×10 mL). Combined organic layers were washed with water and sat. aq. NaCl (10 mL each), dried over Na₂SO₄ and evaporated in vacuo. The residue was purified by flash chromatography on Si cartridge by eluting with 0-80% DCM/MeOH/NH₄OH (90:9:0.5) in DCM to afford the title product (158.4 mg).

LCMS (Method 2), Rt=1.22, ES⁺ m/z 514.3.

### Step 2

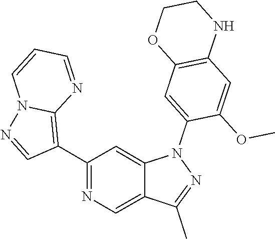 [SMILES: COc1cc2c(cc1-n1nc(C)c3cnc(-c4cnn5cccnc45)cc31)OCCN2]

### 6-Methoxy-7-(3-methyl-6-(pyrazolo[1,5-a]pyrimidin-3-yl)-1H-pyrazolo[4,3-c]pyridin-1-yl)-3,4-dihydro-2H-benzo[b][1,4]oxazine (Example 40)

TFA (0.7 mL, 9.23 mmol) was added to a solution of intermediate example 1—step 1 (158 mg, 0.31 mmol) in DCM (5.0 mL) and stirred at RT overnight. Solvents were removed in vacuo, and the residue loaded on a SCX cartridge, washed with methanol, and eluted with methanolic ammonia (7M) to afford the title product (78.7 mg)

LCMS (Method 3), Rt=2.67 min, ES⁺ m/z 414.1 [M+H]⁺

¹H-NMR (600 MHz, DMSO-d₆) δ: 9.21 (dd, J=6.9, 1.8 Hz, 1H); 9.09 (d J=1.2 Hz, 1H); 8.88 (s, 1H); 8.71 (dd, J=4.0, 1.7 Hz, 1H); 8.14 (d J=1.2 Hz, 1H); 7.15 (dd, J=6.9, 4.1 Hz, 1H); 6.73 (s, 1H); 6.46 (s, 1H); 6.26 (brs, 1H); 4.13 (t, J=3.3 Hz, 2H); 3.62 (s, 3H); 3.36 (m, 2H); 2.60 (s, 3H).

### Examples 41 to 70

The following examples were prepared by a two step process similarly to example 40 starting from the indicated intermediate.

| | | Step 1 | | | |
|---|---|---|---|---|---|
| Ex- | | Inter- | Name of | | |
| am- | | me- | intermediate | Step 2 | |
| ple | Structure/name | diate | Step 1/LCMS | ¹H-NMR | LCMS |
| Ex- am- ple 41 | 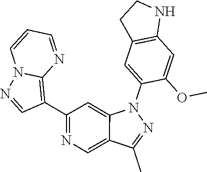 [SMILES: COc1cc2c(cc1-n1nc(C)c3cnc(-c4cnn5cccnc45)cc31)CCN2] | Inter- me- diate 32e | tert-butyl 6-methoxy-5-(3- methyl-6-(pyrazolo[1,5- a]pyrimidin-3-yl)-1H- pyrazolo[4,3-c]pyridin-1- yl)indoline-1-carboxylate (Method 1): Rt = 1.02 min, ES⁺ m/z 498.3 [M + H]⁺ | (500 MHz, DMSO-d₆) δ: 9.21 (d, J = 6.7 Hz, 1H); 9.09 (s, 1H); 8.88 (s, 1H); 8.70 (br s, 1H); 8.13 (s, 1H); 7.15 (m, 1H); 7.05 (m, 1H); 6.41 (s, 1H); 5.94 (br s, 1H); 3.64 (s, 3H); 3.55 (t, J = 7.8 Hz, 2H); 2.93 (t, J = 8.2 Hz, 2H); 2.61 (s, 3H) | (Method 4): Rt = 3.92 min, ES⁺ m/z 398.2 [M + H]⁺ |
| | 1-(6-methoxyindolin-5-yl)-3-methyl-6- | | | | |
| | (pyrazolo[1,5-a]pyrimidin-3-yl)-1H- | | | | |
| | pyrazolo[4,3-c]pyridine | | | | |
| Ex- am- ple 42 | 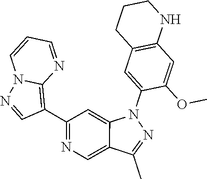 [SMILES: COc1cc2c(cc1-n1nc(C)c3cnc(-c4cnn5cccnc45)cc31)CCCN2] | Inter- me- diate 32g | tert-butyl 7-methoxy-6-(3- methyl-6-(pyrazolo[1,5- a]pyrimidin-3-yl)-1H- pyrazolo[4,3-c]pyridin- 1-yl)-3,4- dihydroquinoline-1(2H)- carboxylate (Method 1): Rt = 1.02 min, ES⁺ m/z 512.3 [M + H]⁺ | (500 MHz, DMSO-d₆) δ: 9.21 (dd, J = 7.0 Hz, J = 1.5 Hz, 1H); 9.08 (d, J = 0.9 Hz, 1H); 8.88 (s, 1H); 8.71 (dd, J = 4.1 Hz, J = 1.7 Hz, 1H); 8.13-8.14 (s, 1H); 7.14 (dd, J = 6.9 Hz, J = 4.1 Hz, 1H); 6.90 (s, 1H); 6.30 (s, 1H); 6.12 (s, 1H); 3.62 (s, 3H); 3.25 (m, 2H); 2.62-2.67 (m, 2H); 2.60 (s, 3H); 1.80-1.85 (m, 2H) | (Method 4): Rt = 4.61 min, ES⁺ m/z 412.2 [M + H]⁺ |
| | 7-methoxy-6-(3-methyl-6-(pyrazolo[1,5- | | | | |
| | a]pyrimidin-3-yl)-1H-pyrazolo[4,3- | | | | |
| | c]pyridin-1-yl)-1,2,3,4- | | | | |
| | tetrahydroquinoline | | | | |
| Ex- am- ple 43 | 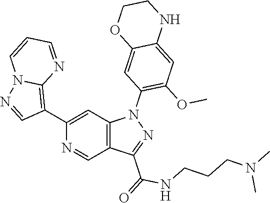 [SMILES: COc1cc2c(cc1-n1nc(C(=O)NCCCN(C)C)c3cnc(-c4cnn5cccnc45)cc31)OCCN2] | Inter- me- diate 37a | tert-butyl 7-(3-((3- (dimethylamino)propyl) carbamoyl)-6-(pyrazolo [1,5-a]pyrimidin-3-yl)-1H- pyrazolo[4,3-c]pyridin- 1-yl)-6-methoxy- 2,3-dihydro-4H- benzo[b][1,4]oxazine-4- carboxylate (Method 2): Rt = 0.83 min, ES⁺ m/z 512.3 [M + H]⁺ | (500 MHz, DMSO-d₆) δ: 9.45 (s, 1H), 9.22 (d, J = 6.9 Hz, 1H), 8.90 (s, 1H), 8.73 (d, J = 4.0 Hz, 1H), 8.69 (t, J = 6.2 Hz, 1H), 8.25 (s, 1H), 7.16 (dd, J = 7.6 Hz, J = 4.6 Hz, 1H), 6.89 (s, 1H), 6.49 (s, 1H), 6.37 (s, 1H), 4.13 (t, J = 4.0 Hz, 2H), 3.64 (s, 3H), 3.33-3.41 (m, overlapped with HDO, 4H), 2.26 (t, J = 5.9 Hz, 2H), 2.14 (s, 6H), 1.67 (quint, J = 6.4 Hz, 2H) | (Method 4): Rt = 3.60 min, ES⁺ m/z 528.2 [M + H]⁺ |
| | N-(3-(dimethylamino)propyl)-1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3-carboxamide | | | | |
| Ex- am- ple 44 | 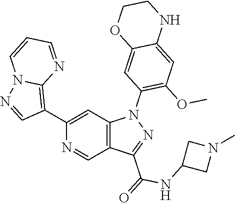 [SMILES: COc1cc2c(cc1-n1nc(C(=O)NC3CN(C)C3)c3cnc(-c4cnn5cccnc45)cc31)OCCN2] | Inter- me- diate 37b | tert-butyl 6-methoxy-7-(3- ((1-methylazetidin-3- yl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.81 min, ES⁺ m/z 612.4 [M + H]⁺ | (500 MHz, DMSO-d₆) δ: 9.43 (s, 1H), 9.23 (dd, J = 0.9, 7.0 Hz, 1H), 8.97 (d, J = 7.6 Hz, 1H), 8.91 (s, 1H), 8.73 (dd, J = 1.2, 3.7 Hz, 1H), 8.25 (s, 1H), 7.17 (dd, J = 4.0, 7.0 Hz, 1H), 6.90 (s, 1H), 6.49 (s, 1H), 6.38 (s, 1H), 4.58- 4.51 (m, 1H), 4.14 (t, J = 4.3 Hz, 2H), 3.63 (s, 3H), 3.60 (br t, J = 7.0 Hz, 2H), 3.39 (br s, 2H), 3.10 (br t, J = 6.9 Hz, 2H), 2.27 (s, 3H) | (Method 4): Rt = 3.79 min, ES⁺ m/z 512.2 [M + H]⁺ |
| | 1-(6-methoxy-3,4-dihydro-2H- | | | | |
| | benzo[b][1,4]oxazin-7-yl)-N-(1- | | | | |
| | methylazetidin-3-yl)-6-(pyrazolo[1,5- | | | | |
| | a]pyrimidin-3-yl)-1H-pyrazolo[4,3- | | | | |
| | c]pyridine-3-carboxamide | | | | |
| Ex- am- ple 45 | 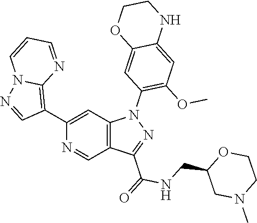 [SMILES: COc1cc2c(cc1-n1nc(C(=O)NC[C@@H]3CN(C)CCO3)c3cnc(-c4cnn5cccnc45)cc31)OCCN2] | Inter- me- diate 37c | tert-butyl (R)-6- methoxy-7-(3-(((4- methylmorpholin-2- yl)methyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.81 min, ES⁺ m/z 656.4 [M + H]⁺ | (500 MHz, DMSO-d₆) δ: 9.46 (s, 1H), 9.23 (d, J = 7.0 Hz, 1H), 8.91 (s, 1H), 8.76-8.72 (m, 1H), 8.54 (br t, J = 5.8 Hz, 1H), 8.25 (s, 1H), 7.17 (dd, J = 4.4, 6.6 Hz, 1H), 6.90 (s, 1H), 6.50 (s, 1H), 6.39 (br s, 1H), 4.14 (br s, 2H), 3.82 (br d, J = 11.6 Hz, 1H), 3.73- 3.67 (m, 1H), 3.64 (s, 3H), 3.52 (br t, J = 11.3 Hz, 1H), 3.45-3.36 (m, 4H), 2.86-2.79 (m, 1H), 2.69-2.62 (m, 1H), 2.24 (br s, 3H), 2.15-2.04 (m, 1H), 1.95- 1.83 (m, 1H) | (Method 4): Rt = 3.85 min, ES⁺ m/z 556.2 [M + H]⁺ |
| | (R)-1-(6-methoxy-3,4-dihydro-2H- | | | | |
| | benzo[b][1,4]oxazin-7-yl)-N-((4- | | | | |
| | methylmorpholin-2-yl)methyl)-6- | | | | |
| | (pyrazolo[1,5-a]pyrimidin-3-yl)-1H- | | | | |
| | pyrazolo[4,3-c]pyridine-3-carboxamide | | | | |
| Ex- am- ple 46 | 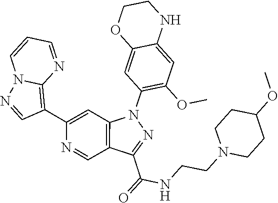 [SMILES: COc1cc2c(cc1-n1nc(C(=O)NCCN3CCC(OC)CC3)c3cnc(-c4cnn5cccnc45)cc31)OCCN2] | Inter- me- diate 37q | tert-butyl 6-methoxy- 7-(3-((2-(4- methoxypiperidin-1- yl)ethyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.95 min, ES⁺ m/z 684.3 [M + H]⁺ | (600 MHz, DMSO-d₆) δ: 9.45- 9.48 (m, 1H), 9.24 (dd, J = 7.0, 1.8 Hz, 1H), 8.92 (s, 1H), 8.74 (dd, J = 4.0, 1.8 Hz, 1H), 8.45 (t, J = 5.8 Hz, 1H), 8.25 (d, J = 1.3 Hz, 1H), 7.15-7.20 (m, 1H), 6.89 (s, 1H), 6.50 (s, 1H), 6.39 (s, 1H), 4.11-4.18 (m, 2H), 3.64 (s, 3H), 3.41-3.46 (m, 2H), 3.38- 3.41 (m, 2H), 3.22 (s, 3H), 3.13- 3.19 (m, 1H), 2.71-2.79 (m, 2H), 2.47-2.54 (m, 2 H; overlapped with DMSO); 2.10-2.19 (m, 2H), 1.83 (br dd, J = 9.0, 3.5 Hz, 2H), 1.37-1.46 (m, 2H) | (Method 4): Rt = 3.83 min, ES⁺ m/z 584.3 [M + H]⁺ |
| | 1-(6-methoxy-3,4-dihydro-2H- | | | | |
| | benzo[b][1,4]oxazin-7-yl)-N-(2-(4- | | | | |
| | methoxypiperidin-1-yl)ethyl)-6- | | | | |
| | (pyrazolo[1,5-a]pyrimidin-3-yl)-1H- | | | | |
| | pyrazolo[4,3-c]pyridine-3-carboxamide | | | | |
| Ex- am- ple 47 | 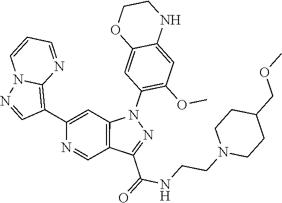 [SMILES: COCC1CCN(CCNC(=O)c2nn(-c3cc4c(cc3OC)NCCO4)c3cc(-c4cnn5cccnc45)ncc23)CC1] | Inter- me- diate 37r | tert-butyl 6-methoxy-7-(3- ((2-(4-(methoxy- methyl)piperidin-1- yl)ethyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.86 min, ES⁺ m/z 698.4 [M + H]⁺ | (600 MHz, DMSO-d₆) δ: 9.46 (d, J = 1.4 Hz, 1H),9.23 (dd, J = 6.8 Hz, 1.8 Hz, 1H), 8.91 (s, 1H), 8.73 (dd, J = 4.1 Hz, 1.6 Hz, 1H), 8.44 (bs, 1H), 8.25 (d, J = 1.5 Hz, 1H), 7.16 (dd, J = 7.0 Hz, 4.2 Hz, 1H), 6.89 (s, 1H), 6.49 (s, 1H), 6.38 (t, J = 2.4 Hz, 1H), 4.13 (t, J = 4.3 Hz, 2H), 3.64 (s, 3H), 3.43 (q, J = 7.3 Hz, 2H), 3.38 (q, J = 4.1 Hz, 2H), 3.32 (s, 3H), 3.15 (d, J = 6.5 Hz, 2H), 2.86-2.96 (m, 2H), 2.47-2.53 | (Method 4): Rt = 3.83 min, ES⁺ m/z 598.2 [M + H]⁺ |
| | 1-(6-methoxy-3,4-dihydro-2H- | | | (overlapped with DMSO, 2H), | |
| | benzo[b][1,4]oxazin-7-yl)-N-(2-(4- | | | 1.96 (bs, 2H), 1.60-1.65 (m, | |
| | (methoxymethyl)piperidin-1-yl)ethyl)-6- | | | 2H), 1.52 (bs, 1H), 1.12-1.22 | |
| | (pyrazolo[1,5-a]pyrimidin-3-yl)-1H- | | | (m, 2H) | |
| | pyrazolo[4,3-c]pyridine-3-carboxamide | | | | |
| Ex- am- ple 48 | 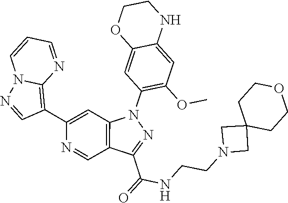 [SMILES: COc1cc2c(cc1-n1nc(C(=O)NCCN3CC4(CCOCC4)C3)c3cnc(-c4cnn5cccnc45)cc31)OCCN2] | Inter- me- diate 37s | tert-butyl 7-(3-((2-(7-oxa- 2-azaspiro[3.5]nonan-2- yl)ethyl)carbamoyl)-6- (pyrazolo[1,5-a] pyrimidin-3- yl)-1H-pyrazolo[4,3- c]pyridin-1-yl)-6-methoxy- 2,3-dihydro-4H- benzo[b][1,4]oxazine-4- carboxylate (Method 1): Rt = 0.84 min, ES⁺ m/z 696.4 [M + H]⁺ | (600 MHz, DMSO-d₆) δ: 9.46 (d, J = 1.4 Hz, 1H),9.23 (dd, J = 6.8 Hz, 1.8 Hz, 1H), 8.91 (s, 1H), 8.73 (dd, J = 4.1 Hz, 1.6 Hz, 1H), 8.44 (bs, 1H), 8.25 (d, J = 1.5 Hz, 1H), 7.16 (dd, J = 7.0 Hz, 4.2 Hz, 1H), 6.89 (s, 1H), 6.49 (s, 1H), 6.38 (t, J = 2.4 Hz, 1H), 4.13 (t, J = 4.3 Hz, 2H), 3.64 (s, 3H), 3.43 (q, J = 7.3 Hz, 2H), 3.38 (q, J = 4.1 Hz, 2H), 3.32 (s, 3H), 3.15 (d, J = 6.5 Hz, 2H), 2.86-2.96 (m, 2H), 2.47-2.53 | (Method 4): Rt = 3.77 min, ES⁺ m/z 596.2 [M + H]⁺ |
| | N-(2-(7-oxa-2-azaspiro[3.5]nonan-2- | | | (overlapped with DMSO, 2H), | |
| | yl)ethyl)-1-(6-methoxy-3,4-dihydro-2H- | | | 1.96 (bs, 2H), 1.60-1.65 (m, | |
| | benzo[b][1,4]oxazin-7-yl)-6- | | | 2H), 1.52 (bs, 1H), 1.12-1.22 | |
| | (pyrazolo[1,5-a]pyrimidin-3-yl)-1H- | | | (m, 2H) | |
| | pyrazolo[4,3-c]pyridine-3-carboxamide | | | | |
| Ex- am- ple 49 | 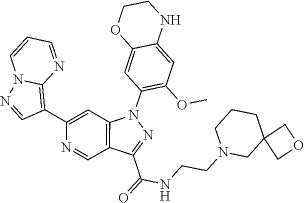 [SMILES: COc1cc2c(cc1-n1nc(C(=O)NCCN3CCCC4(COC4)C3)c3cnc(-c4cnn5cccnc45)cc31)OCCN2] | Inter- me- diate 37t | tert-butyl 7-(3-((2-(2-oxa- 6-azaspiro[3.5]nonan-6- yl)ethyl)carbamoyl)-6- (pyrazolo[1,5-a] pyrimidin-3- yl)-1H-pyrazolo[4,3- c]pyridin-1-yl)-6-methoxy- 2,3-dihydro-4H- benzo[b][1,4]oxazine-4- carboxylate (Method 1): Rt = 0.82 min, ES⁺ m/z 696.4 [M + H]⁺ | (600 MHz, DMSO-d₆) δ: 1H NMR (600 MHz, DMSO-d6) δ ppm: 9.46 (d, J = 1.3 Hz, 1H), 9.23 (dd, J = 7.9, 2.0 Hz, 1H), 8.91 (s, 1H), 8.73 (dd, J = 4.3, 1.8 Hz, 1H), 8.43 (t, J = 6.6 Hz, 1H), 8.25 (d, J = 1.6 Hz, 1H), 7.16 (dd, J = 7.2, 4.3 Hz, 1H), 6.87 (s, 1H), 6.49 (s, 1H), 6.37- 6.39 (m, 1H), 4.26 (d, J = 6.1 Hz, 2H), 4.19 (d, J = 6.1 Hz, 2H), 4.13 (t, J = 4.1 Hz, 2H), 3,.63 (s, 3H), 3.45 (q, J = 7.0 Hz, | (Method 4): Rt = 3.76 min, ES⁺ m/z 596.2 [M + H]⁺ |
| | N-(2-(2-oxa-6-azaspiro[3.5]nonan-6- | | | 2H), 3.37-3.40 (m, 2H), 2.53 | |
| | yl)ethyl)-1-(6-methoxy-3,4-dihydro-2H- | | | (t, J = 6.6 Hz, 2H), 2.42-2.52 | |
| | benzo[b][1,4]oxazin-7-yl)-6- | | | (overlapped with DMSO, 2H), | |
| | (pyrazolo[1,5-a]pyrimidin-3-yl)-1H- | | | 2.32 (bs, 2H), 1.62 (bs, 2H), | |
| | pyrazolo[4,3-c]pyridine-3-carboxamide | | | 1.40-1.46 (m, 2H) | |
| Ex- am- ple 50 | 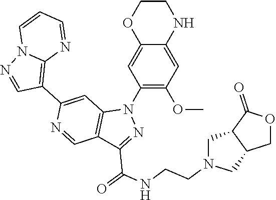 [SMILES: COc1cc2c(cc1-n1nc(C(=O)NCCN3C[C@H]4COC(=O)[C@H]4C3)c3cnc(-c4cnn5cccnc45)cc31)OCCN2] | Inter- me- diate 37u | tert-butyl 6-methoxy-7-(3- ((2-((3aS,6aR)-1- oxotetrahydro-1H-furo [3,4-c]pyrrol-5(3H)- yl)ethyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.88 min, | (500 MHz, DMSO-d₆) δ: 9.46 (d, J = 1.1 Hz, 1H), 9.24 (dd, J = 7.0, 1.7 Hz, 1H), 8.92 (s, 1H), 8.74 (dd, J = 4.0, 1.8 Hz, 1H), 8.56 (t, J = 6.0 Hz, 1H), 8.25 (d, J = 1.1 Hz, 1H), 7.17 (dd, J = 7.0, 4.0 Hz, 1H), 6.89 (s, 1H), 6.49 (s, 1H), 6.38 (s, 1H), 4.43 (t, J = 8.7 Hz, 1H), 4.14 (t, J = 4.3 Hz, 2H), 4.04 (dd, J = 9.0, 3.3 Hz, 1H), 3.63 (s, 3H), 3.44 (m, 2H), 3.39 (m, 2H), 3.10 (m, 2H), 3.02 (m, 2H), 2.66 (m, 1H), 2.53 (m, | (Method 4): Rt = 3.69 min, ES⁺ m/z 596.2 [M + H]⁺ |
| | 1-(6-methoxy-3,4-dihydro-2H- | | ES⁺ m/z 696.3 [M + H]⁺ | overlapped with DMSO, 1H), | |
| | benzo[b][1,4]oxazin-7-yl)-N-(2- | | | 2.38 (m, 1H), 2.33 (m, 1H) | |
| | ((3aS,6aR)-1-oxotetrahydro-1H-furo[3,4- | | | | |
| | c]pyrrol-5(3H)-yl)ethyl)-6-(pyrazolo[1,5- | | | | |
| | a]pyrimidin-3-yl)-1H-pyrazolo[4,3- | | | | |
| | c]pyridine-3-carboxamide | | | | |
| Ex- am- ple 51 | 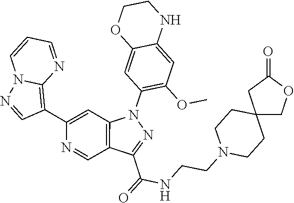 [SMILES: COc1cc2c(cc1-n1nc(C(=O)NCCN3CCC4(CC3)COC(=O)C4)c3cnc(-c4cnn5cccnc45)cc31)OCCN2] | Inter- me- diate 37v | tert-butyl 6-methoxy-7-(3- ((2-(3-oxo-2-oxa-8- azaspiro[4.5]decan-8- yl)ethyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.86 min, ES⁺ m/z 724.4 [M + H]⁺ | (500 MHz, DMSO-d₆) δ: 9.46 (d, J = 0.6 Hz, 1H), 9.23 (dd, J = 6.9, 1.7 Hz, 1H), 8.91 (s, 1H), 8.73 (dd, J = 4.0, 1.5 Hz, 1H), 8.43 (t, J = 5.8 Hz, 1H), 8.24 (d, J = 0.9 Hz, 1H), 7.17 (dd, J = 7.0, 4.0 Hz, 1H), 6.89 (s, 1H), 6.49 (s, 1H), 6.39 (s, 1H), 4.14 (t, J = 4.3 Hz, 2H), 4.04 (s, 2H), 3.63 (s, 3H), 3.44 (q, J = 6.4 Hz, 2H), 3.39 (s, 2H), 2.552.51 (m, 2H), 2.41 (s, 2H), 2.392.29 (m, 2H), 1.631.52 (m, 4H) | (Method 4): Rt = 3.75 min, ES⁺ m/z 624.2 [M + H]⁺ |
| | 1-(6-methoxy-3,4-dihydro-2H- | | | | |
| | benzo[b][1,4]oxazin-7-yl)-N-(2-(3-oxo-2- | | | | |
| | oxa-8-azaspiro[4.5]decan-8-yl)ethyl)-6- | | | | |
| | (pyrazolo[1,5-a]pyrimidin-3-yl)-1H- | | | | |
| | pyrazolo[4,3-c]pyridine-3-carboxamide | | | | |
| Ex- am- ple 52 | 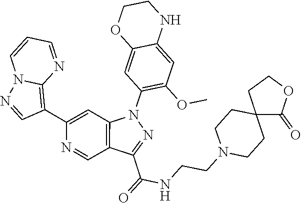 [SMILES: COc1cc2c(cc1-n1nc(C(=O)NCCN3CCC4(CCOC4=O)CC3)c3cnc(-c4cnn5cccnc45)cc31)OCCN2] | Inter- me- diate 37w | tert-butyl 6-methoxy-7-(3- ((2-(1-oxo-2-oxa-8- azaspiro[4.5]decan-8- yl)ethyl)carbamoyl)-6- (pyrazolo[1,5-a] pyrimidin-3- yl)-1H-pyrazolo[4,3- c]pyridin-1-yl)- 2,3-dihydro-4H-benzo [b][1,4]oxazine-4- carboxylate (Method 1): Rt = 0.88 min, ES⁺ m/z 724.4 [M + H]⁺ | (400 MHz, DMSO-d₆) δ: 9.45 (d, J = 1.1 Hz, 1H), 9.22 (dd, J = 7.0 Hz, 1H), 8.90 (s, 1H), 8.72 (dd, J = 4.1, 1.7 Hz, 1H), 8.46 (t, J = 5.9 Hz, 1H), 8.23 (d, J = 1.1 Hz, 1H), 7.16 (dd, J = 7.2, 4,2 Hz, 1H), 6.88 (s, 1H), 6.48 (s, 1H), 6.386.35 (m, 1H), 4.24 (t, J = 7.0 Hz, 2H), 4.13 (t, J = 4.2 Hz, 2H), 3.62 (s, 3H), 3.44 (q, J = 6.7 Hz, 2H), 3.38 (m, 2H), 2.882.80 (m, 2H),2.52 (m, 2H), 2.08-2.14 (m, 4H), 2.08 (s, 1H), | (Method 4): Rt = 3.81 min, ES⁺ m/z 624.2 [M + H]⁺ |
| | 1-(6-methoxy-3,4-dihydro-2H- | | | 1.731.64 (m, 2H), 1.54 (d, | |
| | benzo[b][1,4]oxazin-7-yl)-N-(2-(1-oxo-2- | | | J = 13.1 Hz, 2H) | |
| | oxa-8-azaspiro[4.5]decan-8-yl)ethyl)-6- | | | | |
| | (pyrazolo[1,5-a]pyrimidin-3-yl)-1H- | | | | |
| | pyrazolo[4,3-c]pyridine-3-carboxamide | | | | |
| Ex- am- ple 53 | 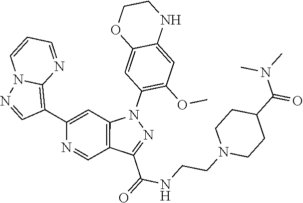 [SMILES: COc1cc2c(cc1-n1nc(C(=O)NCCN3CCC(C(=O)N(C)C)CC3)c3cnc(-c4cnn5cccnc45)cc31)OCCN2] | Inter- me- diate 37m | tert-butyl 7-(3-((2-(4- (dimethylcarbamoyl) piperidin-1-yl)ethyl) carbamoyl)-6-(pyrazolo [1,5-a]pyrimidin-3- yl)-1H-pyrazolo[4,3- c]pyridin-1-yl)-6- methoxy-2,3-dihydro-4H- benzo[b][1,4]oxazine-4- carboxylate (Method 1): Rt = 0.86 min, ES⁺ m/z 725.5 [M + H]⁺ | (600 MHz, DMSO-d₆) δ: 9.46 (s, 1H), 9.22 (d, J = 7.0 Hz, 1H), 8.91 (s, 1H), 8.73 (br d, J = 3.7 Hz, 1H), 8.48 (t, J = 5.4 Hz, 1H), 8.24 (s, 1H), 8.21-8.17 (bs, 1H), 7.16 (dd, J = 6.6, 4.1 Hz, 1H), 6.89 (s, 1H), 6.49 (s, 1H), 6.41- 6.35 (m, 1H), 4.14 (br s, 2H), 3.63 (s, 3H), 3.48-3.43 (overlapping with HDO, 2H), 3.41-3.37 (overlapping with HDO, 2H), 2.99 (s, 3H), 2.98 (m, 2H), 2.79 (s, 3H), 2.56 (m, | (Method 4): Rt = 3.75 min, ES⁺ m/z 625.3 [M + H]⁺ |
| | N-(2-(4-(dimethylcarbamoyl)piperidin-1- | | | 3H), 2.12-2.05 (m, 2H), 1.59 (br | |
| | yl)ethyl)-1-(6-methoxy-3,4-dihydro-2H- | | | s, 4H) | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3-carboxamide | | | | |
| x- am- ple 54 | 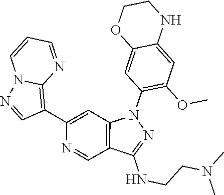 [SMILES: COc1cc2c(cc1-n1nc(NCCN(C)C)c3cnc(-c4cnn5cccnc45)cc31)OCCN2] | Inter- me- diate 31a | tert-butyl 7-(3-((tert- butoxycarbonyl)(2-(di- methylamino)ethyl) amino)-6-(pyrazolo [1,5-a]pyrimidin- 3-yl)-1H-pyrazolo[4,3- c]pyridin-1-yl)-6- methoxy-2,3-dihydro-4H- benzo[b][1,4]oxazine-4- carboxylate (Method 2): Rt = 1.37 min, ES⁺ m/z 686.2 [M + H]⁺ | (500 MHz, DMSO-d₆) δ: 9.09 (d, J = 6.7 Hz, 1H), 9.01 (s, 1H), 8.82 (s, 1H), 8.65-8.67 (m, 1H), 8.00 (s, 1H), 7.52 (br s, 1H), 7.08 (dd, J = 6.9 Hz, J = 4.1 Hz, 1H), 6.72 (s, 1H), 6.48 (s, 1H), 6.20 (br s, 1H), 4.13 (t, J = 4.3 Hz, 2H), 3.66 (s, 3H), 3.43-3.49 (m, 2H), 3.38 (m, 2H), 2.69 (t, J = 6.6 Hz, 2H), 2.32 (s, 6H) | (Method 4): Rt = 3.69 min, ES⁺ m/z 486.1 [M + H]⁺ |
| | N¹-(1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)-N²,N²- | | | | |
| | dimethylethane-1,2-diamine | | | | |
| Ex- am- ple 55 | 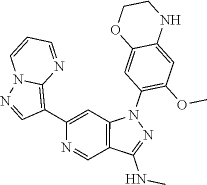 [SMILES: CNc1nn(-c2cc3c(cc2OC)NCCO3)c2cc(-c3cnn4cccnc34)ncc12] | Inter- me- diate 31b | tert-butyl 7-(3-((tert- butoxycarbonyl)(methyl) amino)-6-(pyrazolo[1,5- a]pyrimidin-3-yl)-1H- pyrazolo[4,3-c] pyridin-1-yl)- 6-methoxy-2,3- dihydro-4H- benzo[b][1,4]oxazine-4- carboxylate (Method 2): Rt = 1.41 min, | (500 MHz, DMSO-d₆) δ: 9.19 (d, J = 7.0 Hz, 1H), 8.96 (s, 1H), 8.84 (s, 1H), 8.69 (d, J = 4.0 Hz, 1H), 7.97 (s, 1H), 7.13 (dd, J = 6.9 Hz, J = 4.1 Hz, 1H), 6.71 (s, 1H), 6.58 (d, J = 4.9 Hz, 1H), 6.43 (s, 1H), 6.14 (br s, 1H), 4.12 (t, J = 4.0 Hz, 2H), 3.63 (s, 3H), 3.35 (br s, 2H) 2.88 (d, J = 4.9 Hz, 3H) | (Method 4): Rt = 4.07 min, ES⁺ m/z 429.1 [M + H]⁺ |
| | 1-(6-methoxy-3,4-dihydro-2H- | | ES⁺ m/z 629.3 [M + H]⁺ | | |
| | benzo[b][1,4]oxazin-7-yl)-N-methyl-6- | | | | |
| | (pyrazolo[1,5-a]pyrimidin-3-yl)-1H- | | | | |
| | pyrazolo[4,3-c]pyridin-3-amine | | | | |
| Ex- am- ple 56 | 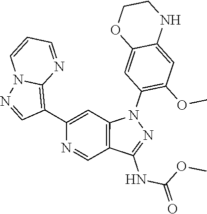 [SMILES: COC(=O)Nc1nn(-c2cc3c(cc2OC)NCCO3)c2cc(-c3cnn4cccnc34)ncc12] | Inter- me- diate 38a | tert-butyl 6-methoxy-7-(3- ((methoxycarbonyl) amino)-6-(pyrazolo [1,5-a]pyrimidin-3- yl)-1H-pyrazolo[4,3- c]pyridin-1-yl)-2,3- dihydro-4H-benzo [b][1,4]oxazine-4- carboxylate (Method 2): Rt = 1.10 min, ES⁺ m/z 573.0 [M + H]⁺ | (500 MHz, DMSO-d₆) δ: 10.46 (bs, 1H), 9.28-9.19 (m, 2H), 8.87 (s, 1H), 8.72 (d, J = 4.1 Hz, 1H), 8.09 (s, 1H), 7.15 (dd, J = 7.1, 4.1 Hz, 1H), 6.74 (s, 1H), 6.47 (s, 1H), 6.28 (bs, 1H), 4.13 (t, J = 4.0 Hz, 2H), 3.76 (s, 3H), 3.63 (s, 3H), 3.37 (bs, 2H). | (Method 4): Rt = 4.18 min, ES⁺ m/z 473.2 [M + H]⁺ |
| | methyl (1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 57 | 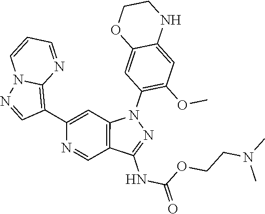 [SMILES: COc1cc2c(cc1-n1nc(NC(=O)OCCN(C)C)c3cnc(-c4cnn5cccnc45)cc31)OCCN2] | Inter- me- diate 38b | tert-butyl 7-(3-(((2- (dimethylamino)ethoxy) carbonyl)amino)- 6-(pyrazolo[1,5-a] pyrimidin-3-yl)-1H- pyrazolo[4,3-c]pyridin- 1-yl)-6-methoxy- 2,3-dihydro-4H- benzo[b][1,4]oxazine-4- carboxylate (Method 2): Rt = 1.12 min, ES⁺ m/z 630.4 [M + H]⁺ | (500 MHz, DMSO-d₆) δ: 10.43 (bs, 1H), 9.25-9.20 (m, 2H), 8.87 (s, 1H), 8.72 (dd, J = 4.1, 1.6 Hz, 1H), 8.09 (bs, 1H), 7.15 (dd, J = 7.0, 4.1 Hz, 1H), 6.74 (s, 1H), 6.47 (s, 1H), 6.28 (bs, 1H), 4.26 (t, J = 5.8 Hz, 2H), 4.13 (t, J = 4.1 Hz, 2H), 3.63 (s, 3H), 3.37 (m, 2H, overlapped with HDO), 2.56 (t, J = 5.8 Hz, 2H), 2.22 (s, 6H). | (Method 4): Rt = 3.36 min, ES⁺ m/z 530.1 [M + H]⁺ |
| | 2-(dimethylamino)ethyl (1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridin-3- | | | | |
| | yl)carbamate | | | | |
| Ex- am- ple 58 | 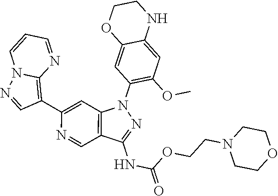 [SMILES: COc1cc2c(cc1-n1nc(NC(=O)OCCN3CCOCC3)c3cnc(-c4cnn5cccnc45)cc31)OCCN2] | Inter- me- diate 38c | tert-butyl 6-methoxy-7-(3- (((2-morpholino- ethoxy)carbonyl) amino)-6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 2): Rt = 1.07 min, ES⁺ m/z 672.2 [M + H]⁺ | (500 MHz, DMSO-d₆) δ: 10.44 (bs, 1H), 9.26 (d, J = 1.1 Hz, 1H), 9.21 (dd, J = 7.0, 4.1 Hz, 1H), 8.89 (s, 1H), 8.71 (dd, J = 4.1, 1.7 Hz, 1H), 8.09 (d, J = 1.1 Hz, 1H), 7.15 (dd, J = 7.1, 4.1 Hz, 1H), 6.74 (s, 1H), 6.47 (s, 1H), 6.27 (bs, 1H), 4.29 (t, J = 5.6 Hz, 2H), 4.13 (t, J = 4.3 Hz, 2H), 3.63 (s, 3H), 3.59 (t, J = 4.5 Hz, 4H), 3.37 (m, 2H, overlapped with HDO), 2.62 (t, J = 5.5 Hz, 2H), 2.47 (bs, | (Method 4): Rt = 3.41 min, ES⁺ m/z 572.1 [M + H]⁺ |
| | 2-morpholinoethyl (1-(6-methoxy-3,4- | | | 4H, overlapped with DMSO) | |
| | dihydro-2H-benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 59 | 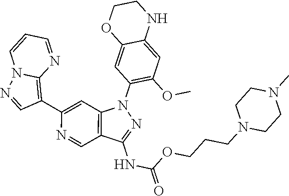 [SMILES: COc1cc2c(cc1-n1nc(NC(=O)OCCCN3CCN(C)CC3)c3cnc(-c4cnn5cccnc45)cc31)OCCN2] | Inter- me- diate 38d | tert-butyl 6-methoxy-7-(3- (((3-(4-methylpiperazin-1- yl)propoxy)carbonyl) amino)-6-(pyrazolo [1,5-a]pyrimidin- 3-yl)-1H-pyrazolo[4,3- c]pyridin-1-yl)- 2,3-dihydro-4H-benzo [b][1,4]oxazine-4- carboxylate (Method 2): Rt = 1.07 min, ES⁺ m/z 699.3 [M + H]⁺ | (500 MHz, DMSO-d₆) δ: 10.38 (bs, 1H), 9.24-9.19 (m, 2H), 8.87 (s, 1H), 8.71 (dd, J = 4.1, 1.6 Hz, 1H), 8.09 (bs, 1H), 7.15 (dd, J = 7.1, 4.1 Hz, 1H), 6.74 (s, 1H), 6.47 (s, 1H), 6.27 (bs, 1H), 4.19 (t, J = 6.6 Hz, 2H), 4.13 (t, J = 4.2 Hz, 2H), 3.63 (s, 3H), 3.37 (bs, 2H, overlapped with HDO), 2.44-2.23 (m, 10H), 2.14 (s, 3H), 1.81 (quint, J = 6.9 Hz, 2H). | (Method 4): Rt = 3.34 min, ES⁺ m/z 599.3 [M + H]⁺ |
| | 3-(4-methylpiperazin-1-yl)propyl (1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 60 | 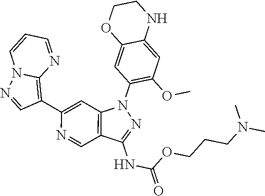 [SMILES: COc1cc2c(cc1-n1nc(NC(=O)OCCCN(C)C)c3cnc(-c4cnn5cccnc45)cc31)OCCN2] | Inter- me- diate 38e | tert-butyl 7-(3-(((3- (dimethylamino)propoxy) carbonyl)amino)-6- (pyrazolo[1,5-a]pyrimidin- 3-yl)-1H-pyrazolo[4,3- c]pyridin-1-yl)-6-methoxy- 2,3-dihydro-4H- benzo[b][1,4]oxazine-4- carboxylate (Method 2): Rt = 1.16 min, ES⁺ m/z 644.4 [M + H]⁺ | (500 MHz, DMSO-d₆) δ: 10.39 (bs, 1H), 9.25-9.19 (m, 2H), 8.87 (s, 1H), 8.71 (dd, J = 4.1, 1.7 Hz, 1H), 8.09 (bs, 1H), 7.15 (dd, J = 7.1, 4.1 Hz, 1H), 6.74 (s, 1H), 6.47 (s, 1H), 6.27 (bs, 1H), 4.19 (t, J = 6.6 Hz, 2H), 4.13 (t, J = 4.2 Hz, 2H), 3.63 (s, 3H), 3.37 (bs, 2H, overlapped with residual water), 2.32 (t, J = 7.0 Hz, 2H), 2.14 (s, 6H), 1.79 (quint, J = 6.9 Hz, 2H). | (Method 4): Rt = 3.45 min, ES⁺ m/z 544.2 [M + H]⁺ |
| | 3-(dimethylamino)propyl (1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridin-3- | | | | |
| | yl)carbamate | | | | |
| Ex- am- ple 61 | 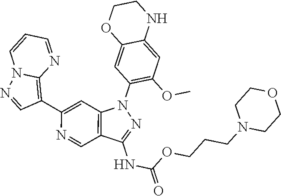 [SMILES: COc1cc2c(cc1-n1nc(NC(=O)OCCCN3CCOCC3)c3cnc(-c4cnn5cccnc45)cc31)OCCN2] | Inter- me- diate 38f | tert-butyl 6-methoxy-7-(3- (((3-morpholino- propoxy)carbonyl) amino)-6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 2): Rt = 1.10 min, ES⁺ m/z 686.4 [M + H]⁺ | (500 MHz, DMSO-d₆) δ: 10.39 (bs, 1H), 9.25-9.20 (m, 2H), 8.87 (s, 1H), 8.71 (dd, J = 4.1, 1.6 Hz, 1H), 8.09 (bs, 1H), 7.15 (dd, J = 7.1, 4.1 Hz, 1H), 6.74 (s, 1H), 6.47 (s, 1H), 6.27 (bs, 1H), 4.21 (t, J = 6.5 Hz, 2H), 4.13 (t, J = 4.2 Hz, 2H), 3.63 (s, 3H), 3.57 (t, J = 4.5 Hz, 4H), 3.37 (bs, 2H), 2.39 (t, J = 7.0 Hz, 2H, overlapped with DMSO satellite signal), 2.36 (bs, 4H, overlapped | (Method 4): Rt = 3.46 min, ES⁺ m/z 586.2 [M + H]⁺ |
| | 3-morpholinopropyl (1-(6-methoxy-3,4- | | | with DMSO satelite signal), | |
| | dihydro-2H-benzo[b][1,4]oxazin-7-yl)-6- | | | 1.83 (quint, J = 7.0 Hz, 2H). | |
| | (pyrazolo[1,5-a]pyrimidin-3-yl)-1H- | | | | |
| | pyrazolo[4,3-c]pyridin-3-yl)carbamate | | | | |
| Ex- am- ple 62 | 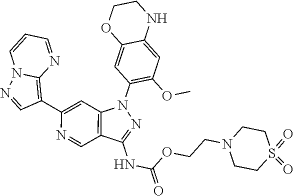 [SMILES: COc1cc2c(cc1-n1nc(NC(=O)OCCN3CCS(=O)(=O)CC3)c3cnc(-c4cnn5cccnc45)cc31)OCCN2] | Inter- me- diate 38g | tert-butyl 7-(3-(((2-(1,1- dioxidothiomorpholino) ethoxy)carbonyl)amino)-6- (pyrazolo[1,5-a]pyrimidin- 3-yl)-1H-pyrazolo[4,3- c]pyridin-1-yl)-6-methoxy- 2,3-dihydro-4H- benzo[b][1,4]oxazine-4- carboxylate (Method 2): Rt = 1.08 min, ES⁺ m/z 720.3 [M + H]⁺ | (500 MHz, DMSO-d₆) δ: 10.43 (bs, 1H), 9.32 (bs, 1H), 9.22 (dd, J = 7.0, 1.2 Hz, 1H), 8.89 (s, 1H), 8.72 (dd, J = 4.1, 1.2 Hz, 1H), 8.10 (bs, 1H), 7.15 (dd, J = 7.0, 4.1 Hz, 1H), 6.74 (s, 1H), 6.47 (s, 1H), 6.28 (bs, 1H), 4.29 (t, J = 5.4 Hz, 2H), 4.13 (t, J = 4.1 Hz, 2H), 3.63 (s, 3H), 3.37 (m, 2H, overlapped with HDO), 3.14 (bs, 4H), 3.08 (bs, 4H), 2.84 (t, J = 5.4 Hz, 2H) | (Method 4): Rt = 3.97 min, ES⁺ m/z 620.2 [M + H]⁺ |
| | 2-(1,1-dioxidothiomorpholino)ethyl (1- | | | | |
| | (6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 63 | 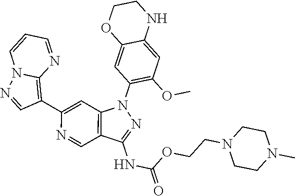 [SMILES: COc1cc2c(cc1-n1nc(NC(=O)OCCN3CCN(C)CC3)c3cnc(-c4cnn5cccnc45)cc31)OCCN2] | Inter- me- diate 38h | tert-butyl 6-methoxy-7-(3- (((2-(4-methylpiperazin-1- yl)ethoxy)carbonyl) amino)-6-(pyrazolo [1,5-a]pyrimidin-3- yl)-1H-pyrazolo[4,3- c]pyridin-1-yl)- 2,3-dihydro-4H-benzo [b][1,4]oxazine-4- carboxylate (Method 2): Rt = 1.04 min, ES⁺ m/z 685.4 [M + H]⁺ | (500 MHz, 353K, DMSO-d₆) δ: 10.03 (bs, 1H), 9.25 (d, J = 0.9 Hz, 1H), 9.12 (dd, J = 7.0, 1.6 Hz, 1H), 8.85 (s, 1H), 8.68 (dd, J = 4.1, 1.6 Hz, 1H), 8.10 (d, J = 0.8 Hz, 1H), 7.10 (dd, J = 7.0, 4.0 Hz, 1H), 6.73 (s, 1H), 6.51 (s, 1H), 5.99 (bs, 1H), 4.29 (t, J = 5.8 Hz, 2H), 4.15 (t, J = 4.5 Hz, 2H), 3.65 (s, 3H), 3.41-3.38 (m, 2H), 2.66 (t, J = 5.8 Hz, 2H), 2.53-2.50 (m, 4H, overlapped | (Method 4): Rt = 3.39 min, ES⁺ m/z 585.2 [M + H]⁺ |
| | 2-(4-methylpiperazin-1-yl)ethyl (1-(6- | | | with DMSO), 2.37-2.33 (m, | |
| | methoxy-3,4-dihydro-2H- | | | 4H), 2.17 (s, 3H). | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 64 | 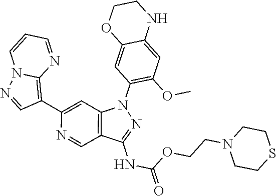 [SMILES: COc1cc2c(cc1-n1nc(NC(=O)OCCN3CCSCC3)c3cnc(-c4cnn5cccnc45)cc31)OCCN2] | Inter- me- diate 38i | tert-butyl 6-methoxy-7-(6- (pyrazolo[1,5-a]pyrimidin- 3-yl)-3-(((2-thiomorpho- linoethoxy)carbonyl) amino)-1H-pyrazolo[4,3- c]pyridin-1-yl)- 2,3-dihydro-4H-benzo [b][1,4]oxazine-4- carboxylate (Method 2): Rt = 1.18 min, ES⁺ m/z 688.2 [M + H]⁺ | (500 MHz, DMSO-d₆) δ = 10.43 (br s, 1H), 9.27 (s, 1H), 9.22 (dd, J = 7.0, 1.5 Hz, 1H), 8.87 (s, 1H), 8.72 (dd, J = 4.0, 1.5 Hz, 1H), 8.09 (s, 1H), 7.15 (dd, J = 6.9, 4.1 Hz, 1H), 6.74 (s, 1H), 6.47 (s, 1H), 6.28 (s, 1H), 4.27 (t, J = 5.6 Hz, 2H), 4.13 (t, J = 4.1 Hz, 2H), 3.63 (s, 3H), 3.37 (br s, 2H), 2.77-2.73 (m, 4H), 2.68- 2.60 (m, 6H) | (Method 4): Rt = 3.56 min, ES⁺ m/z 588.1 [M + H]⁺ |
| | 2-thiomorpholinoethyl (1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridin-3- | | | | |
| | yl)carbamate | | | | |
| Ex- am- ple 65 | 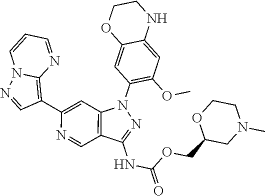 [SMILES: COc1cc2c(cc1-n1nc(NC(=O)OC[C@@H]3CN(C)CCO3)c3cnc(-c4cnn5cccnc45)cc31)OCCN2] | Inter- me- diate 38j | tert-butyl (S)-6- methoxy-7-(3-((((4- methylmorpholin-2- yl)methoxy) carbonyl)amino)-6- (pyrazolo[1,5-a]pyrimidin- 3-yl)-1H-pyrazolo[4,3- c]pyridin-1-yl)- 2,3-dihydro-4H-benzo [b][1,4]oxazine-4- carboxylate (Method 2): Rt = 1.07 min, | (600 MHz, DMSO-d₆) δ: 10.51 (bs, 1H), 9.23-9.21 (m, 2H), 8.87 (s, 1H), 8.72 (dd, J = 4.1, 1.7 Hz, 1H), 8.09 (d, J = 1.2 Hz, 1H), 7.15 (dd, J = 7.2, 4.1 Hz, 1H), 6.74 (s, 1H), 6.47 (s, 1H), 6.28 (t, J = 2.3 Hz, 1H), 4.21-4.12 (m, 4H), 3.84-3-80 (m, 1H), 3.76- 3.71 (m, 1H), 3.63 (s, 3H), 3.53 (td, J = 11.1, 2.6 Hz, 1H), 3.37 (bs, 2H), 2.73 (br.d. J = 11.1 Hz, 1H), 2.61-2.57 (m, 1H, | (Method 4): Rt = 3.44 min, ES⁺ m/z 572.2 [M + H]⁺ |
| | (S)-(4-methylmorpholin-2-yl)methyl (1- | | ES⁺ m/z 672.3 [M + H]⁺ | overlapped with DMSO satellite | |
| | (6-methoxy-3,4-dihydro-2H- | | | signal), 2.19 (s, 3H), 2.01 (td, | |
| | benzo[b][1,4]oxazin-7-yl)-6- | | | J = 11.1, 3.2 Hz, 1H), 1.85 (t, | |
| | (pyrazolo[1,5-a]pyrimidin-3-yl)-1H- | | | J = 10.5 Hz, 1H) | |
| | pyrazolo[4,3-c]pyridin-3-yl)carbamate | | | | |
| Ex- am- ple 66 | 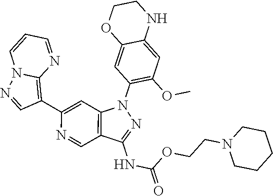 [SMILES: COc1cc2c(cc1-n1nc(NC(=O)OCCN3CCCCC3)c3cnc(-c4cnn5cccnc45)cc31)OCCN2] | Inter- me- diate 38k | tert-butyl 6-methoxy-7-(3- (((2-(piperidin-1-yl) ethoxy)carbonyl) amino)-6-(pyrazolo [1,5-a]pyrimidin-3- yl)-1H-pyrazolo[4,3- c]pyridin-1-yl)- 2,3-dihydro-4H-benzo [b][1,4]oxazine-4- carboxylate (Method 2): Rt = 1.29 min, ES⁺ m/z 670.3 [M + H]⁺ | (500 MHz, 353K, DMSO-d₆) δ: 10.43 (bs, 1H), 9.25 (s, 1H), 9.22 (dd, J = 7.1, 1.6 Hz, 1H), 8.89 (s, 1H), 8.72-8.70 (m, 1H), 8.09 (s, 1H), 7.15 (dd, J = 7.1, 4.0 Hz, 1H), 6.74 (s, 1H), 6.47 (s, 1H), 6.27 (bs, 1H), 4.26 (t, J = 5.8 Hz, 2H), 4.13 (t, J = 4.0 Hz, 2H), 3.63 (s, 3H), 3.37 (bs, overlapped with HDO, 2H), 2.58 (t, J = 5.8 Hz, 2H), 2.42 (bs, 4H), 1.51 (quint, J = 5.5 Hz, 4H), | (Method 4): Rt = 3.54 min, ES⁺ m/z 570.2 [M + H]⁺ |
| | 2-(piperidin-1-yl)ethyl (1-(6-methoxy- | | | 1.42-1.35 (m, 2H) | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridin-3- | | | | |
| | yl)carbamate | | | | |
| Ex- am- ple 67 | 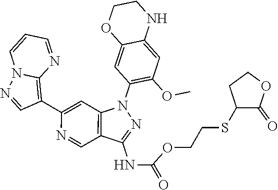 [SMILES: COc1cc2c(cc1-n1nc(NC(=O)OCCSC3CCOC3=O)c3cnc(-c4cnn5cccnc45)cc31)OCCN2] | Inter- me- diate 38w | tert-butyl 6-methoxy-7-(3- (((2-((2-oxotetrahydro- furan-3-yl)thio) ethoxy)carbonyl)amino)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.98 min, ES⁺ m/z 703.1 [M + H]⁺ | (300 MHz, DMSO-d₆) δ: 10.53 (br s, 1H), 9.13-9.31 (m, 2H), 8.86 (s, 1H), 8.65-8.77 (m, 1H), 8.09 (s, 1H), 7.07-7.23 (m, 1H), 6.73 (s, 1H), 6.46 (s, 1H), 6.28 (br s, 1H), 4.20-4.52 (m, 4H), 4.07-4.16 (m, 2H), 4.01 (br t, J = 7.7 Hz, 1H), 3.62 (s, 3H), 3.35-3.44 (m, 2H), 3.05-3.21 (m, 1H), 2.89-3.04 (m, 1H), 2.59-2.78 (m, 1H), 1.97-2.18 (m, 1H) | (Method 4): Rt = 4.51 min, ES⁺ m/z 603.2 [M + H]⁺ |
| | 2-((2-oxotetrahydrofuran-3-yl)thio)ethyl | | | | |
| | (1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 68 | 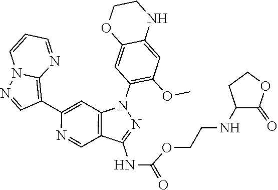 [SMILES: COc1cc2c(cc1-n1nc(NC(=O)OCCNC3CCOC3=O)c3cnc(-c4cnn5cccnc45)cc31)OCCN2] | Inter- me- diate 38y | tert-butyl 6-methoxy-7-(3- (((2-((2-oxotetrahydro- furan-3-yl)amino) ethoxy)carbonyl) amino)-6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.78 min, ES⁺ m/z 686.4 [M + H]⁺ | (600 MHz, DMSO-d₆) δ: 10.48- 10.43 (m, 1H), 9.25 (d, J = 1.1 Hz, 1H), 9.22 (dd, J = 7.0, 1.8 Hz, 1H), 8.87 (s, 1H), 8.72 (dd, J = 4.1, 1.7 Hz, 1H), 8.10 (d, J = 1.1 Hz, 1H), 7.18-7.13 (m, 1H), 6.74 (s, 1H), 6.47 (s, 1H), 6.30-6.26 (m, 1H), 4.32 (dt, J = 8.6, 2.5 Hz, 1H), 4.23 (m, 2H), 4.18-4.26 (m, 1H), 4.16- 4.11 (m, 2H), 3.66 (br dd, J = 8.8, 3.9 Hz, 1H), 3.63 (s, 3H), 3.40- | (Method 4): Rt = 3.59 min, ES⁺ m/z 586.2 [M + H]⁺ |
| | 2-((2-oxotetrahydrofuran-3- | | | 3.36 (m, 2H), 3.07-3.01 (m, 1H), | |
| | yl)amino)ethyl (1-(6-methoxy-3,4- | | | 2.94-2.88 (m, 1H), 2.45 (td, | |
| | dihydro-2H-benzo[b][1,4]oxazin-7-yl)-6- | | | J = 7.5, 5.9 Hz, 1H), 2.02-1.94 | |
| | (pyrazolo[1,5-a]pyrimidin-3-yl)-1H- | | | (m, 1H) | |
| | pyrazolo[4,3-c]pyridin-3-yl)carbamate | | | | |
| Ex- am- ple 69 | 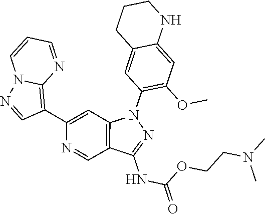 [SMILES: COc1cc2c(cc1-n1nc(NC(=O)OCCN(C)C)c3cnc(-c4cnn5cccnc45)cc31)CCCN2] | Inter- me- diate 38x | tert-butyl 6-(3-(((2- (dimethylamino)ethoxy) carbonyl)amino)-6- (pyrazolo[1,5-a] pyrimidin-3-yl)-1H- pyrazolo[4,3-c]pyridin-1- yl)-7-methoxy-3,4- dihydroquinoline-1(2H)- carboxylate (Method 1): Rt = 0.80 min, ES⁺ m/z 628.2 [M + H]⁺ | (600 MHz, 353K, DMSO-d₆) δ: 10.40 (br s, 1H), 9.21-9.23 (m, 2H), 8.87 (s, 1H), 8.71 (dd, J = 3.9 Hz, J = 1.6 Hz, 1H), 8.08 (d, J = 0.9 Hz, 1H), 7.15 (dd, J = 7.0 Hz, J = 4.0 Hz, 1H), 6.90 (s, 1H), 6.30 (s, 1H), 6.15 (s, 1H), 4.26 (t, J = 5.8 Hz, 2H), 3.62 (s, 3H), 3.25 (m, 2H), 2.65 (t, J = 6.1 Hz, 2H), 2.56 (t, J = 5.8 Hz, 2H), 2.22 (s, 6H), 1.82 (quin, J = 5.9 Hz, 2H) | (Method 4): Rt = 3.63 min, ES⁺ m/z 528.2 [M + H]⁺ |
| | 2-(dimethylamino)ethyl (1-(7-methoxy- | | | | |
| | 1,2,3,4-tetrahydroquinolin-6-yl)-6- | | | | |
| | (pyrazolo[1,5-a]pyrimidin-3-yl)-1H- | | | | |
| | pyrazolo[4,3-c]pyridin-3-yl)carbamate | | | | |
| Ex- am- ple 70 | 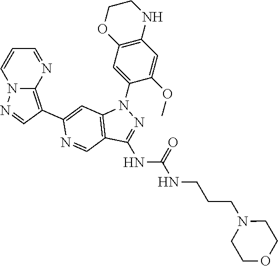 [SMILES: COc1cc2c(cc1-n1nc(NC(=O)NCCCN3CCOCC3)c3cnc(-c4cnn5cccnc45)cc31)OCCN2] | Inter- me- diate 39b | tert-butyl 6-methoxy- 7-(3-(3-(3-morpholino- propyl)ureido)-6- (pyrazolo[1,5-a]pyrimidin- 3-yl)-1H-pyrazolo[4,3- c]pyridin-1-yl)- 2,3-dihydro-4H-benzo [b][1,4]oxazine-4- carboxylate (Method 2): Rt = 1.08 min, ES⁺ m/z 685.3 [M + H]⁺ | (500 MHz, DMSO-d₆) δ: 9.87 (s, 1H), 9.35 (bs, 1H), 9.22 (dd, J = 7.1, 1.6 Hz, 1H), 8.87 (s, 1H), 8.71 (dd, J = 4.0, 1.6 Hz, 1H), 8.06 (s, 1H), 7.74 (bs, 1H), 7.15 (dd, J = 7.1, 4.0 Hz, 1H), 6.78 (s, 1H), 6.46 (s, 1H), 6.26 (bs, 1H), 4.12 (t, J = 4.3 Hz, 2H), 3.63 (s, 3H), 3.51 (t, J = 4.4 Hz, 4H) 3.36 (m, 2H), 3.27 (q, J = 6.2 Hz, 2H), 2.34-2.24 (m, 6H), 1.63 (quint., J = 6.7 Hz, 2H) | (Method 4): Rt = 3.43 min, ES⁺ m/z 585.3 [M + H]⁺ |
| | 1-(1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridin-3-yl)-3-(3- | | | | |
| | morpholinopropyl)urea | | | | |

### Example 71

### Step 1

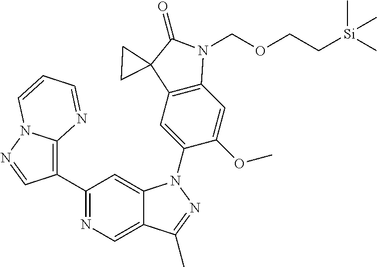 [SMILES: COc1cc2c(cc1-n1nc(C)c3cnc(-c4cnn5cccnc45)cc31)C1(CC1)C(=O)N2COCC[Si](C)(C)C]

### 6′-Methoxy-5′-(3-methyl-6-(pyrazolo[1,5-a]pyrimidin-3-yl)-1H-pyrazolo[4,3-c]pyridin-1-yl)-1′-((2-(trimethylsilyl)ethoxy)methyl)spiro[cyclopropane-1,3′-indolin]-2′-one (Example 71—Step 1)

The title compound was prepared similarly to example 40—step 1 starting from intermediate 34 and 3-(4,4,5,5-tetramethyl-1,3,2-dioxaborolan-2-yl)pyrazolo[1,5-a]pyrimidine.

LCMS (Method 1), Rt=1.32, ES m/z 568.3

### Step 2

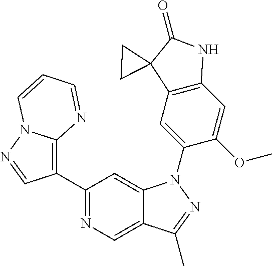 [SMILES: COc1cc2c(cc1-n1nc(C)c3cnc(-c4cnn5cccnc45)cc31)C1(CC1)C(=O)N2]

### 6′-Methoxy-5′-(3-methyl-6-(pyrazolo[1,5-a]pyrimidin-3-yl)-1H-pyrazolo[4,3-c]pyridin-1-yl)spiro[cyclopropane-1,3′-indolin]-2′-one (Example 71)

An ice bath cooled solution of intermediate example 71—step 1 (32.0 mg, 0.05 mmol) in dry DCM (1 mL) was treated with TFA (371 μL, 4.85 mmol) and stirred at RT overnight. RM was evaporated in vacuo and the residue partitioned between DCM (2×10 mL)/NaHCO₃ (15 mL). Combined organic layers were evaporated in vacuo and the residue taken in DCM (1 mL) and treated with 7N methanolic ammonia (200 μL), then stirred at RT for 4 h. RM was diluted with DCM (8 mL) and washed with sat. aq. NaHCO₃ (10 mL), and sat. aq. NaCl (10 mL). Organic layer was evaporated in vacuo and the residue purified by flash chromatography on Si cartridge, by eluting with 0-100% DCM/MeOH/NH₄OH (90:5:0.5) in DCM, to afford the title product (12.5 mg).

LCMS (Method 4): Rt=3.89 min, ES⁺ m/z 438.2 [M+H]⁺

¹H-NMR (600 MHz, DMSO-d₆) δ:10.83 (s, 1H), 9.22 (dd, J=6.9, 1.7 Hz, 1H), 9.12 (d, J=1.1 Hz, 1H), 8.88 (s, 1H), 8.70 (dd, J=4.0, 1.7 Hz, 1H), 8.17 (d, J=1.1 Hz, 1H), 7.14-7.16 (m, 2H), 6.86 (s, 1H), 3.78 (s, 3H), 2.63 (s, 3H), 1.58-1.61 (m, 2H), 1.44-1.47 (m, 2H).

### Example 72

### Step 1

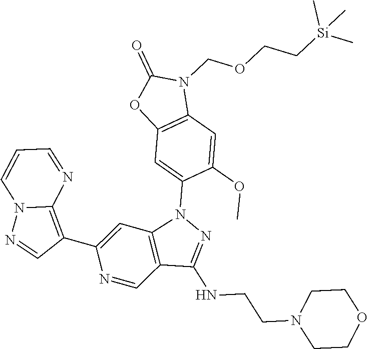 [SMILES: COc1cc2c(cc1-n1nc(NCCN3CCOCC3)c3cnc(-c4cnn5cccnc45)cc31)oc(=O)n2COCC[Si](C)(C)C]

### 5-Methoxy-6-(3-((2-morpholinoethyl)amino)-6-(pyrazolo[1,5-a]pyrimidin-3-yl)-1H-pyrazolo[4,3-c]pyridin-1-yl)-3-((2-(trimethylsilyl)ethoxy)methyl)benzo[d]oxazol-2 (3H)-one (Example 72—Step 1)

The title compound was prepared similarly to intermediate example 72—step 1 starting from intermediate 19 and 3-(4,4,5,5-tetramethyl-1,3,2-dioxaborolan-2-yl)pyrazolo[1,5-a]pyrimidine.

LCMS (Method 1), Rt=0.79, ES⁺ m/z 658.5

### Step 2

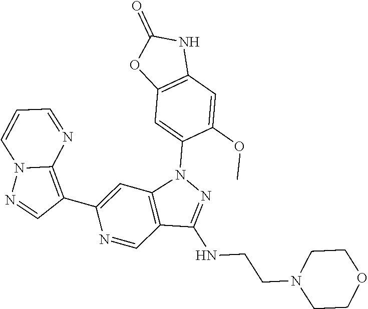 [SMILES: COc1cc2[nH]c(=O)oc2cc1-n1nc(NCCN2CCOCC2)c2cnc(-c3cnn4cccnc34)cc21]

### 5-Methoxy-6-(3-((2-morpholinoethyl)amino)-6-(pyrazolo[1,5-a]pyrimidin-3-yl)-1H-pyrazolo[4,3-c]pyridin-1-yl)benzo[d]oxazol-2 (3H)-one (Example 72)

The title compound was prepared similarly to example 72 (step 2) starting from intermediate example 72—step 1.

LCMS (Method 4): Rt=3.52 min, ES⁺ m/z 528.2 [M+H]⁺

¹H-NMR (500 MHz, DMSO-d₆) δ: 11.87 (s, 1H), 9.20 (d, J=7.1 Hz, 1H), 9.06 (s, 1H), 8.85 (s, 1H), 8.67 (m, 1H), 8.04 (s, 1H), 7.42 (s, 1H), 7.13 (dd, J=7.5, 3.4 Hz, 1H), 7.02 (s, 1H), 6.68 (t, J=5.3 Hz, 1H), 3.82 (s, 3H), 3.56-3.63 (m, 4H), 3.44 (q, J=6.7 Hz, 2H), 2.60-2.65 (m, 2H); 2.42-2.48 (m, 2H, overlapped with DMSO); 2.35-2.38 (m, 2H).

### Example 73

### Step 1

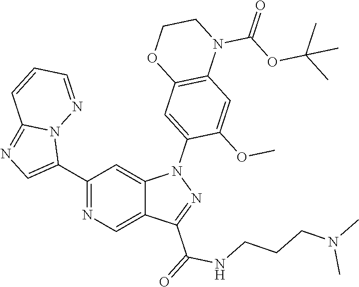 [SMILES: COc1cc2c(cc1-n1nc(C(=O)NCCCN(C)C)c3cnc(-c4cnc5cccnn45)cc31)OCCN2C(=O)OC(C)(C)C]

### tert-Butyl 7-(3-((3-(dimethylamino)propyl)carbamoyl)-6-(imidazo[1,2-b]pyridazin-3-yl)-1H-pyrazolo[4,3-c]pyridin-1-yl)-6-methoxy-2,3-dihydro-4H-benzo[b][1,4]oxazine-4-carboxylate (Example 73—Step 1)

To a degassed mixture of intermediate 37a (70 mg, 0.13 mmol), bis(triphenylphosphine)palladium (II) chloride (14 mg, 19 μmol) in DMF (1 mL), intermediate 12 (270 mg, 0.33 mmol) in DMF (1 mL) was added and RM stirred at 110° C. overnight. After cooling to RT, RM was partitioned between EtOAc (25 mL) and water (15 mL). Aqueous phase was brought to pH 9 with aq. 2N NaOH and extracted with EtOAc (15 mL). Combined organic layers were evaporated in vacuo. The residue was purified by flash chromatography on Si cartridge by eluting with 0-90% DCM/MeOH/NH₄OH (90:9:1.5) in DCM to afford the title product (38 mg).

LCMS (Method 1), Rt=0.86, ES⁺ m/z 628.4

### Step 2

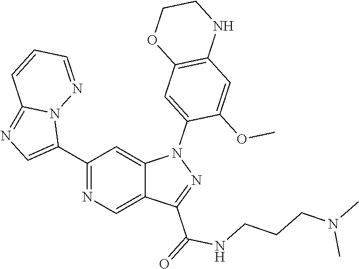 [SMILES: COc1cc2c(cc1-n1nc(C(=O)NCCCN(C)C)c3cnc(-c4cnc5cccnn45)cc31)OCCN2]

### N-(3-(Dimethylamino)propyl)-6-(imidazo[1,2-b]pyridazin-3-yl)-1-(6-methoxy-3,4-dihydro-2H-benzo[b][1,4]oxazin-7-yl)-1H-pyrazolo[4,3-c]pyridine-3-carboxamide (Example 73)

The title compound was prepared similarly to example 40 (step 2).

LCMS (Method 4), Rt=3.94 min, ES⁺ m/z 528.2 [M+H]⁺

¹H-NMR (600 MHz, DMSO-d₆) δ: 9.51 (s, 1H), 8.76 (br s, 2H), 8.59 (s, 1H), 8.52 (s, 1H), 8.30 (d, J=9.2 Hz, 1H), 8.24 (s, 1H), 7.36 (dd, J=9.3, 4.4 Hz, 1H), 6.91 (s, 1H), 6.50 (s, 1H), 4.14 (br s, 2H), 3.67 (s, 3H), 3.41-3.35 (m, 4H), 2.40 (br t, J=7.2 Hz, 2H), 2.23 (s, 6H), 1.78-1.72 (m, 2H).

### Example 74

### Step 1

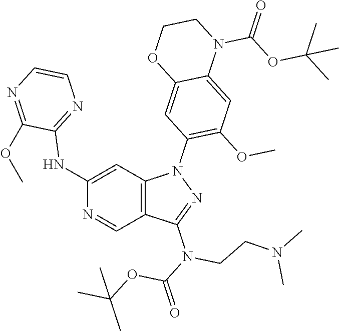 [SMILES: COc1cc2c(cc1-n1nc(N(CCN(C)C)C(=O)OC(C)(C)C)c3cnc(Nc4nccnc4OC)cc31)OCCN2C(=O)OC(C)(C)C]

### tert-Butyl 7-(3-((tert-butoxycarbonyl)(2-(dimethylamino)ethyl)amino)-6-((3-methoxypyrazin-2-yl)amino)-1H-pyrazolo[4,3-c]pyridin-1-yl)-6-methoxy-2,3-dihydro-4H-benzo[b][1,4]oxazine-4-carboxylate (Example 74—Step 1)

A degassed mixture of intermediate 31a (25 mg, 41 μmol), 2-amino-3-methoxypyrazine (6.7 mg, 54 μmol), sodium tert-butoxide (6.0 mg, 62 μmol) and XPhos PdG3 (3.5 mg, 4.1 μmol) in dioxane (600 μL) was stirred at 100° C. overnight. After cooling to RT, RM was chromatographed on Si cartridge by eluting with 0-30% DCM/MeOH/NH₄OH (90:15:1.5) in DCM to afford the title product (11 mg).

LCMS (Method 2): Rt=1.59 min ES⁺ m/z 692.4 [M+H]⁺

### Step 2

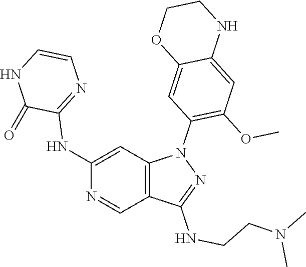 [SMILES: COc1cc2c(cc1-n1nc(NCCN(C)C)c3cnc(Nc4ncc[nH]c4=O)cc31)OCCN2]

### 3-((3-((2-(dimethylamino)ethyl)amino)-1-(6-methoxy-3,4-dihydro-2H-benzo[b][1,4]oxazin-7-yl)-1H-pyrazolo[4,3-c]pyridin-6-yl)amino)pyrazin-2 (1H)-one (Example 74)

A solution of intermediate example 74—Step 1 (11.0 mg, 8 μmol), NaI (3.58 mg, 24 μmol) and TMS-Cl (9.0 μL, 72 μmol) in acetonitrile (0.5 mL) was stirred at 85° C. for 2 h. After cooling to RT, RM was chromatographed on Si cartridge by eluting with 0-100% DCM/MeOH/NH₄OH (90:15:1.5) in DCM to afford the title product (2 mg).

¹H-NMR (600 MHz, DMSO-d₆) δ: 8.74 (d, J=0.9 Hz, 1H), 8.64 (s, 1H), 7.90 (d, J=0.9 Hz, 1H), 6.93 (d, J=4.4 Hz, 1H), 6.86 (d, J=4.4 Hz, 1H), 6.68 (s, 1H), 6.52 (m, 1H), 6.42 (s, 1H), 6.12 (s, 1H), 4.11 (t, J=4.2 Hz, 2H), 3.65 (s, 3H), 2.65 (m, 2H), 2.30 (br s, 6H).

LCMS (Method 4): Rt=4.34, ES⁺ m/z 478.1 [M+H]⁺

### Examples 75 to 76

The following examples were prepared by a two step process similarly to example 74 starting from the indicated intermediate.

| | | Step 1 | | | |
|---|---|---|---|---|---|
| | | | Name of intermediate | Step 2 | |
| Example | Structure/name | Intermediate | Step 1/LCMS | ¹H-NMR | LCMS |
| Example | N-(3-(dimethylamino)propyl)-1-(6- | Intermediate | tert-butyl 7-(3-((3- | (500 MHz, DMSO-d₆) δ: | (Method 4): Rt = |
| 75 | methoxy-3,4-dihydro-2H- | 37a and 2- | (dimethyl- | 9.16 (d, J = 0.9 Hz, 1H), | 3.57, ES⁺ m/z |
| | benzo[b][1,4]oxazin-7-yl)-6-((3-oxo- | amino-3- | amino)propyl)carbamoyl)- | 8.80 (s, 1H), 8.64 (t, | 520.2 [M + H]⁺ |
| | 3,4-dihydropyrazin-2-yl)amino)-1H- | methoxypyrazine | 6-((3-methoxypyrazin-2- | J = 6.0 Hz, 1H), 8.15 (d, | |
| | pyrazolo[4,3-c]pyridine-3-carboxamide | | yl)amino)-1H- | J = 0.9 Hz, 1H), 6.95 (d, | |
| | | | pyrazolo[4,3- | J = 4.3 Hz, 1H), 6.90 (d, | |
| | | | c]pyridin-1-y1)-6- | J = 4.3 Hz, 1H), 6.83 (s, | |
| | | | methoxy-2,3-dihydro- | 1H), 6.47 (s, 1H), 6.35 (s, | |
| | | | 4H- | 1H), 4.13 (t, J = 4.3 Hz, | |
| | | | benzo[b][1,4]oxazine- | 2H), 3.64 (s, 3H), 3.38 | |
| | | | 4-carboxylate | (br s, 2H), 3.35-3.30 (m, | |
| | | | (Method 2): Rt = 1.57 | 2H), 2.29 (t, J = 7.0 Hz, | |
| | | | min, ES⁺ m/z 634.5 | 2H), 2.15 (s, 6H), 1.69 | |
| | | | [M + H]⁺ | (quin, J = 7.1 Hz, 2H) | |
| Example | N-(3-(4,4-difluoropiperidin-1- | Intermediate | N-(3-(4,4- | (500 MHz, DMSO-d₆) δ: | (Method 4): Rt = |
| 76 | yl)propyl)-1-(6-methoxy-3,4-dihydro- | 37d and 2- | difluoropiperidin-1- | 12.20 (br d, J = 4.7 Hz, | 3.78, ES⁺ m/z |
| | 2H-benzo[6][1,4]oxazin-7-yl)-6-((3- | amino-3- | yl)propyl)-1-(6- | 1H), 9.15 (d, J = 1.0 Hz, | 596.2 [M + H]⁺ |
| | oxo-3,4-dihydropyrazin-2-yl)amino)- | methoxypyrazine | methoxy-3,4-dihydro- | 1H), 8.79 (s, 1H), 8.74 | |
| | 1H-pyrazolo[4,3-c]pyridine-3- | | 2H- | (t, J = 5.7 Hz, 1H), 8.13 | |
| | carboxamide | | benzo[b][1,4]oxazin- | (d, J = 1.0 Hz, 1H), 6.93 | |
| | | | 7-yl)-6-((3- | (d, J = 4.0 Hz, 1H), 6.89 | |
| | | | methoxypyrazin-2- | (dd, J = 5.7, 4.7 Hz, 1H), | |
| | | | yl)amino)-1H- | 6.80 (s, 1H), 6.44 (s, | |
| | | | pyrazolo[4,3- | 1H), 6.32 (br s, 1H), | |
| | | | c]pyridine-3- | 4.08-4.14 (m, 2H), 3.61 | |
| | | | carboxamide | (s, 3H), 3.34-3.40 (m, | |
| | | | (Method 2): Rt = 1.13 | 4H), 2.49 (m, 6H, under | |
| | | | min, ES⁺ m/z 610.3 | DMSO), 1.83-2.00 (m, | |
| | | | [M + H]⁺ | 4H), 1.66-1.76 (m, 2H) | |

### Example 77

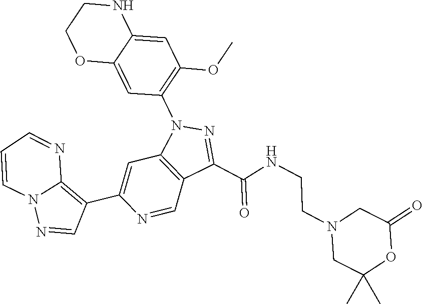 [SMILES: COc1cc2c(cc1-n1nc(C(=O)NCCN3CC(=O)OC(C)(C)C3)c3cnc(-c4cnn5cccnc45)cc31)OCCN2]

### N-(2-(2,2-dimethyl-6-oxomorpholino)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide (Example 77)

The title compound was prepared in a similar manner to Example 1 from Intermediate 45f and 3-(4,4,5,5-tetramethyl-1,3,2-dioxaborolan-2-yl)pyrazolo[1,5-a]pyrimidine.

LCMS (Method 3), Rt=4.51 min, ES⁺ m/z 598.3 [M+H]⁺

¹H-NMR (500 MHz, DMSO-d₆) δ: 9.45 (d, J=1.2 Hz, 1H), 9.24 (dd, J=6.9, 1.7 Hz, 1H), 8.91 (s, 1H), 8.74 (dd, J=4.1, 1.7 Hz, 1H), 8.53 (t, J=6.0 Hz, 1H), 8.25 (d, J=0.9 Hz, 1H), 7.17 (dd, J=7.0, 4.3 Hz, 1H), 6.86 (s, 1H), 6.49 (s, 1H), 6.36-6.41 (m, 1H), 4.14 (t, J=4.3 Hz, 2H), 3.63 (s, 3H), 3.48 (q, J=6.3 Hz, 2H), 3.36-3.42 (m, 2H), 3.29 (s, 2H), 2.57-2.66 (m, 4H), 1.33 (s, 6H).

### Example 78

Step 1

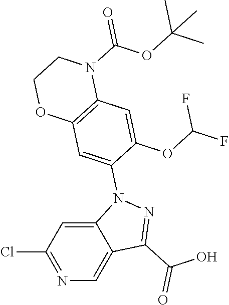 [SMILES: CC(C)(C)OC(=O)N1CCOc2cc(-n3nc(C(=O)O)c4cnc(Cl)cc43)c(OC(F)F)cc21]

### 1-(4-(tert-Butoxycarbonyl)-6-(difluoromethoxy)-3,4-dihydro-2H-benzo[b][1,4]oxazin-7-yl)-6-chloro-1H-pyrazolo[4,3-c]pyridine-3-carboxylic acid (Example 78—Step 1)

Title compound was prepared similarly to Intermediate 24 (Method 1) starting from of 6-chloro-1H-pyrazolo[4,3-c]pyridine-3-carboxylic acid and Intermediate 1.

LCMS (Method 1), Rt=1.22 min ES⁺ m/z 497.1/499.0 [M+H]⁺

### Step 2

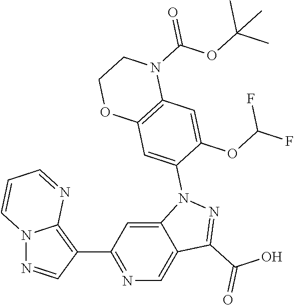 [SMILES: CC(C)(C)OC(=O)N1CCOc2cc(-n3nc(C(=O)O)c4cnc(-c5cnn6cccnc56)cc43)c(OC(F)F)cc21]

### 1-(4-(tert-Butoxycarbonyl)-6-(difluoromethoxy)-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylic acid (Example 78—step 2)

To a degassed mixture of Example 78—step 1 (200 mg, 0.40 mmol), 3-(4,4,5,5-tetramethyl-1,3,2-dioxaborolan-2-yl)pyrazolo[1,5-a]pyrimidine (138 mg, 0.56 mmol) and K₃PO₄ (0.5 M in water, 1.61 mL, 0.81 mmol) in THF/water (5.61 mL), XPhos Pd G3 (19 mg, 22 μmol) was added and RM heated at 80° C. for 1 h. Warm reaction mixture was filtrated and THF evaporated in vacuo. Water was added to RM until solids dissolved, filtered and added with sat. aq. NH₄Cl to form a precipitate that was collected by filtration to give the title product (77 mg) used in the next step without further purifications.

LCMS (Method 1), Rt=1.01 min, ES⁺ m/z 580.2 [M+H]⁺

### Step 3

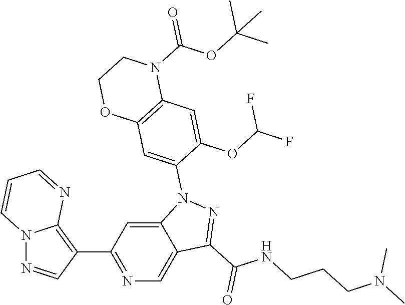 [SMILES: CN(C)CCCNC(=O)c1nn(-c2cc3c(cc2OC(F)F)N(C(=O)OC(C)(C)C)CCO3)c2cc(-c3cnn4cccnc34)ncc12]

### tert-Butyl 6-(difluoromethoxy)-7-(3-((3-(dimethylamino)propyl)carbamoyl)-6-(pyrazolo[1,5-a]pyrimidin-3-yl)-1H-pyrazolo[4,3-c]pyridine-1-yl)-2,3-dihydro-4H-benzo[b][1,4]oxazine-4-carboxylate (Example 78—Step 3)

Title compound was prepared similarly to Intermediate 37a starting from Example 78-step 2 and N′,N′-dimethylpropane-1,3-diamine.

LCMS (Method 1), Rt=0.86 min, ES⁺ m/z 664.3 [M+H]⁺

### Step 4

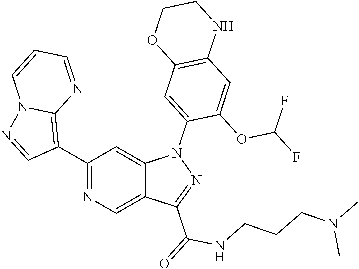 [SMILES: CN(C)CCCNC(=O)c1nn(-c2cc3c(cc2OC(F)F)NCCO3)c2cc(-c3cnn4cccnc34)ncc12]

### 1-(6-(Difluoromethoxy)-3,4-dihydro-2H-benzo[b][1,4]oxazin-7-yl)-N-(3-(dimethylamino)propyl)-6-(pyrazolo[1,5-a]pyrimidin-3-yl)-1H-pyrazolo[4,3-c]pyridine-3-carboxamide (Example 78)

The title compound was prepared similarly to step 2 of example 40 starting from Example 78—step 3.

LCMS (Method 4): Rt=3.92, ES⁺ m/z 564.2 [M+H]⁺

¹H-NMR (500 MHz, DMSO-d₆) δ: 9.48 (d, J=1.2 Hz, 1H), 9.24 (dd, J=7.0, 1.8 Hz, 1H), 8.92 (s, 1H), 8.74 (dd, J=4.1, 1.7 Hz, 1H), 8.70 (t, J=6.0 Hz, 1H), 8.29 (d, J=0.9 Hz, 1H), 7.18 (dd, J=7.0, 4.3 Hz, 1H), 7.06 (s, 1H), 6.95 (t, J=73.9 Hz, 1H), 6.66-6.71 (m, 2H), 4.20 (t, J=4.3 Hz, 2H), 3.39-3.44 (m, 2H), 3.34-3.38 (m, 2H), 2.28 (t, J=7.0 Hz, 2H), 2.14 (s, 6H), 1.70 (quin, J=7.1 Hz, 2H).

### Example 79

### Step 1

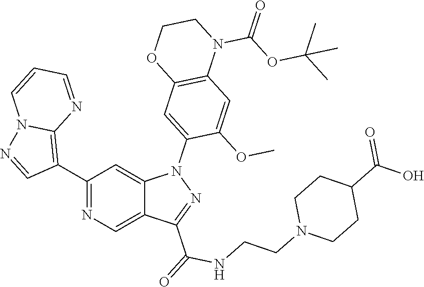 [SMILES: COc1cc2c(cc1-n1nc(C(=O)NCCN3CCC(C(=O)O)CC3)c3cnc(-c4cnn5cccnc45)cc31)OCCN2C(=O)OC(C)(C)C]

### 1-(2-(1-(4-(tert-Butoxycarbonyl)-6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylic acid (Example 79—Step 1)

A degassed mixture of Intermediate 37m-1 (348 mg, 0.55 mmol), 3-(4,4,5,5-tetramethyl-1,3,2-dioxaborolan-2-yl)pyrazolo[1,5-a]pyrimidine (202 mg, 0.82 mmol), K₃PO₄ (233 mg, 1.1 mmol) and XPhos PdG3 (46 mg, 0.055 mmol) in THF/water (2:1, 9 mL) was stirred at 70° C. for 45 min After cooling to RT, RM was partitioned between EtOAc (30 mL) and water (30 mL). After adjusting pH from 9.5 to 6.5, aqueous layer was extracted with DCM/isopropanol mix 4/1 (4×20 mL). Combined organic layers were passed through a phase separator and evaporated in vacuo.

The residue was purified by flash chromatography on Si cartridge by eluting with 0-100% DCM/MeOH:HCO₂H (90:9:2) in DCM to afford the title product (320 mg).

LCMS (Method 1): Rt=0.74 min, ES⁺ m/z 698.4 [M+H]⁺

### Step 2

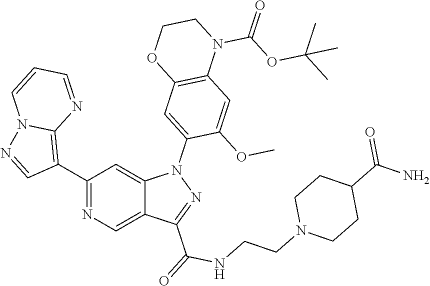 [SMILES: COc1cc2c(cc1-n1nc(C(=O)NCCN3CCC(C(N)=O)CC3)c3cnc(-c4cnn5cccnc45)cc31)OCCN2C(=O)OC(C)(C)C]

### tert-Butyl 7-(3-((2-(4-carbamoylpiperidin-1-yl)ethyl)carbamoyl)-6-(pyrazolo[1,5-a]pyrimidin-3-yl)-1-H-pyrazolo[4,3-c]pyridin-1-yl)-6-methoxy-2,3-dihydro-4H-benzo[b][1,4]oxazine-4-carboxylate (Example 79—step 2)

To a pre-stirred solution of Example 79—step 1 (100 mg, 0.14 mmol), HATU (65.4 mg, 0.17 mmol) and DIPEA (74.9 μL, 0.43 mmol) in DMF (2.0 mL), NH₄Cl (61.3 mg, 0.57 mmol) and DIPEA (99.9 μL, 0.57 mmol) were added and RM stirred at RT for 1 h. A second portion of HATU (15.0 mg, 0.04 mmol) was added and the mixture stirred for further 1 h. Volatiles were removed in vacuo and residue purified by flash chromatography on Si cartridge by eluting with 0-100% DCM/MeOH/NH₄OH (90:9:0.5) in DCM to afford the title product (32 mg).

LCMS (Method 1): Rt=0.74 min, ES⁺ m/z 697.4 [M+H]⁺

### Step 3

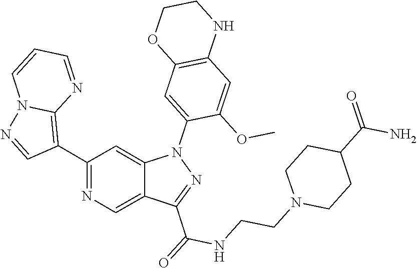 [SMILES: COc1cc2c(cc1-n1nc(C(=O)NCCN3CCC(C(N)=O)CC3)c3cnc(-c4cnn5cccnc45)cc31)OCCN2]

### N-(2-(4-Carbamoylpiperidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide (Example 79)

Title compound was prepared similarly to step 2 of Example 40 starting from Example 79-step 2.

LCMS (Method 4): Rt=3.61, ES⁺ m/z 597.2 [M+H]⁺

¹H-NMR (500 MHz, DMSO-d₆) δ: 9.46 (d, J=0.9 Hz, 1H), 9.23 (dd, J=7.0, 1.8 Hz, 1H), 8.91 (s, 1H), 8.73 (dd, J=4.1, 1.7 Hz, 1H), 8.44 (t, J=5.8 Hz, 1H), 8.24 (d, J=0.9 Hz, 1H), 7.19 (br s, 1H), 7.17 (dd, J=7.0, 4.0 Hz, 1H), 6.89 (s, 1H), 6.69 (br s, 1H), 6.49 (s, 1H), 6.38 (s, 1H), 4.14 (t, J=4.3 Hz, 2H), 3.63 (s, 3H), 3.44 (q, J=6.7 Hz, 2H), 3.39 (m, 2H), 2.93 (m, 2H), 2.48 (m, 2H), 2.00-2.08 (m, 1H), 1.90-1.99 (m, 2H), 1.66 (m, 2H), 1.50-1.58 (m, 2H).

### Example 80

### Step 1

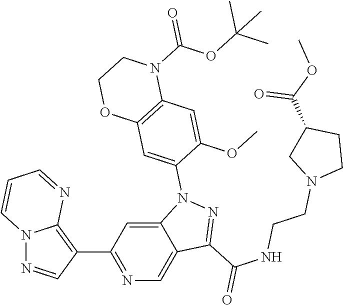 [SMILES: COC(=O)[C@@H]1CCN(CCNC(=O)c2nn(-c3cc4c(cc3OC)N(C(=O)OC(C)(C)C)CCO4)c3cc(-c4cnn5cccnc45)ncc23)C1]

### tert-Butyl (R)-6-methoxy-7-(3-((2-(3-(methoxycarbonyl)pyrrolidin-1-V)ethyl)carbamoyl)-6-(pyrazolo[1,5-a]pyrimidin-3-yl)-1H-pyrazolo[4,3-c]pyridin-1-yl)-2,3-dihydro-4H-benzo[b][1,4]oxazine-4-carboxylate (Example 80—step 1)

Title compound was prepared similarly to Example 1 from Intermediate 42b and 3-(4,4,5,5-tetramethyl-1,3,2-dioxaborolan-2-yl)pyrazolo[1,5-a]pyrimidine.

LCMS (Method 2): Rt=1.17 min, ES⁺ m/z 698.3 [M+H]⁺

### Step 2

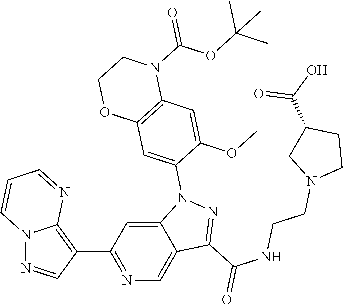 [SMILES: COc1cc2c(cc1-n1nc(C(=O)NCCN3CC[C@@H](C(=O)O)C3)c3cnc(-c4cnn5cccnc45)cc31)OCCN2C(=O)OC(C)(C)C]

### (R)-1-(2-(1-(4-(tert-butoxycarbonyl)-6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylic acid (Example 80—step 2)

A solution of Example 80—step 1 (574 mg, 0.82 mmol) and LiOH (1.00 M in water, 4.11 mL, 4.11 mmol) in THF (10 mL) was stirred at RT overnight. RM was diluted with water and washed with EtOAc, then pH of the aqueous layer adjusted to 5 and aqueous layer extracted with DCM:i-PrOH 8:1 (4×15 mL). Combined organic layers were dried over Na₂SO₄ and evaporated to dryness to afford the title compound (553 mg) that was used in the next step without further purification.

LCMS (Method 1): Rt=0.82 min, ES m/z 684.4 [M+H]⁺

### Step 3

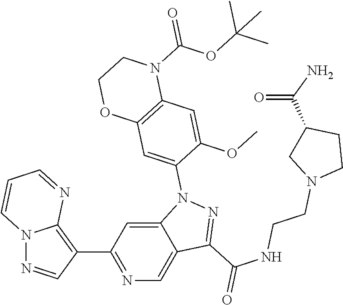 [SMILES: COc1cc2c(cc1-n1nc(C(=O)NCCN3CC[C@@H](C(N)=O)C3)c3cnc(-c4cnn5cccnc45)cc31)OCCN2C(=O)OC(C)(C)C]

### tert-Butyl (R)-7-(3-((2-(3-carbamoylpyrrolidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 80-Step 2)

Title compound was prepared similarly to step 2 of Example 79 starting from Example 80-step 1.

LCMS (Method 2): Rt=1.00 min, ES⁺ m/z 683.4 [M+H]⁺

### Step 4

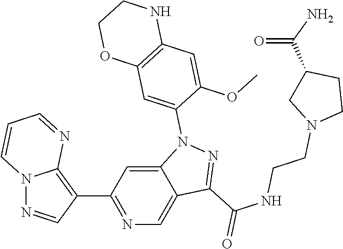 [SMILES: COc1cc2c(cc1-n1nc(C(=O)NCCN3CC[C@@H](C(N)=O)C3)c3cnc(-c4cnn5cccnc45)cc31)OCCN2]

### (R)—N-(2-(3-Carbamoylpyrrolidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide (Example 80)

Title compound was prepared similarly to step 2 of Example 40 starting from Example 80-step 3.

LCMS (Method 4): Rt=3.56, ES⁺ m/z 583.1 [M+H]⁺

¹H-NMR (500 MHz, DMSO-d₆) δ: 9.46 (d, J=0.9 Hz, 1H), 9.24 (dd, J=7.0, 1.5 Hz, 1H), 8.91 (s, 1H), 8.74 (dd, J=4.1, 1.7 Hz, 1H), 8.47 (t, J=5.8 Hz, 1H), 8.23-8.26 (m, 1H), 7.25 (br s, 1H), 7.17 (dd, J=7.0, 4.3 Hz, 1H), 6.89 (s, 1H), 6.76 (br s, 1H), 6.49 (s, 1H), 6.36-6.41 (m, 1H), 4.14 (br t, J=4.1 Hz, 2H), 3.63 (s, 3H), 3.43 (q, J=6.6 Hz, 2H), 3.37-3.41 (m, 2H), 2.84-2.90 (m, 1H), 2.79 (quin, J=7.8 Hz, 1H), 2.66-2.73 (m, 1H), 2.56-2.65 (m, 2H), 2.37-2.48 (m, 2H), 1.87 (q, J=7.3 Hz, 2H).

### Examples 81 to 86

The following examples were prepared by a two steps process similarly to example 40 starting from the indicated intermediate. It is additionally stated if base, catalyst, solvent or temperature were varied.

| | | Step 1 | | | |
|---|---|---|---|---|---|
| Ex- | | | Name of | | |
| am- | | Inter- | intermediate | Step 2 | |
| ple | Structure/name | mediate | Step 1/LCMS | ¹H-NMR | LCMS |
| Ex- am- ple 81 | 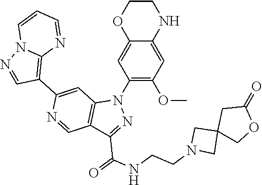 [SMILES: COc1cc2c(cc1-n1nc(C(=O)NCCN3CC4(COC(=O)C4)C3)c3cnc(-c4cnn5cccnc45)cc31)OCCN2] | Inter- mediate 42a | tert-butyl 6- methoxy-7-(3-((2- (5-oxo-6-oxa-2- azaspiro[3.4] octan-2-yl)ethyl) carbamoyl)- 6-(pyrazolo[1,5-a] pyrimidin-3- yl)-1H-pyrazolo [4,3-c]pyridin- 1-yl)-2,3- dihydro-4H-benzo | (500 MHz, DMSO-d₆) δ: 9.45 (d, J = 0.9 Hz, 1H), 9.23 (dd, J = 7.0, 1.8 Hz, 1H), 8.91 (s, 1H), 8.73 (dd, J = 4.1, 1.7 Hz, 1H), 8.46 (t, J = 5.8 Hz, 1H), 8.24 (d, J = 0.9 Hz, 1H), 7.17 (dd, J = 7.1, 4.0 Hz, 1H), 6.89 (s, 1H), 6.49 (s, 1H), 6.38 (s, 1H), 4.35 (s, 2H), 4.14 (t, J = 4.4 Hz, 2H), 3.63 (s, 3H), 3.39 (m, 2H), 3.27 (m, 2H), 3.23 (s, 4H), 2.73 (s, 2H), 2.52-2.60 (m, 2H). | (Method 4): Rt = 3.72 min, ES⁺ m/z 596.3 [M + H]⁺ |
| | 1-(6-methoxy-3,4-dihydro-2H- | | [b][1,4]oxazine- | | |
| | benzo[b][1,4]oxazin-7-yl)-N-(2-(7- | | 4-carboxylate | | |
| | oxo-6-oxa-2-azaspiro[3.4]octan-2- | | (Method 2): Rt = | | |
| | yl)ethyl)-6-(pyrazolo[1,5-a]pyrimidin- | | 1.08 min, | | |
| | 3-yl)-1H-pyrazolo[4,3-c]pyridine-3- | | ES⁺ m/z 696.4 | | |
| | carboxamide | | [M + H]⁺ | | |
| Ex- am- ple 82 | 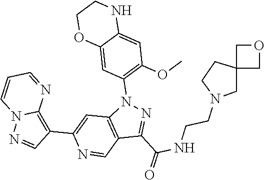 [SMILES: COc1cc2c(cc1-n1nc(C(=O)NCCN3CCC4(COC4)C3)c3cnc(-c4cnn5cccnc45)cc31)OCCN2] | Inter- mediate 45c | tert-butyl 7-(3- ((2-(2-oxa-6- azaspiro[3.4] octan-6-yl)ethyl) carbamoyl)- 6-(pyrazolo[1,5-a] pyrimidin-3- yl)-1H-pyrazolo [4,3-c]pyridin- 1-yl)-6-methoxy- 2,3-dihydro-4H- | (600 MHz, DMSO-d₆) δ: 9.46 (d, J = 0.9 Hz, 1H), 9.23 (dd, J = 7.0, 1.6 Hz, 1H), 8.91 (s, 1H), 8.73 (dd, J = 4.1, 1.7 Hz, 1H), 8.49 (t, J = 6.1 Hz, 1H), 8.24 (d, J = 1.4 Hz, 1H), 7.16 (dd, J = 6.7, 3.9 Hz, 1H), 6.88 (s, 1H), 6.49 (s, 1H), 6.38 (t, J = 2.6 Hz, 1H), 4.48 (d, J = 5.8 Hz, 2H), 4.47 (d, J = 5.8 Hz, 2H), 4.13 (t, J = 4.7 Hz, 2H), 3.63 (s, 3H), 3.41 (q, J = 6.6 Hz, 2H), 3.38- | (Method 4): Rt = 3.71 min, ES⁺ m/z 582.2 [M + H]⁺ |
| | N-(2-(2-oxa-6-azaspiro[3.4]octan-6- | | benzo[b][1,4] | 3.40 (m, 2H), 2.81 (s, 2H), 2.58 | |
| | yl)ethyl)-1-(6-methoxy-3,4-dihydro- | | oxazine-4- | (t, J = 6.7 Hz, 2H), 2.50 (m, 2H, | |
| | 2H-benzo[b][1,4]oxazin-7-yl)-6- | | carboxylate | overlapped with DMSO); 2.02 (t, | |
| | (pyrazolo[1,5-a]pyrimidin-3-yl)-1H- | | | J = 6.9 Hz, 2H). | |
| | pyrazolo[4,3-c]pyridine-3- | | | | |
| | carboxamide | | | | |
| Ex- am- ple 83 | 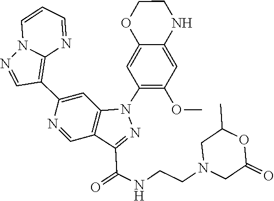 [SMILES: COc1cc2c(cc1-n1nc(C(=O)NCCN3CC(=O)OC(C)C3)c3cnc(-c4cnn5cccnc45)cc31)OCCN2] | Inter- mediate 45e Solvent: 1,2-di- methoxy- ethane in the presence of KF and Pd (PPh₃)₂Cl₂ | tert-butyl 6- methoxy-7-(3-((2- (2-methyl-6-oxo- morpholino)ethyl) carbamoyl)-6- (pyrazolo [1,5-a]pyrimidin- 3-yl)-1H-pyrazolo [4,3-c]pyridin- 1-yl)- 2,3-dihydro-4H- benzo[b][1,4] oxazine- | (500 MHz, DMSO-d₆) δ: 9.45 (d, J = 1.1 Hz, 1H), 9.23 (dd, J = 6.9 Hz, J = 1.7 Hz, 1H), 8.91 (s, 1H), 8.73 (dd, J = 4.0 Hz, J = 1.6 Hz, 1H), 8.56 (t, J = 5.9 Hz, 1H), 8.24 (d, J = 1.1 Hz, 1H), 7.17 (dd, J = 6.9 Hz, J = 4.1 Hz, 1H), 6.87 (s, 1H), 6.49 (s, 1H), 6.38 (s, 1H), 4.59-4.51 (m, 1H), 4.13 (t, J = 4.0 Hz, 2H), 3.62 (s, 3H), 3.58 (d, J = 17.2 Hz, 1H), 3.47 (q, J = 6.3 Hz, 2H), 3.41-3.37 (m, 2H), 3.09 (d, J = 17.2 Hz, 1H), 3.03 (m, 1H), 2.68-2- 54 (m, 2H), 2.30 (dd, J = 12.3, 8.8 Hz, | (Method 4): Rt = 4.49 min, ES⁺ m/z 584.1 [M + H]⁺ |
| | 1-(6-methoxy-3,4-dihydro-2H- | | 4-carboxylate | 1H), 1.25 (d, J = 6.3 Hz, 3H.). | |
| | benzo[b][1,4]oxazin-7-yl)-N-(2-(2- | | (Method 2): Rt = | | |
| | methyl-6-oxomorpholino)ethyl)-6- | | 1.16 min, | | |
| | (pyrazolo[1,5-a]pyrimidin-3-yl)-1H- | | ES⁺ m/z 684.4 | | |
| | pyrazolo[4,3-c]pyridine-3- | | [M + H]⁺ | | |
| | carboxamide | | | | |
| Ex- am- ple 84 | 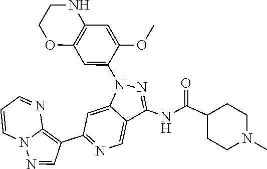 [SMILES: COc1cc2c(cc1-n1nc(NC(=O)C3CCN(C)CC3)c3cnc(-c4cnn5cccnc45)cc31)OCCN2] | Inter- mediate 43 | tert-butyl 6-methoxy- 7-(3-(1-methyl- piperidine-4- carboxamido)-6- (pyrazolo[1,5-a] pyrimidin-3- yl)-1H- pyrazolo[4,3-c] pyridin-1-yl)-2,3- dihydro-4H-benzo | (500 MHz, DMSO-d₆) δ: 10.82 (s, 1H), 9.27 (s, 1H), 9.21 (d, J = 5.9 Hz, 1H), 8.87 (s, 1H), 8.71 (bs, 1H), 8.08 (s, 1H), 7.14-7.16 (m, 1H), 6.75 (s, 1H), 6.47 (s, 1H), 6.28 (bs, 1H), 4.13 (bs, 2H), 3.62 (s, 3H), 3.37 (m, 2H, overlapped with HDO), 2.84 (d, J = 10.4 Hz, 2H), 2.46 (m, 1H, overlapped with dmso), 2.17 (s, 3H), 1.82-1.91 (m, 4H), 1.70-1.77 (m, 2H). | (Method 4): Rt = 3.54 min, ES⁺ m/z 540.2 [M + H]⁺ |
| | N-(1-(6-methoxy-3,4-dihydro-2H- | | [b][1,4]oxazine- | | |
| | benzo[b][1,4]oxazin-7-yl)-6- | | 4-carboxylate | | |
| | (pyrazolo[1,5-a]pyrimidin-3-yl)-1H- | | (Method 2): Rt = | | |
| | pyrazolo[4,3-c]pyridin-3-yl)-1- | | 1.10 min, | | |
| | methylpiperidine-4-carboxamide | | ES⁺ m/z 640.4 | | |
| | | | [M + H]⁺ | | |
| Ex- am- ple 85 | 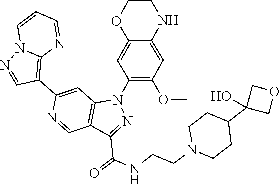 [SMILES: COc1cc2c(cc1-n1nc(C(=O)NCCN3CCC(C4(O)COC4)CC3)c3cnc(-c4cnn5cccnc45)cc31)OCCN2] | Inter- mediate 45b | tert-butyl 7-(3-((2-(4-(3- hydroxyoxetan- 3-yl)piperidin- 1-yl)ethyl) carbamoyl)-6- (pyrazolo[1,5- a]pyrimidin-3- yl)-1H-pyrazolo [4,3-c]pyridin- 1-yl)-6-methoxy- 2,3-dihydro-4H- | (500 MHz, DMSO-d₆) δ: 9.46 (d, J = 0.9 Hz, 1H), 9.23 (dd, J = 7.0, 1.5 Hz, 1H), 8.91 (s, 1H), 8.73 (dd, J = 4.1, 1.7 Hz, 1H), 8.44 (t, J = 5.8 Hz, 1H), 8.24 (d, J = 1.2 Hz, 1H), 7.17 (dd, J = 7.0, 4.0 Hz, 1H), 6.89 (s, 1H), 6.49 (s, 1H), 6.38 (s, 1H), 5.45 (s, 1H), 4.40 (d, J = 6.7 Hz, 2H), 4.34 (d, J = 6.4 Hz, 2H), 4.14 (t, J = 4.3 Hz, 2H), 3.63 (s, 3H), 3.45 (q, J = 6.5 Hz, 2H), 3.39 (m, 2H), 2.97-3.04 (m, 2H), 2.52 (m, 2H), 1.92 (br t, J = 11.3 Hz, 2H), 1.51- | (Method 4): Rt = 3.72 min, ES⁺ m/z 626.3 [M + H]⁺ |
| | N-(2-(4-(3-hydroxyoxetan-3- | | benzo[b][1,4] | 1.61 (m, 3H), 1.29-1.39 (m, 2H) | |
| | yl)piperidin-1-yl)ethyl)-1-(6-methoxy- | | oxazine- | | |
| | 3,4-dihydro-2H-benzo[b][1,4]oxazin- | | 4-carboxylate | | |
| | 7-yl)-6-(pyrazolo[1,5-a]pyrimidin-3- | | (Method 2): Rt = | | |
| | yl)-1H-pyrazolo[4,3-c]pyridine-3- | | 1.03 min, | | |
| | carboxamide | | ES⁺ m/z 726.5 | | |
| | | | [M + H]⁺ | | |
| Ex- am- ple 86 | 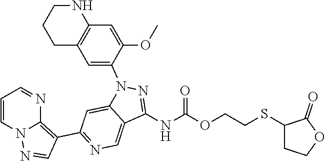 [SMILES: COc1cc2c(cc1-n1nc(NC(=O)OCCSC3CCOC3=O)c3cnc(-c4cnn5cccnc45)cc31)CCCN2] | Inter- mediate- 44 | tert-butyl 7- methoxy-6-(3-(((2- ((2-oxotetra- hydrofuran-3-yl) thio)ethoxy) carbonyl)amino)- 6-(pyrazolo[1,5- a]pyrimidin-3-yl)- 1H-pyrazolo[4,3- c]pyridin-1-yl)-3,4- | (500 MHz, DMSO-d₆) δ: 10.49 (br s, 1H), 9.24 (d, J = 0.6 Hz, 1H), 9.21 (dd, J = 7.0, 1.8 Hz, 1H), 8.87 (s, 1H), 8.71 (dd, J = 4.0, 1.6 Hz, 1H), 8.08 (d, J = 0.9 Hz, 1H), 7.15 (dd, J = 7.0, 4.3 Hz, 1H), 6.90 (s, 1H), 6.30 (s, 1H), 6.16 (m, 1H), 4.43 (dt, J = 12.6, 6.5 Hz, 1H), 4.31-4.38 (m, 2H), 4.25- 4.30 (m, 1H), 4.02 (dd, J = 8.6, 6.7 Hz, 1H), 3.62 (s, 3H), 3.25 (br t, J = 6.3 | (Method 4): Rt = 5.66 min, ES⁺ m/z 601.1 [M + H]⁺ |
| | 2-((2-oxotetrahydrofuran-3- | | dihydroquinoline- | Hz, 2H), 3.14 (dt, J = 14.0, 6.7 Hz, | |
| | yl)thio)ethyl (1-(7-methoxy-1,2,3,4- | | 1(2H)-carboxylate | 1H), 2.98 (dt, J = 14.0, 6.4 Hz, 1H), | |
| | tetrahydroquinolin-6-yl)-6- | | (Method 1): Rt = | 2.68 (m, 1H), 2.65 (t, J = 6.4 Hz, 2H), | |
| | (pyrazolo[1,5-a]pyrimidin-3-yl)-1H- | | 1.01 min, | 2.09 (m, 1H), 1.82 (m, 2H) | |
| | pyrazolo[4,3-c]pyridin-3-yl)carbamate | | ES⁺ m/z 701.3 | | |
| | | | [M + H]⁺ | | |

### Pharmacological Activity of the Compounds of the Invention (1-86).

### Biochemical Potency on JAK1, JAK2, JAK3 and Tyk2

### Assay Principle

The objective of this study was to assess the capability of compounds to inhibit all 4 JAK isoforms activity in a cell-free environment. Assay for JAK 1, JAK 2, JAK 3 and TYK2 were performed by Time-resolved fluorescence resonance energy transfer (TR-FRET) technology. It consists in the interaction of two labelled binding partners detected by the energy transfer from an excited donor to an acceptor dye and measurement of light emission by the acceptor dye. LANCE Ultra kinase assay was used. In presence of JAK 1, JAK 2, JAK 3 and TYK2 kinases and ATP (corresponding to Kim), the ULight peptide substrate (LANCE Ulight-JAK-1 (Tyr1023) Peptide, Perkin Elmer, TRF0121) is phosphorylated. It is then captured by Eu-anti-phospho-substrate antibody (LANCE Eu-W1024 Anti-phosphotyrosine (PT66), Perkin Elmer, AD0069), which bring the Eu-chelate donor and ULight acceptor dyes into close proximity. Upon excitation at 320 nm, the Eu-chelate transfers its energy to the ULight dye, resulting in a fluorescent light emission at 665 nm.

### Compound Testing

Serial dilutions of compounds in pure DMSO are prepared from 10 mM DMSO stock solutions. Compounds were tested in 384-well plate for 11 consecutive 5-fold dilutions starting from 20 μM highest concentration (20 μM-2 pM). 200 nL of compound were transferred from mother plate to test plate by using Mosquito (TTP labtech). Assay was performed in 384-well Perkin Elmer test plate in 20 μL assay volume (kinase reaction) and 40 μL total volume (stopping reagent and antibody detection reagents). In 10 μL of substrate solution (peptide+ATP) 30/50/20/10 nM of peptide and 20/0.7/0.2/12 μM of ATP were added for JAK 1, JAK 2, JAK 3 and TYK2 respectively. 10 μL of enzyme solution was added to kinase reaction at these concentrations: 0.15/0.083/0.025/0.144 ng/μL of JAK 1, JAK 2, JAK 3 and TYK2 respectively. After shaking and 1.5 h of incubation at r.t., 20 μL of Stop (10 μL EDTA) and Detection mixture (10 μL Europium-anti-phospho antibody, final: 0.5 nM) were added. Reading was performed after 1 h of incubation on a EnVision 2104 reader (Perkin Elmer).

Calculation of IC50 data, curves and QC analysis was performed by using Excel tool and GraphPadPrism software, v9. Briefly, individual concentration-effect curves are generated by plotting the logarithm of the tested concentration of tested compounds (X) vs. corresponding percent inhibition values (Y) using least squares (ordinary) fit. Best fit IC50 values are calculated using Log(inhibitor) vs. normalized response—Variable slope equation, where Y=100/(1+10\hat{ }((Log IC50−X)*HillSlope)). QC criteria parameters (Z′, S:B, R2, HillSlope) were checked for every IC50 curve. Calculation of IC50 data, curves and QC analysis were made using Excel tools and GraphPadPrism software. QC criteria parameters: Z′≥0.5, Hill Slope range 0.5 to 5, S:B>2.

Compounds according to the invention showed values in terms of pIC50 higher than 6 with respect to their inhibitory activity on all JAK isoforms corresponding to ≤1 μM in terms of inhibitory concentration. Most compounds preferably showed values higher than 7.3, even more preferably higher than 8.3, at least with respect to their inhibitory activity on JAK1; corresponding to ≤50 nM, even more preferably ≤5 nM, in terms of inhibitory concentration.

Data for compounds 1-76 are reported in the table hereinbelow

| Example No | JAK1 | JAK2 | JAK3 | TYK2 |
|---|---|---|---|---|
| 1 | +++ | +++ | +++ | ++ |
| 2 | +++ | +++ | +++ | ++ |
| 3 | +++ | +++ | +++ | ++ |
| 4 | +++ | +++ | +++ | +++ |
| 5 | +++ | +++ | +++ | ++ |
| 6 | +++ | +++ | +++ | +++ |
| 7 | +++ | +++ | +++ | +++ |
| 8 | +++ | +++ | +++ | + |
| 9 | ++ | +++ | ++ | + |
| 10 | +++ | +++ | +++ | ++ |
| 11 | +++ | +++ | +++ | ++ |
| 12 | ++ | ++ | ++ | + |
| 13 | +++ | +++ | +++ | ++ |
| 14 | +++ | +++ | +++ | ++ |
| 15 | +++ | +++ | +++ | ++ |
| 16 | +++ | +++ | +++ | ++ |
| 17 | +++ | +++ | +++ | +++ |
| 18 | +++ | +++ | +++ | ++ |
| 19 | +++ | +++ | +++ | ++ |
| 20 | +++ | +++ | +++ | +++ |
| 21 | +++ | +++ | +++ | +++ |
| 22 | +++ | +++ | +++ | +++ |
| 23 | +++ | +++ | +++ | +++ |
| 24 | +++ | +++ | +++ | ++ |
| 25 | ++ | ++ | +++ | ++ |
| 26 | +++ | +++ | +++ | ++ |
| 27 | +++ | +++ | +++ | ++ |
| 28 | +++ | +++ | +++ | +++ |
| 29 | +++ | +++ | +++ | +++ |
| 30 | +++ | +++ | +++ | ++ |
| 31 | +++ | +++ | +++ | +++ |
| 32 | +++ | +++ | +++ | +++ |
| 33 | +++ | +++ | +++ | +++ |
| 34 | +++ | +++ | +++ | ++ |
| 35 | +++ | +++ | +++ | +++ |
| 36 | +++ | +++ | +++ | ++ |
| 37 | +++ | +++ | +++ | +++ |
| 38 | +++ | +++ | +++ | +++ |
| 39 | +++ | +++ | ++ | ++ |
| 40 | +++ | +++ | +++ | ++ |
| 41 | ++ | +++ | ++ | ++ |
| 42 | +++ | +++ | +++ | ++ |
| 43 | +++ | +++ | +++ | ++ |
| 44 | +++ | +++ | +++ | ++ |
| 45 | +++ | +++ | +++ | ++ |
| 46 | +++ | +++ | +++ | ++ |
| 47 | +++ | +++ | +++ | ++ |
| 48 | +++ | +++ | +++ | ++ |
| 49 | +++ | +++ | +++ | ++ |
| 50 | +++ | +++ | +++ | ++ |
| 51 | +++ | +++ | +++ | ++ |
| 52 | +++ | +++ | +++ | ++ |
| 53 | +++ | +++ | +++ | ++ |
| 54 | +++ | +++ | +++ | ++ |
| 55 | +++ | +++ | +++ | ++ |
| 56 | +++ | +++ | +++ | ++ |
| 57 | +++ | +++ | +++ | ++ |
| 58 | +++ | +++ | +++ | +++ |
| 59 | +++ | +++ | +++ | ++ |
| 60 | +++ | +++ | +++ | ++ |
| 61 | +++ | +++ | +++ | ++ |
| 62 | +++ | +++ | +++ | ++ |
| 63 | +++ | +++ | +++ | ++ |
| 64 | +++ | +++ | +++ | ++ |
| 65 | +++ | +++ | +++ | ++ |
| 66 | +++ | +++ | +++ | ++ |
| 67 | +++ | +++ | +++ | ++ |
| 68 | +++ | +++ | +++ | ++ |
| 69 | +++ | +++ | +++ | ++ |
| 70 | +++ | +++ | +++ | ++ |
| 71 | ++ | ++ | ++ | + |
| 72 | +++ | +++ | +++ | +++ |
| 73 | ++ | +++ | +++ | ++ |
| 74 | +++ | +++ | +++ | +++ |
| 75 | +++ | +++ | +++ | ++ |
| 76 | +++ | +++ | +++ | ++ |
| 77 | +++ | +++ | +++ | + |
| 78 | +++ | +++ | +++ | ++ |
| 79 | +++ | +++ | +++ | ++ |
| 80 | +++ | +++ | +++ | ++ |
| 81 | +++ | +++ | +++ | ++ |
| 82 | +++ | +++ | +++ | ++ |
| 83 | +++ | +++ | +++ | + |
| 84 | ++ | +++ | +++ | ++ |
| 85 | +++ | +++ | +++ | ++ |
| 86 | +++ | +++ | +++ | ++ |

The compounds are classified in the table above in term of potency with respect to their inhibitory activity on JAK1, JAK2, JAK3 and Tyk2 isoforms according to the following classification criterion:

$$++ + : {pIC}_{50} \geq 8.3$$

$$++ : 8.3 > {pIC}_{50} \geq 7.3$$

$$+ : {pIC}_{50} < 7.3$$

Inhibition of IL-13 induced pSTAT6 in BEAS

BEAS-2B human cell line was seeded (100.000 cells/well) and incubated for 48 h at 37° C., 5% CO2, 95% humidity. Compounds were added and incubated 30 min followed by IL-13 as trigger. After 30 min incubation, cells were lysed and pSTAT6 determined by Fastscan phospho-stat6 (Tyr641) sandwich ELISA kit (cell signaling). Inhibitors were tested at 11 consecutive 5-fold dilutions starting from 10 μM (10 μM-40 pM) in duplicate. Calculation of IC50 data, curves and QC analysis were made using Excel tools and GraphPadPrism software. QC criteria parameters: Z′≥0.35, Hill Slope range 0.5 to 5, S:B>2.

| Example No | pIC₅₀ (BEAS) |
|---|---|
| 1 | §§§ |
| 2 | §§§ |
| 3 | §§§ |
| 4 | §§§ |
| 5 | §§§ |
| 6 | §§§§ |
| 7 | §§§ |
| 8 | §§§ |
| 9 | §§§ |
| 10 | §§§ |
| 11 | §§§ |
| 12 | §§ |
| 13 | §§§ |
| 14 | §§§ |
| 15 | §§§§ |
| 16 | §§§§ |
| 17 | §§§ |
| 18 | §§§ |
| 19 | §§§ |
| 20 | §§§ |
| 21 | §§§ |
| 22 | §§§ |
| 23 | §§§ |
| 24 | §§ |
| 25 | §§§ |
| 26 | §§§ |
| 27 | §§§ |
| 28 | §§§§ |
| 29 | §§§§ |
| 30 | §§§ |
| 31 | §§§ |
| 32 | §§§ |
| 33 | §§§ |
| 34 | §§§ |
| 35 | §§§ |
| 36 | §§§§ |
| 37 | §§§ |
| 38 | §§§ |
| 39 | §§§ |
| 40 | §§§ |
| 41 | §§§ |
| 42 | §§§§ |
| 43 | §§§ |
| 44 | §§§ |
| 45 | §§§ |
| 46 | §§§§ |
| 47 | §§§ |
| 48 | §§§§ |
| 49 | §§§ |
| 50 | §§§ |
| 51 | §§§§ |
| 52 | §§§§ |
| 53 | §§§ |
| 54 | §§§ |
| 55 | §§§ |
| 56 | §§§ |
| 57 | §§§ |
| 58 | §§§ |
| 59 | §§§ |
| 60 | §§§ |
| 61 | §§§ |
| 62 | §§§ |
| 63 | §§§ |
| 64 | §§§ |
| 65 | §§§ |
| 66 | §§§ |
| 67 | §§§ |
| 68 | §§§ |
| 69 | §§§ |
| 70 | §§§ |
| 71 | §§§ |
| 72 | §§ |
| 73 | §§§ |
| 74 | §§ |
| 75 | §§ |
| 76 | §§§ |
| 77 | §§§ |
| 78 | §§§ |
| 79 | §§ |
| 80 | §§ |
| 81 | §§§ |
| 82 | §§§ |
| 83 | §§ |
| 84 | §§§ |
| 85 | §§§ |
| 86 | §§§ |

The compounds are classified in the table above in term of potency with respect to their functional activity in BEAS according to the following classification criterion:

$$§§§§ : {pIC}_{50} \geq 8.3$$

$$§§§ : 8.3 > {pIC}_{50} \geq 7.3$$

$$§§ : 7.3 > {pIC}_{50} \geq 6.3$$

$$§ : {pIC}_{50} < 6.3$$

Where a numerical limit or range is stated herein, the endpoints are included. Also, all values and subranges within a numerical limit or range are specifically included when not explicitly written out. As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

1: A compound of formula I 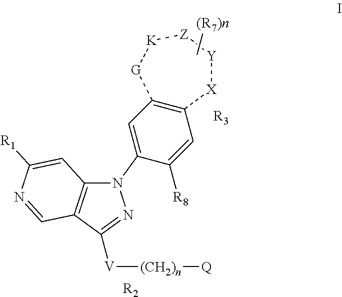
wherein:
R₁ is a heteroaryl group selected from the group consisting of imidazo[1,2-b]pyridazin-3-yl, pyrazolo[1,5-a]pyrimidin-3-yl and 3-oxo-(3,4-dihydropyrazin-2-yl)amino;
R₂ is a group of formula 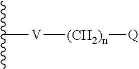
that is a substituent group linked to the molecule scaffold,
wherein
V is a bond or is a divalent group selected from the group consisting of O, S, N(R₆), C(O)N(R₆), N(R₆)C(O); N(R₆)C(O)O; N(R₆)S(O)₂; and N(R₆)C(O)N(R₆);
Q is selected from the group consisting of H, (C₁-C₆)alkyl, (C₁-C₆)hydroxyalkyl, (C₁-C₆)alkoxy, —(CH₂)ₘNR₄R₅, (C₃-C₈)cycloalkyl, (C₃-C₁₀)heterocycloalkyl; —S—(C₃-C₆)heterocycloalkyl, and —N(R₆)—(C₃-C₆)heterocycloalkyl; wherein said (C₃-C₈)cycloalkyl and (C₃-C₁₀)heterocycloalkyl are further optionally substituted by one or more substituent selected from the group consisting of —OH, oxo, (C₁-C₁₀)alkyl, (C₁-C₆)alkoxy; halogen, (C₁-C₆)haloalkyl, alkanoyl, (C₁-C₆)hydroxyalkyl, (C₁-C₆)alkoxy(C₁-C₆)alkyl, —N(R₆)(CH₂)ₘC(O)NR₄R₅, —(CO)NR₄R₅, —(CH₂)ₘNR₄R₅, (C₃-C₈)cycloalkyl(C₁-C₆)alkyl; (C₃-C₆)heterocycloalkyl(C₁-C₆)alkyl, (C₃-C₆)heterocycloalkyl and hydroxyl-(C₃-C₆)heterocycloalkyl;
R₃ is a bicyclic moiety which is a substituent group linked to the scaffold graphically represented by 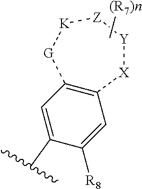
wherein the dashed line [symbol] indicates a single or a double bond;
X is selected from the group consisting of N, S, and C;
Y is selected from the group consisting of Cand N;
Z is selected from the group consisting of C, N, and O;
K is a bond or selected from the group consisting of O, C, and S;
G is a bond or selected from the group consisting of C and O; 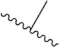
is the point of attachment of a substituent group to the rest of the molecule;
n and m are in each occurrence independently 0, 1, 2, 3 or 4;
R₄ and R₅ are each independently selected from the group consisting of
—H,
(C₁-C₆)alkyl,
(C₁-C₆)haloalkyl, and
(C₃-C₆)heterocycloalkyl;
R₆ is in each occurrence independently selected from the group consisting of H, (C₁-C₆)alkyl and (C₁-C₆)hydroxyalkyl;
R₇ is one or more group in each occurrence independently selected from the group consisting of —OH, oxo, (C₁-C₆)alkyl, halogen, (C₁-C₆)haloalkyl, (C₁-C₆)hydroxyalkyl, —(CH₂)ₘNR₄R₅, (C₁-C₆)alkyl-S(O)₂— and (C₁-C₆)alkyl-S(O)₂N(R₆)—; and
R₈ is selected from the group consisting of (C₁-C₆)alkoxy and (C₁-C₆)haloalkoxy;
a single enantiomer, a diastereoisomer, a mixture thereof in any proportion,
or a pharmaceutically acceptable salt or solvate thereof.

2: The compound, single enantiomer, diastereoisomer, mixture thereof, or pharmaceutically acceptable salt or solvate thereof, according to claim 1
wherein the bicyclic moiety R₃ 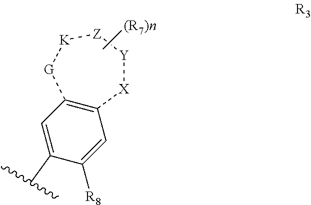
is selected from the group consisting of

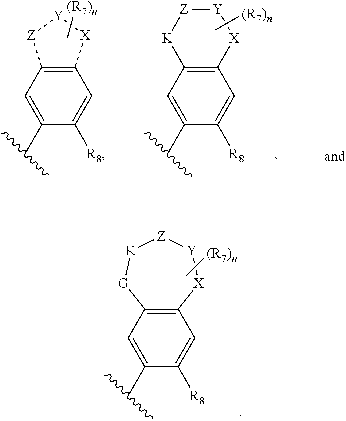

3: The compound, single enantiomer, diastereoisomer, mixture thereof, or pharmaceutically acceptable salt or solvate thereof, of according to claim 2
wherein the bicyclic moiety R₃ is selected from the group consisting of J₁-J₁₃

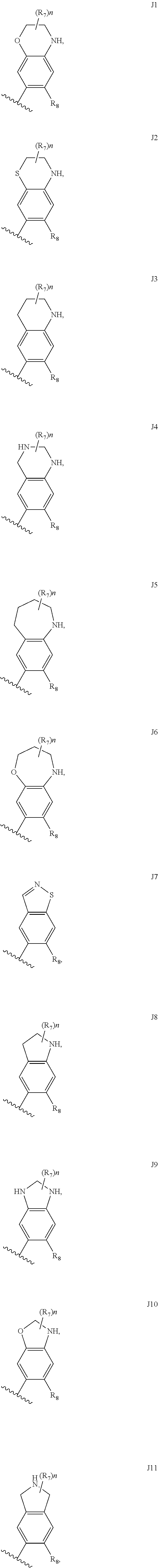

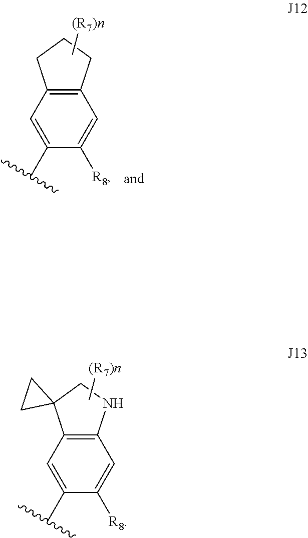

4: The compound, single enantiomer, diastereoisomer, mixture thereof, or pharmaceutically acceptable salt or solvate thereof, according to claim 3
wherein
R₃ is J1 and R₈ is methoxy;
the compound being thus represented by the formula Ia 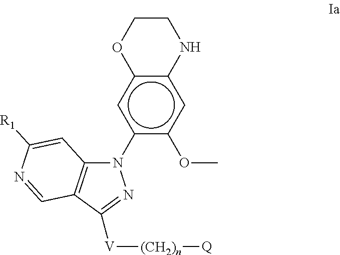
wherein
R₁ is pyrazolo[1,5-a]pyrimidin-3-yl or (3-oxo-3,4-dihydropyrazin-2-yl)amino;
V is a divalent group selected from the group consisting of C(O)N(R₆) and N(R₆)C(O)O;
Q is selected from the group consisting of (C₁-C₆)alkyl, (C₁-C₆)alkoxy, —(CH₂)ₘNR₄R₅, (C₃-C₈)cycloalkyl and (C₃-C₁₂)heterocycloalkyl; wherein said (C₃-C₈)cycloalkyl and (C₃-C₁₂)heterocycloalkyl are further optionally substituted by one or more substituent selected from the group consisting of oxo, (C₁-C₁₀)alkyl, halogen, (C₁-C₆)hydroxyalkyl, —(CO)NR₄R₅; (C₃-C₈)cycloalkyl(C₁-C₆)alkyl; (C₃-C₆)heterocycloalkyl;
n and m are in each occurrence independently 0, 1, 2, 3 or 4;
R₄ and R₅ are in each occurrence independently selected from the group consisting of
—H,
(C₁-C₆)alkyl, and
(C₁-C₆)haloalkyl.

5: The compound, single enantiomer, diastereoisomer, mixture thereof, or pharmaceutically acceptable salt or solvate thereof, according to claim 4 wherein
V is N(R₆)C(O)O;
Q is selected from the group consisting of (C₁-C₆)alkyl, (C₁-C₆)alkoxy, —(CH₂)ₘNR₄R₅, and (C₃-C₆)heterocycloalkyl being a group 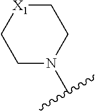
wherein X₁ is selected from the group consisting of CH₂, O, S, NH, NCH₃, (C═O) and S(═O)₂.

6: The compound, single enantiomer, diastereoisomer, mixture thereof, or pharmaceutically acceptable salt or solvate thereof, according to claim 4 wherein
V is C(O)N(R₆),
Q is selected from the group consisting of —(CH₂)ₘNR₄R₅, and (C₃-C₆)heterocycloalkyl being a group 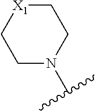
wherein X₁ is selected from the group consisting of CHR₉, O, S, NH, NCH₃, CF₂, (C₁-C₆) alkoxy and (C₁-C₆)alkoxy(C₁-C₆)alkyl, wherein R₉ is H or —(CO)NR₄R₅.

7: The compound, single enantiomer, diastereoisomer, mixture thereof, or pharmaceutically acceptable salt or solvate thereof, according to claim 4 wherein
Q is (C₃-C₆)heterocycloalkyl, —S—(C₃-C₆)heterocycloalkyl or —N(R₆)—(C₃-C₆)heterocycloalkyl selected from the group consisting of:

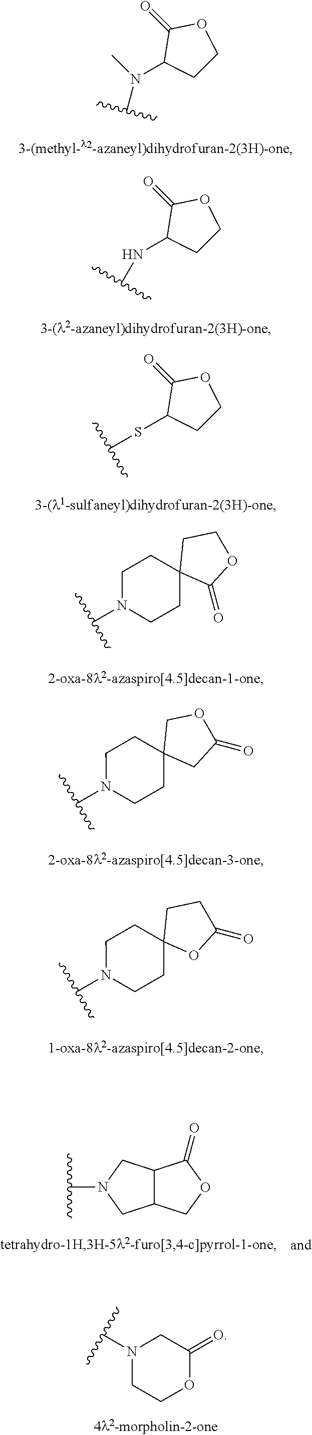

8: The compound, single enantiomer, diastereoisomer, mixture thereof, or pharmaceutically acceptable salt or solvate thereof, according to claim 1, wherein the compound is selected from the group consisting of:
6-(difluoromethoxy)-7-(3-methyl-6-(pyrazolo[1,5-a]pyrimidin-3-yl)-1H-pyrazolo[4,3-c]pyridin-1-yl)-3,4-dihydro-2H-benzo[b][1,4]oxazine,
1-(6-methoxy-2-(methylsulfonyl)isoindolin-5-yl)-3-methyl-6-(pyrazolo[1,5-a]pyrimidin-3-yl)-1H-pyrazolo[4,3-c]pyridine,
8-methoxy-7-(3-methyl-6-(pyrazolo[1,5-a]pyrimidin-3-yl)-1H-pyrazolo[4,3-c]pyridin-1-yl)-1,3,4,5-tetrahydro-2H-benzo[b]azepin-2-one,
7-methoxy-6-(3-methyl-6-(pyrazolo[1,5-a]pyrimidin-3-yl)-1H-pyrazolo[4,3-c]pyridin-1-yl)-3,4-dihydroquinolin-2 (1H)-one,
7-methoxy-6-(3-methyl-6-(pyrazolo[1,5-a]pyrimidin-3-yl)-1H-pyrazolo[4,3-c]pyridin-1-yl)-2,3-dihydroquinolin-4 (1H)-one,
6-methoxy-7-(3-methyl-6-(pyrazolo[1,5-a]pyrimidin-3-yl)-1H-pyrazolo[4,3-c]pyridin-1-yl)-3,4-dihydro-2H-benzo[b][1,4]thiazine,
6-methoxy-7-(3-methyl-6-(pyrazolo[1,5-a]pyrimidin-3-yl)-1H-pyrazolo[4,3-c]pyridin-1-yl)-2H-benzo[b][1,4]thiazin-3 (4H)-one,
6-methoxy-7-(3-methyl-6-(pyrazolo[1,5-a]pyrimidin-3-yl)-1H-pyrazolo[4,3-c]pyridin-1-yl)-3,4-dihydro-2H-benzo[b][1,4]thiazine 1,1-dioxide,
7-methoxy-8-(3-methyl-6-(pyrazolo[1,5-a]pyrimidin-3-yl)-1H-pyrazolo[4,3-c]pyridin-1-yl)-2,3,4,5-tetrahydrobenzo[b][1,4]oxazepine,
6-methoxy-5-(3-methyl-6-(pyrazolo[1,5-a]pyrimidin-3-yl)-1H-pyrazolo[4,3-c]pyridin-1-yl)indolin-2-one,
2,2-difluoro-6-methoxy-7-(3-methyl-6-(pyrazolo[1,5-a]pyrimidin-3-yl)-1H-pyrazolo[4,3-c]pyridin-1-yl)-2H-benzo[b][1,4]oxazin-3 (4H)-one,
7-methoxy-3-methyl-6-(3-methyl-6-(pyrazolo[1,5-a]pyrimidin-3-yl)-1H-pyrazolo[4,3-c]pyridin-1-yl)-3,4-dihydroquinazolin-2 (1H)-one,
1-(6-methoxy-3,4-dihydro-2H-benzo[b][1,4]oxazin-7-yl)-6-(pyrazolo[1,5-a]pyrimidin-3-yl)-N-(1-((tetrahydro-2H-pyran-4-yl)methyl)azetidin-3-yl)-1H-pyrazolo[4,3-c]pyridine-3-carboxamide,
N-(3-(4,4-difluoropiperidin-1-yl)propyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(2-oxo-1-oxa-8-azaspiro[4.5]decan-8-yl)ethyl)-6-(pyrazolo[1,5-a]pyrimidin-3-yl)-1H-pyrazolo[4,3-c]pyridine-3-carboxamide,
N-(3-(dimethylamino)propyl)-1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]thiazin-7-yl)-N-(1-methylazetidin-3-yl)-6-(pyrazolo[1,5-a]pyrimidin-3-yl)-1H-pyrazolo[4,3-c]pyridine-3-carboxamide,
(R)-1-(6-methoxy-3,4-dihydro-2H-benzo[b][1,4]thiazin-7-yl)-N-((4-methylmorpholin-2-yl)methyl)-6-(pyrazolo[1,5-a]pyrimidin-3-yl)-1H-pyrazolo[4,3-c]pyridine-3-carboxamide,
3-(2-oxomorpholino)propyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
2-(methyl(2-oxotetrahydrofuran-3-yl)amino)ethyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
(R)-1-(6-methoxy-3-oxo-3,4-dihydro-2H-benzo[b][1,4]thiazin-7-yl)-N-((4-methylmorpholin-2-yl)methyl)-6-(pyrazolo[1,5-a]pyrimidin-3-yl)-1H-pyrazolo[4,3-c]pyridine-3-carboxamide,
(S)-1-(6-methoxy-3-oxo-3,4-dihydro-2H-benzo[b][1,4]thiazin-7-yl)-N-((4-methylmorpholin-2-yl)methyl)-6-(pyrazolo[1,5-a]pyrimidin-3-yl)-1H-pyrazolo[4,3-c]pyridine-3-carboxamide,
N-(3-(dimethylamino)propyl)-1-(6-methoxy-3-oxo-3,4-dihydro-2H-benzo[b][1,4]thiazin-7-yl)-6-(pyrazolo[1,5-a]pyrimidin-3-yl)-1H-pyrazolo[4,3-c]pyridine-3-carboxamide,
N-(3-(dimethylamino)propyl)-1-(7-methoxy-2-oxo-1,2,3,4-tetrahydroquinolin-6-yl)-6-(pyrazolo[1,5-a]pyrimidin-3-yl)-1H-pyrazolo[4,3-c]pyridine-3-carboxamide,
N-(3-(dimethylamino)propyl)-1-(7-methoxy-1,2,3,4-tetrahydroquinolin-6-yl)-6-(pyrazolo[1,5-a]pyrimidin-3-yl)-1H-pyrazolo[4,3-c]pyridine-3-carboxamide,
7-methoxy-6-(3-(methylamino)-6-(pyrazolo[1,5-a]pyrimidin-3-yl)-1H-pyrazolo[4,3-c]pyridin-1-yl)-3,4-dihydroquinolin-2 (1H)-one,
2-(dimethylamino)ethyl (1-(7-methoxy-2-oxo-1,2,3,4-tetrahydroquinolin-6-yl)-6-(pyrazolo[1,5-a]pyrimidin-3-yl)-1H-pyrazolo[4,3-c]pyridin-3-yl)carbamate,
2-(dimethylamino)ethyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
2-morpholinoethyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
3-(4-methylpiperazin-1-yl)propyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
2-(piperidin-1-yl)ethyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
(R)-1-methylpyrrolidin-3-yl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
(S)-1-methylpyrrolidin-3-yl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
(4-(dimethylamino)tetrahydro-2H-pyran-4-yl)methyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
(R)-2-(3-methoxypyrrolidin-1-yl)ethyl (1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamate,
3-(1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)-1,1-dimethylurea,
6-methoxy-7-(3-((2-(4-methylpiperazin-1-yl)ethyl)amino)-6-(pyrazolo[1,5-a]pyrimidin-3-yl)-1H-pyrazolo[4,3-c]pyridin-1-yl)-2H-benzo[b][1,4]thiazin-3 (4H)-one,
7-(3-((2-(dimethylamino)ethyl)amino)-6-(pyrazolo[1,5-a]pyrimidin-3-yl)-1H-pyrazolo[4,3-c]pyridin-1-yl)-6-methoxy-2H-benzo[b][1,4]thiazin-3 (4H)-one,
1-(6-methoxybenzo[d]isothiazol-5-yl)-N-(2-morpholinoethyl)-6-(pyrazolo[1,5-a]pyrimidin-3-yl)-1H-pyrazolo[4,3-c]pyridin-3-amine,
6-methoxy-7-(3-methyl-6-(pyrazolo[1,5-a]pyrimidin-3-yl)-1H-pyrazolo[4,3-c]pyridin-1-yl)-3,4-dihydro-2H-benzo[b][1,4]oxazine,
1-(6-methoxyindolin-5-yl)-3-methyl-6-(pyrazolo[1,5-a]pyrimidin-3-yl)-1H-pyrazolo[4,3-c]pyridine,
7-methoxy-6-(3-methyl-6-(pyrazolo[1,5-a]pyrimidin-3-yl)-1H-pyrazolo[4,3-c]pyridin-1-yl)-1,2,3,4-tetrahydroquinoline,
N-(3-(dimethylamino)propyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(1-methylazetidin-3-yl)-6-(pyrazolo[1,5-a]pyrimidin-3-yl)-1H-pyrazolo[4,3-c]pyridine-3-carboxamide,
(R)-1-(6-methoxy-3,4-dihydro-2H-benzo[b][1,4]oxazin-7-yl)-N-((4-methylmorpholin-2-yl)methyl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(4-methoxypiperidin-1-yl)ethyl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(4-(methoxymethyl)piperidin-1-yl)ethyl)-6-(pyrazolo[1,5-a]pyrimidin-3-yl)-1H-pyrazolo[4,3-c]pyridine-3-carboxamide,
N-(2-(7-oxa-2-azaspiro[3.5]nonan-2-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
N-(2-(2-oxa-6-azaspiro[3.5]nonan-6-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-((3aS,6aR)-1-oxotetrahydro-1H-furo[3,4-c]pyrrol-5 (3H)-yl)ethyl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(3-oxo-2-oxa-8-azaspiro[4.5]decan-8-yl)ethyl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(1-oxo-2-oxa-8-azaspiro[4.5]decan-8-yl)ethyl)-6-(pyrazolo[1,5-a]pyrimidin-3-yl)-1H-pyrazolo[4,3-c]pyridine-3-carboxamide,
N-(2-(4-(dimethylcarbamoyl)piperidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
N1-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)-N2,N2-dimethylethane-1,2-diamine,
1-(6-methoxy-3,4-dihydro-2H-benzo[b][1,4]oxazin-7-yl)-N-methyl-6-(pyrazolo[1,5-a]pyrimidin-3-yl)-1H-pyrazolo[4,3-c]pyridin-3-amine,
methyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-(dimethylamino)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-morpholinoethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
3-(4-methylpiperazin-1-yl)propyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
3-(dimethylamino)propyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
3-morpholinopropyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-(1,1-dioxidothiomorpholino)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-(4-methylpiperazin-1-yl)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-thiomorpholinoethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
(S)-(4-methylmorpholin-2-yl)methyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-(piperidin-1-yl)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-((2-oxotetrahydrofuran-3-yl)thio)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-((2-oxotetrahydrofuran-3-yl)amino)ethyl (1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamate,
2-(dimethylamino)ethyl (1-(7-methoxy-1,2,3,4-tetrahydroquinolin-6-yl)-6-(pyrazolo[1,5-a]pyrimidin-3-yl)-1H-pyrazolo[4,3-c]pyridin-3-yl)carbamate,
1-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)-3-(3-morpholinopropyl)urea,
6′-methoxy-5′-(3-methyl-6-(pyrazolo[1,5-a]pyrimidin-3-yl)-1H-pyrazolo[4,3-c]pyridin-1-yl)spiro[cyclopropane-1,3′-indolin]-2′-one,
5-methoxy-6-(3-((2-morpholinoethyl)amino)-6-(pyrazolo[1,5-a]pyrimidin-3-yl)-1H-pyrazolo[4,3-c]pyridin-1-yl)benzo[d]oxazol-2 (3H)-one,
N-(3-(dimethylamino)propyl)-6-(imidazo[1,2-b]pyridazin-3-yl)-1-(6-methoxy-3,4-dihydro-2H-benzo[b][1,4]oxazin-7-yl)-1H-pyrazolo[4,3-c]pyridine-3-carboxamide,
3-((3-((2-(dimethylamino)ethyl)amino)-1-(6-methoxy-3,4-dihydro-2H-benzo[b][1,4]oxazin-7-yl)-1H-pyrazolo[4,3-c]pyridin-6-yl)amino)pyrazin-2 (1H)-one,
N-(3-(dimethylamino)propyl)-1-(6-methoxy-3,4-dihydro-2H-benzo[b][1,4]oxazin-7-yl)-6-((3-oxo-3,4-dihydropyrazin-2-yl)amino)-1H-pyrazolo[4,3-c]pyridine-3-carboxamide,
N-(3-(4,4-difluoropiperidin-1-yl)propyl)-1-(6-methoxy-3,4-dihydro-2H-benzo[b][1,4]oxazin-7-yl)-6-((3-oxo-3,4-dihydropyrazin-2-yl)amino)-1H-pyrazolo[4,3-c]pyridine-3-carboxamide,
N-(2-(2,2-dimethyl-6-oxomorpholino)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-(difluoromethoxy)-3,4-dihydro-2H-benzo[b][1,4]oxazin-7-yl)-N-(3-(dimethylamino)propyl)-6-(pyrazolo[1,5-a]pyrimidin-3-yl)-1H-pyrazolo[4,3-c]pyridine-3-carboxamide,
N-(2-(4-carbamoylpiperidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
(R)—N-(2-(3-carbamoylpyrrolidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(7-oxo-6-oxa-2-azaspiro[3.4]octan-2-yl)ethyl)-6-(pyrazolo[1,5-a]pyrimidin-3-yl)-1H-pyrazolo[4,3-c]pyridine-3-carboxamide,
N-(2-(2-oxa-6-azaspiro[3.4]octan-6-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide,
1-(6-methoxy-3,4-dihydro-2H-benzo[b][1,4]oxazin-7-yl)-N-(2-(2-methyl-6-oxomorpholino)ethyl)-6-(pyrazolo[1,5-a]pyrimidin-3-yl)-1H-pyrazolo[4,3-c]pyridine-3-carboxamide,
N-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)-1-methylpiperidine-4-carboxamide,
N-(2-(4-(3-hydroxyoxetan-3-yl)piperidin-1-yl)ethyl)-1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamide, and
2-((2-oxotetrahydrofuran-3-yl)thio)ethyl (1-(7-methoxy-1,2,3,4-tetrahydroquinolin-6-yl)-6-(pyrazolo[1,5-a]pyrimidin-3-yl)-1H-pyrazolo[4,3-c]pyridin-3-yl)carbamate.

9: A pharmaceutical composition comprising the compound, single enantiomer, diastereoisomer, mixture thereof, or pharmaceutically acceptable salt or solvate thereof, according to claim 1 in admixture with at least one pharmaceutically acceptable carrier or excipient.

10: The pharmaceutical composition according to claim 9, which is formulated as inhalable powders, propellant-containing metering aerosols, or propellant-free inhalable formulations, to be administered by inhalation.

11: A device comprising the pharmaceutical composition according to claim 10, which is a single- or multi-dose dry powder inhaler, a metered dose inhaler, or a soft mist nebulizer.

12. (canceled)

13: A method for treating a pulmonary disease selected from the group consisting of asthma, chronic obstructive pulmonary disease (COPD), idiopathic pulmonary fibrosis (IPF), acute lung injury and acute respiratory distress syndrome (ARDS), the method comprising administering the pharmaceutical composition of claim 9 to a subject in need thereof.

14: A composition comprising:
the compound, single enantiomer, diastereoisomer, mixture thereof, or pharmaceutically acceptable salt or solvate thereof, according to claim 1, and
one or more active ingredients selected from the group consisting of a beta2-agonist, an antimuscarinic agent, a corticosteroid, a mitogen-activated kinase (P38 MAP kinase) inhibitor, a PI3K inhibitors (phosphoinositide 3-kinase), a nuclear factor kappa-B kinase subunit beta inhibitor (IKK2), a Rho kinase inhibitor (ROCKi), a human neutrophil elastase (HNE) inhibitor, a phosphodiesterase 4 (PDE4) inhibitor, a leukotriene modulator, a non-steroidal anti-inflammatory agent (NSAIDs), and a mucus regulator.
